(19) United States
(12) Patent Application Publication
(10) Pub. No.: US 2017/0183677 A1
(43) Pub. Date: Jun. 29, 2017

(54) AGRONOMIC TRAIT MODIFICATION USING GUIDE RNA/CAS ENDONUCLEASE SYSTEMS AND METHODS OF USE

(71) Applicant: PIONEER HI-BRED INTERNATIONAL, INC., Johnston, IA (US)

(72) Inventors: HUIRONG GAO, JOHNSTON, IA (US); XIAOMU NIU, JOHNSTON, IA (US); JINRUI SHI, JOHNSTON, IA (US)

(21) Appl. No.: 15/323,772

(22) PCT Filed: Jul. 13, 2015
(86) PCT No.: PCT/US15/40143

§ 371 Date: Jan. 4, 2017

Related U.S. Application Data
(60) Provisional application No. 62/023,239, filed on Jul. 11, 2014.

(51) Int. Cl. C12N 15/82; C12N 9/88

(52) U.S. Cl. CPC C12N 15/8249; C12N 15/8213; C12N 15/8271; C12N 15/8273; C12N 15/8261; C12N 9/88; C12Y 404/01014; C12Y 301/03012

(57) ABSTRACT

Compositions and methods are provided for agronomic trait modification of a target sequence in the genome of a plant or plant cell. The methods and compositions employ a guide RNA/Cas endonuclease system to provide an effective system for modifying or altering target sites within a genomic region of a plant, plant cell or seed to provide improvement in a desirable agronomic trait such as drought, yield, and stress tolerance. Breeding methods for selecting plants utilizing a two component RNA guide and Cas endonuclease system are also disclosed. Compositions and methods are also provided for editing a nucleotide sequence in the genome of a cell.

### CROSS REFERENCE TO RELATED APPLICATION

This application claims priority to U.S. Provisional Application No. 62/023,239, filed Jul. 11, 2014, which is hereby incorporated by reference in its entirety.

### FIELD

The invention relates to the field of plant molecular biology, in particular, to methods for altering the genome of a plant cell.

### BACKGROUND

Recombinant DNA technology has made it possible to insert foreign DNA sequences into the genome of an organism, thus, altering the organism's phenotype. The most commonly used plant transformation methods are Agrobacterium infection and biolistic particle bombardment in which transgenes integrate into a plant genome in a random fashion and in an unpredictable copy number. Thus, efforts are undertaken to control transgene integration in plants.

One method for inserting or modifying a DNA sequence involves homologous DNA recombination by introducing a transgenic DNA sequence flanked by sequences homologous to the genomic target.

Although several approaches have been developed to target a specific site for modification in the genome of a plant, there still remains a need for more efficient and effective methods for producing a fertile plant, having an altered genome comprising specific modifications in a defined region of the genome of the plant.

### SUMMARY

Compositions and methods are provided employing a guide RNA/Cas endonuclease system in plants for genome modification of a target sequence (involved in improving an agronomic trait in the plant) in the genome of a plant or plant cell, for selecting plants, for gene editing, and for inserting a polynucleotide of interest into the genome of a plant. The methods and compositions employ a guide RNA/Cas endonuclease system to provide for an effective system for modifying or altering target sites and nucleotide of interest within the genome of a plant, plant cell or seed. Once a genomic target site is identified, a variety of methods can be employed to further modify the target sites such that they contain a variety of polynucleotides of interest. Breeding methods and methods for selecting plants utilizing a two component RNA guide and Cas endonuclease system are also disclosed. Also provided are nucleic acid constructs, plants, plant cells, explants, seeds and grain having the guide RNA/Cas endonuclease system. Compositions and methods are also provided employing a guide polynucleotide/Cas endonuclease system for genome modification of a target sequence in the genome of a cell or organism, for gene editing, and for inserting or deleting a polynucleotide of interest into or from the genome of a cell or organism. The methods and compositions employ a guide polynucleotide/Cas endonuclease system to provide for an effective system for modifying or altering target sites and editing nucleotide sequences of interest within the genome of a cell, wherein the guide polynucleotide is comprised of a RNA sequence, a DNA sequence, or a DNA-RNA combination sequence.

In an embodiment, a method of improving an agronomic trait of a plant, the method comprising providing a guide RNA that targets a polynucleotide involved in improving one or more agronomic characteristics of the plant in association with a Cas endonuclease that creates a double strand break at the polynucleotide and generating the plant, wherein the plant exhibits an improvement in the agronomic trait. In an embodiment, a donor polynucleotide that comprises one or more nucleotide changes as compared to a corresponding endogenous unmodified genomic DNA is disclosed. In an embodiment, the donor polynucleotide does not encode a full-length protein. In an embodiment, the donor polynucleotide comprises a heterologous regulatory element. In an embodiment, the regulatory element comprises a promoter. In an embodiment, the regulatory element comprises an enhancer element. In an embodiment, the enhancer element is plant derived. In an embodiment, the polynucleotide is selected from the group consisting of a regulatory element, 5′-UTR, intron, exon, coding sequence, and a promoter. In an embodiment, the heterologous regulatory element is from the same plant species as the polynucleotide involved in improving one or more agronomic characteristics of the plant. In an embodiment, the guide RNA targets the polynucleotide selected from the group consisting of polynucleotide sequences involved in the expression of ZmArgos8, ZmACS6, ZmSRTF18, ZmXERICO1, trehalose 6 phosphate phosphatase (T6PP), and ZmSTPP3. In an embodiment, the agronomic characteristic is selected from the group consisting of abiotic stress tolerance. In an embodiment, the abiotic stress tolerance is drought or nutrient deficiency. In an embodiment, the agronomic characteristic is an increase in yield or an increase in drought tolerance. In an embodiment, the Cas9 endonuclease creates the double strand break in a coding region of the polynucleotide. In an embodiment, the plant is selected from the group consisting of maize, soybean, rice, wheat, sorghum, brassica, sunflower, and camelina.

A method of improving grain yield of a maize plant, the method includes providing a guide RNA that targets a polynucleotide involved in ethylene biosynthesis or ethylene signaling, the guide RNA acts in association with a Cas endonuclease that creates a double strand break at the polynucleotide and generating the plant, wherein the maize plant exhibits improved grain yield. In an embodiment, the donor polynucleotide comprises one or more nucleotide changes as compared to a corresponding endogenous unmodified genomic DNA of the polynucleotide involved in ethylene biosynthesis or ethylene signaling. In an embodiment, the polynucleotide is a maize ACC synthase. In an embodiment, the polynucleotide is maize ARGOS. In an embodiment, the expression of the maize ACC synthase is reduced as compared to a control maize plant. In an embodiment, the maize ARGOS is increased as compared to a control maize plant. In an embodiment, the maize ARGOS is increased by inserting a heterologous regulatory element.

A method of improving grain yield or nitrogen use efficieny of a maize plant, the method includes providing a guide RNA that targets a genomic region regulating the expression of a polynucleotide encoding a serine threonine protein phosphatase, the guide RNA acts in association with a Cas endonuclease that creates a double strand break at the genomic region and generating the maize plant, wherein the maize plant exhibits improved grain yield or nitrogen use efficiency. In an embodiment, the serine threonine protein phosphatase is ZmSTPP3. In an embodiment, expression of ZmSTPP3 is increased as compared to a control maize plant.

In an embodiment, expression of ZmSTPP3 is increased by inserting a heterologous regulatory element. In an embodiment, heterologous regulatory element is a moderate constitutive promoter. In an embodiment, the heterologous regulatory element is maize derived.

A method of improving grain yield or nitrogen use efficieny of a maize plant, the method includes providing a guide RNA that targets a genomic region of the maize plant to introduce one or more changes to a polynucleotide thereby generating a dominant phenotype of reduced male fertility, the guide RNA acting in association with a Cas endonuclease that creates a double strand break at the genomic region and generating the maize plant, wherein the maize plant exhibits reduced male fertility and thereby improved grain yield or nitrogen use efficiency when fertilized by a maize plant comprising a plurality of fertile pollen. In an embodiment, the reduced male fertility In an embodiment, the maize plant is an elite inbred or hybrid maize plant. In an embodiment, the MS44 polypeptide has a mutation at a position that corresponds to a signal peptide cleavage site. In an embodiment, the signal peptide cleavage site is at about amino acid position 38 or 39 of the unprocessed MS44 polypeptide.

A method of improving grain yield or nitrogen use efficieny of a crop plant, the method includes providing a guide RNA that targets a genomic region of the plant to introduce one or more changes to a polynucleotide encoding a polypeptide that is at least 70% identical to SEQ ID NO: 554, thereby generating a dominant phenotype of reduced male fertility, the guide RNA acting in association with a Cas endonuclease that creates a double strand break at the genomic region and generating the plant, wherein the plant exhibits reduced male fertility and thereby improved grain yield or nitrogen use efficiency when fertilized by a fertile plant comprising a plurality of fertile pollen. In an embodiment, the plant is selected from the group consisting of rice, wheat, and sorghum. In an embodiment, the plant is of an elite variety that is transformable. In an embodiment, the MS44 polypeptide has a mutation at a position that corresponds to a signal peptide cleavage site. In an embodiment, the plant is grown in a reduced nitrogen environment. In an embodiment, the polypeptide is about 90% identical to SEQ ID NO: 554

In an embodiment, the of the disclosure, the method comprises a method for selecting a plant comprising an altered target site in its plant genome, the method comprising: a) obtaining a first plant comprising at least one Cas endonuclease capable of introducing a double strand break at a target site in the plant genome; b) obtaining a second plant comprising a guide RNA that is capable of forming a complex with the Cas endonuclease of (a), c) crossing the first plant of (a) with the second plant of (b); d) evaluating the progeny of (c) for an alteration in the target site and e) selecting a progeny plant that possesses the desired alteration of said target site.

In another embodiment, the method comprises, a method for selecting a plant comprising an altered target site in its plant genome, the method comprising selecting at least one progeny plant that comprises an alteration at a target site in its plant genome, wherein said progeny plant was obtained by crossing a first plant comprising at least one Cas endonuclease with a second plant comprising a guide RNA, wherein said Cas endonuclease is capable of introducing a double strand break at said target site.

The plant in these embodiments is a monocot or a dicot. More specifically, the monocot is selected from the group consisting of maize, rice, sorghum, rye, barley, wheat, millet, oats, sugarcane, turfgrass, or switchgrass. The dicot is selected from the group consisting of soybean, canola, alfalfa, sunflower, cotton, tobacco, peanut, potato, tobacco, Arabidopsis, or safflower.

In some embodiments, the target site is located in the gene sequence of an acetolactate synthase.

In another embodiment the disclosure comprises a plant, plant part, or seed, comprising a recombinant DNA construct, said recombinant DNA construct comprising a promoter operably linked to a nucleotide sequence encoding a plant optimized Cas9 endonuclease, wherein said plant optimized Cas9 endonuclease is capable of binding to and creating a double strand break in a genomic target sequence said plant genome.

In another embodiment the plant comprises a recombinant DNA construct and a guide RNA, wherein said recombinant DNA construct comprises a promoter operably linked to a nucleotide sequence encoding a plant optimized Cas9 endonuclease, wherein said plant optimized Cas9 endonuclease and guide RNA are capable of forming a complex and creating a double strand break in a genomic target sequence said plant genome.

In another embodiment, the recombinant DNA construct comprises a promoter operably linked to a nucleotide sequence encoding a plant optimized Cas9 endonuclease, wherein said plant optimized Cas9 endonuclease is capable of binding to and creating a double strand break in a genomic target sequence said plant genome.

In another embodiment, the recombinant DNA construct comprises a promoter operably linked to a nucleotide sequence expressing a guide RNA, wherein said guide RNA is capable of forming a complex with a plant optimized Cas9 endonuclease, and wherein said complex is capable of binding to and creating a double strand break in a genomic target sequence said plant genome.

In another embodiment, the method comprises a method for selecting a male sterile or male fertile plant, the method comprising selecting at least one progeny plant that comprises an alteration at a genomic target site located in a male fertility gene locus, wherein said progeny plant is obtained by crossing a first plant expressing a Cas9 endonuclease to a second plant comprising a guide RNA, wherein said Cas endonuclease is capable of introducing a double strand break at said genomic target site.

In another embodiment, the method comprises a method for producing a male sterile or male fertile plant, the method comprising: a) obtaining a first plant comprising at least one Cas endonuclease capable of introducing a double strand break at a genomic target site located in a male fertility gene locus in the plant genome; b) obtaining a second plant comprising a guide RNA that is capable of forming a complex with the Cas endonuclease of (a),c) crossing the first plant of (a) with the second plant of (b); d) evaluating the progeny of (c) for an alteration in the target site; and e) selecting a progeny plant that is male sterile or male fertile. Male fertility genes can be selected from,

Compositions and methods are also provided for editing a nucleotide sequence in the genome of a cell. In one embodiment, the disclosure describes a method for editing a nucleotide sequence in the genome of a plant cell, the method comprising providing a guide RNA, a polynucleotide modification template, and at least one maize optimized Cas9 endonuclease to a plant cell, wherein the maize optimized Cas9 endonuclease is capable of introducing a double-strand break at a target site in the plant genome, wherein said polynucleotide modification template includes at least one nucleotide modification of said nucleotide sequence. The nucleotide to be edited (the nucleotide sequence of interest) can be located within or outside a target site that is recognized and cleaved by a Cas endonuclease. Cells include, but are not limited to, human, animal, bacterial, fungal, insect, and plant cells as well as plants and seeds produced by the methods described herein.

A method of providing an additional expression profile for an endogenous polynucleotide of a plant cell while maintaining the original endogenous expression pattern, the method comprising providing a heterologous regulatory element in an upstream region of the endogenous polynucleotide such that the native expression pattern of the original gene is maintained by providing functional terminator sequences Additional embodiments of the methods and compositions of the present invention are disclosed herein.

### BRIEF DESCRIPTION OF THE DRAWINGS AND THE SEQUENCE LISTING

The disclosure is more fully understood from the following detailed description and the accompanying drawings and Sequence Listing, which form a part of this application. The sequence descriptions and sequence listing (file name BB2394_SeqListing.txt”, created Jul. 4, 2011 and 548 kb) attached hereto comply with the rules governing nucleotide and amino acid sequence disclosures in patent applications as set forth in 37 C.F.R. §§1.821-1.825. The sequence descriptions contain the three letter codes for amino acids as defined in 37 C.F.R. §§1.821-1.825, which are incorporated herein by reference.

### FIGURES

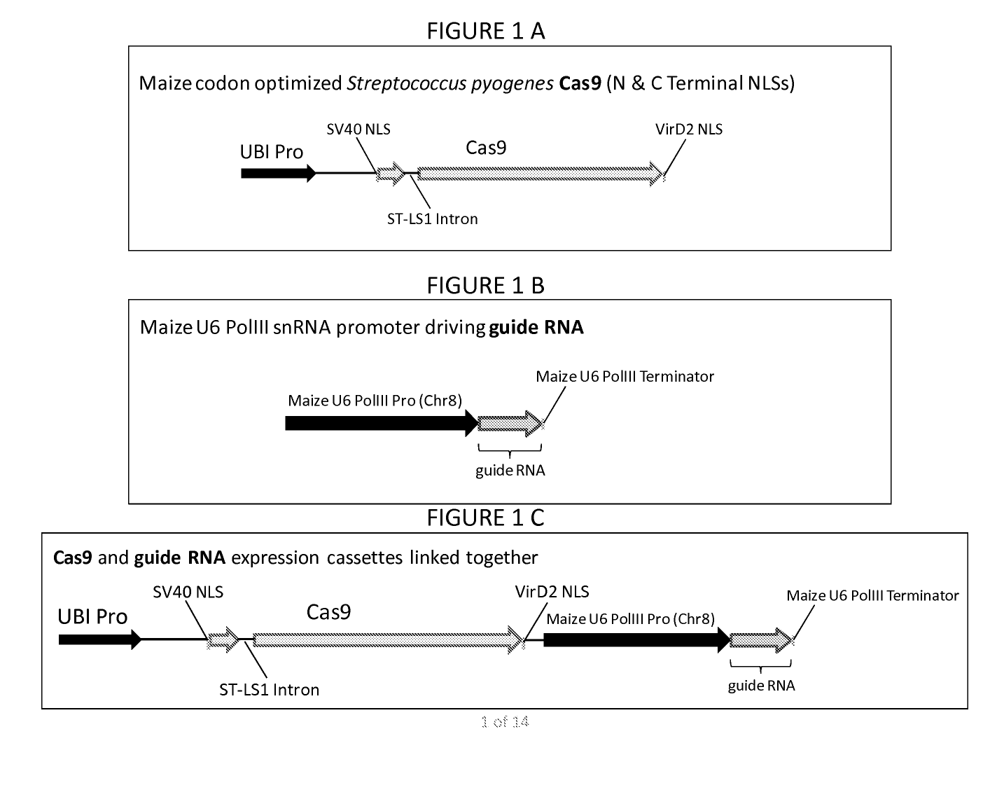

FIG. 1A shows a maize optimized Cas9 gene (encoding a Cas9 endonuclease) containing a potato ST-LS1 intron, a SV40 amino terminal nuclear localization sequence (NLS), and a VirD2 carboxyl terminal NLS, operably linked to a plant ubiquitin promoter (SEQ ID NO: 5). The maize optimized Cas9 gene (just Cas9 coding sequence, no NLSs) corresponds to nucleotide positions 2037-2411 and 2601-6329 of SEQ ID NO: 5 with the potato intron residing at positions 2412-2600 of SEQ ID NO: 5.SV40 NLS is at positions 2010-2036 of SEQ ID NO: 5. VirD2 NLS is at positions 6330-6386 of SEQ ID NO: 5. FIG. 1B shows a long guide RNA operably linked to a maize U6 polymerase III promoter terminating with a maize U6 terminator (SEQ ID NO: 12). The long guide RNA containing the variable targeting domain corresponding to the maize LIGCas-3 target site (SEQ ID NO: 8) is transcribed from/corresponds to positions 1001-1094 of SEQ ID NO: 12. FIG. 1 C shows the maize optimized Cas9 and long guide RNA expression cassettes combined on a single vector DNA (SEQ ID NO: 102).

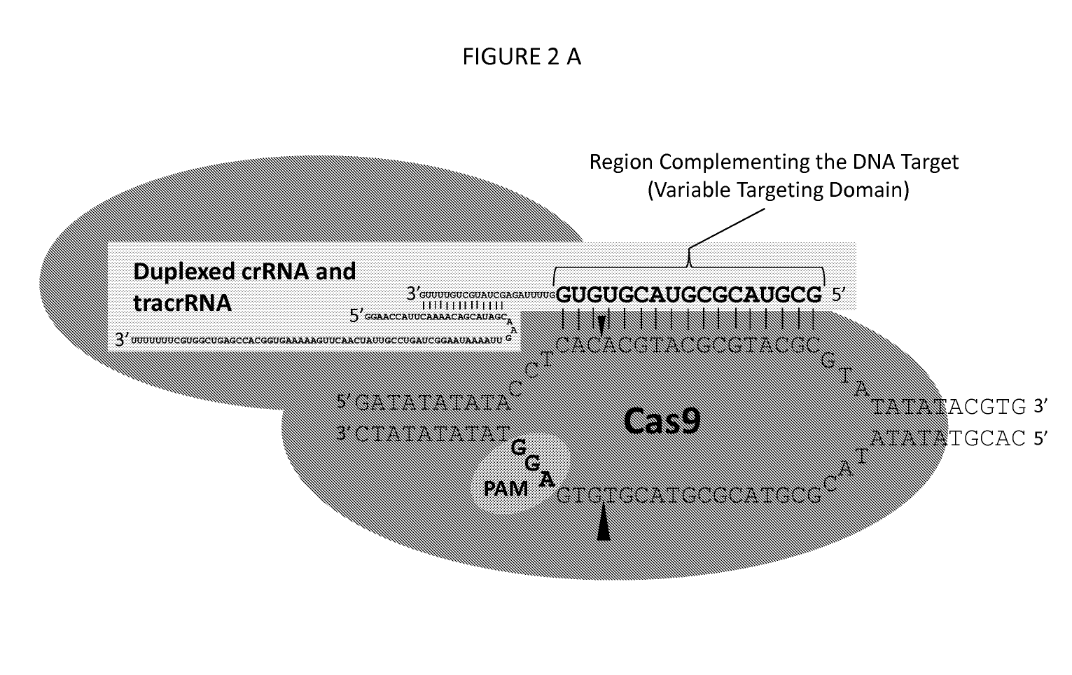

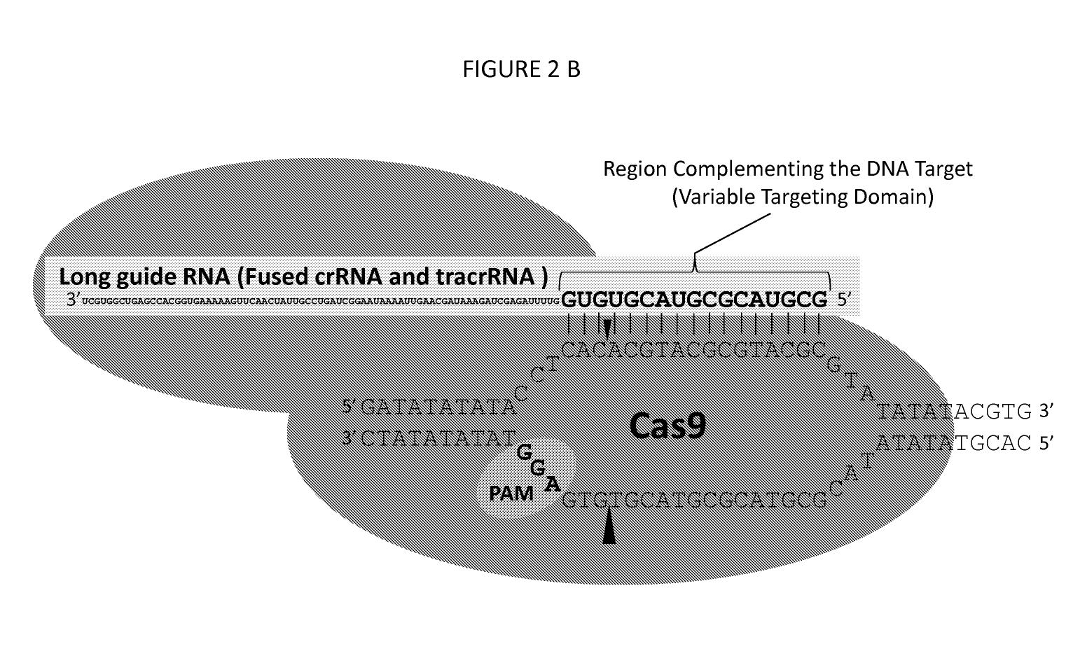

FIG. 2A illustrates the duplexed crRNA (SEQ ID NO:6)-tracrRNA (SEQ ID NO:7)/Cas9 endonuclease system and target DNA complex relative to the appropriately oriented PAM sequence at the maize LIGCas-3 (SEQ ID NO: 18) target site with triangles pointing towards the expected site of cleavage on both sense and anti-sense DNA strands. FIG. 2B illustrates the guide RNA/Cas9 endonuclease complex interacting with the genomic target site relative to the appropriately oriented PAM sequence (GGA) at the maize genomic LIGCas-3 target site (SEQ ID NO:18). The guide RNA (shown as boxed-in in light gray, SEQ ID NO: 8) is a fusion between a crRNA and tracrRNA and comprises a variable targeting domain that is complementary to one DNA strand of the double strand DNA genomic target site. The Cas9 endonuclease is shown in dark gray. Triangles point towards the expected site of DNA cleavage on both sense and anti-sense DNA strands.

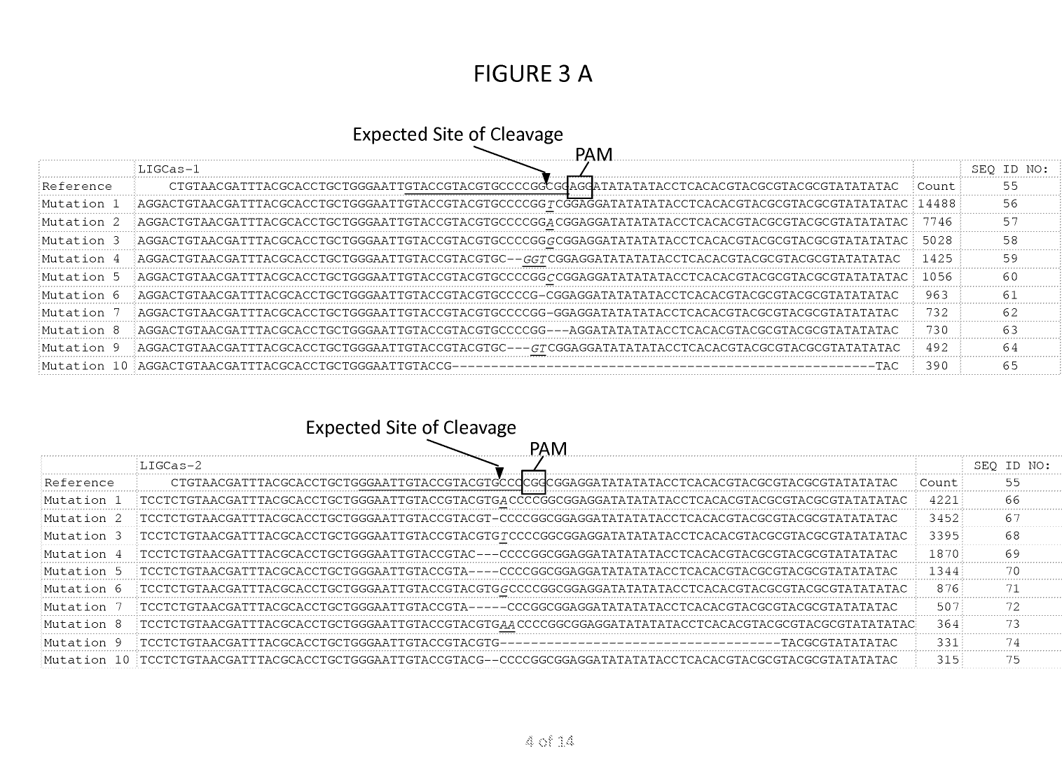

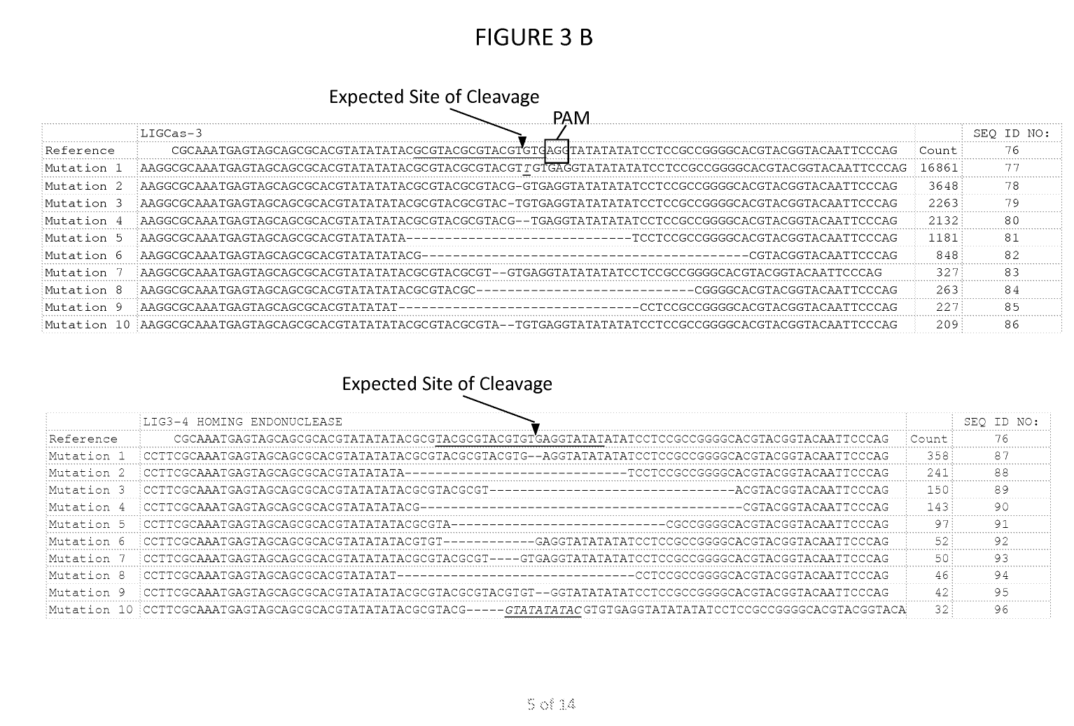

FIG. 3A-3B shows an alignment and count of the top 10 most frequent NHEJ mutations induced by the maize optimized guide RNA/Cas endonuclease system described herein compared to a LIG3-4 homing endonuclease control at the maize genomic Liguleless 1 locus. The mutations were identified by deep sequencing. The reference sequence represents the unmodified locus with each target site underlined. The PAM sequence and expected site of cleavage are also indicated. Deletions or insertions as a result of imperfect NHEJ are shown by a “−” or an italicized underlined nucleotide, respectively. The reference and mutations 1-10 of the LIGCas-1 target site correspond to SEQ ID NOs: 55-65, respectively. The reference and mutations 1-10 of the LIGCas-2 correspond to SEQ ID NOs: 55, 65-75, respectively. The reference and mutations 1-10 of the LIGCas-3 correspond to SEQ ID NOs: 76-86, respectively. The reference and mutations 1-10 of the LIG3-4 homing endonuclease target site correspond to SEQ ID NOs: 76, 87-96, respectively.

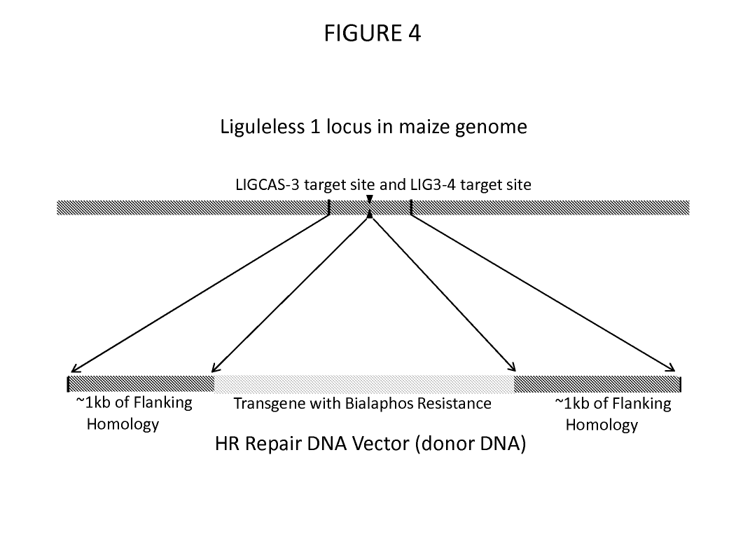

FIG. 4 illustrates how the homologous recombination (HR) repair DNA vector (SEQ ID NO: 97) was constructed. To promote site-specific transgene insertion by homologous recombination, the transgene (shown in light gray) was flanked on either side by approximately 1 kb of DNA with homology to the maize genomic regions immediately adjacent to the LIGCas3 and LIG3-4 homing endonuclease expected sites of cleavage.

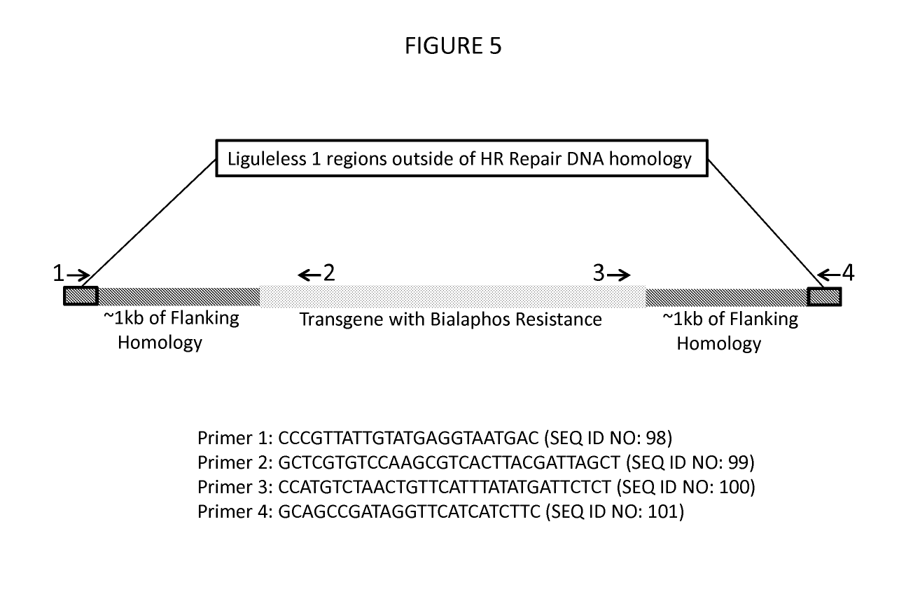

FIG. 5 illustrates how genomic DNA extracted from stable transformants was screened for site-specific transgene insertion by PCR. Genomic primers (corresponding to SEQ ID NOs: 98 and 101) within the Liguleless 1 locus were designed outside of the regions used in constructing the HR repair DNA vector (SEQ ID NO: 97) and were paired with primers inside the transgene (corresponding to SEQ ID NOs: 99 and 100) to facilitate PCR detection of unique genomic DNA junctions created by appropriately oriented site-specific transgene integration.

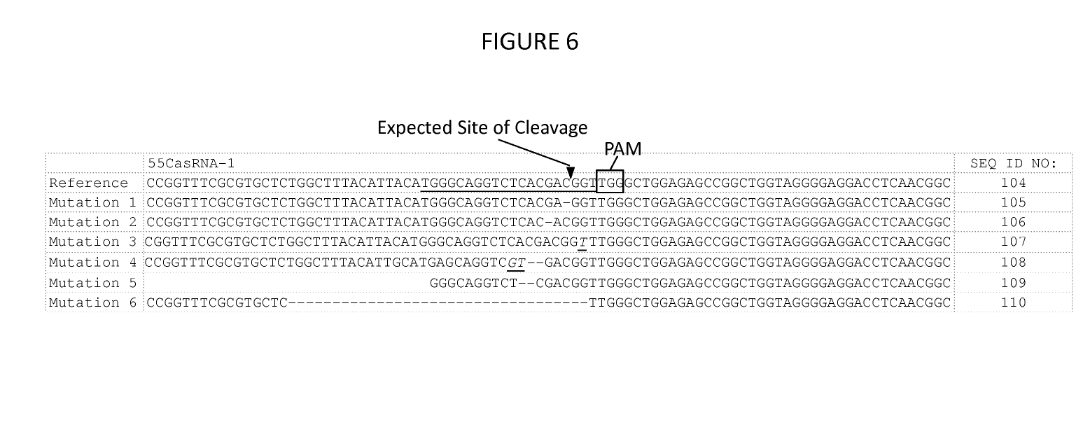

FIG. 6 shows an alignment of the NHEJ mutations induced by the maize optimized guide RNA/Cas endonuclease system, described herein, when the short guide RNA was delivered directly as RNA. The mutations were identified by deep sequencing. The reference illustrates the unmodified locus with the genomic target site underlined. The PAM sequence and expected site of cleavage are also indicated. Deletions or insertions as a result of imperfect NHEJ are shown by a “−” or an italicized underlined nucleotide, respectively. The reference and mutations 1-6 for 55CasRNA-1 correspond to SEQ ID NOs: 104-110, respectively.

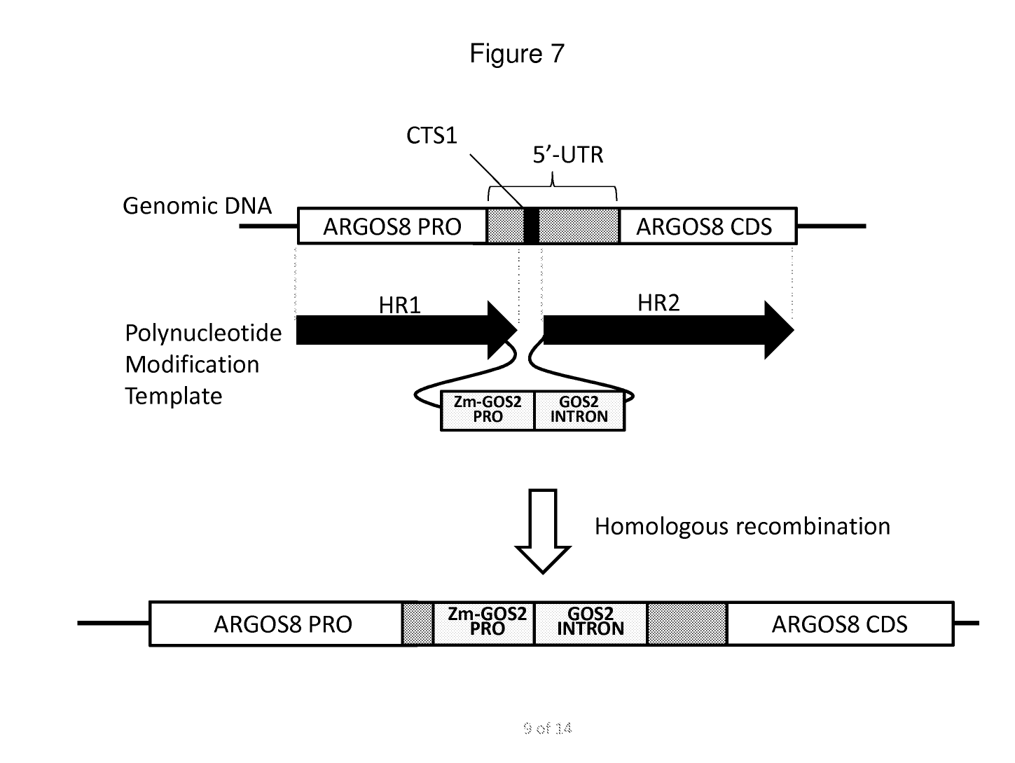

FIG. 7. Schematic representation of Zm-GOS2 PRO:GOS2 INTRON insertion in the 5′-UTR of maize ARGOS8 gene by targeting the guide RNA/Cas9 target sequence 1 (CTS1, SEQ ID NO: 1) with the gRNA1/Cas9 endonuclease system, described herein. HR1 and HR2 indicate homologous recombination regions.

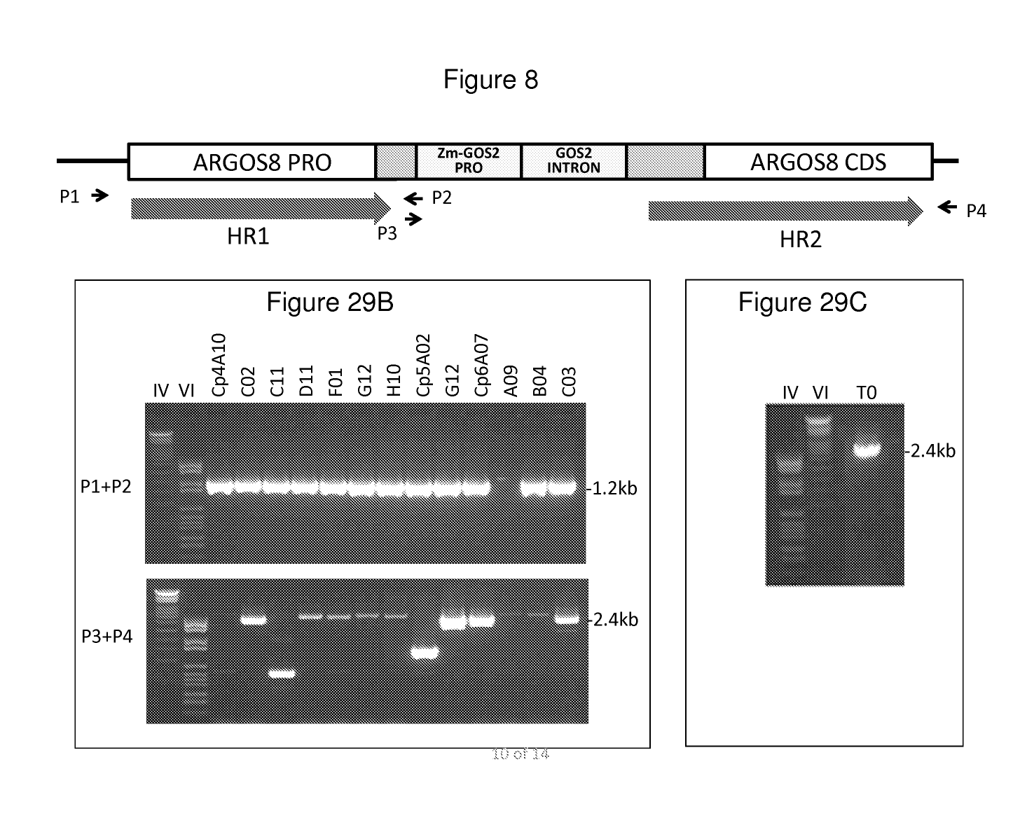

FIG. 8A-8C. Identification and analysis of Zm-GOS2 PRO:GOS2 INTRON insertion events in maize plants. (A) Schematic representation of Zm-GOS2 PRO:GOS2 INTRON insertion in the 5′-UTR of Zm-ARGOS8. CTS1 was targeted with the gRNA1/Cas9 endonuclease system, described herein. HR1 and HR2 indicate homologous recombination regions. P1 to P4 indicate PCR primers. (B) PCR screening of PMI-resistance calli to identify insertion events. PCR results are shown for 13 representative calli. The left and right junction PCRs were carried out with the primer pair P1+P2 and P3+P4, respectively. (C) PCR analysis of a T0 plant. A PCR product with the expected size (2.4 kb, Lane T0) was amplified with the primer P3 and P4.

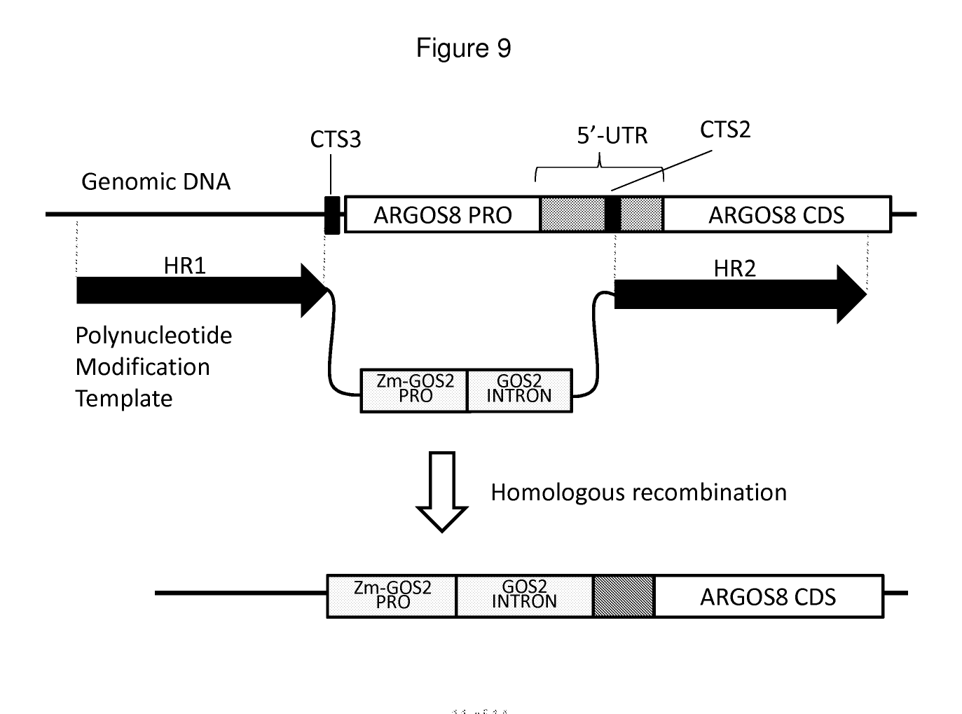

FIG. 9. Schematic representation of Zm-ARGOS8 promoter substitution with Zm-GOS2 PRO:GOS2 INTRON by targeting CTS3 (SEQ ID NO: 3) and CTS2 (SEQ ID NO:2). HR1 and HR2 indicate homologous recombination regions.

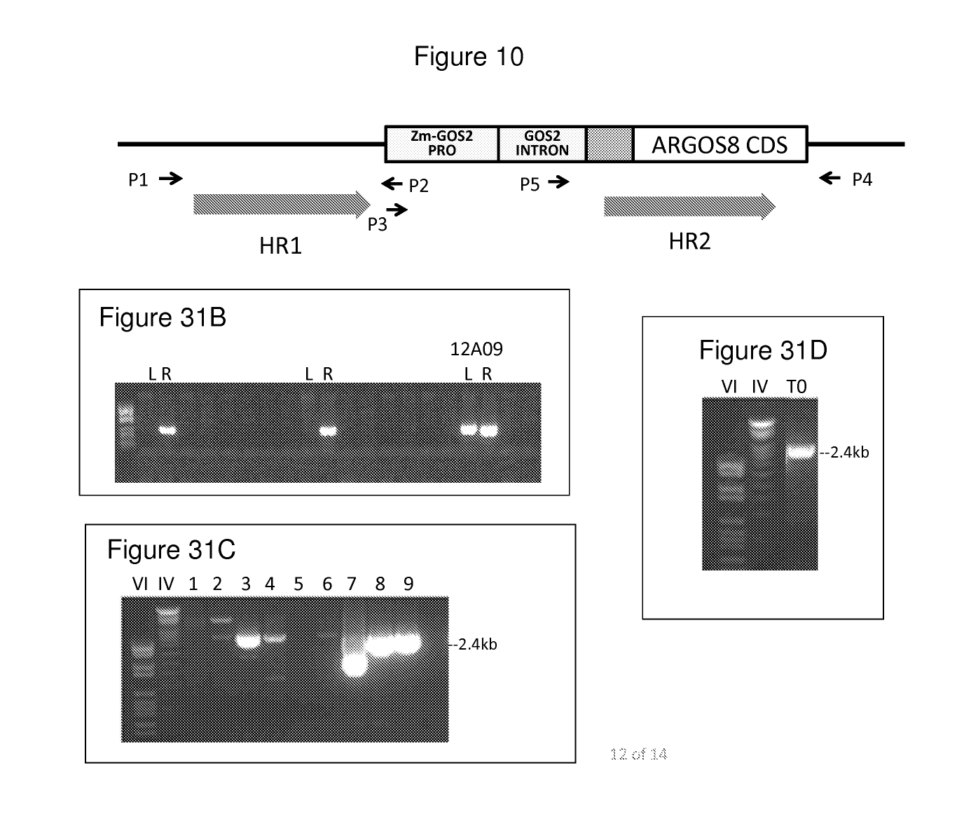

FIG. 10A-10D. Substitution of the native promoter of the ARGOS8 gene with Zm-GOS2 PRO:GOS2 INTRON in maize plants. (A) Schematic representation of the Zm-GOS2 PRO:GOS2 INTRON:ARGOS8 allele generated by promoter swap. Two guide RNA/Cas9 target sites, CTS3 (SEQ ID NO:3) and CTS2 (SEQ ID NO:2), were targeted with a gRNA3/gRNA2/Cas9 system. HR1 and HR2 indicate homologous recombination regions. P1 to P5 indicate PCR primers. (B) PCR screening of PMI-resistance calli to identify swap events. PCR results are shown for representative calli. One callus sample, 12A09, is positive for both left junction (L, primer P1+P2) and right junction (R, primer P5+P4) PCR, indicating that 12A09 is a swap event. (C) PCR analysis of the callus events identified in primary screening. PCR products with the expected size (2.4 kb) were amplified using the primer P3 and P4 from event #3, 4, 6, 8 and 9, indicating presence of the Zm-GOS2 PRO:GOS2 INTRON:ARGOS8 allele. (D) PCR analysis of a T0 plant. A PCR product with the expected size (2.4 kb, Lane T0) was amplified with the primer P3 and P4.

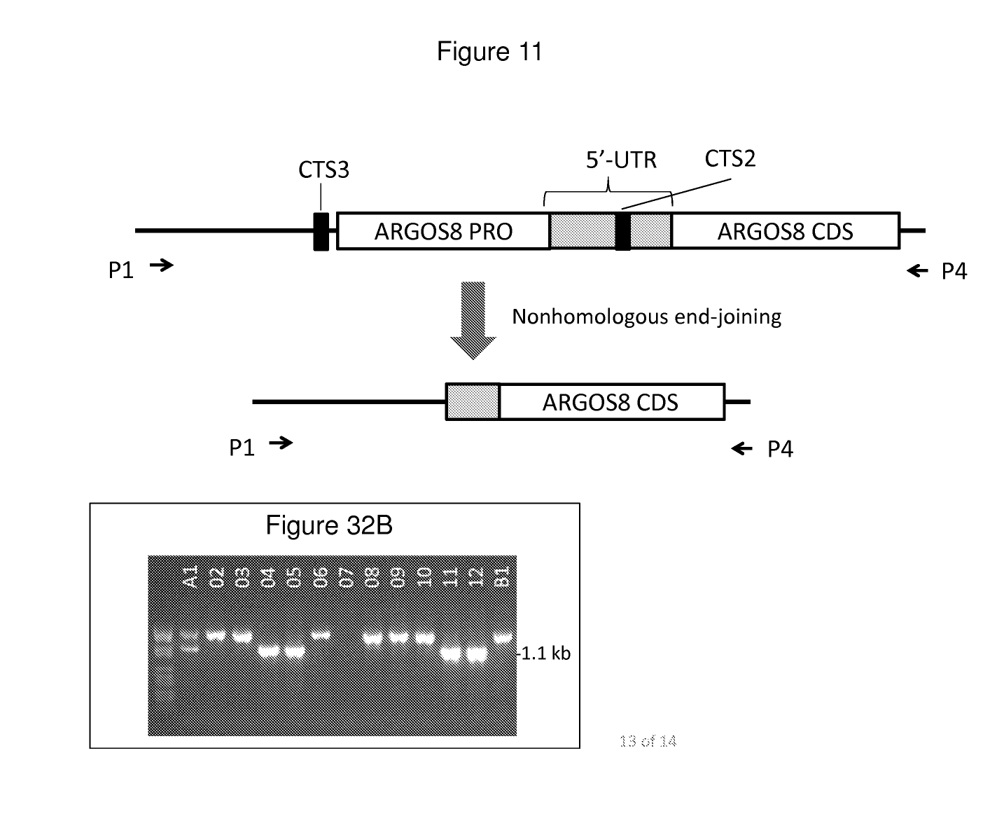

FIG. 11A-11B. Deletion of the native promoter of the ARGOS8 gene in maize plants. (A) Schematic representation of promoter deletion. Two guide RNA's and a Cas9 endonuclease system, referred to as a gRNA3/gRNA2/Cas9 system, were used to target the CTS3 and CTS2 sites in Zm-ARGOS8. P1 and P4 indicate PCR primers for deletion event screening. (B) PCR screening of PMI-resistance calli to identify deletion events. PCR results are shown for 15 representative calli. A 1.1-kp PCR product indicates deletion of the CTS3/CTS2 fragment.

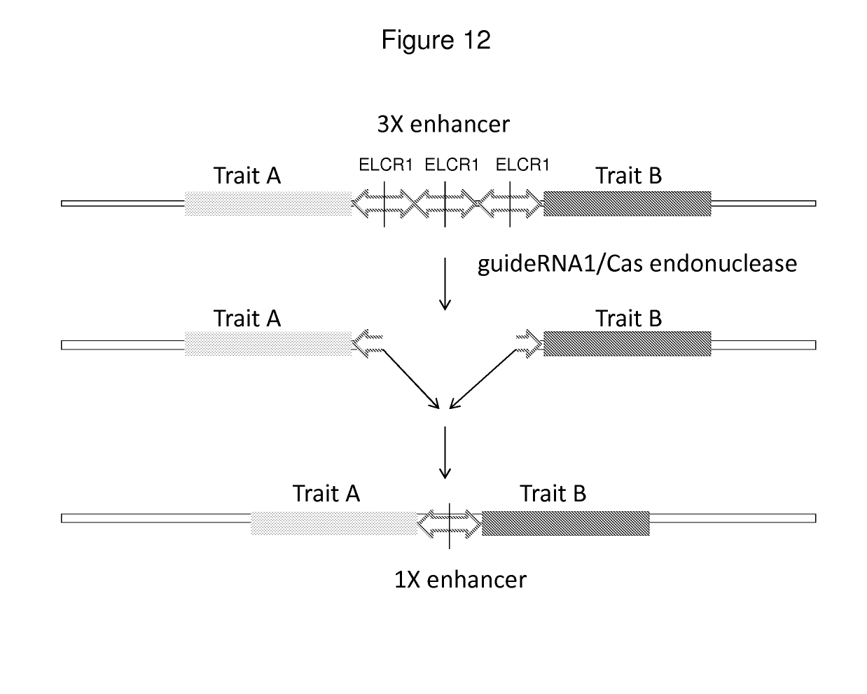

FIG. 12. Schematic representation of enhancer element deletions using the guide RNA/Cas9 target sequence. The enhancer element to be deleted can be, but is not limited to, a 35S enhancer element.

### SEQUENCES

SEQ ID NO: 1 is the nucleotide sequence of the Cas9 gene from Streptococcus pyogenes M1 GAS (SF370).

SEQ ID NO: 2 is the nucleotide sequence of the potato ST-LS1 intron.

SEQ ID NO: 3 is the amino acid sequence of SV40 amino N-terminal.

SEQ ID NO: 4 is the amino acid sequence of Agrobacterium tumefaciens bipartite VirD2 T-DNA border endonuclease carboxyl terminal.

SEQ ID NO: 5 is the nucleotide sequence of an expression cassette expressing the maize optimized Cas9.

SEQ ID NO: 6 is the nucleotide sequence of crRNA containing the LIGCas-3 target sequence in the variable targeting domain.

SEQ ID NO: 7 is the nucleotide sequence of the tracrRNA.

SEQ ID NO: 8 is the nucleotide sequence of a long guide RNA containing the LIGCas-3 target sequence in the variable targeting domain.

SEQ ID NO: 9 is the nucleotide sequence of the Chromosome 8 maize U6 polymerase III promoter.

SEQ ID NO: 10 list two copies of the nucleotide sequence of the maize U6 polymerase III terminator.

SEQ ID NO: 11 is the nucleotide sequence of the maize optimized short guide RNA containing the LIGCas-3 variable targeting domain.

SEQ ID NO: 12 is the nucleotide sequence of the maize optimized long guide RNA expression cassette containing the LIGCas-3 variable targeting domain.

SEQ ID NO: 13 is the nucleotide sequence of the Maize genomic target site MS26Cas-1 plus PAM sequence.

SEQ ID NO: 14 is the nucleotide sequence of the Maize genomic target site MS26Cas-2 plus PAM sequence.

SEQ ID NO: 15 is the nucleotide sequence of the Maize genomic target site MS26Cas-3 plus PAM sequence.

SEQ ID NO: 16 is the nucleotide sequence of the Maize genomic target site LIGCas-2 plus PAM sequence.

SEQ ID NO: 17 is the nucleotide sequence of the Maize genomic target site LIGCas-3 plus PAM sequence.

SEQ ID NO: 18 is the nucleotide sequence of the Maize genomic target site LIGCas-4 plus PAM sequence.

SEQ ID NO: 19 is the nucleotide sequence of the Maize genomic target site MS45Cas-1 plus PAM sequence.

SEQ ID NO: 20 is the nucleotide sequence of the Maize genomic target site MS45Cas-2 plus PAM sequence.

SEQ ID NO: 21 is the nucleotide sequence of the Maize genomic target site MS45Cas-3 plus PAM sequence.

SEQ ID NO: 22 is the nucleotide sequence of the Maize genomic target site ALSCas-1 plus PAM sequence.

SEQ ID NO: 23 is the nucleotide sequence of the Maize genomic target site ALSCas-2 plus PAM sequence.

SEQ ID NO: 24 is the nucleotide sequence of the Maize genomic target site ALSCas-3 plus PAM sequence.

SEQ ID NO: 25 is the nucleotide sequence of the Maize genomic target site EPSPSCas-1 plus PAM sequence.

SEQ ID NO: 26 is the nucleotide sequence of the Maize genomic target site EPSPSCas-2 plus PAM sequence.

SEQ ID NO: 27 is the nucleotide sequence of the Maize genomic target site EPSPSCas-3 plus PAM sequence.

SEQ ID NOs: 28-52 are the nucleotide sequence of target site specific forward primers for primary PCR.

SEQ ID NO: 53 is the nucleotide sequence of the forward primer for secondary PCR.

SEQ ID NO: 54 is the nucleotide sequence of Reverse primer for secondary PCR SEQ ID NO: 55 is the nucleotide sequence of the unmodified reference sequence for LIGCas-1 and LIGCas-2 locus.

SEQ ID NOs: 56-65 are the nucleotide sequences of mutations 1-10 for LIGCas-1.

SEQ ID NOs: 66-75 are the nucleotide sequences of mutations 1-10 for LIGCas-2.

SEQ ID NO: 76 is the nucleotide sequence of the unmodified reference sequence for the LIGCas-3 and LIG3-4 homing endonuclease locus.

SEQ ID NOs: 77-86 are the nucleotide sequences of mutations 1-10 for LIGCas-3.

SEQ ID NOs: 88-96 are the nucleotide sequences of mutations 1-10 for LIG3-4 homing endonuclease locus.

SEQ ID NO: 97 is the nucleotide sequence of a donor vector referred to as an HR Repair DNA.

SEQ ID NO: 98 is the nucleotide sequence of forward PCR primer for site-specific transgene insertion at junction 1.

SEQ ID NO: 99 is the nucleotide sequence of reverse PCR primer for site-specific transgene insertion at junction 1.

SEQ ID NO: 100 is the nucleotide sequence of forward PCR primer for site-specific transgene insertion at junction 2.

SEQ ID NO: 101 is the nucleotide sequence of reverse PCR primer for site-specific transgene insertion at junction 2.

SEQ ID NO: 102 is the nucleotide sequence of the linked Cas9 endonuclease and LIGCas-3 long guide RNA expression cassettes

SEQ ID NO: 103 is the nucleotide sequence of Maize genomic target site 55CasRNA-1 plus PAM sequence.

SEQ ID NO: 104 is the nucleotide sequence of the unmodified reference sequence for 55CasRNA-1 locus.

SEQ ID NOs: 105-110 are the nucleotide sequences of mutations 1-6 for 55CasRNA-1.

SEQ ID NO: 111 is the nucleotide sequence of LIG3-4 homing endonuclease target site

SEQ ID NO: 112 is the nucleotide sequence of LIG3-4 homing endonuclease coding sequence.

SEQ ID NO: 113 is the nucleotide sequence of the MS26++ homing endonuclease target site.

SEQ ID NO: 114 is the nucleotide sequence of MS26++ homing endonuclease coding sequence

SEQ ID NO: 115 is the nucleotide sequence of the soybean codon optimized Cas9 gene.

SEQ ID NO: 116 is the nucleotide sequence of the soybean constitutive promoter GM-EF1A2.

SEQ ID NO: 117 is the nucleotide sequence of linker SV40 NLS.

SEQ ID NO: 118 is the amino acid sequence of soybean optimized Cas9 with a SV40 NLS.

SEQ ID NO: 119 is the nucleotide sequence of vector QC782.

SEQ ID NO: 120 is the nucleotide sequence of soybean U6 polymerase III promoter described herein, GM-U6-13.1 PRO.

SEQ ID NO: 121 is a nucleotide sequence of a guide RNA.

SEQ ID NO: 122 is the nucleotide sequence of vector QC783.

SEQ ID NO: 123 is the nucleotide sequence of vector QC815.

SEQ ID NO: 124 is the nucleotide sequence of a Cas9 endonuclease (cas9-2) from S. pyogenes.

SEQ ID NO: 125 is the nucleotide sequence of the DD20CR1 soybean target site

SEQ ID NO: 126 is the nucleotide sequence of the DD20CR2 soybean target site

SEQ ID NO: 127 is the nucleotide sequence of the DD43CR1 soybean target site

SEQ ID NO: 128 is the nucleotide sequence of the DD43CR2 soybean target site

SEQ ID NO: 129 is the nucleotide sequence of the DD20 sequence.

SEQ ID NO: 130 is the nucleotide sequence of the complementary DD20 sequence.

SEQ ID NO: 131 is the nucleotide sequence of DD43 sequence.

SEQ ID NO: 132 is the nucleotide sequence of the DD43 complementary sequence.

SEQ ID NO: 133-141 are primer sequences.

SEQ ID NO: 142 is the nucleotide sequence of the DD20CR1 PCR amplicon.

SEQ ID NO: 143 is the nucleotide sequence of the DD20CR2 PCR amplicon.

SEQ ID NO: 144 is the nucleotide sequence of the DD43CR1 PCR amplicon.

SEQ ID NO: 145 is the nucleotide sequence of the DD43CR2 PCR amplicon.

SEQ ID NO: 146 is the nucleotide sequence of the DD43CR2 PCR amplicon.

SEQ ID NO: 147-156 are the nucleotide sequence of mutations 1 to 10 for the DD20CR1 target site

SEQ ID NO: 157-166 are the nucleotide sequence of mutations 1 to 10 for the DD20CR2 target site

SEQ ID NO: 167-176 are the nucleotide sequence of mutations 1 to 10 for the DD43CR1 target site

SEQ ID NO: 177-191 are the nucleotide sequence of mutations 1 to 10 for the DD43CR2 target site.

SEQ ID NO: 192 is the amino acid sequence of a maize optimized version of the Cas9 protein.

SEQ ID NO: 193 is the nucleotide sequence of the maize optimized version of the Cas9 gene of SEQ ID NO: 192.

SEQ ID NO: 194 is the DNA version of guide RNA (EPSPS sgRNA).

SEQ ID NO: 195 is the EPSPS polynucleotide modification template.

SEQ ID NO: 196 is a nucleotide fragment comprising the TIPS nucleotide modifications.

SEQ ID NO: 197-204 are primer sequences.

SEQ ID NO: 205-208 are nucleotide fragments shown in FIG. 14.

SEQ ID NO: 209 is an example of a TIPS edited EPSPS nucleotide sequence fragment shown in FIG. 17.

SEQ ID NO: 210 is an example of a Wild-type EPSPS nucleotide sequence fragment shown in FIG. 17.

SEQ ID NO: 211 is the nucleotide sequence of a maize enolpyruvylshikimate-3-phosphate synthase (epsps) locus

SEQ ID NO: 212 is the nucleotide sequence of a Cas9 endonuclease (genbank CS571758.1) from S. thermophiles.

SEQ ID NO: 213 is the nucleotide sequence of a Cas9 endonuclease (genbank CS571770.1) from S. thermophiles.

SEQ ID NO: 214 is the nucleotide sequence of a Cas9 endonuclease (genbank CS571785.1) from S. agalactiae.

SEQ ID NO: 215 is the nucleotide sequence of a Cas9 endonuclease, (genbank CS571790.1) from S. agalactiae.

SEQ ID NO: 216 is the nucleotide sequence of a Cas9 endonuclease (genbank CS571790.1) from S. mutant.

SEQ ID NOs: 217-228 are primer and probe nucleotide sequences described in Example 17.

SEQ ID NOs: 229 is the nucleotide sequence of the MHP14Cas1 target site.

SEQ ID NOs: 230 is the nucleotide sequence of the MHP14Cas3 target site.

SEQ ID NOs: 231 is the nucleotide sequence of the TS8Cas1 target site.

SEQ ID NOs: 232 is the nucleotide sequence of the TS8Cas2 target site.

SEQ ID NOs: 233 is the nucleotide sequence of the TS9Cas2 target site.

SEQ ID NOs: 234 is the nucleotide sequence of the TS9Cas3 target site.

SEQ ID NOs: 235 is the nucleotide sequence of the TS10Cas1 target site.

SEQ ID NOs: 236 is the nucleotide sequence of the TS10Cas3 target site.

SEQ ID NOs: 237-244 are the nucleotide sequences shown in FIG. 19A-D.

SEQ ID NOs: 245-252 are the nucleotide sequences of the guide RNA expression cassettes described in Example 18.

SEQ ID NOs: 253-260 are the nucleotide sequences of donor DNA expression cassettes described in Example 18.

SEQ ID NOs: 261-270 are the nucleotide sequences of the primers described in Example 18.

SEQ ID NOs: 271-294 are the nucleotide sequences of the primers and probes described in Example 18.

SEQ ID NO: 295 is the nucleotide sequence of GM-U6-13.1 PRO, a soybean U6 polymerase III promoter described herein, SEQ ID NOs: 298, 300, 301 and 303 are the nucleotide sequences of the linked guideRNA/Cas9 expression cassettes.

SEQ ID NOs: 299 and 302 are the nucleotide sequences of the donor DNA expression cassettes.

SEQ ID NOs: 271-294 are the nucleotide sequences of the primers and probes described in Example 18.

SEQ ID NO: 304 is the nucleotide sequence of the DD20 qPCR amplicon.

SEQ ID NO: 305 is the nucleotide sequence of the DD43 qPCR amplicon.

SEQ ID NOs: 306-328 are the nucleotide sequences of the primers and probes described herein.

SEQ ID NOs: 329-334 are the nucleotide sequences of PCR amplicons described herein.

SEQ ID NO: 335 is the nucleotide sequence of a soybean genomic region comprising the DD20CR1 target site.

SEQ ID NO: 364 is the nucleotide sequence of a soybean genomic region comprising the DD20CR2 target site.

SEQ ID NO: 386 is the nucleotide sequence of a soybean genomic region comprising the DD43CR1 target site.

SEQ ID NOs: 336-363, 365-385 and 387-414 are the nucleotide sequences of shown in FIG. 26 A-C.

SEQ ID NOs: 415-444 are the nucleotide sequences of NHEJ mutations recovered based on the crRNA/tracrRNA/Cas endonuclease system shown in FIG. 27A-C.

SEQ ID NO: 445-447 are the nucleotide sequence of the LIGCas-1, LIGCas2 and LIGCas3 crRNA expression cassettes, respectively.

SEQ ID NO: 448 is the nucleotide sequence of the tracrRNA expression cassette.

SEQ ID NO: 449 is the nucleotide sequence of LIGCas-2 forward primer for primary PCR

SEQ ID NO: 450 is the nucleotide sequence of LIGCas-3 forward primer for primary PCR.

SEQ ID NO: 451 is the nucleotide sequence of the maize genomic Cas9 endonuclease target site Zm-ARGOS8-CTS1.

SEQ ID NO: 452 is the nucleotide sequence of the maize genomic Cas9 endonuclease target site Zm-ARGOS8-CTS2.

SEQ ID NO: 453 is the nucleotide sequence of the maize genomic Cas9 endonuclease target site Zm-ARGOS8-CTS3

SEQ ID NOs: 454-458 are the nucleotide sequence of primers P1, P2, P3, P4, P5, respectively.

SEQ ID NO: 459 is the nucleotide sequence of a Primer Binding Site (PBS), a sequence to facilitate event screening.

SEQ ID NO: 460 is the nucleotide sequence of the Zm-GOS2 PRO-GOS2 INTRON, the maize GOS2 promoter and GOS2 intron1 including the promoter, 5′-UTR1, INTRON1 and 5′-UTR2.

SEQ ID NO:461 is the nucleotide sequence of the maize Zm-ARGOS8 promoter.

SEQ ID NO:462 is the nucleotide sequence of the maize Zm-ARGOS8 5′-UTR.

SEQ ID NO:463 is the nucleotide sequence of the maize Zm-ARGOS8 codon sequence

SEQ ID NO:464 is the nucleotide sequence of the maize Zm-GOS2 gene, including promoter, 5′-UTR, CDS, 3′-UTR and introns.

SEQ ID NO:465 is the nucleotide sequence of the maize Zm-GOS2 PRO promoter.

SEQ ID NO:466 is the nucleotide sequence of the maize GOS2 INTRON, maize GOS2 5′-UTR1 and intron1 and 5′-UTR2.

SEQ ID NOs: 467-468, 490-491, 503-504 are the nucleotide sequence of the soybean genomic Cas endonuclease target sequences soy EPSPS-CR1, soy EPSPS-CR2, soy EPSPS-CR4, soy EPSPS-CR5, soy EPSPS-CR6, soy EPSPS-CR7, respectively

SEQ ID NO:469 is the nucleotide sequence of the soybean U6 small nuclear RNA promoter GM-U6-13.1.

SEQ ID NOs:470, 471 are the nucleotide sequences of the QC868, QC879 plasmids, respectively.

SEQ ID NOs:472, 473, 492, 493, 494, 505, 506, 507 are the nucleotide sequences of the RTW1013A, RTW1012A, RTW1199, RTW1200, RTW1190A, RTW1201, RTW1202, RTW1192A respectively.

SEQ ID NOs:474-488, 495-402, 508-512 are the nucleotide sequences of primers and probes.

SEQ ID NO: 489 is the nucleotide sequence of the soybean codon optimized Cas9.

SEQ ID NO: 513 is the nucleotide sequence of the 35S enhancer.

SEQ ID NO: 514 is the nucleotide sequence of the 35S-CRTS for gRNA1 at 163-181 (including pam at 3′end).

SEQ ID NO: 515 is the nucleotide sequence of the 35S-CRTS for gRNA2 at 295-319 (including pam at 3′end).

SEQ ID NO: 516 is the nucleotide sequence of the 35S-CRT for gRNA3 at 331-350 (including pam at 3′end).

SEQ ID NO: 517 is the nucleotide sequence of the EPSPS-K90R template.

SEQ ID NO: 518 is the nucleotide sequence of the EPSPS-IME template. S

SEQ ID NO: 519 is the nucleotide sequence of the EPSPS-Tspliced template.

SEQ ID NO: 520 is the amino acid sequence of ZM-RAP2.7 peptide

SEQ ID NO: 521 is the nucleotide sequence zM-RAP2.7 coding DNA sequence

SEQ ID NOs: 522 is the amino acid sequence of ZM-NPK1B peptide

SEQ ID NO: 523 is the nucleotide sequence of the ZM-NPK1B coding DNA sequence

SEQ ID NOs: 524 is the nucleotide sequence of the RAB17 promoter

SEQ ID NOs: 525 is the amino acid sequence of the Maize FTM1.

SEQ ID NO: 526 is the nucleotide sequence of the Maize FTM1 coding DNA sequence.

SEQ ID NOs: 527-532 are nucleotide sequences.

SEQ ID NOS: 551-553 are guide RNA targets for a male fertility reduction gene.

SEQ ID NO: 554 is a polypeptide involved in maize male fertility.

### DETAILED DESCRIPTION OF THE INVENTION

The present disclosure now will be described more fully hereinafter with reference to the accompanying drawings, in which some, but not all embodiments of the disclosure are shown. These should not be construed as limited to the embodiments set forth herein. Like numbers refer to like elements throughout.

Although specific terms are employed herein, they are used in a generic and descriptive sense only and not for purposes of limitation.

### I. Overview

Compositions and methods are provided for genome modification of a target sequence in the genome of a plant or plant cell, for selecting plants, for gene editing, and for inserting a polynucleotide of interest into the genome of a plant. The methods employ a guide RNA/Cas endonuclease system, wherein the Cas endonuclease is guided by the guide RNA to recognize and optionally introduce a double strand break at a specific target site into the genome of a cell. The guide RNA/Cas endonuclease system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest.

Breeding methods utilizing a two component guide RNA/Cas endonuclease system are also disclosed. Compositions and methods are also provided for editing a nucleotide sequence in the genome of a cell. The nucleotide sequence to be edited (the nucleotide sequence of interest) can be located within or outside a target site that is recognized by a Cas endonuclease.

### II. Guide RNA/Cas Endonuclease Systems

a. CRISPR Loci

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

CRISPR loci were first recognized in E. coli (Ishino et al. (1987) J. Bacterial. 169:5429-5433; Nakata et al. (1989) J. Bacterial. 171:3553-3556). Similar interspersed short sequence repeats have been identified in Haloferax mediterranei, Streptococcus pyogenes, Anabaena, and Mycobacterium tuberculosis (Groenen et al. (1993) Mol. Microbiol. 10:1057-1065; Hoe et al. (1999) Emerg. Infect. Dis. 5:254-263; Masepohl et al. (1996) Biochim. Biophys. Acta 1307:26-30; Mojica et al. (1995) Mol. Microbiol. 17:85-93). The CRISPR loci differ from other SSRs by the structure of the repeats, which have been termed short regularly spaced repeats (SRSRs) (Janssen et al. (2002) OMICS J. Integ. Biol. 6:23-33; Mojica et al. (2000) Mol. Microbiol. 36:244-246). The repeats are short elements that occur in clusters, that are always regularly spaced by variable sequences of constant length (Mojica et al. (2000) Mol. Microbiol. 36:244-246).

b. Cas Genes, Cas Endonucleases

As used herein, the term “Cas gene” refers to a gene that is generally coupled, associated or close to or in the vicinity of flanking CRISPR loci. The terms “Cas gene”, “CRISPR-associated (Cas) gene” are used interchangeably herein. A comprehensive review of the Cas protein family is presented in Haft et al. (2005) Computational Biology, PLoS Comput Biol 1(6): e60. doi:10.1371/journal.pcbi.0010060.

As described therein, 41 CRISPR-associated (Cas) gene families are described, in addition to the four previously known gene families. It shows that CRISPR systems belong to different classes, with different repeat patterns, sets of genes, and species ranges. The number of Cas genes at a given CRISPR locus can vary between species.

As used herein, the term “Cas endonuclease” refers to a Cas protein encoded by a Cas gene, wherein said Cas protein is capable of introducing a double strand break into a DNA target sequence. The Cas endonuclease is guided by the guide polynucleotide to recognize and optionally introduce a double strand break at a specific target site into the genome of a cell. As used herein, the tem “guide polynucleotide/Cas endonuclease system” refers to a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is approximately oriented at the 3′ end of the target sequence (FIG. 2A, FIG. 2B).

In one embodiment, the Cas endonuclease gene is a Cas9 endonuclease, such as but not limited to, Cas9 genes listed in SEQ ID NOs: 462, 474, 489, 494, 499, 505, and 518 of WO2007/025097 published Mar. 1, 2007, and incorporated herein by reference. In another embodiment, the Cas endonuclease gene is plant, maize or soybean optimized Cas9 endonuclease (FIG. 1 A). In another embodiment, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

In one embodiment, the Cas endonuclease gene is a Cas9 endonuclease gene of SEQ ID NO:1, 124, 212, 213, 214, 215, 216, 193 or nucleotides 2037-6329 of SEQ ID NO:5, or any functional fragment or variant thereof.

The terms “functional fragment”, “fragment that is functionally equivalent” and “functionally equivalent fragment” are used interchangeably herein. These terms refer to a portion or subsequence of the Cas endonuclease sequence of the present invention in which the ability to create a double-strand break is retained.

The terms “functional variant”, “Variant that is functionally equivalent” and “functionally equivalent variant” are used interchangeably herein. These terms refer to a variant of the Cas endonuclease of the present invention in which the ability create a double-strand break is retained. Fragments and variants can be obtained via methods such as site-directed mutagenesis and synthetic construction.

In one embodiment, the Cas endonuclease gene is a plant codon optimized streptococcus pyogenes Cas9 gene that can recognize any genomic sequence of the form N(12-30)NGG can in principle be targeted.

In one embodiment, the Cas endonuclease is introduced directly into a cell by any method known in the art, for example, but not limited to transient introduction methods, transfection and/or topical application.

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more. (patent application WO-PCT PCT/US12/30061 filed on Mar. 22, 2012) Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI-for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

TAL effector nucleases are a new class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, Fokl. The unique, modular TAL effector DNA binding domain allows for the design of proteins with potentially any given DNA recognition specificity (Miller et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs consist of an engineered DNA-binding zinc finger domain linked to a non-specific endonuclease domain, for example nuclease domain from a Type IIs endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

c. Guide RNA/Cas Endonuclease System

Bacteria and archaea have evolved adaptive immune defenses termed clustered regularly interspaced short palindromic repeats (CRISPR)/CRISPR-associated (Cas) systems that use short RNA to direct degradation of foreign nucleic acids (Prashant Mali et al.). The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target (FIG. 2 B).

As used herein, the term “guide RNA” refers to a synthetic fusion of two RNA molecules, a crRNA (CRISPR RNA) comprising a variable targeting domain, and a tracrRNA (FIG. 2 B). In one embodiment, the guide RNA comprises a variable targeting domain of 12 to 30 nucleotide sequences and a RNA fragment that can interact with a Cas endonuclease.

As used herein, the term “guide polynucleotide”, refers to a polynucleotide sequence that can form a complex with a Cas endonuclease and enables the Cas endonuclease to recognize and optionally cleave a DNA target site. The guide polynucleotide can be comprised of a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotride that solely comprises ribonucleic acids is also referred to as a “guide RNA”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. In one embodiment, the size of the fragment of the cRNA naturally occurring in Bacteria and Archaea that is present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides. In some embodiments the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides In one embodiment, the RNA that guides the RNA/Cas9 endonuclease complex, is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some embodiments the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). In one embodiment of the disclosure, the single guide RNA comprises a cRNA or cRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site.

The term “variable targeting domain” or “VT domain” is used interchangeably herein and refers to a nucleotide sequence that is complementary to one strand (nucleotide sequence) of a double strand DNA target site (FIGS. 2 A and 2 B). The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and refers to a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In one embodiment, the guide RNA and Cas endonuclease are capable of forming a complex that enables the Cas endonuclease to introduce a double strand break at a DNA target site

In one embodiment of the invention the variable target domain is 12, 13, 14, 15, 16, 17, 18, 19, 20, 21, 22, 23, 24, 25, 26, 27, 28, 29 or 30 nucleotides in length.

In one embodiment of the disclosure, the guide RNA comprises a cRNA (or cRNA fragment) and a tracrRNA (or tracfRNA fragment) of the type II CRISPR/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site.

In one embodiment the guide RNA can be introduced into a plant or plant cell directly using any method known in the art such as, but not limited to, particle bombardment or topical applications.

In another embodiment the guide RNA can be introduced indirectly by introducing a recombinant DNA molecule comprising the corresponding guide DNA sequence operably linked to a plant specific promoter (as shown in FIG. 1 B) that is capable of transcribing the guide RNA in said plant cell. The term “corresponding guide DNA” refers to a DNA molecule that is identical to the RNA molecule but has a “T” substituted for each “U” of the RNA molecule.

In some embodiments, the guide RNA is introduced via particle bombardment or Agrobacterium transformation of a recombinant DNA construct comprising the corresponding guide DNA operably linked to a plant U6 polymerase III promoter.

In one embodiment, the RNA that guides the RNA/Cas9 endonuclease complex, is a duplexed RNA comprising a duplex crRNA-tracrRNA (as shown in FIG. 2B). One advantage of using a guide RNA versus a duplexed crRNA-tracrRNA is that only one expression cassette needs to be made to express the fused guide RNA.

### III. Target Sites for Cas Endonucleases

The terms “target site”, “target sequence”, “target DNA”, “target locus”, “genomic target site”, “genomic target sequence”, and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a plant and is at the endogenous or native position of that target sequence in the genome of the plant.

In one embodiments, the target site can be similar to a DNA recognition site or target site that that is specifically recognized and/or bound by a double-strand break inducing agent such as a LIG3-4 endonuclease (US patent publication 2009-0133152 A1 (published May 21, 2009) or a MS26++ meganuclease (U.S. patent application Ser. No. 13/526,912 filed Jun. 19, 2012).

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for modifying a plant genomic target site are disclosed herein. In one embodiment, a method for modifying a target site in the genome of a plant cell comprises introducing a guide RNA into a plant cell having a Cas endonuclease, wherein said guide RNA and Cas endonuclease are capable of forming a complex that enables the Cas endonuclease to introduce a double strand break at said target site.

Also provided is a method for modifying a target site in the genome of a plant cell, the method comprising introducing a guide RNA and a Cas endonuclease into said plant, wherein said guide RNA and Cas endonuclease are capable of forming a complex that enables the Cas endonuclease to introduce a double strand break at said target site.

Further provided is a method for modifying a target site in the genome of a plant cell, the method comprising introducing a guide RNA and a donor DNA into a plant cell having a Cas endonuclease, wherein said guide RNA and Cas endonuclease are capable of forming a complex that enables the Cas endonuclease to introduce a double strand break at said target site, wherein said donor DNA comprises a polynucleotide of interest.

Further provided is a method for modifying a target site in the genome of a plant cell, the method comprising: a) introducing into a plant cell a guide RNA comprising a variable targeting domain and a Cas endonuclease, wherein said guide RNA and Cas endonuclease are capable of forming a complex that enables the Cas endonuclease to introduce a double strand break at said target site; and, b) identifying at least one plant cell that has a modification at said target, wherein the modification includes at least one deletion or substitution of one or more nucleotides in said target site.

Further provided, a method for modifying a target DNA sequence in the genome of a plant cell, the method comprising: a) introducing into a plant cell a first recombinant DNA construct capable of expressing a guide RNA and a second recombinant DNA construct capable of expressing a Cas endonuclease, wherein said guide RNA and Cas endonuclease are capable of forming a complex that enables the Cas endonuclease to introduce a double strand break at said target site; and, b) identifying at least one plant cell that has a modification at said target, wherein the modification includes at least one deletion or substitution of one or more nucleotides in said target site.

The length of the target site can vary, and includes, for example, target sites that are at least 12, 13, 14, 15, 16, 17, 18, 19, 20, 21, 22, 23, 24, 25, 26, 27, 28, 29, or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs.

Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the double-strand break of a target site by an endonuclease are known in the art and generally measure the overall activity and specificity of the agent on DNA substrates containing recognition sites.

### IV. Methods for Integrating a Polynucleotide of Interest into a Plant Genomic Target Site that is Recognized by a Guide RNA/Cas System

Various methods and compositions can be employed to obtain a plant having a polynucleotide of interest inserted in a target site for a Cas endonuclease. Such methods can employ homologous recombination to provide integration of the polynucleotide of Interest at the target site. In one method provided, a polynucleotide of interest is provided to the plant cell in a donor DNA construct. As used herein, “donor DNA” is a DNA construct that comprises a polynucleotide of Interest to be inserted into the target site of a cas endonuclease. The donor DNA construct further comprises a first and a second region of homology that flank the polynucleotide of Interest. The first and second regions of homology of the donor DNA share homology to a first and a second genomic region, respectively, present in or flanking the target site of the plant genome. By “homology” is meant DNA sequences that are similar. For example, a “region of homology to a genomic region” that is found on the donor DNA is a region of DNA that has a similar sequence to a given “genomic region” in the plant genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction.

As used herein, a “genomic region” is a segment of a chromosome in the genome of a plant cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

Polynucleotides of interest and/or traits can be stacked together in a complex trait locus as described in US-2013-0263324-A1, published 3 Oct. 2013 and in PCT/US13/22891, published Jan. 24, 2013, both applications are hereby incorporated by reference. The guide polynucleotide/Cas9 endonuclease system described herein provides for an efficient system to generate double strand breaks and allows for traits to be stacked in a complex trait locus.

In one embodiment, the guide polynucleotide/Cas endonuclease system is used for introducing one or more polynucleotides of interest or one or more traits of interest into one or more target sites by providing one or more guide polynucleotides, one Cas endonuclease, and optionally one or more donor DNAs to a plant cell. A fertile plant can be produced from that plant cell that comprises an alteration at said one or more target sites, wherein the alteration is selected from the group consisting of (i) replacement of at least one nucleotide, (ii) a deletion of at least one nucleotide, (iii) an insertion of at least one nucleotide, and (iv) any combination of (i)-(iii). Plants comprising these altered target sites can be crossed with plants comprising at least one gene or trait of interest in the same complex trait locus, thereby further stacking traits in said complex trait locus. (see also US-2013-0263324-A1, published 3 Oct. 2013 and in PCT/US13/22891, published Jan. 24, 2013).

In one embodiment, the method comprises a method for producing in a plant a complex trait locus comprising at least two altered target sequences in a genomic 1o region of interest, said method comprising: (a) selecting a genomic region in a plant, wherein the genomic region comprises a first target sequence and a second target sequence; (b) contacting at least one plant cell with at least a first guide polynucleotide, a second polynucleotide, and optionally at least one donor DNA, and a Cas endonuclease, wherein the first and second guide polynucleotide and the Cas endonuclease can form a complex that enables the Cas endonuclease to introduce a double strand break in at least a first and a second target sequence; (c) identifying a cell from (b) comprising a first alteration at the first target sequence and a second alteration at the second target sequence; and (d) recovering a first fertile plant from the cell of (c) said fertile plant comprising the first alteration and the second alteration, wherein the first alteration and the second alteration are physically linked.

In one embodiment, the method comprises a method for producing in a plant a complex trait locus comprising at least two altered target sequences in a genomic region of interest, said method comprising: (a) selecting a genomic region in a plant, wherein the genomic region comprises a first target sequence and a second target sequence; (b) contacting at least one plant cell with a first guide polynucleotide, a Cas endonuclease, and optionally a first donor DNA, wherein the first guide polynucleotide and the Cas endonuclease can form a complex that enables the Cas endonuclease to introduce a double strand break a first target sequence; (c) identifying a cell from (b) comprising a first alteration at the first target sequence; (d) recovering a first fertile plant from the cell of (c), said first fertile plant comprising the first alteration; (e) contacting at least one plant cell with a second guide polynucleotide, a Cas endonuclease and optionally a second Donor DNA; (f) identifying a cell from (e) comprising a second alteration at the second target sequence; (g) recovering a second fertile plant from the cell of (f), said second fertile plant comprising the second alteration; and, (h) obtaining a fertile progeny plant from the second fertile plant of (g), said fertile progeny plant comprising the first alteration and the second alteration, wherein the first alteration and the second alteration are physically linked.

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the plant genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” refers to the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS(0027-8424), 111 (10), p. E924-E932.

Alteration of the genome of a plant cell, for example, through homologous recombination (HR), is a powerful tool for genetic engineering. Despite the low frequency of homologous recombination in higher plants, there are a few examples of successful homologous recombination of plant endogenous genes. The parameters for homologous recombination in plants have primarily been investigated by rescuing introduced truncated selectable marker genes. In these experiments, the homologous DNA fragments were typically between 0.3 kb to 2 kb. Observed frequencies for homologous recombination were on the order of 10⁻⁴ to 10⁻⁵. See, for example, Halfter et al., (1992) Mol Gen Genet 231:186-93; Offringa et al., (1990) EMBO J 9:3077-84; Offringa et al., (1993) Proc. Natl. Acad. Sci. USA 90:7346-50; Paszkowski et al., (1988) EMBO J 7:4021-6; Hourda and Paszkowski, (1994) Mol Gen Genet 243:106-11; and Risseeuw et al., (1995) Plant J 7:109-19.

Homologous recombination has been demonstrated in insects. In Drosophila, Dray and Gloor found that as little as 3 kb of total template:target homology sufficed to copy a large non-homologous segment of DNA into the target with reasonable efficiency (Dray and Gloor, (1997) Genetics 147:689-99). Using FLP-mediated DNA integration at a target FRT in Drosophila, Golic et al., showed integration was approximately 10-fold more efficient when the donor and target shared 4.1 kb of homology as compared to 1.1 kb of homology (Golic et al., (1997) Nucleic Acids Res 25:3665). Data from Drosophila indicates that 2-4 kb of homology is sufficient for efficient targeting, but there is some evidence that much less homology may suffice, on the order of about 30 bp to about 100 bp (Nassif and Engels, (1993) Proc. Natl. Acad. Sci. USA 90:1262-6; Keeler and Gloor, (1997) Mol Cell Biol 17:627-34).

Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al., (2000) Nucleic Acids Res 28:e97). Targeted gene replacement has also been demonstrated in the ciliate Tetrahymena thermophila (Gaertig et al., (1994) Nucleic Acids Res 22:5391-8). In mammals, homologous recombination has been most successful in the mouse using pluripotent embryonic stem cell lines (ES) that can be grown in culture, transformed, selected and introduced into a mouse embryo. Embryos bearing inserted transgenic ES cells develop as genetically offspring. By interbreeding siblings, homozygous mice carrying the selected genes can be obtained. An overview of the process is provided in Watson et al., (1992) Recombinant DNA, 2nd Ed., (Scientific American Books distributed by WH Freeman & Co.); Capecchi, (1989) Trends Genet 5:70-6; and Bronson, (1994) J Biol Chem 269:27155-8. Homologous recombination in mammals other than mouse has been limited by the lack of stem cells capable of being transplanted to oocytes or developing embryos. However, McCreath et al., Nature 405:1066-9 (2000) reported successful homologous recombination in sheep by transformation and selection in primary embryo fibroblast cells.

Error-prone DNA repair mechanisms can produce mutations at double-strand break sites. The nonhomologous end-joining (NHEJ) pathways are the most common repair mechanism to bring the broken ends together (Bleuyard et al., (2006) DNA Repair 5:1-12). The structural integrity of chromosomes is typically preserved by the repair, but deletions, insertions, or other rearrangements are possible. The two ends of one double-strand break are the most prevalent substrates of NHEJ (Kirik et al., (2000) EMBO J 19:5562-6), however if two different double-strand breaks occur, the free ends from different breaks can be ligated and result in chromosomal deletions (Siebert and Puchta, (2002) Plant Cell 14:1121-31), or chromosomal translocations between different chromosomes (Pacher et al., (2007) Genetics 175:21-9).

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

Once a double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements are possible (Siebert and Puchta, (2002) Plant Cell 14:1121-31; Pacher et al., (2007) Genetics 175:21-9).

Alternatively, the double-strand break can be repaired by homologous recombination between homologous DNA sequences. Once the sequence around the double-strand break is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

DNA double-strand breaks appear to be an effective factor to stimulate homologous recombination pathways (Puchta et al., (1995) Plant Mol Biol 28:281-92; Tzfira and White, (2005) Trends Biotechnol 23:567-9; Puchta, (2005) J Exp Bot 56:1-14). Using DNA-breaking agents, a two- to nine-fold increase of homologous recombination was observed between artificially constructed homologous DNA repeats in plants (Puchta et al., (1995) Plant Mol Biol 28:281-92). In maize protoplasts, experiments with linear DNA molecules demonstrated enhanced homologous recombination between plasmids (Lyznik et al., (1991) Mol Gen Genet 230:209-18).

In one embodiment provided herein, the method comprises contacting a plant cell with the donor DNA and the endonuclease. Once a double-strand break is introduced in the target site by the endonuclease, the first and second regions of homology of the donor DNA can undergo homologous recombination with their corresponding genomic regions of homology resulting in exchange of DNA between the donor and the genome. As such, the provided methods result in the integration of the polynucleotide of interest of the donor DNA into the double-strand break in the target site in the plant genome, thereby altering the original target site and producing an altered genomic target site.

The donor DNA may be introduced by any means known in the art. For example, a plant having a target site is provided. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Zinc finger nucleases are engineered endonucleases with altered specificities, for example by fusion of an engineered DNA binding domain to an endonuclease, for example, Fokl (Durai et al., (2005) Nucleic Acids Res 33:5978-90; Mani et al., (2005) Biochem Biophys Res Comm 335:447-57). Wright et al., and Lloyd et al., reported a high frequency mutagenesis at a DNA target site integrated into tobacco or Arabidopsis chromosomal DNA using zinc-finger nucleases (Wright et al., (2005) Plant J 44:693-705; Lloyd et al., (2005) Proc. Natl. Acad. Sci. USA 102:2232-7). Using a designed zinc-finger nuclease recognizing a tobacco endogenous acetolactate synthase (ALS) gene locus, a mutated ALS gene known to confer resistance to imidazolinone and sulphonylurea herbicides was introduced to replace the endogenous ALS gene at frequencies exceeding 2% of transformed cells (Townsend et al., (2009) Nature 459:442-5). The knock-out of an endogenous gene and the expression of a transgene can be achieved simultaneously by gene targeting. The IPK1 gene, which encodes inositol-1,3,4,5,6-pentakisphosphate 2-kinase needed in the final step of phytate biosythesis in maize seeds, was targeted using a designed zinc-finger nuclease to insert via homologous recombination a PAT gene, which encodes phosphinothricin acetyl transferase tolerance to glufosinate ammonium herbicides such as bialaphos. The disruption of the IPK1 gene with the insertion of the PAT gene resulted in both herbicide tolerance and the expected alteration of the inositol phosphate profile in developing seeds (Shukla et al., (2009) Nature 459:437-41).

Another approach uses protein engineering of existing homing endonucleases to alter their target specificities. Homing endonucleases, such as I-SceI or I-CreI, bind to and cleave relatively long DNA recognition sequences (18 bp and 22 bp, respectively). These sequences are predicted to naturally occur infrequently in a genome, typically only 1 or 2 sites/genome. The cleavage specificity of a homing endonuclease can be changed by rational design of amino acid substitutions at the DNA binding domain and/or combinatorial assembly and selection of mutated monomers (see, for example, Arnould et al., (2006) J Mol Biol 355:443-58; Ashworth et al., (2006) Nature 441:656-9; Doyon et al., (2006) J Am Chem Soc 128:2477-84; Rosen et al., (2006) Nucleic Acids Res 34:4791-800; and Smith et al., (2006) Nucleic Acids Res 34:e149; Lyznik et al., (2009) U.S. Patent Application Publication No. 20090133152A1; Smith et al., (2007) U.S. Patent Application Publication No. 20070117128A1). Engineered meganucleases have been demonstrated that can cleave cognate mutant sites without broadening their specificity. An artificial recognition site specific to the wild type yeast I-SceI homing nuclease was introduced in maize genome and mutations of the recognition sequence were detected in 1% of analyzed F1 plants when a transgenic I-SceI was introduced by crossing and activated by gene excision (Yang et al., (2009) Plant Mol Biol 70:669-79). More practically, the maize liguleless locus was targeted using an engineered single-chain endonuclease designed based on the I-CreI meganuclease sequence. Mutations of the selected liguleless locus recognition sequence were detected in 3% of the T0 transgenic plants when the designed homing nuclease was introduced by Agrobacterium-mediated transformation of immature embryos (Gao et al., (2010) Plant J 61:176-87).

Polynucleotides of interest are further described herein and are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly.

### V. Genome Editing Using the Guide RNA/Cas Endonuclease System

As described herein, the guide RNA/Cas endonuclease system can be used in combination with a co-delivered polynucleotide modification template to allow for editing of a genomic nucleotide sequence of interest. Also, as described herein, for each embodiment that uses a guide RNA/Cas endonuclease system, a similar guide polynucleotide/Cas endonuclease system can be deployed where the guide polynucleotide does not solely comprise ribonucleic acids but wherein the guide polynucleotide comprises a combination of RNA-DNA molecules or solely comprise DNA molecules.

While numerous double-strand break-making systems exist, their practical applications for gene editing may be restricted due to the relatively low frequency of induced double-strand breaks (DSBs). To date, many genome modification methods rely on the homologous recombination system. Homologous recombination (HR) can provide molecular means for finding genomic DNA sequences of interest and modifying them according to the experimental specifications. Homologous recombination takes place in plant somatic cells at low frequency. The process can be enhanced to a practical level for genome engineering by introducing double-strand breaks (DSBs) at selected endonuclease target sites. The challenge has been to efficiently make DSBs at genomic sites of interest since there is a bias in the directionality of information transfer between two interacting DNA molecules (the broken one acts as an acceptor of genetic information). Described herein is the use of a guide RNA/Cas system which provides flexible genome cleavage specificity and results in a high frequency of double-strand breaks at a DNA target site, thereby enabling efficient gene editing in a nucletotide sequence of interest, wherein the nucleotide sequence of interest to be edited can be located within or outside the target site recognized and cleaved by a Cas endonuclease.

The term “polynucleotide modification template” refers to a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In one embodiment, the disclosure describes a method for editing a nucleotide sequence in the genome of a cell, the method comprising providing a guide RNA, a polynucleotide modification template, and at least one Cas endonuclease to a cell, wherein the Cas endonuclease is capable of introducing a double-strand break at a target sequence in the genome of said cell, wherein said polynucleotide modification template includes at least one nucleotide modification of said nucleotide sequence. Cells include, but are not limited to, human, animal, bacterial, fungal, insect, and plant cells as well as plants and seeds produced by the methods described herein. The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

In another embodiment, the disclosure describes a method for editing a nucleotide sequence in the genome of a plant cell, the method comprising introducing a guide RNA, a polynucleotide modification template, and at least one maize optimized Cas9 endonuclease into a plant cell, wherein the maize optimized Cas9 endonuclease is capable of introducing a double-strand break at a moCas9 target sequence (bases 25-44 of SEQ ID NO:209) in the plant genome, wherein said polynucleotide modification template includes at least one nucleotide modification of said nucleotide sequence.

In another embodiment, the disclosure describes a method for editing a nucleotide sequence in the genome of a cell, the method comprising providing a guide RNA, a polynucleotide modification template and at least one Cas endonuclease to a cell, wherein said guide RNA and Cas endonuclease are capable of forming a complex that enables the Cas endonuclease to introduce a double strand break at a target site, wherein said polynucleotide modification template comprises at least one nucleotide modification of said nucleotide sequence.

The nucleotide sequence to be edited can be a sequence that is endogenous, artificial, pre-existing, or transgenic to the cell that is being edited. For example, the nucleotide sequence in the genome of a cell can be a transgene that is stably incorporated into the genome of a cell. Editing of such transgene may result in a further desired phenotype or genotype. The nucleotide sequence in the genome of a cell can also be a mutated or pre-existing sequence that was either endogenous or artificial from origin such as an endogenous gene or a mutated gene of interest.

### Promoter Modifications Using the Guide Polynucleotide/Cas Endonuclease System

A regulatory element generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located. The nucleic acid molecule regulated by a regulatory element does not necessarily have to encode a functional peptide or polypeptide, e.g., the regulatory element can modulate the expression of a short interfering RNA or an anti-sense RNA.

An enhancer element is any nucleic acid molecule that increases transcription of a nucleic acid molecule when functionally linked to a promoter regardless of its relative position. An enhancer may be an innate element of the promoter or a heterologous element inserted to enhance the level or tissue-specificity of a promoter.

A repressor (also sometimes called herein silencer) is defined as any nucleic acid molecule which inhibits the transcription when functionally linked to a promoter regardless of relative position.

“Promoter” generally refers to a nucleic acid fragment capable of controlling transcription of another nucleic acid fragment. A promoter generally includes a core promoter (also known as minimal promoter) sequence. Generally, a core promoter includes a TATA box and a GC rich region associated with a CAAT box or a CCAAT box. These elements act to bind RNA polymerase II to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” generally refers to a promoter whose activity is determined by developmental events.

“Constitutive promoter” generally refers to promoters active in all or most tissues or cell types of a plant at all or most developing stages. As with other promoters classified as “constitutive” (e.g. ubiquitin), some variation in absolute levels of expression can exist among different tissues or stages. The term “constitutive promoter” or “tissue-independent” are used interchangeably herein.

The promoter nucleotide sequences and methods disclosed herein are useful in regulating constitutive expression of any heterologous nucleotide sequences in a host plant in order to alter the phenotype of a plant.

A “heterologous nucleotide sequence” generally refers to a sequence that is not naturally occurring with the plant promoter sequence of the disclosure. While this nucleotide sequence is heterologous to the promoter sequence, it may be homologous, or native, or heterologous, or foreign, to the plant host. However, it is recognized that the instant promoters may be used with their native coding sequences to increase or decrease expression resulting in a change in phenotype in the transformed seed. The terms “heterologous nucleotide sequence”, “heterologous sequence”, “heterologous nucleic acid fragment”, and “heterologous nucleic acid sequence” are used interchangeably herein.

The present disclosure encompasses recombinant DNA constructs comprising functional fragments of the promoter sequences disclosed herein. A “functional fragment” refer to a portion or subsequence of the promoter sequence of the present disclosure in which the ability to initiate transcription or drive gene expression (such as to produce a certain phenotype) is retained. Fragments can be obtained via methods such as site-directed mutagenesis and synthetic construction. As with the provided promoter sequences described herein, the functional fragments operate to promote the expression of an operably linked heterologous nucleotide sequence, forming a recombinant DNA construct (also, a chimeric gene). For example, the fragment can be used in the design of recombinant DNA constructs to produce the desired phenotype in a transformed plant. Recombinant DNA constructs can be designed for use in co-suppression or antisense by linking a promoter fragment in the appropriate orientation relative to a heterologous nucleotide sequence.

In one embodiment the nucleotide sequence to be modified can be a promoter wherein the editing of the promoter comprises replacing the promoter (also referred to as a “promoter swap” or “promoter replacement”) or promoter fragment with a different promoter (also referred to as replacement promoter) or promoter fragment (also referred to as replacement promoter fragment), wherein the promoter replacement results in any one of the following or any one combination of the following: an increased promoter activity, an increased promoter tissue specificity, a decreased promoter activity, a decreased promoter tissue specificity, a new promoter activity, an inducible promoter activity, an extended window of gene expression, a modification of the timing or developmental progress of gene expression in the same cell layer or other cell layer (such as but not limiting to extending the timing of gene expression in the tapetum of maize anthers (U.S. Pat. No. 5,837,850 issued Nov. 17, 1998), a mutation of DNA binding elements and/or a deletion or addition of DNA binding elements. The promoter (or promoter fragment) to be modified can be a promoter (or promoter fragment) that is endogenous, artificial, pre-existing, or transgenic to the cell that is being edited. The replacement promoter (or replacement promoter fragment) can be a promoter (or promoter fragment) that is endogenous, artificial, pre-existing, or transgenic to the cell that is being edited.

In one embodiment the nucleotide sequence can be a promoter wherein the editing of the promoter comprises replacing an ARGOS 8 promoter with a Zea mays GOS2 PRO:GOS2-intron promoter.

In one embodiment the nucleotide sequence can be a promoter wherein the editing of the promoter comprises replacing a native EPSPS1 promoter from with a soybean ubiquitin promoter.

In one embodiment the nucleotide sequence can be a promoter wherein the editing of the promoter comprises replacing an endogenous maize NPK1 promoter with a stress inducible maize RAB17 promoter.

In one embodiment the nucleotide sequence can be a promoter wherein the promoter to be edited is selected from the group comprising Zea mays-PEPC1 promoter (Kausch et al, Plant Molecular Biology, 45: 1-15, 2001), Zea mays Ubiquitin promoter (UBI1ZM PRO, Christensen et al, plant Molecular Biology 18: 675-689, 1992), Zea mays-Rootmet2 promoter (U.S. Pat. No. 7,214,855), Rice actin promoter (OS-ACTIN PRO, U.S. Pat. No. 5,641,876; McElroy et al, The Plant Cell, Vol 2, 163-171, February 1990), Sorghum RCC3 promoter (US 2012/0210463 filed on 13 Feb. 2012), Zea mays-GOS2 promoter (U.S. Pat. No. 6,504,083), Zea mays-ACO2 promoter (U.S. application Ser. No. 14/210,711 filed 14 Mar. 2014) or Zea mays-oleosin promoter (U.S. Pat. No. 8,466,341 B2).

In another embodiment, the guide polynucleotide/Cas endonuclease system can be used in combination with a co-delivered polynucleotide modification template or donor DNA sequence to allow for the insertion of a promoter or promoter element into a genomic nucleotide sequence of interest, wherein the promoter insertion (or promoter element insertion) results in any one of the following or any one combination of the following: an increased promoter activity (increased promoter strength), an increased promoter tissue specificity, a decreased promoter activity, a decreased promoter tissue specificity, a new promoter activity, an inducible promoter activity, an extended window of gene expression, a modification of the timing or developmental progress of gene expression a mutation of DNA binding elements and/or an addition of DNA binding elements. Promoter elements to be inserted can be, but are not limited to, promoter core elements (such as, but not limited to, a CAAT box, a CCAAT box, a Pribnow box, a and/or TATA box, translational regulation sequences and/or a repressor system for inducible expression (such as TET operator repressor/operator/inducer elements, or SulphonylUrea (Su) repressor/operator/inducer elements. The dehydration-responsive element (DRE) was first identified as a cis-acting promoter element in the promoter of the drought-responsive gene rd29A, which contains a 9 bp conserved core sequence, TACCGACAT (Yamaguchi-Shinozaki, K., and Shinozaki, K. (1994) Plant Cell 6, 251-264). Insertion of DRE into an endogenous promoter may confer a drought inducible expression of the downstream gene. Another example is ABA-responsive elements (ABREs) which contains a (C/T)ACGTGGC consensus sequence found to be present in numerous ABA and/or stress-regulated genes (Busk P. K., Pages M. (1998) Plant Mol. Biol. 37:425-435). Insertion of 35S enhancer or MMV enhancer into an endogenous promoter region will increase gene expression (U.S. Pat. No. 5,196,525). The promoter (or promoter element) to be inserted can be a promoter (or promoter element) that is endogenous, artificial, pre-existing, or transgenic to the cell that is being edited.

In one embodiment, the guide polynucleotide/Cas endonuclease system can be used to insert an enhancer element, such as but not limited to a Cauliflower Mosaic Virus 35 S enhancer, in front of an endogenous FMT1 promoter to enhance expression of the FTM1.

In one embodiment, the guide polynucleotide/Cas endonuclease system can be used to insert a component of the TET operator repressor/operator/inducer system, or a component of the sulphonylUrea (Su) repressor/operator/inducer system into plant genomes to generate or control inducible expression systems.

In another embodiment, the guide polynucleotide/Cas endonuclease system can be used to allow for the deletion of a promoter or promoter element, wherein the promoter deletion (or promoter element deletion) results in any one of the following or any one combination of the following: a permanently inactivated gene locus, an increased promoter activity (increased promoter strength), an increased promoter tissue specificity, a decreased promoter activity, a decreased promoter tissue specificity, a new promoter activity, an inducible promoter activity, an extended window of gene expression, a modification of the timing or developmental progress of gene expression, a mutation of DNA binding elements and/or an addition of DNA binding elements. Promoter elements to be deleted can be, but are not limited to, promoter core elements, promoter enhancer elements or 35 S enhancer elements (as described in Example 32) The promoter or promoter fragment to be deleted can be endogenous, artificial, pre-existing, or transgenic to the cell that is being edited.

In one embodiment, the guide polynucleotide/Cas endonuclease system can be used to delete the ARGOS 8 promoter present in a maize genome as described herein.

In one embodiment, the guide polynucleotide/Cas endonuclease system can be used to delete a 35S enhancer element present in a plant genome as described herein.

### Terminator Modifications Using the Guide Polynucleotide/Cas Endonuclease System

In one embodiment the nucleotide sequence to be modified can be a terminator wherein the editing of the terminator comprises replacing the terminator (also referred to as a “terminator swap” or “terminator replacement”) or terminator fragment with a different terminator (also referred to as replacement terminator) or terminator fragment (also referred to as replacement terminator fragment), wherein the terminator replacement results in any one of the following or any one combination of the following: an increased terminator activity, an increased terminator tissue specificity, a decreased terminator activity, a decreased terminator tissue specificity, a mutation of DNA binding elements and/or a deletion or addition of DNA binding elements.” The terminator (or terminator fragment) to be modified can be a terminator (or terminator fragment) that is endogenous, artificial, pre-existing, or transgenic to the cell that is being edited. The replacement terminator (or replacement terminator fragment) can be a terminator (or terminator fragment) that is endogenous, artificial, pre-existing, or transgenic to the cell that is being edited.

In one embodiment the nucleotide sequence to be modified can be a terminator wherein the terminator to be edited is selected from the group comprising terminators from maize Argos 8 or SRTF18 genes, or other terminators, such as potato PinII terminator, sorghum actin terminator (SB-ACTIN TERM, WO 2013/184537 A1 published December 2013), sorghum SB-GKAF TERM (WO2013019461), rice T28 terminator (OS-T28 TERM, WO 2013/012729 A2), AT-T9 TERM (WO 2013/012729 A2) or GZ-W64A TERM (U.S. Pat. No. 7,053,282).

In one embodiment, the guide polynucleotide/Cas endonuclease system can be used in combination with a co-delivered polynucleotide modification template or donor DNA sequence to allow for the insertion of a terminator or terminator element into a genomic nucleotide sequence of interest, wherein the terminator insertion (or terminator element insertion) results in any one of the following or any one combination of the following: an increased terminator activity (increased terminator strength), an increased terminator tissue specificity, a decreased terminator activity, a decreased terminator tissue specificity, a mutation of DNA binding elements and/or an addition of DNA binding elements.

The terminator (or terminator element) to be inserted can be a terminator (or terminator element) that is endogenous, artificial, pre-existing, or transgenic to the cell that is being edited.

In another embodiment, the guide polynucleotide/Cas endonuclease system can be used to allow for the deletion of a terminator or terminator element, wherein the terminator deletion (or terminator element deletion) results in any one of the following or any one combination of the following: an increased terminator activity (increased terminator strength), an increased terminator tissue specificity, a decreased terminator activity, a decreased terminator tissue specificity, a mutation of DNA binding elements and/or an addition of DNA binding elements. The terminator or terminator fragment to be deleted can be endogenous, artificial, pre-existing, or transgenic to the cell that is being edited.

### Regulatory Sequence Modifications Using the Guide Polynucleotide/Cas Endonuclease System

In one embodiment, the guide polynucleotide/Cas endonuclease system can be used to modify or replace a regulatory sequence in the genome of a cell. A regulatory sequence is a segment of a nucleic acid molecule which is capable of increasing or decreasing the expression of specific genes within an organism and/or is capable of altering tissue specific expression of genes within an organism. Examples of regulatory sequences include, but are not limited to, 3′ UTR (untranslated region) region, 5′ UTR region, transcription activators, transcriptional enhancers transcriptions repressors, translational repressors, splicing factors, miRNAs, siRNA, artificial miRNAs, promoter elements, CAMV 35 S enhancer, MMV enhancer elements (PCT/US14/23451 filed Mar. 11, 2013), SECIS elements, polyadenylation signals, and polyubiquitination sites. In some embodiments the editing (modification) or replacement of a regulatory element results in altered protein translation, RNA cleavage, RNA splicing, transcriptional termination or post translational modification. In one embodiment, regulatory elements can be identified within a promoter and these regulatory elements can be edited or modified do to optimize these regulatory elements for up or down regulation of the promoter.

In one embodiment, the genomic sequence of interest to be modified is a polyubiquitination site, wherein the modification of the polyubiquitination sites results in a modified rate of protein degradation. The ubiquitin tag condemns proteins to be degraded by proteasomes or autophagy. Proteasome inhibitors are known to cause a protein overproduction. Modifications made to a DNA sequence encoding a protein of interest can result in at least one amino acid modification of the protein of interest, wherein said modification allows for the polyubiquitination of the protein (a post translational modification) resulting in a modification of the protein degradation

In one embodiment, the genomic sequence of interest to be modified is a an intron or UTR site, wherein the modification consist of inserting at least one microRNA into said intron or UTR site, wherein expression of the gene comprising the intron or UTR site also results in expression of said microRNA, which in turn can silence any gene targeted by the microRNA without disrupting the gene expression of the native/transgene comprising said intron.

In one embodiment, the guide polynucleotide/Cas endonuclease system can be used to allow for the deletion or mutation of a Zinc Finger transcription factor, wherein the deletion or mutation of the Zinc Finger transcription factor results in or allows for the creation of a dominant negative Zinc Finger transcription factor mutant (Li et al 2013 Rice zinc finger protein DST enhances grain production through controlling Gn1a/OsCKX2 expression PNAS 110:3167-3172). Insertion of a single base pair downstream zinc finger domain will result in a frame shift and produces a new protein which still can bind to DNA without transcription activity. The mutant protein will compete to bind to cytokinin oxidase gene promoters and block the expression of cytokinin oxidase gene. Reduction of cytokinin oxidase gene expression will increase cytokinin level and promote panicle growth in rice and ear growth in maize, and increase yield under normal and stress conditions.

Modifications of Splicing Sites and/or Introducing Alternate Splicing Sites Using the Guide Polynucleotide/Cas Endonuclease System

Protein synthesis utilizes mRNA molecules that emerge from pre-mRNA molecules subjected to the maturation process. The pre-mRNA molecules are capped, spliced and stabilized by addition of polyA tails. Eukaryotic cells developed a complex process of splicing that result in alternative variants of the original pre-mRNA molecules. Some of them may not produce functional templates for protein synthesis. In maize cells, the splicing process is affected by splicing sites at the exon-intron junction sites. An example of a canonical splice site is AGGT. Gene coding sequences can contains a number of alternate splicing sites that may affect the overall efficiency of the pre-mRNA maturation process and as such may limit the protein accumulation in cells. The guide polynucleotide/Cas endonuclease system can be used in combination with a co-delivered polynucleotide modification template to edit a gene of interest to introduce a canonical splice site at a described junction.

In one embodiment, the nucleotide sequence of interest to be modified is a maize EPSPS gene, wherein the modification of the gene consists of eliminating alternative splicing sites resulting in enhanced production of the functional gene transcripts and gene products (proteins).

In one embodiment, the nucleotide sequence of interest to be modified is a gene, wherein the modification of the gene consists of editing the intron borders of alternatively spliced genes to alter the accumulation of splice variants.

### Modifications or Replacements of Nucleotide Sequences Encoding a Protein of Interest Using the Guide Polynucleotide/Cas Endonuclease System

In one embodiment, the guide polynucleotide/Cas endonuclease system can be used to modify or replace a coding sequence in the genome of a cell, wherein the modification or replacement results in any one of the following, or any one combination of the following: an increased protein (enzyme) activity, an increased protein functionality, a decreased protein activity, a decreased protein functionality, a site specific mutation, a protein domain swap, a protein knock-out (for example due to the introduction of DNA binding elements and/or a deletion or addition of DNA binding elements, a new protein functionality, a modified protein functionality.

In one embodiment the protein knockout is due to the introduction of a stop codon into the coding sequence of interest.

In one embodiment the protein knockout is due to the deletion of a start codon into the coding sequence of interest.

Amino Acid and/or Protein Fusions Using the Guide Polynucleotide/Cas Endonuclease System

In one embodiment, the guide polynucleotide/Cas endonuclease system can be used with or without a co-delivered polynucleotide sequence to fuse a first coding sequence encoding a first protein to a second coding sequence encoding a second protein in the genome of a cell, wherein the protein fusion results in any one of the following or any one combination of the following: an increased protein (enzyme) activity, an increased protein functionality, a decreased protein activity, a decreased protein functionality, a new protein functionality, a modified protein functionality, a new protein localization, a new timing of protein expression, a modified protein expression pattern, a chimeric protein, or a modified protein with dominant phenotype functionality.

In one embodiment, the guide polynucleotide/Cas endonuclease system can be used with or without a co-delivered polynucleotide sequence to fuse a first coding sequence encoding a chloroplast localization signal to a second coding sequence encoding a protein of interest, wherein the protein fusion results in targeting the protein of interest to the chloroplast.

In one embodiment, the guide polynucleotide/Cas endonuclease system can be used with or without a co-delivered polynucleotide sequence to fuse a first coding sequence encoding a chloroplast localization signal to a second coding sequence encoding a protein of interest, wherein the protein fusion results in targeting the protein of interest to the chloroplast.

In one embodiment, the guide polynucleotide/Cas endonuclease system can be used with or without a co-delivered polynucleotide sequence to fuse a first coding sequence encoding to a second coding sequence, wherein the protein fusion results in a modified protein with dominant phenotype functionality

Gene Silencing by Expressing an Inverted Repeat into a Gene of Interest Using the Guide Polynucleotide/Cas Endonuclease System

In one embodiment, the guide polynucleotide/Cas endonuclease system can be used in combination with a co-delivered polynucleotide sequence to insert an inverted gene fragment into a gene of interest in the genome of an organism, wherein the insertion of the inverted gene fragment can allow for an in-vivo creation of an inverted repeat (hairpin) and results in the silencing of said endogenous gene.

In one embodiment the insertion of the inverted gene fragment can result in the formation of an in-vivo created inverted repeat (hairpin) in a native (or modified) promoter of a gene and/or in a native 5′ end of the native gene. The inverted gene fragment can further comprise an intron which can result in an enhanced silencing of the targeted gene.

### Genome Deletion for Trait Locus Characterization

Trait mapping in plant breeding often results in the detection of chromosomal regions housing one or more genes controlling expression of a trait of interest. For a qualitative trait, the guide polynucleotide/Cas endonuclease system can be used to eliminate candidate genes in the identified chromosomal regions to determine if deletion of the gene affects expression of the trait. For quantitative traits, expression of a trait of interest is governed by multiple quantitative trait loci (QTL) of varying effect-size, complexity, and statistical significance across one or more chromosomes. In cases of negative effect or deleterious QTL regions affecting a complex trait, the guide polynucleotide/Cas endonuclease system can be used to eliminate whole regions delimited by marker-assisted fine mapping, and to target specific regions for their selective elimination or rearrangement. Similarly, presence/absence variation (PAV) or copy number variation (CNV) can be manipulated with selective genome deletion using the guide polynucleotide/Cas endonuclease system.

In one embodiment, the region of interest can be flanked by two independent guide polynucleotide/CAS endonuclease target sequences. Cutting would be done concurrently. The deletion event would be the repair of the two chromosomal ends without the region of interest. Alternative results would include inversions of the region of interest, mutations at the cut sites and duplication of the region of interest.

### VI. Methods for Identifying at Least One Plant Cell Comprising in its Genome a Polynucleotide of Interest Integrated at the Target Site

Further provided are methods for identifying at least one plant cell comprising in its genome a polynucleotide of Interest integrated at the target site. A variety of methods are available for identifying those plant cells with insertion into the genome at or near to the target site without using a screenable marker phenotype. Such methods can be viewed as directly analyzing a target sequence to detect any change in the target sequence, including but not limited to PCR methods, sequencing methods, nuclease digestion, Southern blots, and any combination thereof. See, for example, U.S. patent application Ser. No. 12/147,834, herein incorporated by reference in its entirety. The method also comprises recovering a plant from the plant cell comprising a polynucleotide of Interest integrated into its genome. The plant may be sterile or fertile. It is recognized that any polynucleotide of interest can be provided, integrated into the plant genome at the target site, and expressed in a plant.

Polynucleotides of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly.

Polynucleotides/polypeptides of interest include, but are not limited to, herbicide-tolerance coding sequences, insecticidal coding sequences, nematicidal coding sequences, antimicrobial coding sequences, antifungal coding sequences, antiviral coding sequences, abiotic and biotic stress tolerance coding sequences, or sequences modifying plant traits such as yield, grain quality, nutrient content, starch quality and quantity, nitrogen fixation and/or utilization, and oil content and/or composition. More specific polynucleotides of interest include, but are not limited to, genes that improve crop yield, polypeptides that improve desirability of crops, genes encoding proteins conferring resistance to abiotic stress, such as drought, nitrogen, temperature, salinity, toxic metals or trace elements, or those conferring resistance to toxins such as pesticides and herbicides, or to biotic stress, such as attacks by fungi, viruses, bacteria, insects, and nematodes, and development of diseases associated with these organisms. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, fertility or sterility, grain characteristics, and commercial products. Genes of interest include, generally, those involved in oil, starch, carbohydrate, or nutrient metabolism as well as those affecting kernel size, sucrose loading, and the like.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Commercial traits can also be encoded on a polynucleotide of interest that could increase for example, starch for ethanol production, or provide expression of proteins. Another commercial use of transformed plants is the production of polymers and bioplastics such as described in U.S. Pat. No. 5,602,321. Genes such as β-Ketothiolase, PHBase (polyhydroxybutyrate synthase), and acetoacetyl-CoA reductase (see Schubert et al. (1988) J. Bacteriol. 170:5837-5847) facilitate expression of polyhydroxyalkanoates (PHAs).

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Polynucleotides that improve crop yield include dwarfing genes, such as Rht1 and Rht2 (Peng et al. (1999) Nature 400:256-261), and those that increase plant growth, such as ammonium-inducible glutamate dehydrogenase. Polynucleotides that improve desirability of crops include, for example, those that allow plants to have reduced saturated fat content, those that boost the nutritional value of plants, and those that increase grain protein. Polynucleotides that improve salt tolerance are those that increase or allow plant growth in an environment of higher salinity than the native environment of the plant into which the salt-tolerant gene(s) has been introduced.

Polynucleotides/polypeptides that influence amino acid biosynthesis include, for example, anthranilate synthase (AS; EC 4.1.3.27) which catalyzes the first reaction branching from the aromatic amino acid pathway to the biosynthesis of tryptophan in plants, fungi, and bacteria. In plants, the chemical processes for the biosynthesis of tryptophan are compartmentalized in the chloroplast. See, for example, US Pub. No. 20080050506, herein incorporated by reference. Additional sequences of interest include Chorismate Pyruvate Lyase (CPL) which refers to a gene encoding an enzyme which catalyzes the conversion of chorismate to pyruvate and pHBA. The most well characterized CPL gene has been isolated from E. coli and bears the GenBank accession number M96268. See, U.S. Pat. No. 7,361,811, herein incorporated by reference.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS), in particular the sulfonylurea-type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes known in the art. See, for example, U.S. Pat. Nos. 7,626,077, 5,310,667, 5,866,775, 6,225,114, 6,248,876, 7,169,970, 6,867,293, and U.S. Provisional Application No. 61/401,456, each of which is herein incorporated by reference. The bargene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male fertility genes such as MS26 (see for example U.S. Pat. Nos. 7,098,388, 7,517,975, 7,612,251), MS45 (see for example U.S. Pat. Nos. 5,478,369, 6,265,640) or MSCA1 (see for example U.S. Pat. No. 7,919,676). Maize plants (Zea mays L.) can be bred by both self-pollination and cross-pollination techniques. Maize has male flowers, located on the tassel, and female flowers, located on the ear, on the same plant. It can self-pollinate (“selfing”) or cross pollinate. Natural pollination occurs in maize when wind blows pollen from the tassels to the silks that protrude from the tops of the incipient ears. Pollination may be readily controlled by techniques known to those of skill in the art. The development of maize hybrids requires the development of homozygous inbred lines, the crossing of these lines, and the evaluation of the crosses. Pedigree breeding and recurrent selections are two of the breeding methods used to develop inbred lines from populations. Breeding programs combine desirable traits from two or more inbred lines or various broad-based sources into breeding pools from which new inbred lines are developed by selfing and selection of desired phenotypes. A hybrid maize variety is the cross of two such inbred lines, each of which may have one or more desirable characteristics lacked by the other or which complement the other. The new inbreds are crossed with other inbred lines and the hybrids from these crosses are evaluated to determine which have commercial potential. The hybrid progeny of the first generation is designated F1. The F1 hybrid is more vigorous than its inbred parents. This hybrid vigor, or heterosis, can be manifested in many ways, including increased vegetative growth and increased yield.

Hybrid maize seed can be produced by a male sterility system incorporating manual detasseling. To produce hybrid seed, the male tassel is removed from the growing female inbred parent, which can be planted in various alternating row patterns with the male inbred parent. Consequently, providing that there is sufficient isolation from sources of foreign maize pollen, the ears of the female inbred will be fertilized only with pollen from the male inbred. The resulting seed is therefore hybrid (F1) and will form hybrid plants.

Field variation impacting plant development can result in plants tasseling after manual detasseling of the female parent is completed. Or, a female inbred plant tassel may not be completely removed during the detasseling process. In any event, the result is that the female plant will successfully shed pollen and some female plants will be self-pollinated. This will result in seed of the female inbred being harvested along with the hybrid seed which is normally produced. Female inbred seed does not exhibit heterosis and therefore is not as productive as F1 seed. In addition, the presence of female inbred seed can represent a germplasm security risk for the company producing the hybrid.

Alternatively, the female inbred can be mechanically detasseled by machine. Mechanical detasseling is approximately as reliable as hand detasseling, but is faster and less costly. However, most detasseling machines produce more damage to the plants than hand detasseling. Thus, no form of detasseling is presently entirely satisfactory, and a need continues to exist for alternatives which further reduce production costs and to eliminate self-pollination of the female parent in the production of hybrid seed.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as glufosinate ammonium, bromoxynil, imidazolinones, and 2,4-dichlorophenoxyacetate (2,4-D). See for example, Yarranton, (1992) Curr Opin Biotech 3:506-11; Christopherson et al., (1992) Proc. Natl. Acad. Sci. USA 89:6314-8; Yao et al., (1992) Cell 71:63-72; Reznikoff, (1992) Mol Microbiol 6:2419-22; Hu et al., (1987) Cell 48:555-66; Brown et al., (1987) Cell 49:603-12; Figge et al., (1988) Cell 52:713-22; Deuschle et al., (1989) Proc. Natl. Acad. Sci. USA 86:5400-4; Fuerst et al., (1989) Proc. Natl. Acad. Sci. USA 86:2549-53; Deuschle et al., (1990) Science 248:480-3; Gossen, (1993) Ph.D. Thesis, University of Heidelberg; Reines et al., (1993) Proc. Natl. Acad. Sci. USA 90:1917-21; Labow et al., (1990) Mol Cell Biol 10:3343-56; Zambretti et al., (1992) Proc. Natl. Acad. Sci. USA 89:3952-6; Baim et al., (1991) Proc. Natl. Acad. Sci. USA 88:5072-6; Wyborski et al., (1991) Nucleic Acids Res 19:4647-53; Hillen and Wissman, (1989) Topics Mol Struc Biol 10:143-62; Degenkolb et al., (1991) Antimicrob Agents Chemother 35:1591-5; Kleinschnidt et al., (1988) Biochemistry 27:1094-104; Bonin, (1993) Ph.D. Thesis, University of Heidelberg; Gossen et al., (1992) Proc. Natl. Acad. Sci. USA 89:5547-51; Oliva et al., (1992) Antimicrob Agents Chemother 36:913-9; Hlavka et al., (1985) Handbook of Experimental Pharmacology, Vol. 78 (Springer-Verlag, Berlin); Gill et al., (1988) Nature 334:721-4. Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

The transgenes, recombinant DNA molecules, DNA sequences of interest, and polynucleotides of interest can be comprise one or more DNA sequences for gene silencing. Methods for gene silencing involving the expression of DNA sequences in plant are known in the art include, but are not limited to, cosuppression, antisense suppression, double-stranded RNA (dsRNA) interference, hairpin RNA (hpRNA) interference, intron-containing hairpin RNA (ihpRNA) interference, transcriptional gene silencing, and micro RNA (miRNA) interference

As used herein, “nucleic acid” means a polynucleotide and includes a single or a double-stranded polymer of deoxyribonucleotide or ribonucleotide bases.

Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA that is single- or double-stranded, optionally containing synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

“Open reading frame” is abbreviated ORF.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of genes to produce the desired phenotype in a transformed plant. genes can be designed for use in suppression by linking a nucleic acid fragment or subfragment thereof, whether or not it encodes an active enzyme, in the sense or antisense orientation relative to a plant promoter sequence.

The term “conserved domain” or “motif” means a set of amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid fragments wherein changes in one or more amino acids or nucleotide bases do not affect the function of the molecule, such as the ability to mediate gene expression or to produce a certain phenotype. These terms also refer to modification(s) of nucleic acid fragments that do not substantially alter the functional properties of the resulting nucleic acid fragment relative to the initial, unmodified fragment. These modifications include deletion, substitution, and/or insertion of one or more nucleotides in the nucleic acid fragment.

Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, Wis.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

Unless otherwise stated, sequence identity/similarity values provided herein refer to the value obtained using GAP Version 10 (GCG, Accelrys, San Diego, Calif.) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present invention, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

“Gene” refers to a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the invention, the mutated gene comprises an alteration that results from a guide polynucleotide/Cas endonuclease system as disclosed herein. A mutated plant is a plant comprising a mutated gene.

As used herein, a “targeted mutation” is a mutation in a native gene that was made by altering a target sequence within the native gene using a method involving a double-strand-break-inducing agent that is capable of inducing a double-strand break in the DNA of the target sequence as disclosed herein or known in the art.

In one embodiment, the targeted mutation is the result of a guideRNA/Cas endonuclease induced gene editing as described herein. The guide RNA/Cas endonuclease induced targeted mutation can occur in a nucleotide sequence that is located within or outside a genomic target site that is recognized and cleaved by a Cas endonuclease.

The term “genome” as it applies to a plant cells encompasses not only chromosomal DNA found within the nucleus, but organelle DNA found within subcellular components (e.g., mitochondria, or plastid) of the cell.

A “codon-modified gene” or “codon-preferred gene” or “codon-optimized gene” is a gene having its frequency of codon usage designed to mimic the frequency of preferred codon usage of the host cell.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to: promoters, translation leader sequences, 5′ untranslated sequences, 3′ untranslated sequences, introns, polyadenylation target sequences, RNA processing sites, effector binding sites, and stem-loop structures.

“A plant-optimized nucleotide sequence” is nucleotide sequence that has been optimized for increased expression in plants, particularly for increased expression in plants or in one or more plants of interest. For example, a plant-optimized nucleotide sequence can be synthesized by modifying a nucleotide sequence encoding a protein such as, for example, double-strand-break-inducing agent (e.g., an endonuclease) as disclosed herein, using one or more plant-preferred codons for improved expression. See, for example, Campbell and Gowri (1990) Plant Physiol. 92:1-11 for a discussion of host-preferred codon usage.

Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present invention comprises one or more of such sequence modifications.

“Promoter” refers to a DNA sequence capable of controlling the expression of a coding sequence or functional RNA. The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”.

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels. Since patterns of expression of a chimeric gene (or genes) introduced into a plant are controlled using promoters, there is an ongoing interest in the isolation of novel promoters which are capable of controlling the expression of a chimeric gene or (genes) at certain levels in specific tissue types or at specific plant developmental stages.

Some embodiments of the inventions relate to newly discovered U6 RNA polymerase III promoters, GM-U6-13.1 (SEQ ID NO: 120) as described in Example 12 and GM-U6-9.1 (SEQ ID NO: 295) described in Example 19.

“Translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript. A RNA transcript is referred to as the mature RNA when it is a RNA sequence derived from post-transcriptional processing of the primary transcript. “Messenger RNA” or “mRNA” refers to the RNA that is without introns and that can be translated into protein by the cell. “cDNA” refers to a DNA that is complementary to, and synthesized from, a mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, N.Y. (1989). Transformation methods are well known to those skilled in the art and are described infra.

“PCR” or “polymerase chain reaction” is a technique for the synthesis of specific DNA segments and consists of a series of repetitive denaturation, annealing, and extension cycles. Typically, a double-stranded DNA is heat denatured, and two primers complementary to the 3′ boundaries of the target segment are annealed to the DNA at low temperature, and then extended at an intermediate temperature. One set of these three consecutive steps is referred to as a “cycle”.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to an extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector containing a gene and having elements in addition to the gene that facilitates transformation of a particular host cell. “Expression cassette” refers to a specific vector containing a gene and having elements in addition to the gene that allow for expression of that gene in a host.

The terms “recombinant DNA molecule”, “recombinant construct”, “expression construct”, “construct”, “construct”, and “recombinant DNA construct” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “expression”, as used herein, refers to the production of a functional end-product (e.g., an mRNA, guide RNA, or a protein) in either precursor or mature form.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA).

“Mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

“Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or other DNA-containing organelle, of a host organism resulting in gene expression without integration or stable inheritance. Host organisms containing the transformed nucleic acid fragments are referred to as “transgenic” organisms.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different genes of interest.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to roots, stems, shoots, leaves, pollens, seeds, tumor tissue and various forms of cells and culture (e.g., single cells, protoplasts, embryos, and callus tissue). The plant tissue may be in plant or in a plant organ, tissue or cell culture. The term “plant organ” refers to plant tissue or a group of tissues that constitute a morphologically and functionally distinct part of a plant. The term “genome” refers to the entire complement of genetic material (genes and non-coding sequences) that is present in each cell of an organism, or virus or organelle; and/or a complete set of chromosomes inherited as a (haploid) unit from one parent. “Progeny” comprises any subsequent generation of a plant.

In certain embodiments of the invention, a fertile plant is a plant that produces viable male and female gametes and is self-fertile. Such a self-fertile plant can produce a progeny plant without the contribution from any other plant of a gamete and the genetic material contained therein. Other embodiments of the invention can involve the use of a plant that is not self-fertile because the plant does not produce male gametes, or female gametes, or both, that are viable or otherwise capable of fertilization. As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female fertile (but male sterile) plant can produce viable progeny when crossed with a male fertile plant.

A “centimorgan” (cM) or “map unit” is the distance between two linked genes, markers, target sites, loci, or any pair thereof, wherein 1% of the products of meiosis are recombinant. Thus, a centimorgan is equivalent to a distance equal to an 1% average recombination frequency between the two linked genes, markers, target sites, loci, or any pair thereof.

Breeding Methods and Methods for Selecting Plants Utilizing a Two Component RNA Guide and Cas Endonuclease System

The present invention finds use in the breeding of plants comprising one or more transgenic traits. Most commonly, transgenic traits are randomly inserted throughout the plant genome as a consequence of transformation systems based on Agrobacterium, biolistics, or other commonly used procedures. More recently, gene targeting protocols have been developed that enable directed transgene insertion. One important technology, site-specific integration (SSI) enables the targeting of a transgene to the same chromosomal location as a previously inserted transgene. Custom-designed meganucleases and custom-designed zinc finger meganucleases allow researchers to design nucleases to target specific chromosomal locations, and these reagents allow the targeting of transgenes at the chromosomal site cleaved by these nucleases.

The currently used systems for precision genetic engineering of eukaryotic genomes, e.g. plant genomes, rely upon homing endonucleases, meganucleases, zinc finger nucleases, and transcription activator-like effector nucleases (TALENs), which require de novo protein engineering for every new target locus. The highly specific, RNA-directed DNA nuclease, guide RNA/Cas9 endonuclease system described herein, is more easily customizable and therefore more useful when modification of many different target sequences is the goal. This invention takes further advantage of the two component nature of the guide RNA/Cas system, with its constant protein component, the Cas endonucleae, and its variable and easily reprogrammable targeting component, the guide RNA or the crRNA.

The guide RNA/Cas system described herein is especially useful for genome engineering, especially plant genome engineering, in circumstances where nuclease off-target cutting can be toxic to the targeted cells. In one embodiment of the guide RNA/Cas system described herein, the constant component, in the form of an expression-optimized Cas9 gene, is stably integrated into the target genome, e.g. plant genome. Expression of the Cas9 gene is under control of a promoter, e.g. plant promoter, which can be a constitutive promoter, tissue-specific promoter or inducible promoter, e.g. temperature-inducible, stress-inducible, developmental stage inducible, or chemically inducible promoter. In the absence of the variable component, i.e. the guide RNA or crRNA, the Cas9 protein is not able to cut DNA and therefore its presence in the plant cell should have little or no consequence. Hence a key advantage of the guide RNA/Cas system described herein is the ability to create and maintain a cell line or transgenic organism capable of efficient expression of the Cas9 protein with little or no consequence to cell viability. In order to induce cutting at desired genomic sites to achieve targeted genetic modifications, guide RNAs or crRNAs can be introduced by a variety of methods into cells containing the stably-integrated and expressed cas9 gene. For example, guide RNAs or crRNAs can be chemically or enzymatically synthesized, and introduced into the Cas9 expressing cells via direct delivery methods such a particle bombardment or electroporation.

Alternatively, genes capable of efficiently expressing guide RNAs or crRNAs in the target cells can be synthesized chemically, enzymatically or in a biological system, and these genes can be introduced into the Cas9 expressing cells via direct delivery methods such a particle bombardment, electroporation or biological delivery methods such as Agrobacterium mediated DNA delivery.

One embodiment of the disclosure is a method for selecting a plant comprising an altered target site in its plant genome, the method comprising: a) obtaining a first plant comprising at least one Cas endonuclease capable of introducing a double strand break at a target site in the plant genome; b) obtaining a second plant comprising a guide RNA that is capable of forming a complex with the Cas endonuclease of (a), c) crossing the first plant of (a) with the second plant of (b); d) evaluating the progeny of (c) for an alteration in the target site and e) selecting a progeny plant that possesses the desired alteration of said target site.

Another embodiment of the disclosure is a method for selecting a plant comprising an altered target site in its plant genome, the method comprising: a) obtaining a first plant comprising at least one Cas endonuclease capable of introducing a double strand break at a target site in the plant genome; b) obtaining a second plant comprising a guide RNA and a donor DNA, wherein said guide RNA is capable of forming a complex with the Cas endonuclease of (a), wherein said donor DNA comprises a polynucleotide of interest; c) crossing the first plant of (a) with the second plant of (b); d) evaluating the progeny of (c) for an alteration in the target site and e) selecting a progeny plant that comprises the polynucleotide of interest inserted at said target site.

Another embodiment of the disclosure is a method for selecting a plant comprising an altered target site in its plant genome, the method comprising selecting at least one progeny plant that comprises an alteration at a target site in its plant genome, wherein said progeny plant was obtained by crossing a first plant expressing at least one Cas endonuclease to a second plant comprising a guide RNA and a donor DNA, wherein said Cas endonuclease is capable of introducing a double strand break at said target site, wherein said donor DNA comprises a polynucleotide of interest.

As disclosed herein, a guide RNA/Cas system mediating gene targeting can be used in methods for directing transgene insertion and/or for producing complex transgenic trait loci comprising multiple transgenes in a fashion similar as disclosed in WO2013/0198888 (published Aug. 1, 2013) where instead of using a double strand break inducing agent to introduce a gene of interest, a guide RNA/Cas system or a guide polynucleotide/Cas system as disclosed herein is used. In one embodiment, a complex transgenic trait locus is a genomic locus that has multiple transgenes genetically linked to each other. By inserting independent transgenes within 0.1, 0.2, 0.3, 04, 0.5, 1, 2, or even 5 centimorgans (cM) from each other, the transgenes can be bred as a single genetic locus (see, for example, U.S. patent application Ser. No. 13/427,138) or PCT application PCT/US2012/030061. After selecting a plant comprising a transgene, plants containing (at least) one transgenes can be crossed to form an F1 that contains both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for northern leaf blight resistance. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identify the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates containing target sites.

Sufficient homology or sequence identity indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bp. The amount of homology can also described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc); and, Tijssen (1993) Laboratory Techniques in Biochemistry and Molecular Biology—Hybridization with Nucleic Acid Probes, (Elsevier, New York).

A variety of methods are known for the introduction of nucleotide sequences and polypeptides into an organism, including, for example, transformation, sexual crossing, and the introduction of the polypeptide, DNA, or mRNA into the cell.

Methods for contacting, providing, and/or introducing a composition into various organisms are known and include but are not limited to, stable transformation methods, transient transformation methods, virus-mediated methods, and sexual breeding. Stable transformation indicates that the introduced polynucleotide integrates into the genome of the organism and is capable of being inherited by progeny thereof. Transient transformation indicates that the introduced composition is only temporarily expressed or present in the organism.

Protocols for introducing polynucleotides and polypeptides into plants may vary depending on the type of plant or plant cell targeted for transformation, such as monocot or dicot. Suitable methods of introducing polynucleotides and polypeptides into plant cells and subsequent insertion into the plant genome include microinjection (Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6, Agrobacterium-mediated transformation (U.S. Pat. Nos. 5,563,055 and 5,981,840), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin); McCabe et al., (1988) Biotechnology 6:923-6; Weissinger et al., (1988) Ann Rev Genet 22:421-77; Sanford et al., (1987) Particulate Science and Technology 5:27-37 (onion); Christou et al., (1988) Plant Physiol 87:671-4 (soybean); Finer and McMullen, (1991) In Vitro Cell Dev Biol 27P:175-82 (soybean); Singh et al., (1998) Theor Appl Genet 96:319-24 (soybean); Datta et al., (1990) Biotechnology 8:736-40 (rice); Klein et al., (1988) Proc. Natl. Acad. Sci. USA 85:4305-9 (maize); Klein et al., (1988) Biotechnology 6:559-63 (maize); U.S. Pat. Nos. 5,240,855; 5,322,783 and 5,324,646; Klein et al., (1988) Plant Physiol 91:440-4 (maize); Fromm et al., (1990) Biotechnology 8:833-9 (maize); Hooykaas-Van Slogteren et al., (1984) Nature 311:763-4; U.S. Pat. No. 5,736,369 (cereals); Bytebier et al., (1987) Proc. Natl. Acad. Sci. USA 84:5345-9 (Liliaceae); De Wet et al., (1985) in The Experimental Manipulation of Ovule Tissues, ed. Chapman et al., (Longman, N.Y.), pp. 197-209 (pollen); Kaeppler et al., (1990) Plant Cell Rep 9:415-8) and Kaeppler et al., (1992) Theor Appl Genet 84:560-6 (whisker-mediated transformation); D'Halluin et al., (1992) Plant Cell 4:1495-505 (electroporation); Li et al., (1993) Plant Cell Rep 12:250-5; Christou and Ford (1995) Annals Botany 75:407-13 (rice) and Osjoda et al., (1996) Nat Biotechnol 14:745-50 (maize via Agrobacterium tumefaciens).

Alternatively, polynucleotides may be introduced into plants by contacting plants with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931. Transient transformation methods include, but are not limited to, the introduction of polypeptides, such as a double-strand break inducing agent, directly into the organism, the introduction of polynucleotides such as DNA and/or RNA polynucleotides, and the introduction of the RNA transcript, such as an mRNA encoding a double-strand break inducing agent, into the organism. Such methods include, for example, microinjection or particle bombardment. See, for example Crossway et al., (1986) Mol Gen Genet 202:179-85; Nomura et al., (1986) Plant Sci 44:53-8; Hepler et al., (1994) Proc. Natl. Acad. Sci. USA 91:2176-80; and, Hush et al., (1994) J Cell Sci 107:775-84.

The term “dicot” refers to the subclass of angiosperm plants also knows as “dicotyledoneae” and includes reference to whole plants, plant organs (e.g., leaves, stems, roots, etc.), seeds, plant cells, and progeny of the same. Plant cell, as used herein includes, without limitation, seeds, suspension cultures, embryos, meristematic regions, callus tissue, leaves, roots, shoots, gametophytes, sporophytes, pollen, and microspores.

The term “crossed” or “cross” or “crossing” in the context of this invention means the fusion of gametes via pollination to produce progeny (i.e., cells, seeds, or plants). The term encompasses both sexual crosses (the pollination of one plant by another) and selfing (self-pollination, i.e., when the pollen and ovule are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene or a selected allele of a marker or QTL.

Standard DNA isolation, purification, molecular cloning, vector construction, and verification/characterization methods are well established, see, for example Sambrook et al., (1989) Molecular Cloning: A Laboratory Manual, (Cold Spring Harbor Laboratory Press, NY). Vectors and constructs include circular plasmids, and linear polynucleotides, comprising a polynucleotide of interest and optionally other components including linkers, adapters, regulatory regions, introns, restriction sites, enhancers, insulators, selectable markers, nucleotide sequences of interest, promoters, and/or other sites that aid in vector construction or analysis. In some examples a recognition site and/or target site can be contained within an intron, coding sequence, 5′ UTRs, 3′ UTRs, and/or regulatory regions.

The present invention further provides expression constructs for expressing in a plant, plant cell, or plant part a guide RNA/cas system that is capable of binding to and creating a double strand break in a target site. In one embodiment, the expression constructs of the invention comprise a promoter operably linked to a nucleotide sequence encoding a cas gene and a promoter operably linked to a guide RNA of the present invention. The promoter is capable of driving expression of an operably linked nucleotide sequence in a plant cell.

A promoter is a region of DNA involved in recognition and binding of RNA polymerase and other proteins to initiate transcription. A plant promoter is a promoter capable of initiating transcription in a plant cell, for a review of plant promoters, see, Potenza et al., (2004) In Vitro Cell Dev Biol 40:1-22. Constitutive promoters include, for example, the core promoter of the Rsyn7 promoter and other constitutive promoters disclosed in WO99/43838 and U.S. Pat. No. 6,072,050; the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; Christensen et al., (1992) Plant Mol Biol 18:675-89); pEMU (Last et al, (1991) Theor Appl Genet 81:581-8); MAS (Velten et al, (1984) EMBO J 3:2723-30); ALS promoter (U.S. Pat. No. 5,659,026), and the like. Other constitutive promoters are described in, for example, U.S. Pat. Nos. 5,608,149; 5,608,144; 5,604,121; 5,569,597; 5,466,785; 5,399,680; 5,268,463; 5,608,142 and 6,177,611. In some examples an inducible promoter may be used. Pathogen-inducible promoters induced following infection by a pathogen include, but are not limited to those regulating expression of PR proteins, SAR proteins, beta-1,3-glucanase, chitinase, etc.

Chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzene sulfonamide herbicide safeners (De Veylder et al., (1997) Plant Cell Physiol 38:568-77), the maize GST promoter (GST-II-27, WO93/01294), activated by hydrophobic electrophilic compounds used as pre-emergent herbicides, and the tobacco PR-la promoter (Ono et al., (2004) Biosci Biotechnol Biochem 68:803-7) activated by salicylic acid. Other chemical-regulated promoters include steroid-responsive promoters (see, for example, the glucocorticoid-inducible promoter (Schena et al., (1991) Proc. Natl. Acad. Sci. USA 88:10421-5; McNellis et al., (1998) Plant J 14:247-257); tetracycline-inducible and tetracycline-repressible promoters (Gatz et al., (1991) Mol Gen Genet 227:229-37; U.S. Pat. Nos. 5,814,618 and 5,789,156).

Tissue-preferred promoters can be utilized to target enhanced expression within a particular plant tissue. Tissue-preferred promoters include, for example, Kawamata et al., (1997) Plant Cell Physiol 38:792-803; Hansen et al., (1997) Mol Gen Genet 254:337-43; Russell et al., (1997) Transgenic Res 6:157-68; Rinehart et al., (1996) Plant Physiol 112:1331-41; Van Camp et al., (1996) Plant Physiol 112:525-35; Canevascini et al., (1996) Plant Physiol 112:513-524; Lam, (1994) Results Probl Cell Differ 20:181-96; and Guevara-Garcia et al., (1993) Plant J 4:495-505. Leaf-preferred promoters include, for example, Yamamoto et al., (1997) Plant J 12:255-65; Kwon et al., (1994) Plant Physiol 105:357-67; Yamamoto et al., (1994) Plant Cell Physiol 35:773-8; Gotor et al., (1993) Plant J 3:509-18; Orozco et al., (1993) Plant Mol Biol 23:1129-38; Matsuoka et al., (1993) Proc. Natl. Acad. Sci. USA 90:9586-90; Simpson et al., (1958) EMBO J 4:2723-9; Timko et al., (1988) Nature 318:57-8. Root-preferred promoters include, for example, Hire et al., (1992) Plant Mol Biol 20:207-18 (soybean root-specific glutamine synthase gene); Miao et al., (1991) Plant Cell 3:11-22 (cytosolic glutamine synthase (GS)); Keller and Baumgartner, (1991) Plant Cell 3:1051-61 (root-specific control element in the GRP 1.8 gene of French bean); Sanger et al., (1990) Plant Mol Biol 14:433-43 (root-specific promoter of A. tumefaciens mannopine synthase (MAS)); Bogusz et al., (1990) Plant Cell 2:633-41 (root-specific promoters isolated from Parasponia andersoni and Trema tomentosa); Leach and Aoyagi, (1991) Plant Sci 79:69-76 (A. rhizogenes rolC and rolD root-inducing genes); Teeri et al., (1989) EMBO J 8:343-50 (Agrobacterium wound-induced TR1′ and TR2′ genes); VfENOD-GRP3 gene promoter (Kuster et al., (1995) Plant Mol Biol 29:759-72); and rolB promoter (Capana et al., (1994) Plant Mol Biol 25:681-91; phaseolin gene (Murai et al., (1983) Science 23:476-82; Sengopta-Gopalen et al., (1988) Proc. Natl. Acad. Sci. USA 82:3320-4). See also, U.S. Pat. Nos. 5,837,876; 5,750,386; 5,633,363; 5,459,252; 5,401,836; 5,110,732 and 5,023,179.

Seed-preferred promoters include both seed-specific promoters active during seed development, as well as seed-germinating promoters active during seed germination. See, Thompson et al., (1989) BioEssays 10:108. Seed-preferred promoters include, but are not limited to, Cim1 (cytokinin-induced message); cZ19B1 (maize 19 kDa zein); and milps (myo-inositol-1-phosphate synthase); (WO00/11177; and U.S. Pat. No. 6,225,529). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, and nuc1. See also, WO00/12733, where seed-preferred promoters from END1 and END2 genes are disclosed.

A phenotypic marker is a screenable or selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as glufosinate ammonium, bromoxynil, imidazolinones, and 2,4-dichlorophenoxyacetate (2,4-D). See for example, Yarranton, (1992) Curr Opin Biotech 3:506-11; Christopherson et al., (1992) Proc. Natl. Acad. Sci. USA 89:6314-8; Yao et al., (1992) Cell 71:63-72; Reznikoff, (1992) Mol Microbiol 6:2419-22; Hu et al., (1987) Cell 48:555-66; Brown et al., (1987) Cell 49:603-12; Figge et al., (1988) Cell 52:713-22; Deuschle et al., (1989) Proc. Natl. Acad. Sci. USA 86:5400-4; Fuerst et al., (1989) Proc. Natl. Acad. Sci. USA 86:2549-53; Deuschle et al., (1990) Science 248:480-3; Gossen, (1993) Ph.D. Thesis, University of Heidelberg; Reines et al., (1993) Proc. Natl. Acad. Sci. USA 90:1917-21; Labow et al., (1990) Mol Cell Biol 10:3343-56; Zambretti et al., (1992) Proc. Natl. Acad. Sci. USA 89:3952-6; Baim et al., (1991) Proc. Natl. Acad. Sci. USA 88:5072-6; Wyborski et al., (1991) Nucleic Acids Res 19:4647-53; Hillen and Wissman, (1989) Topics Mol Struc Biol 10:143-62; Degenkolb et al., (1991) Antimicrob Agents Chemother 35:1591-5; Kleinschnidt et al., (1988) Biochemistry 27:1094-104; Bonin, (1993) Ph.D. Thesis, University of Heidelberg; Gossen et al., (1992) Proc. Natl. Acad. Sci. USA 89:5547-51; Oliva et al., (1992) Antimicrob Agents Chemother 36:913-9; Hlavka et al., (1985) Handbook of Experimental Pharmacology, Vol. 78 (Springer-Verlag, Berlin); Gill et al., (1988) Nature 334:721-4.

The cells having the introduced sequence may be grown or regenerated into plants using conventional conditions, see for example, McCormick et al., (1986) Plant Cell Rep 5:81-4. These plants may then be grown, and either pollinated with the same transformed strain or with a different transformed or untransformed strain, and the resulting progeny having the desired characteristic and/or comprising the introduced polynucleotide or polypeptide identified. Two or more generations may be grown to ensure that the polynucleotide is stably maintained and inherited, and seeds harvested.

Any plant can be used, including monocot and dicot plants. Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum aestivum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses. Examples of dicot plants that can be used include, but are not limited to, soybean (Glycine max), canola (Brassica napus and B. campestris), alfalfa (Medicago sativa), tobacco (Nicotiana tabacum), Arabidopsis (Arabidopsis thaliana), sunflower (Helianthus annuus), cotton (Gossypium arboreum), and peanut (Arachis hypogaea), tomato (Solanum lycopersicum), potato (Solanum tuberosum) etc.

The transgenes, recombinant DNA molecules, DNA sequences of interest, and polynucleotides of interest can comprise one or more genes of interest. Such genes of interest can encode, for example, a protein that provides agronomic advantage to the plant.

Marker Assisted Selection and Breeding of Plants

A primary motivation for development of molecular markers in crop species is the potential for increased efficiency in plant breeding through marker assisted selection (MAS). Genetic marker alleles, or alternatively, quantitative trait loci (QTL alleles, are used to identify plants that contain a desired genotype at one or more loci, and that are expected to transfer the desired genotype, along with a desired phenotype to their progeny. Genetic marker alleles (or QTL alleles) can be used to identify plants that contain a desired genotype at one locus, or at several unlinked or linked loci (e.g., a haplotype), and that would be expected to transfer the desired genotype, along with a desired phenotype to their progeny. It will be appreciated that for the purposes of MAS, the term marker can encompass both marker and QTL loci.

After a desired phenotype and a polymorphic chromosomal locus, e.g., a marker locus or QTL, are determined to segregate together, it is possible to use those polymorphic loci to select for alleles corresponding to the desired phenotype—a process called marker-assisted selection (MAS). In brief, a nucleic acid corresponding to the marker nucleic acid is detected in a biological sample from a plant to be selected. This detection can take the form of hybridization of a probe nucleic acid to a marker, e.g., using allele-specific hybridization, southern blot analysis, northern blot analysis, in situ hybridization, hybridization of primers followed by PCR amplification of a region of the marker or the like. A variety of procedures for detecting markers are well known in the art. After the presence (or absence) of a particular marker in the biological sample is verified, the plant is selected, i.e., used to make progeny plants by selective breeding.

Plant breeders need to combine traits of interest with genes for high yield and other desirable traits to develop improved plant varieties. Screening for large numbers of samples can be expensive, time consuming, and unreliable. Use of markers, and/or genetically-linked nucleic acids is an effective method for selecting plant having the desired traits in breeding programs. For example, one advantage of marker-assisted selection over field evaluations is that MAS can be done at any time of year regardless of the growing season. Moreover, environmental effects are irrelevant to marker-assisted selection.

When a population is segregating for multiple loci affecting one or multiple traits, the efficiency of MAS compared to phenotypic screening becomes even greater because all the loci can be processed in the lab together from a single sample of DNA.

The DNA repair mechanisms of cells are the basis of transformation to introduce extraneous DNA or induce mutations on endogenous genes. DNA homologous recombination is a specialized way of DNA repair that the cells repair DNA damages using a homologous sequence. In plants, DNA homologous recombination happens at frequencies too low to be used in transformation until it has been found that the process can be stimulated by DNA double-strand breaks (Bibikova et al., (2001) Mol. Cell Biol. 21:289-297; Puchta and Baltimore, (2003) Science 300:763; Wright et al., (2005) Plant J. 44:693-705).

The meaning of abbreviations is as follows: “sec” means second(s), “min” means minute(s), “h” means hour(s), “d” means day(s), “PL” means microliter(s), “mL” means milliliter(s), “L” means liter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kb” means kilobase(s).

Also, as described herein, for each example or embodiment that cites a guide RNA, a similar guide polynucleotide can be designed wherein the guide polynucleotide does not solely comprise ribonucleic acids but wherein the guide polynucleotide comprises a combination of RNA-DNA molecules or solely comprises DNA molecules.

A method for editing a nucleotide sequence in the genome of a cell, the method comprising introducing a guide polynucleotide, a Cas endonuclease, and optionally a polynucleotide modification template, into a cell, wherein said guide RNA and Cas endonuclease are capable of forming a complex that enables the Cas endonuclease to introduce a double strand break at a target site in the genome of said cell, wherein said polynucleotide modification template comprises at least one nucleotide modification of said nucleotide sequence.

The method of embodiment 53, wherein the nucleotide sequence in the genome of a cell is selected from the group consisting of a promoter sequence, a terminator sequence, a regulatory element sequence, a splice site, a coding sequence, a polyubiquitination site, an intron site and an intron enhancing motif.

A method for editing a promoter sequence in the genome of a cell, the method comprising introducing a guide polynucleotide, a polynucleotide modification template and at least one Cas endonuclease into a cell, wherein said guide RNA and Cas endonuclease are capable of forming a complex that enables the Cas endonuclease to introduce a double strand break at a target site in the genome of said cell, wherein said polynucleotide modification template comprises at least one nucleotide modification of said nucleotide sequence.

A method for replacing a first promoter sequence in a cell, the method comprising introducing a guide RNA, a polynucleotide modification template, and a Cas endonuclease into said cell, wherein said guide RNA and Cas endonuclease are capable of forming a complex that enables the Cas endonuclease to introduce a double strand break at a target site in the genome of said cell, wherein said polynucleotide modification template comprises a second promoter or second promoter fragment that is different from said first promoter sequence.

The method of embodiment 56, wherein the replacement of the first promoter sequence results in any one of the following, or any one combination of the following: an increased promoter activity, an increased promoter tissue specificity, a decreased promoter activity, a decreased promoter tissue specificity, a new promoter activity, an inducible promoter activity, an extended window of gene expression, or a modification of the timing or developmental progress of gene expression in the same cell layer or other cell layer

The method of embodiment 56, wherein the first promoter sequence is selected from the group consisting of Zea mays ARGOS 8 promoter, a soybean EPSPS1 promoter, a maize EPSPS promoter, maize NPK1 promoter, wherein the second promoter sequence is selected from the group consisting of a Zea mays GOS2 PRO:GOS2-intron promoter, a soybean ubiquitin promoter, a stress inducible maize RAB17 promoter, a Zea mays-PEPC1 promoter, a Zea mays Ubiquitin promoter, a Zea mays-Rootmet2 promoter, a rice actin promoter, a sorghum RCC3 promoter, a Zea mays-GOS2 promoter, a Zea mays-ACO2 promoter and a Zea mays oleosin promoter.

A method for deleting a promoter sequence in the genome of a cell, the method comprising introducing a guide polynucleotide, a Cas endonuclease into a cell, wherein said guide RNA and Cas endonuclease are capable of forming a complex that enables the Cas endonuclease to introduce a double strand break in at least one target site located inside or outside said promoter sequence.

A method for inserting a promoter or a promoter element in the genome of a cell, the method comprising introducing a guide polynucleotide, a polynucleotide modification template comprising the promoter or the promoter element, and a Cas endonuclease into a cell, wherein said guide RNA and Cas endonuclease are capable of forming a complex that enables the Cas endonuclease to introduce a double strand break at a target site in the genome of said cell.

The method of embodiment 60, wherein the insertion of the promoter or promoter element results in any one of the following, or any one combination of the following: an increased promoter activity, an increased promoter tissue specificity, a decreased promoter activity, a decreased promoter tissue specificity, a new promoter activity, an inducible promoter activity, an extended window of gene expression, a modification of the timing or developmental progress of gene expression, a mutation of DNA binding elements, or an addition of DNA binding elements.

A method for editing a Zinc Finger transcription factor, the method comprising introducing a guide polynucleotide, a Cas endonuclease, and optionally a polynucleotide modification template, into a cell, wherein the Cas endonuclease introduces a double-strand break at a target site in the genome of said cell, wherein said polynucleotide modification template comprises at least one nucleotide modification or deletion of said Zinc Finger transcription factor, wherein the deletion or modification of said Zinc Finger transcription factor results in the creation of a dominant negative Zinc Finger transcription factor mutant.

A method for creating a fusion protein, the method comprising introducing a guide polynucleotide, a Cas endonuclease, and a polynucleotide modification template, into a cell, wherein the Cas endonuclease introduces a double-strand break at a target site located inside or outside a first coding sequence in the genome of said cell, wherein said polynucleotide modification template comprises a second coding sequence encoding a protein of interest, wherein the protein fusion results in any one of the following, or any one combination of the following: a targeting of the fusion protein to the chloroplast of said cell, an increased protein activity, an increased protein functionality, a decreased protein activity, a decreased protein functionality, a new protein functionality, a modified protein functionality, a new protein localization, a new timing of protein expression, a modified protein expression pattern, a chimeric protein, or a modified protein with dominant phenotype functionality.

In an embodiment, maize corn root worm (crw1) mutation (WO2014047505A1, incorporated herein by reference) can be engineered using guided cas9 technology disclosed herein.

In an embodiment, maize corn root worm (crw2) mutation (WO2014047508A1, incorporated herein by reference) can be engineered using guided cas9 technology disclosed herein.

### EXAMPLES

The present invention is further defined in the following Examples, in which parts and percentages are by weight and degrees are Celsius, unless otherwise stated. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Such modifications are also intended to fall within the scope of the appended claims.

### Example 1

### Maize Optimized Expression Cassettes for Guide RNA/Cas Endonuclease Based Genome Modification in Maize Plants

For genome engineering applications, the type II CRISPR/Cas system minimally requires the Cas9 protein and a duplexed crRNA/tracrRNA molecule or a synthetically fused crRNA and tracrRNA (guide RNA) molecule for DNA target site recognition and cleavage (Gasiunas et al. (2012) Proc. Natl. Acad. Sci. USA 109:E2579-86, Jinek et al. (2012) Science 337:816-21, Mali et al. (2013) Science 339:823-26, and Cong et al. (2013) Science 339:819-23). Described herein is a guideRNA/Cas endonuclease system that is based on the type II CRISPR/Cas system and consists of a Cas endonuclease and a guide RNA (or duplexed crRNA and tracrRNA) that together can form a complex that recognizes a genomic target site in a plant and introduces a double-strand-break into said target site.

To test the guide RNA/Cas endonuclease system in maize, the Cas9 gene from Streptococcus pyogenes M1 GAS (SF370) (SEQ ID NO: 1) was maize codon optimized per standard techniques known in the art and the potato ST-LS1 intron (SEQ ID NO: 2) was introduced in order to eliminate its expression in E. coli and Agrobacterium (FIG. 1 A). To facilitate nuclear localization of the Cas9 protein in maize cells, Simian virus 40 (SV40) monopartite amino terminal nuclear localization signal (MAPKKKRKV, SEQ ID NO: 3) and Agrobacterium tumefaciens bipartite VirD2 T-DNA border endonuclease carboxyl terminal nuclear localization signal (KRPRDRHDGELGGRKRAR, SEQ ID NO: 4) were incorporated at the amino and carboxyl-termini of the Cas9 open reading frame (FIG. 1 A), respectively. The maize optimized Cas9 gene was operably linked to a maize constitutive or regulated promoter by standard molecular biological techniques. An example of the maize optimized Cas9 expression cassette (SEQ ID NO: 5) is illustrated in FIG. 1 A. FIG. 1A shows a maize optimized Cas9 gene containing the ST-LS1 intron, SV40 amino terminal nuclear localization signal (NLS) and VirD2 carboxyl terminal NLS driven by a plant Ubiquitin promoter.

The second component recommended to form a functional guide RNA/Cas endonuclease system for genome engineering applications is a duplex of the crRNA and tracrRNA molecules or a synthetic fusing of the crRNA and tracrRNA molecules, a guide RNA. To confer efficient guide RNA expression (or expression of the duplexed crRNA and tracrRNA) in maize, the maize U6 polymerase III promoter (SEQ ID NO: 9) and maize U6 polymerase III terminator (first 8 bases of SEQ ID NO: 10) residing on chromosome 8 were isolated and operably fused to the termini of a guide RNA (FIG. 1 B) using standard molecular biology techniques. Two different guide RNA configurations were developed for testing in maize, a short guide RNA (SEQ ID NO: 11) based on Jinek et al. (2012) Science 337:816-21 and a long guide RNA (SEQ ID NO: 8) based on Mali et al. (2013) Science 339:823-26. An example expression cassette (SEQ ID NO: 12) is shown in FIG. 1 B which illustrates a maize U6 polymerase III promoter driving expression of a long guide RNA terminated with a U6 polymerase III terminator.

As shown in FIGS. 2 A and 2B, the guide RNA or crRNA molecule also need to contain a region complementary to one strand of the double strand DNA target (referred to as the variable targeting domain) that is approximately 12-30 nucleotides in length and upstream of a PAM sequence (5′NGG3′ on antisense strand of FIG. 2A-2B, corresponding to 5′CCN3′ on sense strand of FIG. 2A-2B) for target site recognition and cleavage (Gasiunas et al. (2012) Proc. Natl. Acad. Sci. USA 109:E2579-86, Jinek et al. (2012) Science 337:816-21, Mali et al. (2013) Science 339:823-26, and Cong et al. (2013) Science 339:819-23). To facilitate the rapid introduction of maize genomic DNA target sequences into the crRNA or guide RNA expression constructs, two Type IIS BbsI restriction endonuclease target sites were introduced in an inverted tandem orientation with cleavage orientated in an outward direction as described in Cong et al. (2013) Science 339:819-23. Upon cleavage, the Type IIS restriction endonuclease excises its target sites from the crRNA or guide RNA expression plasmid, generating overhangs allowing for the in-frame directional cloning of duplexed oligos containing the desired maize genomic DNA target site into the variable targeting domain. In this example, only target sequences starting with a G nucleotide were used to promote favorable polymerase III expression of the guide RNA or crRNA.

Expression of both the Cas endonuclease gene and the guide RNA then allows for the formation of the guide RNA/Cas complex depicted in FIG. 2 B (SEQ ID NO: 8). Alternatively, expression of the Cas endonucleases gene, crRNA, and tracrRNA allow for the formation of the crRNA/tracrRNA/Cas complex as depicted in FIG. 2 A, (SEQ ID NOs: 6-7).

### Example 2

### The Guide RNA/Cas Endonuclease System May be Multiplexed to Simultaneously Target Multiple Chromosomal Loci in Maize for Mutagenesis by Imperfect Non-Homologous End-Joining

To test if multiple chromosomal loci may be simultaneously mutagenized with the guide RNA/maize optimized Cas endonuclease system described herein, the long guide RNA expression cassettes targeting the MS26Cas-2 target site (SEQ ID NO: 14), the LIGCas-3 target site (SEQ ID NO: 18) and the MS45Cas-2 target site (SEQ ID NO: 20), were co-transformed into maize embryos either in duplex or in triplex along with the Cas9 endonuclease expression cassette and examined by deep sequencing for the presence of imprecise NHEJ mutations as described in Example 2.

Hi-II maize embryos co-transformed with the Cas9 expression cassette and the corresponding guide RNA expression cassette singly served as a positive control and embryos transformed with only the Cas9 expression cassette served as a negative control.

### Example 3

### Delivery Methods for Plant Genome Editing Using the Guide RNA/Cas Endonuclease System

This example describes methods to deliver or maintain and express the Cas9 endonuclease and guide RNA (or individual crRNA and tracrRNAs) into, or within plants, respectively, to enable directed DNA modification or gene insertion via homologous recombination. More specifically this example describes a variety of methods which include, but are not limited to, delivery of the Cas9 endonuclease as a DNA, RNA (5′-capped and polyadenylated) or protein molecule. In addition, the guide RNA may be delivered as a DNA or RNA molecule.

Shown in Example 2, a high mutation frequency was observed when Cas9 endonuclease and guide RNA were delivered as DNA vectors by biolistic transformation of immature corn embryos. Other embodiments of this disclosure can be to deliver the Cas9 endonuclease as a DNA, RNA or protein and the guide RNA as a DNA or RNA molecule or as a duplex crRNA/tracrRNA molecule as RNA or DNA or a combination.

Delivery of the Cas9 (as DNA vector) and guide RNA (as DNA vector) example can also be accomplished by co-delivering these DNA cassettes on a single or multiple Agrobacterium vectors and transforming plant tissues by Agrobacterium mediated transformation. In addition, a vector containing a constitutive, tissue-specific or conditionally regulated Cas9 gene can be first delivered to plant cells to allow for stable integration into the plant genome to establish a plant line that contains only the Cas9 gene in the plant genome. In this example, single or multiple guide RNAs, or single or multiple crRNA and a tracrRNA can be delivered as either DNA or RNA, or combination, to the plant line containing the genome-integrated version of the Cas9 gene for the purpose of generating mutations or promoting homologous recombination when HR repair DNA vectors for targeted integration are co-delivered with the guide RNAs. As extension of this example, plant line containing the genome-integrated version of the Cas9 gene and a tracrRNA as a DNA molecule can also be established. In this example single or multiple crRNA molecules can be delivered as RNA or DNA to promote the generation of mutations or to promote homologous recombination when HR repair DNA vectors for targeted integration are co-delivered with crRNA molecule(s) enabling the targeted mutagenesis or homologous recombination at single or multiple sites in the plant genome.

### Example 4

### Components of the Guide RNA/Cas Endonuclease System Delivered Directly as RNA in Plants

This example illustrates the use of the methods as described herein and configuration of Example 7 [Cas9 (DNA vector), guide RNA (RNA)] for modification or mutagenesis of chromosomal loci in plants. The maize optimized Cas9 endonuclease expression cassette described in Example 1 was co-delivered by particle gun as described in Example 2 along with single stranded RNA molecules (synthesized by Integrated DNA Technologies, Inc.) constituting a short guide RNA targeting the maize locus and sequence shown. Embryos transformed with only the Cas9 expression cassette or short guide RNA molecules served as negative controls. Seven days post-bombardment, the immature embryos were harvested and analyzed by deep sequencing for NHEJ mutations as described in Example 2. Mutations not present in the negative controls were found at the site (FIG. 6, corresponding to SEQ ID NOs: 104-110). These mutations were similar to those found in Examples 2, 3, 4 and 6. This data indicates that component(s) of the maize optimized guide RNA/Cas endonuclease system described herein may be delivered directly as RNA.

TABLE 1
Maize genomic target site and location
for short guide RNA delivered as RNA.

| | | Guide | | Maize | | SEQ |
|---|---|---|---|---|---|---|
| | | RNA | Desig- | Target | PAM | ID |
| Locus | Location | Used | nation | Site | Sequence | NO |
| 55 | Chr.1: | Short | 55CasRNA-1 | TGGGCA | TGG | 103 |
| | 51.78 cM | | | GGTCTC | | |
| | | | | ACGACG | | |
| | | | | GT | | |

### Example 5

### Creation of Rare Cutting Engineered Meganucleases

### LIG3-4 Meganuclease and LIG3-4 Intended Recognition Sequence

An endogenous maize genomic target site comprising the LIG3-4 intended recognition sequence (SEQ ID NO: 111) was selected for design of a rare-cutting double-strand break inducing agent (SEQ ID NO: 112) as described in US patent publication 2009-0133152 A1 (published May 21, 2009). The LIG3-4 intended recognition sequence is a 22 bp polynucleotide having the following sequence:

(SEQ ID NO: 111)
ATATACCTCACACGTACGCGTA.

### MS 26++ Meganuclease

An endogenous maize genomic target site designated “TS-MS26” (SEQ ID NO: 113) was selected for design of a custom double-strand break inducing agent MS26++ as described in U.S. patent application Ser. No. 13/526,912 filed Jun. 19, 2012). The TS-MS26 target site is a 22 bp polynucleotide positioned 62 bps from the 5′ end of the fifth exon of the maize MS26 gene and having the following sequence: gatggtgacgtaĉgtgccctac (SEQ ID NO: 113). The double strand break site and overhang region is underlined, the enzyme cuts after C13, as indicated by the ̂. Plant optimized nucleotide sequences for an engineered endonuclease (SEQ ID NO: 114) encoding an engineered MS26++ endonuclease were designed to bind and make double-strand breaks at the selected TS-MS26 target site.

### Example 6

### Transformation of Maize Immature Embryos

Transformation can be accomplished by various methods known to be effective in plants, including particle-mediated delivery, Agrobacterium-mediated transformation, PEG-mediated delivery, and electroporation.

a. Particle-Mediated Delivery

Transformation of maize immature embryos using particle delivery is performed as follows. Media recipes follow below.

The ears are husked and surface sterilized in 30% Clorox bleach plus 0.5% Micro detergent for 20 minutes, and rinsed two times with sterile water. The immature embryos are isolated and placed embryo axis side down (scutellum side up), 25 embryos per plate, on 560Y medium for 4 hours and then aligned within the 2.5-cm target zone in preparation for bombardment. Alternatively, isolated embryos are placed on 560 L (Initiation medium) and placed in the dark at temperatures ranging from 26° C. to 37° C. for 8 to 24 hours prior to placing on 560Y for 4 hours at 26° C. prior to bombardment as described above.

Plasmids containing the double strand brake inducing agent and donor DNA are constructed using standard molecular biology techniques and co-bombarded with plasmids containing the developmental genes ODP2 (AP2 domain transcription factor ODP2 (Ovule development protein 2); US20090328252 A1) and Wushel (US2011/0167516).

The plasmids and DNA of interest are precipitated onto 0.6 m (average diameter) gold pellets using a water-soluble cationic lipid Tfx™-50 (Cat# E1811, Promega, Madison, Wis., USA) as follows. DNA solution is prepared on ice using 1 g of plasmid DNA and optionally other constructs for co-bombardment such as 50 ng (0.5 μl) of each plasmid containing the developmental genes ODP2 (AP2 domain transcription factor ODP2 (Ovule development protein 2); US20090328252 A1) and Wushel. To the pre-mixed DNA, 20 μl of prepared gold particles (15 mg/ml) and 5 μl Tfx-50 is added in water and mixed carefully. Gold particles are pelleted in a microfuge at 10,000 rpm for 1 min and supernatant is removed. The resulting pellet is carefully rinsed with 100 ml of 100% EtOH without resuspending the pellet and the EtOH rinse is carefully removed. 105 μl of 100% EtOH is added and the particles are resuspended by brief sonication. Then, 10 μl is spotted onto the center of each macrocarrier and allowed to dry about 2 minutes before bombardment.

Alternatively, the plasmids and DNA of interest are precipitated onto 1.1 m (average diameter) tungsten pellets using a calcium chloride (CaCl₂) precipitation procedure by mixing 100 μl prepared tungsten particles in water, 10 μl (1 μg) DNA in Tris EDTA buffer (1 μg total DNA), 100 μl 2.5 M CaCl2, and 10 μl 0.1 M spermidine. Each reagent is added sequentially to the tungsten particle suspension, with mixing. The final mixture is sonicated briefly and allowed to incubate under constant vortexing for 10 minutes. After the precipitation period, the tubes are centrifuged briefly, liquid is removed, and the particles are washed with 500 ml 100% ethanol, followed by a 30 second centrifugation. Again, the liquid is removed, and 105 μl 100% ethanol is added to the final tungsten particle pellet. For particle gun bombardment, the tungsten/DNA particles are briefly sonicated. 10 μl of the tungsten/DNA particles is spotted onto the center of each macrocarrier, after which the spotted particles are allowed to dry about 2 minutes before bombardment.

The sample plates are bombarded at level #4 with a Biorad Helium Gun. All samples receive a single shot at 450 PSI, with a total of ten aliquots taken from each tube of prepared particles/DNA.

Following bombardment, the embryos are incubated on 560P (maintenance medium) for 12 to 48 hours at temperatures ranging from 26° C. to 367° C., and then placed at 26 C. After 5 to 7 days the embryos are transferred to 560R selection medium containing 3 mg/liter Bialaphos, and subcultured every 2 weeks at 26 C. After approximately 10 weeks of selection, selection-resistant callus clones are transferred to 288 J medium to initiate plant regeneration. Following somatic embryo maturation (2-4 weeks), well-developed somatic embryos are transferred to medium for germination and transferred to a lighted culture room. Approximately 7-10 days later, developing plantlets are transferred to 272V hormone-free medium in tubes for 7-10 days until plantlets are well established. Plants are then transferred to inserts in flats (equivalent to a 2.5″ pot) containing potting soil and grown for 1 week in a growth chamber, subsequently grown an additional 1-2 weeks in the greenhouse, then transferred to Classic 600 pots (1.6 gallon) and grown to maturity. Plants are monitored and scored for transformation efficiency, and/or modification of regenerative capabilities.

Initiation medium (560 L) comprises 4.0 g/l N6 basal salts (SIGMA C-1416), 1.0 ml/l Eriksson's Vitamin Mix (1000×SIGMA-1511), 0.5 mg/l thiamine HCl, 20.0 g/l sucrose, 1.0 mg/l 2,4-D, and 2.88 g/l L-proline (brought to volume with D-I H2O following adjustment to pH 5.8 with KOH); 2.0 g/l Gelrite (added after bringing to volume with D-I H2O); and 8.5 mg/l silver nitrate (added after sterilizing the medium and cooling to room temperature).

Maintenance medium (560P) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose, 2.0 mg/l 2,4-D, and 0.69 g/l L-proline (brought to volume with D-I H2O following adjustment to pH 5.8 with KOH); 3.0 g/l Gelrite (added after bringing to volume with D-I H2O); and 0.85 mg/l silver nitrate (added after sterilizing the medium and cooling to room temperature).

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H2O following adjustment to pH 5.8 with KOH); 2.0 g/l Gelrite (added after bringing to volume with D-I H2O); and 8.5 mg/l silver nitrate (added after sterilizing the medium and cooling to room temperature).

Selection medium (560R) comprises 4.0 g/l N6 basal salts (SIGMA C-1416), 1.0 ml/I Eriksson's Vitamin Mix (1000×SIGMA-1511), 0.5 mg/l thiamine HCl, 30.0 g/I sucrose, and 2.0 mg/l 2,4-D (brought to volume with D-I H2O following adjustment to pH 5.8 with KOH); 3.0 g/l Gelrite (added after bringing to volume with D-I H2O); and 0.85 mg/l silver nitrate and 3.0 mg/l bialaphos (both added after sterilizing the medium and cooling to room temperature).

Plant regeneration medium (288 J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H2O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H2O after adjusting to pH 5.6); 3.0 g/l Gelrite (added after bringing to volume with D-I H2O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H2O), 0.1 g/l myo-inositol, and 40.0 g/l sucrose (brought to volume with polished D-I H2O after adjusting pH to 5.6); and 6 g/l bacto-agar (added after bringing to volume with polished D-I H2O), sterilized and cooled to 60° C.

b. Agrobacterium-Mediated Transformation

Agrobacterium-mediated transformation was performed essentially as described in Djukanovic et al. (2006) Plant Biotech J 4:345-57. Briefly, 10-12 day old immature embryos (0.8-2.5 mm in size) were dissected from sterilized kernels and placed into liquid medium (4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 68.5 g/L sucrose, 36.0 g/L glucose, pH 5.2). After embryo collection, the medium was replaced with 1 ml Agrobacterium at a concentration of 0.35-0.45 OD550. Maize embryos were incubated with Agrobacterium for 5 min at room temperature, then the mixture was poured onto a media plate containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 30.0 g/L sucrose, 0.85 mg/L silver nitrate, 0.1 nM acetosyringone, and 3.0 g/L Gelrite, pH 5.8. Embryos were incubated axis down, in the dark for 3 days at 200, then incubated 4 days in the dark at 280, then transferred onto new media plates containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.69 g/L L-proline, 30.0 g/L sucrose, 0.5 g/L MES buffer, 0.85 mg/L silver nitrate, 3.0 mg/L Bialaphos, 100 mg/L carbenicillin, and 6.0 g/L agar, pH 5.8. Embryos were subcultured every three weeks until transgenic events were identified. Somatic embryogenesis was induced by transferring a small amount of tissue onto regeneration medium (4.3 g/L MS salts (Gibco 11117), 5.0 ml/L MS Vitamins Stock Solution, 100 mg/L myo-inositol, 0.1 μM ABA, 1 mg/L IAA, 0.5 mg/L zeatin, 60.0 g/L sucrose, 1.5 mg/L Bialaphos, 100 mg/L carbenicillin, 3.0 g/L Gelrite, pH 5.6) and incubation in the dark for two weeks at 28° C. All material with visible shoots and roots were transferred onto media containing 4.3 g/L MS salts (Gibco 11117), 5.0 ml/L MS Vitamins Stock Solution, 100 mg/L myo-inositol, 40.0 g/L sucrose, 1.5 g/L Gelrite, pH 5.6, and incubated under artificial light at 280. One week later, plantlets were moved into glass tubes containing the same medium and grown until they were sampled and/or transplanted into soil.

### Example 7

### Transient Expression of BBM Enhances Transformation

Parameters of the transformation protocol can be modified to ensure that the BBM activity is transient. One such method involves precipitating the BBM-containing plasmid in a manner that allows for transcription and expression, but precludes subsequent release of the DNA, for example, by using the chemical PEI. In one example, the BBM plasmid is precipitated onto gold particles with PEI, while the transgenic expression cassette (UBI::moPAT˜GFPm::PinII; moPAT is the maize optimized PAT gene) to be integrated is precipitated onto gold particles using the standard calcium chloride method.

Briefly, gold particles were coated with PEI as follows. First, the gold particles were washed. Thirty-five mg of gold particles, 1.0 in average diameter (A.S.I. #162-0010), were weighed out in a microcentrifuge tube, and 1.2 ml absolute EtOH was added and vortexed for one minute. The tube was incubated for 15 minutes at room temperature and then centrifuged at high speed using a microfuge for 15 minutes at 4° C. The supernatant was discarded and a fresh 1.2 ml aliquot of ethanol (EtOH) was added, vortexed for one minute, centrifuged for one minute, and the supernatant again discarded (this is repeated twice). A fresh 1.2 ml aliquot of EtOH was added, and this suspension (gold particles in EtOH) was stored at −20° C. for weeks. To coat particles with polyethylimine (PEI; Sigma #P3143), 250 μl of the washed gold particle/EtOH mix was centrifuged and the EtOH discarded. The particles were washed once in 100 μl ddH2O to remove residual ethanol, 250 μl of 0.25 mM PEI was added, followed by a pulse-sonication to suspend the particles and then the tube was plunged into a dry ice/EtOH bath to flash-freeze the suspension, which was then lyophilized overnight. At this point, dry, coated particles could be stored at −80° C. for at least 3 weeks. Before use, the particles were rinsed 3 times with 250 μl aliquots of 2.5 mM HEPES buffer, pH 7.1, with 1×pulse-sonication, and then a quick vortex before each centrifugation. The particles were then suspended in a final volume of 250 μl HEPES buffer. A 25 μl aliquot of the particles was added to fresh tubes before attaching DNA. To attach uncoated DNA, the particles were pulse-sonicated, then 1 μg of DNA (in 5 μl water) was added, followed by mixing by pipetting up and down a few times with a Pipetteman and incubated for 10 minutes. The particles were spun briefly (i.e. 10 seconds), the supernatant removed, and 60 μl EtOH added. The particles with PEI-precipitated DNA-1 were washed twice in 60 μl of EtOH. The particles were centrifuged, the supernatant discarded, and the particles were resuspended in 45 μl water. To attach the second DNA (DNA-2), precipitation using TFX-50 was used. The 45 μl of particles/DNA-1 suspension was briefly sonicated, and then 5 μl of 100 ng/μl of DNA-2 and 2.5 μl of TFX-50 were added. The solution was placed on a rotary shaker for 10 minutes, centrifuged at 10,000 g for 1 minute. The supernatant was removed, and the particles resuspended in 60 μl of EtOH. The solution was spotted onto macrocarriers and the gold particles onto which DNA-1 and DNA-2 had been sequentially attached were delivered into scutellar cells of 10 DAP Hi-II immature embryos using a standard protocol for the PDS-1000. For this experiment, the DNA-1 plasmid contained a UBI::RFP::pinII expression cassette, and DNA-2 contained a UBI::CFP::pinII expression cassette. Two days after bombardment, transient expression of both the CFP and RFP fluorescent markers was observed as numerous red & blue cells on the surface of the immature embryo. The embryos were then placed on non-selective culture medium and allowed to grow for 3 weeks before scoring for stable colonies. After this 3-week period, 10 multicellular, stably-expressing blue colonies were observed, in comparison to only one red colony. This demonstrated that PEI-precipitation could be used to effectively introduce DNA for transient expression while dramatically reducing integration of the PEI-introduced DNA and thus reducing the recovery of RFP-expressing transgenic events. In this manner, PEI-precipitation can be used to deliver transient expression of BBM and/or WUS2.

For example, the particles are first coated with UBI::BBM::pinII using PEI, then coated with UBI::moPAT˜YFP using TFX-50, and then bombarded into scutellar cells on the surface of immature embryos. PEI-mediated precipitation results in a high frequency of transiently expressing cells on the surface of the immature embryo and extremely low frequencies of recovery of stable transformants (relative to the TFX-50 method). Thus, it is expected that the PEI-precipitated BBM cassette expresses transiently and stimulates a burst of embryogenic growth on the bombarded surface of the tissue (i.e. the scutellar surface), but this plasmid will not integrate. The PAT˜GFP plasmid released from the Ca++/gold particles is expected to integrate and express the selectable marker at a frequency that results in substantially improved recovery of transgenic events. As a control treatment, PEI-precipitated particles containing a UBI::GUS::pinII (instead of BBM) are mixed with the PAT˜GFP/Ca++ particles. Immature embryos from both treatments are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS).

As an alternative method, the BBM plasmid is precipitated onto gold particles with PEI, and then introduced into scutellar cells on the surface of immature embryos, and subsequent transient expression of the BBM gene elicits a rapid proliferation of embryogenic growth. During this period of induced growth, the explants are treated with Agrobacterium using standard methods for maize (see Example 1), with T-DNA delivery into the cell introducing a transgenic expression cassette such as UBI::moPAT˜GFPm::pinII. After co-cultivation, explants are allowed to recover on normal culture medium, and then are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS).

It may be desirable to “kick start” callus growth by transiently expressing the BBM and/or WUS2 polynucleotide products. This can be done by delivering BBM and WUS2 5′-capped polyadenylated RNA, expression cassettes containing BBM and WUS2 DNA, or BBM and/or WUS2 proteins. All of these molecules can be delivered using a biolistics particle gun. For example 5′-capped polyadenylated BBM and/or WUS2 RNA can easily be made in vitro using Ambion's mMessage mMachine kit. RNA is co-delivered along with DNA containing a polynucleotide of interest and a marker used for selection/screening such as Ubi::moPAT˜GFPm::PinII. It is expected that the cells receiving the RNA will immediately begin dividing more rapidly and a large portion of these will have integrated the agronomic gene. These events can further be validated as being transgenic clonal colonies because they will also express the PAT˜GFP fusion protein (and thus will display green fluorescence under appropriate illumination). Plants regenerated from these embryos can then be screened for the presence of the polynucleotide of interest.

### Example 8

### Modifying the ARGOS8 Gene to Improve Drought Tolerance and Nitrogen Use Efficiency in Maize Plants

ARGOS is a negative regulator for ethylene responses in plants (WO 2013/066805 A1, published 10 May 2013). ARGOS proteins target the ethylene signal transduction pathway. When over-expressed in maize plants, ARGOS reduces plant sensitivity to ethylene and promotes organ growth, leading to increased drought tolerance (DRT) and improved nitrogen use efficiency (NUE) ((WO 2013/066805 A1, published 10 May 2013). To achieve optimal ethylene sensitivity, promoters have been tested for driving Zm-ARGOS8 over-expression in transgenic maize plants. Field trials showed that a maize promoter, Zm-GOS2 PRO:GOS2 INTRON (SEQ ID NO:460, U.S. Pat. No. 6,504,083 patent issued on Jan. 7, 2003; Zm-GOS2 is a maize homologous gene of rice GOS2. Rice GOS2 stands for Gene from Oryza Sativa 2), provided a favorable expression level and tissue coverage for Zm-ARGOS8 and the transgenic plants have a higher grain yield than non-transgenic controls under drought stress and low nitrogen conditions (WO 2013/066805 A1, published 10 May 2013). However, these transgenic plants contain two ARGOS8 genes, the endogenous gene and the transgene. ARGOS8 protein levels, therefore, are determined by these two genes. Because the endogenous ARGOS8 gene varies in sequence and the expression level among different inbred lines, the ARGOS8 protein level will be different when the transgene is integrated into different inbreds. Here we present a mutagenization (gene editing) method to modify the promoter region of the endogenous ARGOS8 gene to attain desired expression patterns and eliminate the need for a transgene.

The promoter Zm-GOS2 PRO:GOS2 INTRON (SEQ ID NO:460; U.S. Pat. No. 6,504,083 patent issued on Jan. 7, 2003) was inserted into the 5′-UTR of Zm-ARGOS8 (SEQ ID NO:462) by using a guideRNA/Cas9 system. The Zm-GOS2 PRO:GOS2 INTRON fragment also included a primer binding site (SEQ ID NO:459) at its 5′ end to facilitate event screening with PCR. We also substituted the native promoter of Zm-ARGOS8 (SEQ ID NO:461) with Zm-GOS2 PRO::GOS2 INTRON (SEQ ID NO:460). Resulted maize lines carry a new ARGOS8 allele whose expression levels and tissue specificity will differ from the native form. We expect that these lines will recapitulate the phenotype of increased drought tolerance and improved NUE as observed in the Zm-GOS2 PRO:Zm-ARGOS8 transgenic plants (WO 2013/066805 A1, published 10 May 2013). These maize lines are different from those conventional transgenic events: (1) there is only one ARGOS8 gene in the genome; (2) this modified version of Zm-ARGOS8 resides at its native locus; (3) the ARGOS8 protein level and the tissue specificity of gene expression are entirely controlled by the edited allele. The DNA reagents used during the mutagenization, such as guideRNA, Cas9endonuclease, transformation selection marker and other DNA fragments are not required for function of the newly generated ARGOS8 allele and can be eliminated from the genome by segregation through standard breeding methods. Because the promoter Zm-GOS2 PRO:GOS2 INTRON was copied from maize GOS2 gene (SEQ ID NO:464) and inserted into the ARGOS8 locus through homologous recombination, this ARGOS8 allele is indistinguishable from natural mutant alleles.

A. Insertion of Zea mays-GOS2 PRO:GOS2 INTRON into Maize-ARGOS 8 Promoter

To insert Zm-GOS2 PRO:GOS2 INTRON into the 5′-UTR of maize ARGOS8 gene, a guideRNA construct, gRNA1, was made using maize U6 promoter and terminator as described herein. The 5′-end of the guide RNA contained a 19-bp variable targeting domain targeting the genomic target sequence 1 (CTS1; SEQ ID NO; 451) in the 5′-UTR of Zm-ARGOS8 (FIG. 7). A polynucleotide modification template containing the Zm-GOS2 PRO:GOS2 INTRON that was flanked by two genomic DNA fragments (HR1 and HR2, 370 and 430-bp in length, respectively) derived from the upstream and downstream region of the CTS1 (FIG. 7). The gRNA1 construct, the polynucleotide modification template, a Cas9 cassette and transformation selection marker phosphomannose isomerase (PMI) were introduced into maize immature embryo cells by using a particle bombardment method. PMI-resistant calli were screened with PCR for Zm-GOS2 PRO:GOS2 INTRON insertion (FIGS. 8A and 8B). Multiple callus events were identified and plants were regenerated. The insertion events were confirmed by amplifying the Zm-ARGOS8 region in T0 plants with PCR (FIG. 8C) and sequencing the PCR products.

B. Replacement of Zm-ARGOS 8 Promoter with Zm-GOS2 PRO:GOS2 INTRON Promoter (Promoter Swap).

To substitute (replace) the native promoter of Zm-ARGOS8 with Zm-GOS2 PRO:GOS2 INTRON, a guide RNA construct, gRNA3, was made for targeting the genomic target site CTS3 (SEQ ID NO:453), located 710-bp upstream of the Zm-ARGOS8 start codon (FIG. 9). Another guide RNA, gRNA2, was designed to target the genomic target site CTS2 (SEQ ID NO:452) located in the 5′-UTR of Zm-ARGOSO8 (FIG. 9). The polynucleotide modification template contained a 400-bp genomic DNA fragment derived from the upstream region of CTS3, Zm-GOS2 PRO:GOS2 INTRON and a 360-bp genomic DNA fragment derived from the downstream region of CTS2 (FIG. 9). The gRNA3 and gRNA2, the Cas9 cassette, the polynucleotide modification template and the PMI selection marker were used to transform immature embryo cells. Multiple promoter swap (promoter replacement) events were identified by PCR screening of the PMI-resistance calli and plants were regenerated. The swap events were confirmed by PCR analysis of the Zm-ARGOS8 region in T0 plants (FIG. 10D).

### C. Deletion of Zm-ARGOS 8 Promoter

To delete the promoter of Zm-ARGOS8, we screened the PMI-resistance calli obtained from the above gRNA3/gRNA2 experiment to look for events that produce a 1.1-kb PCR product (FIG. 11A). Multiple deletion events were identified (FIG. 11 B) and plants were regenerated. The deletion events were confirmed by amplifying the Zm-ARGOS8 region in T0 plants with PCR and sequencing of the PCR products.

TABLE 2
Argos8 cas9 variants

| ARGOS8 | | |
|---|---|---|
| variants | Line | Nature of modification |
| ARGOS8-cm1 | CML-189 | GOS2 PRO insertion in 5′-UTR (CTS1) |
| ARGOS8-cm3 | CML-664 | GOS2 Promoter swap (CTS3 & CTS2) |
| ARGOS8-cm4 | CML-232 | GOS2 PRO swap (CTS3 & CTS2) and |
| | | GR2HT to allele conversion |
| ARGOS8-cm6 | CML-422 | GOS2 PRO insertion (CTS2) & GR2HT |
| | | to allele conversion |
| ARGOS8-cm6 | CML-527 | GOS2 PRO insertion (CTS2) & GR2HT |
| | | to allele conversion |

Native ARGOS8 gene does not express in leaves. However GOS2 expression pattern includes leaves. ARGOS8 expression in heterozygous and homozygous plants were measured and homozygous variants showed higher gene expression than the corresponding heterozygous variant. ACC treatment enhances brace root emergence and growth in GR2HT wild-type (WT) plants. ACC-treated ARGOS8-cm1 homozygous plants produced fewer brace roots than WT, demonstrating reduced ethylene sensitivity.

### Example 9

### Enhancer Element Deletions Using the GuideRNA/Cas Endonuclease System

The guide RNA/Cas endonuclease system described herein can be used to allow for the deletion of a promoter element from either a transgenic (pre-existing, artificial) or endogenous gene. Promoter elements, such enhancer elements, or often introduced in promoters driving gene expression cassettes in multiple copies (3×=3 copies of enhancer element, FIG. 11) for trait gene testing or to produce transgenic plants expressing specific trait. Enhancer elements can be, but are not limited to, a 35S enhancer element (Benfey et al, EMBO J, August 1989; 8(8): 2195-2202, SEQ ID NO:513). In some plants (events), the enhancer elements can cause an unwanted phenotype, a yield drag, or a change in expression pattern of the trait of interest that is not desired. For example, as shown in FIG. 11, a plant comprising multiple enhancer elements (3 copies, 3×) in its genomic DNA located between two trait cassettes (Trait A and Trait B) was characterized to show an unwanted phenotype. It is desired to remove the extra copies of the enhancer element while keeping the trait gene cassettes intact at their integrated genomic location. The guide RNA/Cas endonuclease system described herein can be used to removing the unwanted enhancing element from the plant genome. A guide RNA can be designed to contain a variable targeting region targeting a target site sequence of 12-30 bps adjacent to a NGG (PAM) in the enhancer. If a Gas endonuclease target site sequence is present in all copies of the enhancer elements (such as the three Cas endonuclease target sites 35S-CRTS1 (SEQ ID NO:514), 35S-CRTS2 (SEQ ID NO:515), 35S-CRTS3 (SEQ ID NO:516)), only one guide RNA is needed to guide the Cas endonuclease to the target sites and induce a double strand break in all the enhancer elements at once. The Cas endonuclease can make cleavage to remove one or multiple enhancers. The guideRNA/Cas endonuclease system can introduced by either agrobacterium or particle gun bombardment. Alternatively, two different guide RNAs (targeting tow different genomic target sites) can be used to remove all 3×enhancer elements from the genome of an organism, in a manner similar to the removal of a (transgenic or endogenous) promoter described herein.

### Example 10

### Shortening Maturity Via Manipulation of Early Flowering Phenotype with ZmRap2.7 Down-Regulation Using the Guide RNA/Cas Endonuclease System

Overall plant maturity can be shortened by modulating the flowering time phenotype of plants through modulation of a maize ZmRap2.7 gene. Shortening of plant maturity can be obtained by an early flowering phenotype.

RAP2.7 is an acronym for Related to APETALA 2.7. RAPL means RAP2.7 LIKE and RAP2.7 functions as an AP2-family transcription factor that suppresses floral transition (SEQ ID NOs:520 and 521). Transgenic phenotype upon silencing or knock-down of Rap2.7 resulted in early flowering, reduced plant height, but surprisingly developed normal ear and tassel as compared the wild-type plants (PCT/US14/26279 application, filed Mar. 13, 2014). The guide RNA/Cas endonuclease system described herein can be used to target and induce a double strand break at a Cas endonuclease target site located within the RAP2.7 gene. Plants comprising NHEJ within the RAP2.7 gene can be selected and evaluated for the presence of a shortened maturity phenotype.

### Example 11

### Modulating Expression of a Maize NPK1B Gene for Engineering Frost Tolerance in Maize Using a Guide RNA/Cas Endonuclease System

Nicotiana Protein Kinase1 (NPK1) is a mitogen activated protein kinase kinase that is involved in cytokinesis regulation and oxidative stress signal transduction. The ZM-NPK1B (SEQ ID NO: 522 and SEQ ID NO: 523) which has about 70% amino acid similarity to rice NPKL3 has been tested for frost tolerance in maize seedlings and reproductive stages (PCT/US14/26279 application, filed Mar. 13, 2014). Transgenic seedlings and plants comprising a ZM-NPK1B driven by an inducible promoter Rab17, had significantly higher frost tolerance than control seedlings and control plants. The gene seemed inducted after cold acclimation and during −3° C. treatment period in most of the events but at low levels. (PCT/US14/26279 application, filed Mar. 13, 2014).

A guide RNA/Cas endonuclease system described herein can be used to replace the endogenous promoter of NPK1 gene, with a stress-inducible promoter such as the maize RAB17 promoter stages (SEQ ID NO: 524; PCT/US14/26279 application, filed Mar. 13, 2014), thus modulate NPK1B expression in a stress-responsive manner and provide frost tolerance to the modulated maize plants.

### Example 12

### Shortening Maturity Via Manipulation of Early Flowering Phenotype with FTM1 Expression Using a Guide RNA/Cas Endonuclease Systems

Overall plant maturity can shortened by modulating the flowering time phenotype of plants through expressing a transgene. Such a phenotype modification can also be achieved with additional transgenes or through a breeding approach.

FTM1 stands for Floral Transition MADS 1 transcription factor (SEQ ID NOs: 525 and 526). It is a MADS Box transcriptional factor and induces floral transition. Upon expression of FTM1 under a constitutive promoter, transgenic plants exhibited early flowering and shortened maturity, but surprisingly ear and tassel developed normally as compared to the wild-type plants (PCT/US14/26279 application, filed Mar. 13, 2014).

FTM1-expressing maize plants demonstrated that by manipulating a floral transition gene, time to flowering can be reduced significantly, leading to a shortened maturity for the plant. As maturity can be generally described as time from seeding to harvest, a shorter maturity is desired for ensuring that a crop can finish in the northern continental dry climatic environment (PCT/US14/26279 application, filed Mar. 13, 2014).

A guide RNA/Cas endonuclease system described herein can be used to introduce enhancer elements such as the CaMV35S enhancers (Benfey et al, EMBO J, August 1989; 8(8): 2195-2202, SEQ ID NO:512), specifically targeted in front of the endogenous promoter of FTM1, in order to enhance the expression of FTM1 while preserving most of the tissue and temporal specificities of native expression, providing shortened maturity to the modulated plants.

### Example 13

### Inserting Inducible Responsive Elements in Plant Genomes

Inducible expression systems controlled by an external stimulus are desirable for functional analysis of cellular proteins as well as trait development as changes in the expression level of the gene of interest can lead to an accompanying phenotype modification. Ideally such a system would not only mediate an “on/off” status for gene expression but would also permit limited expression of a gene at a defined level.

The guide RNA/Cas endonuclease system described herein can be used to introduce components of repressor/operator/inducer systems to regulate gene expression of an organism. Repressor/operator/inducer systems and their components are well known I the art (US 2003/0186281 published Oct. 2, 2003; U.S. Pat. No. 6,271,348). For example, nut not limited to, components of the tetracycline (Tc) resistance system of E. coli have been found to function in eukaryotic cells and have been used to regulate gene expression (U.S. Pat. No. 6,271,348). Nucleotide sequences of tet operators of different classes are known in the art see for example: classA, calssB, classC, classD, classE TET operator sequences listes as SEQ ID NOs:11-15 of U.S. Pat. No. 6,271,348.

Components of a sulfonylurea-responsive repressor system (as described in U.S. Pat. No. 8,257,956, issued on Sep. 4, 2012) can also be introduced into plant genomes to generate a pressor/operator/inducer systems into said plant where polypeptides can specifically bind to an operator, wherein the specific binding is regulated by a sulfonylurea compound.

### Example 14

### Engineering Drought Tolerance and Nitrogen Use Efficiency into Maize Via Gene Silencing by Expressing an Inverted Repeat into an ACS6 Gene Using the Guide RNA/Cas Endonuclease System

ACC (1-aminocyclopropane-1-carboxylic acid) synthase (ACS) genes encode enzymes that catalyze the rate limiting step in ethylene biosynthesis. A construct containing one of the maize ACS genes, ZM-ACS6, in an inverted repeat configuration, has been extensively tested for improved abiotic stress tolerance in maize (PCT/US2010/051358, filed Oct. 4, 2010; PCT/US2010/031008, filed Apr. 14, 2010). Multiple transgenic maize events containing a ZM-ACS6 RNAi sequence driven by a ubiquitin constitutive promoter had reduced ethylene emission, and a concomitant increase in grain yield relative to controls under both drought and low nitrogen field conditions (Plant Biotechnology Journal: 12 Mar. 2014, DOI: 10.1111/pbi.12172).

In one embodiment, the guide RNA/Cas endonuclease system can be used in combination with a co-delivered polynucleotide sequence to insert an inverted ZM-ACS6 gene fragment into the genome of maize, wherein the insertion of the inverted gene fragment allows for the in-vivo creation of an inverted repeat (hairpin) and results in the silencing of the endogenous ethylene biosynthesis gene.

In an embodiment the insertion of the inverted gene fragment can result in the formation of an in-vivo created inverted repeat (hairpin) in a native (or modified) promoter of an ACS6 gene and/or in a native 5′ end of the native ACS6 gene. The inverted gene fragment can further comprise an intron which can result in an enhanced silencing of the targeted ethylene biosynthetic gene.

### Example 15

### Modulating the Expression of Endogenous Serine Threonine Protein Phosphatase (STPP)

In an embodiment, expression level of an endogenous STPP present in plants is modulated. For example, as disclosed in US 20140259225 (incorporated herein by reference), a maize STPP is modulated by selectively affecting on or more regulatory elements present in the promoter region of the maize endogenous STPP. In an embodiment, the endogenous regulatory region driving the expression of a polynucleotide encoding a STPP3 polypeptide comprising SEQ ID NO: 1 of US20140259225 is edited by guided cas9 technology disclosed herein. In another embodiment, the endogenous regulatory region driving the expression of a polynucleotide encoding a STPP comprising a sequence selected from the group consisting of SEQ ID NOS: 1-8 of US20140259225 is edited by guided cas9 technology disclosed herein. Allelic differences in the promoter or other regulatory regions controlling the endogenous expression of STPP in maize or another target plant are within one of ordinary skill in the art to identify and design appropriate guide RNAs based on the teachings and guidance provided in the present disclosure and those available in the general genome editing literature.

In an embodiment, the native promoter element including the TATA box or an equivalent signature motif is replaced with another desirable promoter, e.g., a moderate constitutive promoter or a tissue preferred promoter in a promoter swap approach disclosed herein. In another embodiment, one or more enhancer elements are inserted upstream to the coding sequence of STPP. In an embodiment, the enhancer element is plant derived.

### Example 16

### Increasing Agronomic Characteristics of a Maize Plant by Altering Dominant Male Fertility

In an embodiment, plant yield is improved by modulating male fertility. For example, a mutation in a nucleotide sequence that reduces male fertility in a nuclear dominant fashion was disclosed in US 20150167013 (incorporated herein by reference).

In an embodiment, the reduction of male fertility or rendering the plant male sterile is effected by a single nucleotide substitution from G to an A at position 118 relative to the first Met codon of SEQ ID NO: 13 of US20150167013, resulting in an amino acid change at amino acid 37, from Alanine to Threonine in the protein encoded by the MS44 gene (MS44 polypeptide or MS44 protein), for example the dominant mutant allele represented by SEQ ID NO: 15 encoding SEQ ID NO: 14 of US20150167013. Single base change in the maize MS44 gene can result in a dominant male sterility phenotype. A codon change for a single amino acid at position 38 or 39 of the secretory signal cleavage site is able to generate the observed phenotype of reduced male fertility. Using Cas9 and guide RNA technology disclosed herein, such mutations and others can readily be introduced into a wild type plant. An exemplary gRNA target site, GCGCGCCGGACCCCAGCGCGG (SED ID NO: 551), about 70-bp downstream from these amino acid residues, can be used with Cas9 nuclease to introduce modified coding sequences, and recreate the dominant mutations needed for male sterility. Additional guide RNA sites exist surrounding these residues, such as GCCTCGTCTTGTGGGGGCTGG (SEQ ID NO: 552), about 115 bp upstream; or GCTTACAGCAGTTGGCTTGG (SEQ ID NO: 553), about 200 bp downstream. These sites can be used to engineer changes with Cas9 as small as a single base change in those residues. As an example, the codon for Alanine at position 38 can be changed to Valine (from GCG to ACG). In another example, Glutamic acid at position 39 can be changed to Proline (from CAG to CCG). Both of these changes can result in dominant sterility or a reduction in male fertility in a nuclear dominant manner. Similarly, other mutations that render dominant male sterility or reduction in male fertility can be incorporated into a plant genome using the guided RNA and cas9 technology provided herein. Other genome editing technologies such as zinc finger nucleases, TALENs, custom meganucleases and oligonucleobase approaches can also be used.

Allelic differences in the coding or other regulatory regions controlling the endogenous expression of any of the genes disclosed herein for maize or another target plant are within one of ordinary skill in the art to identify and design appropriate guide RNAs based on the teachings and guidance provided in the present disclosure and those available in the general genome editing literature.

### Example 17

### Modulating the Expression of a Drought Protein in Plants

In an embodiment, expression level of an endogenous XERICO gene present in plants is modulated to increase drought tolerance. For example, as disclosed in WO2013056000A1 (incorporated herein by reference), a maize XERICO gene is modulated by selectively affecting on or more regulatory elements present in the promoter region of the maize endogenous XERICO gene. In an embodiment, the endogenous regulatory region driving the expression of a polynucleotide encoding a XERICO polypeptide comprising a sequence selected from the group consisting of SEQ ID NO: 2 (ZmXERICOI), SEQ ID NO:m4 (ZmXERIC02), or SEQ ID NO: 6 (ZmXERICOIA), all SEQ IDs of WO2013056000A1, is edited by guided cas9 technology disclosed herein. In another embodiment, the endogenous regulatory region driving the expression of a polynucleotide encoding a XERICO protein is edited by guided cas9 technology disclosed herein to replace the endogenous promoter with a heterologous regulatory element, such as for example, GOS2 or a rice actin promoter element. Allelic differences in the promoter or other regulatory regions controlling the endogenous expression of XERICO in maize or another target plant are within one of ordinary skill in the art to identify and design appropriate guide RNAs based on the teachings and guidance provided in the present disclosure and those available in the general genome editing literature.

In an embodiment, the native promoter element including the TATA box or an equivalent signature motif is replaced with another desirable promoter, e.g., a moderate constitutive promoter or a tissue preferred promoter in a promoter swap approach disclosed herein. In another embodiment, one or more enhancer elements are inserted upstream to the coding sequence of XERICO. In an embodiment, the enhancer element is plant derived.

### Example 18

### Modulating the Expression of an Endogenous Gene while Maintaining the Native Expression Pattern

In an embodiment, affecting the endogenous gene expression of a native gene may not be beneficial, for example eliminating the expression of an endogenous expression pattern of a native gene. In certain instances, it may be desirable to maintain the endogenous expression pattern of the endogenous gene, while modulating the expression by providing additional or different expressions through a heterologous regulatory element. For example, a heterologous promoter sequence is inserted in an upstream region of the native gene that does not affect the endogenous expression pattern. In embodiment, such an insertion can be accomplished by providing a heterologous regulatory cassette that includes a promoter element and a terminator and inserted in the untranslated region of the native gene. In an embodiment, a new heterologous promoter element is included as part of a non-enhancing intron and with sufficient space between the new inserted promoter and the native promoter such that the expression pattern of the native promoter is substantially preserved and the inserted heterologous promoter provides additional expression pattern for the endogenous gene. In an embodiment, the heterologous promoter can be an inducible promoter.

1. A method of improving an agronomic trait of a plant, the method comprising providing a guide RNA that targets a polynucleotide involved in improving one or more agronomic characteristics of the plant in association with a Cas endonuclease that creates a double strand break at the polynucleotide and generating the plant, wherein the plant exhibits an improvement in the agronomic trait.

2. The method of claim 1, further comprising a donor polynucleotide that comprises one or more nucleotide changes as compared to a corresponding endogenous unmodified genomic DNA.

3. The method of claim 2, wherein the donor polynucleotide does not encode a full-length protein.

4. The method of claim 2, wherein the donor polynucleotide comprises a heterologous regulatory element.

5. The method of claim 4, wherein the regulatory element comprises a promoter.

6. The method of claim 4, wherein the regulatory element comprises an enhancer element.

7. The method of claim 6, wherein the enhancer element is plant derived.

8. The method of claim 1, wherein the polynucleotide is selected from the group consisting of a regulatory element, 5′-UTR, intron, exon, coding sequence, and a promoter.

9. The method of claim 4, wherein the heterologous regulatory element is from the same plant species as the polynucleotide involved in improving one or more agronomic characteristics of the plant.

10. The method of claim 1, wherein the guide RNA targets the polynucleotide selected from the group consisting of polynucleotide sequences involved in the expression of ZmArgos8, ZmACS6, ZmSRTF18, ZmXERICO1, trehalose 6 phosphate phosphatase (T6PP), and ZmSTPP3.

11. The method of claim 1, wherein the agronomic characteristics is selected from the group consisting of abiotic stress tolerance.

12. The method of claim 11, wherein the abiotic stress tolerance is drought or nutrient deficiency.

13. The method of claim 1, wherein the agronomic characteristic is an increase in yield or an increase in drought tolerance.

14. The method of claim 1, wherein Cas9 endonuclease creates the double strand break in a coding region of the polynucleotide.

15. The method of claim 1, where in the plant is selected from the group consisting of maize, soybean, rice, wheat, sorghum, brassica, sunflower, and camelina.

16. A method of improving grain yield of a maize plant, the method comprising providing a guide RNA that targets a polynucleotide involved in ethylene biosynthesis or ethylene signaling, the guide RNA acts in association with a Cas endonuclease that creates a double strand break at the polynucleotide and generating the plant, wherein the maize plant exhibits improved grain yield.

17. The method of claim 16, further comprising a donor polynucleotide that comprises one or more nucleotide changes as compared to a corresponding endogenous unmodified genomic DNA of the polynucleotide involved in ethylene biosynthesis or ethylene signaling.

18. The method of claim 16, wherein the polynucleotide is a maize ACC synthase.

19. The method of claim 16, wherein the polynucleotide is maize ARGOS.

20. The method of claim 18, wherein the expression of the maize ACC synthase is reduced as compared to a control maize plant.

21. The method of claim 19, wherein the expression of the maize ARGOS is increased as compared to a control maize plant.

22. The method of claim 21, wherein the expression of the maize ARGOS is increased by inserting a heterologous regulatory element.

23. The method of claim 22, wherein the heterologous regulatory element is a moderate constitutive promoter.

24. The method of claim 22, wherein the heterologous regulatory element is maize derived.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. The method of claim 16, wherein the polynucleotide sequence in the genome of a cell is selected from the group consisting of a promoter sequence, a terminator sequence, a regulatory element sequence, a splice site, a coding sequence, a polyubiquitination site, an intron site and an intron enhancing motif.

43. A method for editing a regulatory sequence involved in the expression of a gene for abiotic stress tolerance, in the genome of a cell, the method comprising introducing a guide polynucleotide, a polynucleotide modification template and at least one Cas endonuclease into a cell, wherein said guide RNA and Cas endonuclease are capable of forming a complex that enables the Cas endonuclease to introduce a double strand break at a target site in the genome of said cell, wherein said polynucleotide modification template comprises at least one nucleotide modification of said nucleotide sequence.

44. A method for replacing a first regulatory sequence modulating the expression of a gene involved in agronomic trait in a cell, the method comprising introducing a guide RNA, a polynucleotide modification template, and a Cas endonuclease into said cell, wherein said guide RNA and Cas endonuclease are capable of forming a complex that enables the Cas endonuclease to introduce a double strand break at a target site in the genome of said cell, wherein said polynucleotide modification template comprises a second promoter or second promoter fragment that is different from said first promoter sequence.

45. The method of claim 44, wherein the replacement of the first regulatory sequence results in any one of the following, or any one combination of the following: an increased promoter activity, an increased promoter tissue specificity, a decreased promoter activity, a decreased promoter tissue specificity, a new promoter activity, an inducible promoter activity, an extended window of gene expression, or a modification of the timing or developmental progress of gene expression in the same cell layer or other cell layer.

46. The method of claim 45, wherein the first regulatory sequence is selected from the group consisting of Zea mays ARGOS 8 promoter, maize NPK1 promoter, wherein the second promoter sequence is selected from the group consisting of a Zea mays GOS2 PRO:GOS2-intron promoter, a soybean ubiquitin promoter, a stress inducible maize RAB17 promoter, a Zea mays-PEPC1 promoter, a Zea mays Ubiquitin promoter, a Zea mays-Rootmet2 promoter, a rice actin promoter, a sorghum RCC3 promoter, a Zea mays-GOS2 promoter, a Zea mays-ACO2 promoter, and a Zea mays oleosin promoter.

47. (canceled)

48. (canceled)

49. (canceled)

50. (canceled)

51. (canceled)


SEQUENCE LISTING (554 sequences)

SEQ ID NO: 1 - DNA, 4107 bp, Streptococcus pyogenes M1 GAS (SF370)
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 2 - DNA, 189 bp, Solanum tuberosum
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcag

SEQ ID NO: 3 - protein, 9 aa, Simian virus 40
MAPKKKRKV

SEQ ID NO: 4 - protein, 18 aa, Agrobacterium tumefaciens
KRPRDRHDGELGGRKRAR

SEQ ID NO: 5 - DNA, 6717 bp, Artificial Sequence
  Maize optimized Cas9 expression cassette
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggcaccgaagaagaagcgcaaggtgatggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagacaagagaccacgggaccgccacgatggcgagctgggaggccgcaagcgggcaaggtaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcc

SEQ ID NO: 6 - RNA, 39 nt, Artificial Sequence
  crRNA containing the LIGCas-3 target sequence in the variable targeting domain
gcguacgcguacgugugguuuuagagcuaugcuguuuug

SEQ ID NO: 7 - RNA, 86 nt, Streptococcus pyogenes M1 GAS (SF370)
  tracRNA (1)..(86)
ggaaccauucaaaacagcauagcaaguuaaaauaaggcuaguccguuaucaacuugaaaaaguggcaccgagucggugcuuuuuuu

SEQ ID NO: 8 - RNA, 94 nt, Artificial Sequence
  Long guide RNA containing the LIGCas-3 target sequence in the variable targeting domain
gcguacgcguacgugugguuuuagagcuagaaauagcaaguuaaaauaaggcuaguccguuaucaacuugaaaaaguggcaccgagucggugcu

SEQ ID NO: 9 - DNA, 1000 bp, Zea mays
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 10 - DNA, 16 bp, Zea mays
tttttttttttttttt

SEQ ID NO: 11 - RNA, 59 nt, Artificial Sequence
  Short guide RNA containing the LIGCas-3 variable targeting domain
gcguacgcguacgugugguuuuagagcuagaaauagcaaguuaaaauaaggcuaguccg

SEQ ID NO: 12 - DNA, 1102 bp, Artificial Sequence
  Maize optimized long guide RNA expression cassette containing the LIGCas-3 variable targeting domain
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgcgtacgcgtacgtgtggttttagagctagaaatagcaagttaaaataaggctagtccgttatcaacttgaaaaagtggcaccgagtcggtgcttttttttt

SEQ ID NO: 13 - DNA, 27 bp, Zea mays
gtactccatccgccccatcgagtaggg

SEQ ID NO: 14 - DNA, 24 bp, Zea mays
gcacgtacgtcaccatcccgccgg

SEQ ID NO: 15 - DNA, 24 bp, Zea mays
gacgtacgtgccctactcgatggg

SEQ ID NO: 16 - DNA, 24 bp, Zea mays
gtaccgtacgtgccccggcggagg

SEQ ID NO: 17 - DNA, 24 bp, Zea mays
ggaattgtaccgtacgtgccccgg

SEQ ID NO: 18 - DNA, 20 bp, Zea mays
gcgtacgcgtacgtgtgagg

SEQ ID NO: 19 - DNA, 22 bp, Zea mays
gctggccgaggtcgactaccgg

SEQ ID NO: 20 - DNA, 23 bp, Zea mays
ggccgaggtcgactaccggccgg

SEQ ID NO: 21 - DNA, 23 bp, Zea mays
ggcgcgagctcgtgcttcaccgg

SEQ ID NO: 22 - DNA, 21 bp, Zea mays
ggtgccaatcatgcgtcgcgg

SEQ ID NO: 23 - DNA, 20 bp, Zea mays
ggtcgccatcacgggacagg

SEQ ID NO: 24 - DNA, 24 bp, Zea mays
gtcgcggcacctgtcccgtgatgg

SEQ ID NO: 25 - DNA, 23 bp, Zea mays
ggaatgctggaactgcaatgcgg

SEQ ID NO: 26 - DNA, 24 bp, Zea mays
gcagctcttcttggggaatgctgg

SEQ ID NO: 27 - DNA, 23 bp, Zea mays
gcagtaacagctgctgtcaatgg

SEQ ID NO: 28 - DNA, 56 bp, Artificial Sequence
  MS26Cas-1 forward primer
ctacactctttccctacacgacgctcttccgatctaggaccggaagctcgccgcgt

SEQ ID NO: 29 - DNA, 54 bp, Artificial Sequence
  MS26Cas-1 and MS26Cas-3 reverse primer
caagcagaagacggcatacgagctcttccgatcttcctggaggacgacgtgctg

SEQ ID NO: 30 - DNA, 59 bp, Artificial Sequence
  MS26Cas-2 forward primer
ctacactctttccctacacgacgctcttccgatctaaggtcctggaggacgacgtgctg

SEQ ID NO: 31 - DNA, 51 bp, Artificial Sequence
  MS26Cas-2 and MS26 meganuclease reverse primer
caagcagaagacggcatacgagctcttccgatctccggaagctcgccgcgt

SEQ ID NO: 32 - DNA, 56 bp, Artificial Sequence
  MS26Cas-3 forward primer
ctacactctttccctacacgacgctcttccgatcttcctccggaagctcgccgcgt

SEQ ID NO: 33 - DNA, 59 bp, Artificial Sequence
  MS26 Meganuclease forward primer
ctacactctttccctacacgacgctcttccgatctttcctcctggaggacgacgtgctg

SEQ ID NO: 34 - DNA, 63 bp, Artificial Sequence
  LIGCas-1 forward primer
ctacactctttccctacacgacgctcttccgatctaggactgtaacgatttacgcacctgctg

SEQ ID NO: 35 - DNA, 58 bp, Artificial Sequence
  LIGCas-1 and LIGCas-2 reverse primer
caagcagaagacggcatacgagctcttccgatctgcaaatgagtagcagcgcacgtat

SEQ ID NO: 36 - DNA, 63 bp, Artificial Sequence
  LIGCas-2 forward primer
ctacactctttccctacacgacgctcttccgatcttcctctgtaacgatttacgcacctgctg

SEQ ID NO: 37 - DNA, 60 bp, Artificial Sequence
  LIGCas-3 forward primer
ctacactctttccctacacgacgctcttccgatctaaggcgcaaatgagtagcagcgcac

SEQ ID NO: 38 - DNA, 57 bp, Artificial Sequence
  LIGCas-3 and LIG3-4 meganuclease reverse primer
caagcagaagacggcatacgagctcttccgatctcacctgctgggaattgtaccgta

SEQ ID NO: 39 - DNA, 60 bp, Artificial Sequence
  LIG3-4 meganuclease forward primer
ctacactctttccctacacgacgctcttccgatctccttcgcaaatgagtagcagcgcac

SEQ ID NO: 40 - DNA, 58 bp, Artificial Sequence
  MS45Cas-1 forward primer
ctacactctttccctacacgacgctcttccgatctaggaggacccgttcggcctcagt

SEQ ID NO: 41 - DNA, 54 bp, Artificial Sequence
  MS45Cas-1, MS45Cas-2 and MS45Cas-3 reverse primer
caagcagaagacggcatacgagctcttccgatctgccggctggcattgtctctg

SEQ ID NO: 42 - DNA, 58 bp, Artificial Sequence
  MS45Cas-2 forward primer
ctacactctttccctacacgacgctcttccgatcttcctggacccgttcggcctcagt

SEQ ID NO: 43 - DNA, 58 bp, Artificial Sequence
  MS45Cas-3 forward primer
ctacactctttccctacacgacgctcttccgatctgaagggacccgttcggcctcagt

SEQ ID NO: 44 - DNA, 58 bp, Artificial Sequence
  ALSCas-1 forward primer
ctacactctttccctacacgacgctcttccgatctaaggcgacgatgggcgtctcctg

SEQ ID NO: 45 - DNA, 53 bp, Artificial Sequence
  ALSCas-1, ALSCas-2 and ALSCas-3 reverse primer
caagcagaagacggcatacgagctcttccgatctgcgtctgcatcgccacctc

SEQ ID NO: 46 - DNA, 58 bp, Artificial Sequence
  ALSCas-2 forward primer
ctacactctttccctacacgacgctcttccgatctttcccgacgatgggcgtctcctg

SEQ ID NO: 47 - DNA, 58 bp, Artificial Sequence
  ALSCas-3 forward primer
ctacactctttccctacacgacgctcttccgatctggaacgacgatgggcgtctcctg

SEQ ID NO: 48 - DNA, 63 bp, Artificial Sequence
  EPSPSCas-1 forward primer
ctacactctttccctacacgacgctcttccgatctggaagaggaaacatacgttgcatttcca

SEQ ID NO: 49 - DNA, 57 bp, Artificial Sequence
  PSPSCas-1 and EPSPSCas-3 reverse primer
caagcagaagacggcatacgagctcttccgatctggtggaaagttcccagttgagga

SEQ ID NO: 50 - DNA, 62 bp, Artificial Sequence
  PSPSCas-2 forward primer
ctacactctttccctacacgacgctcttccgatctaagcggtggaaagttcccagttgagga

SEQ ID NO: 51 - DNA, 58 bp, Artificial Sequence
  EPSPSCas-2 reverse primer
caagcagaagacggcatacgagctcttccgatctgaggaaacatacgttgcatttcca

SEQ ID NO: 52 - DNA, 63 bp, Artificial Sequence
  EPSPSCas-3 forward primer
ctacactctttccctacacgacgctcttccgatctccttgaggaaacatacgttgcatttcca

SEQ ID NO: 53 - DNA, 43 bp, Artificial Sequence
  Forward primer for secondary PCR
aatgatacggcgaccaccgagatctacactctttccctacacg

SEQ ID NO: 54 - DNA, 18 bp, Artificial Sequence
  Reverse primer for secondary PCR
caagcagaagacggcata

SEQ ID NO: 55 - DNA, 93 bp, Zea mays
ctgtaacgatttacgcacctgctgggaattgtaccgtacgtgccccggcggaggatatatatacctcacacgtacgcgtacgcgtatatatac

SEQ ID NO: 56 - DNA, 98 bp, Artificial sequence
  Mutation 1 for LIGCas-1 locus
aggactgtaacgatttacgcacctgctgggaattgtaccgtacgtgccccggtcggaggatatatatacctcacacgtacgcgtacgcgtatatatac

SEQ ID NO: 57 - DNA, 98 bp, Artificial sequence
  Mutation 2 for LIGCas-1 locus
aggactgtaacgatttacgcacctgctgggaattgtaccgtacgtgccccggacggaggatatatatacctcacacgtacgcgtacgcgtatatatac

SEQ ID NO: 58 - DNA, 98 bp, Artificial sequence
  Mutation 3 for LIGCas-1 locus
aggactgtaacgatttacgcacctgctgggaattgtaccgtacgtgccccgggcggaggatatatatacctcacacgtacgcgtacgcgtatatatac

SEQ ID NO: 59 - DNA, 95 bp, Artificial sequence
  Mutation 4 for LIGCas-1 locus
aggactgtaacgatttacgcacctgctgggaattgtaccgtacgtgcggtcggaggatatatatacctcacacgtacgcgtacgcgtatatatac

SEQ ID NO: 60 - DNA, 98 bp, Artificial Sequence
  Mutation 5 for LIGCas-1 locus
aggactgtaacgatttacgcacctgctgggaattgtaccgtacgtgccccggccggaggatatatatacctcacacgtacgcgtacgcgtatatatac

SEQ ID NO: 61 - DNA, 96 bp, Artificial sequence
  Mutation 6 for LIGCas-1 locus
aggactgtaacgatttacgcacctgctgggaattgtaccgtacgtgccccgcggaggatatatatacctcacacgtacgcgtacgcgtatatatac

SEQ ID NO: 62 - DNA, 96 bp, Artificial Sequence
  Mutation 7 for LIGCas-1 locus
aggactgtaacgatttacgcacctgctgggaattgtaccgtacgtgccccggggaggatatatatacctcacacgtacgcgtacgcgtatatatac

SEQ ID NO: 63 - DNA, 94 bp, Artificial Sequence
  Mutation 8 for LIGCas-1 locus
aggactgtaacgatttacgcacctgctgggaattgtaccgtacgtgccccggaggatatatatacctcacacgtacgcgtacgcgtatatatac

SEQ ID NO: 64 - DNA, 94 bp, Artificial Sequence
  Mutation 8 for LIGCas-1 locus
aggactgtaacgatttacgcacctgctgggaattgtaccgtacgtgcgtcggaggatatatatacctcacacgtacgcgtacgcgtatatatac

SEQ ID NO: 65 - DNA, 43 bp, Artificial Sequence
  Mutation 10 for LIGCas-1 locus
aggactgtaacgatttacgcacctgctgggaattgtaccgtac

SEQ ID NO: 66 - DNA, 98 bp, Artificial Sequence
  Mutation 1 for LIGCas-2 locus
tcctctgtaacgatttacgcacctgctgggaattgtaccgtacgtgaccccggcggaggatatatatacctcacacgtacgcgtacgcgtatatatac

SEQ ID NO: 67 - DNA, 96 bp, Artificial Sequence
  Mutation 2 for LIGCas-2 locus
tcctctgtaacgatttacgcacctgctgggaattgtaccgtacgtccccggcggaggatatatatacctcacacgtacgcgtacgcgtatatatac

SEQ ID NO: 68 - DNA, 98 bp, Artificial Sequence
  Mutation 3 for LIGCas-2 locus
tcctctgtaacgatttacgcacctgctgggaattgtaccgtacgtgtccccggcggaggatatatatacctcacacgtacgcgtacgcgtatatatac

SEQ ID NO: 69 - DNA, 94 bp, Artificial Sequence
  Mutation 4 for LIGCas-2 locus
tcctctgtaacgatttacgcacctgctgggaattgtaccgtacccccggcggaggatatatatacctcacacgtacgcgtacgcgtatatatac

SEQ ID NO: 70 - DNA, 93 bp, Artificial Sequence
  Mutation 5 for LIGCas-2 locus
tcctctgtaacgatttacgcacctgctgggaattgtaccgtaccccggcggaggatatatatacctcacacgtacgcgtacgcgtatatatac

SEQ ID NO: 71 - DNA, 98 bp, Artificial Sequence
  Mutation 6 for LIGCas-2 locus
tcctctgtaacgatttacgcacctgctgggaattgtaccgtacgtggccccggcggaggatatatatacctcacacgtacgcgtacgcgtatatatac

SEQ ID NO: 72 - DNA, 92 bp, Artificial Sequence
  Mutation 7  for LIGCas-2 locus
tcctctgtaacgatttacgcacctgctgggaattgtaccgtacccggcggaggatatatatacctcacacgtacgcgtacgcgtatatatac

SEQ ID NO: 73 - DNA, 99 bp, Artificial Sequence
  Mutation 8 for LIGCas-2 locus
tcctctgtaacgatttacgcacctgctgggaattgtaccgtacgtgaaccccggcggaggatatatatacctcacacgtacgcgtacgcgtatatatac

SEQ ID NO: 74 - DNA, 61 bp, Artificial Sequence
  Mutation 9 for LIGCas-2 locus
tcctctgtaacgatttacgcacctgctgggaattgtaccgtacgtgtacgcgtatatatac

SEQ ID NO: 75 - DNA, 95 bp, Artificial Sequence
  Mutation 10 for LIGCas-2 locus
tcctctgtaacgatttacgcacctgctgggaattgtaccgtacgccccggcggaggatatatatacctcacacgtacgcgtacgcgtatatatac

SEQ ID NO: 76 - DNA, 93 bp, Zea mays
cgcaaatgagtagcagcgcacgtatatatacgcgtacgcgtacgtgtgaggtatatatatcctccgccggggcacgtacggtacaattcccag

SEQ ID NO: 77 - DNA, 98 bp, Artificial Sequence
  Mutation 1 for LIGCas-3 locus
aaggcgcaaatgagtagcagcgcacgtatatatacgcgtacgcgtacgttgtgaggtatatatatcctccgccggggcacgtacggtacaattcccag

SEQ ID NO: 78 - DNA, 96 bp, Artificial Sequence
  Mutation 2 for LIGCas-3 locus
aaggcgcaaatgagtagcagcgcacgtatatatacgcgtacgcgtacggtgaggtatatatatcctccgccggggcacgtacggtacaattcccag

SEQ ID NO: 79 - DNA, 96 bp, Artificial Sequence
  Mutation 3 for LIGCas-3 locus
aaggcgcaaatgagtagcagcgcacgtatatatacgcgtacgcgtactgtgaggtatatatatcctccgccggggcacgtacggtacaattcccag

SEQ ID NO: 80 - DNA, 95 bp, Artificial Sequence
  Mutation 4 for LIGCas-3 locus
aaggcgcaaatgagtagcagcgcacgtatatatacgcgtacgcgtacgtgaggtatatatatcctccgccggggcacgtacggtacaattcccag

SEQ ID NO: 81 - DNA, 68 bp, Artificial Sequence
  Mutation 5 for LIGCas-3 locus
aaggcgcaaatgagtagcagcgcacgtatatatatcctccgccggggcacgtacggtacaattcccag

SEQ ID NO: 82 - DNA, 55 bp, Artificial Sequence
  Mutation 6 for LIGCas-3 locus
aaggcgcaaatgagtagcagcgcacgtatatatacgcgtacggtacaattcccag

SEQ ID NO: 83 - DNA, 93 bp, Artificial Sequence
  Mutation 7 for LIGCas-3 locus
aaggcgcaaatgagtagcagcgcacgtatatatacgcgtacgcgtgtgaggtatatatatcctccgccggggcacgtacggtacaattcccag

SEQ ID NO: 84 - DNA, 69 bp, Artificial Sequence
  Mutation 8 for LIGCas-3 locus
aaggcgcaaatgagtagcagcgcacgtatatatacgcgtacgccggggcacgtacggtacaattcccag

SEQ ID NO: 85 - DNA, 66 bp, Artificial Sequence
  Mutation 9 for LIGCas-3 locus
aaggcgcaaatgagtagcagcgcacgtatatatcctccgccggggcacgtacggtacaattcccag

SEQ ID NO: 86 - DNA, 95 bp, Artificial Sequence
  Mutation 10 for LIGCas-3 locus
aaggcgcaaatgagtagcagcgcacgtatatatacgcgtacgcgtatgtgaggtatatatatcctccgccggggcacgtacggtacaattcccag

SEQ ID NO: 87 - DNA, 95 bp, Artificial Sequence
  Mutation 1 for LIG3-4 homing endonuclease locus
ccttcgcaaatgagtagcagcgcacgtatatatacgcgtacgcgtacgtgaggtatatatatcctccgccggggcacgtacggtacaattcccag

SEQ ID NO: 88 - DNA, 68 bp, Artificial Sequence
  Mutation 2 for LIG3-4 homing endonuclease locus
ccttcgcaaatgagtagcagcgcacgtatatatatcctccgccggggcacgtacggtacaattcccag

SEQ ID NO: 89 - DNA, 65 bp, Artificial Sequence
  Mutation 3 for LIG3-4 homing endonuclease locus
ccttcgcaaatgagtagcagcgcacgtatatatacgcgtacgcgtacgtacggtacaattcccag

SEQ ID NO: 90 - DNA, 55 bp, Artificial Sequence
  Mutation 4 for LIG3-4 homing endonuclease locus
ccttcgcaaatgagtagcagcgcacgtatatatacgcgtacggtacaattcccag

SEQ ID NO: 91 - DNA, 69 bp, Artificial Sequence
  Mutation 5 for LIG3-4 homing endonuclease locus
ccttcgcaaatgagtagcagcgcacgtatatatacgcgtacgccggggcacgtacggtacaattcccag

SEQ ID NO: 92 - DNA, 85 bp, Artificial Sequence
  Mutation 6 for LIG3-4 homing endonuclease locus
ccttcgcaaatgagtagcagcgcacgtatatatacgtgtgaggtatatatatcctccgccggggcacgtacggtacaattcccag

SEQ ID NO: 93 - DNA, 93 bp, Artificial Sequence
  Mutation 7 for LIG3-4 homing endonuclease locus
ccttcgcaaatgagtagcagcgcacgtatatatacgcgtacgcgtgtgaggtatatatatcctccgccggggcacgtacggtacaattcccag

SEQ ID NO: 94 - DNA, 66 bp, Artificial Sequence
  Mutation 8 for LIG3-4 homing endonuclease locus
ccttcgcaaatgagtagcagcgcacgtatatatcctccgccggggcacgtacggtacaattcccag

SEQ ID NO: 95 - DNA, 95 bp, Artificial Sequence
  Mutation 9 for LIG3-4 homing endonuclease locus
ccttcgcaaatgagtagcagcgcacgtatatatacgcgtacgcgtacgtgtggtatatatatcctccgccggggcacgtacggtacaattcccag

SEQ ID NO: 96 - DNA, 102 bp, Artificial Sequence
  Mutation 10 for LIG3-4 homing endonuclease locus
ccttcgcaaatgagtagcagcgcacgtatatatacgcgtacggtatatatacgtgtgaggtatatatatcctccgccggggcacgtacggtacaattcccag

SEQ ID NO: 97 - DNA, 5424 bp, Artificial Sequence
  donor DNA -HR Repair DNA
cccatagaaaactgtgtgctataatacaccaaaaggaaagcaaagtgaaaaggaaactttgaatagccaagaagactcggagtgcttcacgccttcacctatcccacataggtgatgagctaagagtaaaatgtagattctctcgagtactgaatattgcctgcacttttccttgcagtaaatacacctttaatccatgacgagagtccactctttgagtccgtcttgagattcttccattgatcatacaacatgacctcgaagtcctgatggagaacaacttatataattaaaactacaatacagaaagttcctgacaattaaaacctttggtggtggcatgccgtaggttaaaaaaaatagataatgacaacacaactggagacacgctctttgccgagtgctcacacgtttgctgagagcgagcactcggcaaatatatgatttgccgaataccaccctcctcggcaaaacaatacactaggcaaaaaggtagtttcccatcaccatgatgcccgccgttaatgtaccttctatgccgagtatgttggcgctcagcaaagagatcgttaccggcgtttgtttcaccaagagctctttgacgagtgtggcacacgacaaaaccttttgccgagtgtaattagtcgtttgccaagtgactggtgcagttggcaaaggagtcgtttattatgtgtgggcaaaatgatatatggtgccagttagggctagcaaattaaaggggggggggggggggttaggttgaagaaggtgacgagtaataaggtctcggacggccgcgcgcatatatatcagatccgatccaatggcacacggtgcaaacgaaaagcacgaaatttccaccagcttaattagggagagaaaaatagagcaccagctgatgagtgaatgaatgagatagacgggacacagagggtccagcaggctagcctactctggccgccctaaatagaagtcagtgccgtgacgacgcgcaaacttcttttgatcggctgcggaaataatatactgtaacgatttacgcacctgctgggaattgtaccgtacgtgccccggcggaggatatatatacctcacacaagggcgaattgtactagttagttagctagtcggtcctagatgccgtaatcattagctaatcgtaagtgacgcttggacacgagcggcttgagctaggaacctacgaagtcatcggaatcagctcaggtgtacagaagttcctatactttctggagaataggaacttcggaataggaacttcgtatacgctagggccgcattcgcaaaacacacctagactagatttgttttgctaacccaattgatattaattatatatgattaatatttatatgtatatggatttggttaatgaaatgcatctggttcatcaaagaattataaagacacgtgacattcatttaggataagaaatatggatgatctctttctcttttattcagataactagtaattacacataacacacaactttgatgcccacattatagtgattagcatgtcactatgtgtgcatccttttatttcatacattaattaagttggccaatccagaagatggacaagtctaggtttcgactcagatctgcgtcaccgggcgcaccgggcgcggcggggccggcagctcgaagtcgcgctgccagaagccgacgtcgtgccagccgccgtgcttgtagccggcggcgcggagggtgccgcgggcggtgtagccgagggcctcgtggaggcgcacggacgggtcgttcgggaggccgatcacggccaccacggacttgaagccctgggcctccatgctcttgaggaggtgggtgtagagggtggagccgaggccgaggcgctggtggcggtgggacacgtacacggtggactccacggtccagtcgtaggcgttgcgggccttccacgggccggcgtaggcgatgccggccaccacgccctccacctcggccacgagccacgggtagcggtcctggaggcgctccaggtcgtcgatccactcctgcggggtctgcggctcggtgcggaagttcacggtggaggtctcgatgtagtggttcacgatgtcgcacacggcggccatgtcggcggcggtggccgggcggatctcgacggggcggcgctcgggggacatggtgtcgtgtggatcccggtggatctgaagttcctatactttctagagaataggaacttcggaataggaacttcgctagcgaattgatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagaggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggctagcggcgaattcgcccttgtacgcgtacgcgtatatatacgtgcgctgctactcatttgcgcgggaatacagctcagtctgctgtgcgctgcaggatgtacatacatacatgcgcaggtgcaaagtctacgcgcgcgggcaatgcaagcccctggcgtagttgggccatgactgagatcacgcctcatggtcatggaacgaaacaccgcgtccggccgggctgcccctggcgtcacgcgggaggcagctgctagcgttagcgtacgtacccaccgtctcgtacacaccaccgcagggagagagaagagcgatgcaatgcacatgtacagcatccgcatcatgcatagatactcatatcttcaaggccacacatgcagcagtgtcgtacgctacgttgtttcaacggaggaggaggatacatacatagacacccacagccagcctagcatatagcagatagcatacggactcccgggtgaggaaaaatggagggcgaaccaaaccaaccacaaagaagcagcagcagcagcagcagcagctgcggctgctatcaccactcaccaactccaattaaagatctctctctctctctctactggccggccctgtcagtgccagcgcccggtttgttgctagctgagctgcgggcgtcgctcttagatatagcccaaaactcactccaccaccactcgttccatggaaccctagaccaaaagtactcgcgctctcggccctcgctctcgccctctccctctccgcagcaaaagagatccggccggccgagaagggcgcgcgctagctgcccggctactagctggcgcccgcccgcgcatatatctgtgtcatcgccatcacccacaccatggcccggccggccaacaccgccgtattagctctgtctgtcgctcgtccacctgcgaccgactgagcgatcgatctccaccgagctctccgctaagcgctgtccttgccgccgtcctcccctccgtcccctacgcatccatttccgtgtgctcgtgtgtgcgcgcgcgggcactcctgctcctgctccctccggcccctcctcccctcccaggctcccagctagccgcgcccgcccgcgcgacctgcacctgcacagatcgggcggccgggccgaccgatcgatcgagat

SEQ ID NO: 98 - DNA, 24 bp, Artificial Sequence
  Forward PCR primer
cccgttattgtatgaggtaatgac

SEQ ID NO: 99 - DNA, 31 bp, Artificial Sequence
  Reverse PCR primer for site-specific transgene insertion at junction 1
gctcgtgtccaagcgtcacttacgattagct

SEQ ID NO: 100 - DNA, 32 bp, Artificial Sequence
  Forward PCR primer for site-specific transgene insertion at junction 2
ccatgtctaactgttcatttatatgattctct

SEQ ID NO: 101 - DNA, 24 bp, Artificial Sequence
  Reverse PCR primer for site-specific transgene insertion at junction 2
gcagccgataggttcatcatcttc

SEQ ID NO: 102 - DNA, 7850 bp, Artificial Sequence
  Linked Cas9 and LIGCas-3 long guide RNA expression cassettes
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggcaccgaagaagaagcgcaaggtgatggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagacaagagaccacgggaccgccacgatggcgagctgggaggccgcaagcgggcaaggtaggtaccgttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccccccctcgaggtcgacggtatcgataagctttgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgcgtacgcgtacgtgtggttttagagctagaaatagcaagttaaaataaggctagtccgttatcaacttgaaaaagtggcaccgagtcggtgcttttttttt

SEQ ID NO: 103 - DNA, 23 bp, Zea mays
tgggcaggtctcacgacggttgg

SEQ ID NO: 104 - DNA, 91 bp, Zea mays
ccggtttcgcgtgctctggctttacattacatgggcaggtctcacgacggttgggctggagagccggctggtaggggaggacctcaacggc

SEQ ID NO: 105 - DNA, 90 bp, Artificial Sequence
  Mutation 1 for 55CasRNA-1 locus
ccggtttcgcgtgctctggctttacattacatgggcaggtctcacgaggttgggctggagagccggctggtaggggaggacctcaacggc

SEQ ID NO: 106 - DNA, 90 bp, Artificial Sequence
  Mutation 2 for 55CasRNA-1 locus
ccggtttcgcgtgctctggctttacattacatgggcaggtctcacacggttgggctggagagccggctggtaggggaggacctcaacggc

SEQ ID NO: 107 - DNA, 92 bp, Artificial Sequence
  Mutation 3 for 55CasRNA-1 locus
ccggtttcgcgtgctctggctttacattacatgggcaggtctcacgacggtttgggctggagagccggctggtaggggaggacctcaacggc

SEQ ID NO: 108 - DNA, 89 bp, Artificial Sequence
  Mutation 4 for 55CasRNA-1 locus
ccggtttcgcgtgctctggctttacattgcatgagcaggtcgtgacggttgggctggagagccggctggtaggggaggacctcaacggc

SEQ ID NO: 109 - DNA, 57 bp, Artificial Sequence
  Mutation 5 for 55CasRNA-1 locus
gggcaggtctcgacggttgggctggagagccggctggtaggggaggacctcaacggc

SEQ ID NO: 110 - DNA, 57 bp, Artificial Sequence
  Mutation 6 for 55CasRNA-1 locus
ccggtttcgcgtgctcttgggctggagagccggctggtaggggaggacctcaacggc

SEQ ID NO: 111 - DNA, 22 bp, Zea mays
atatacctcacacgtacgcgta

SEQ ID NO: 112 - DNA, 1053 bp, Zea mays
atgaacaccaagtacaacaaggagttcctgctctacctggccggcttcgtggacggcgacggctccatcaaggcgcagatcaagccgaaccagtcctgcaagttcaagcaccagctctccctgaccttccaggtgacccagaagacgcagaggcgctggttcctcgacaagctggtcgacgagatcggggtgggctacgtctacgaccgcgggtcggtgtccgactacgagctctcccagatcaagcccctgcacaacttcctcacccagctccagccgttcctcaagctgaagcagaagcaggcgaacctcgtcctgaagatcatcgagcagctcccctcggccaaggagtccccggacaagttcctggaggtgtgcacgtgggtcgaccagatcgcggccctcaacgacagcaagacccgcaagacgacctcggagacggtgcgggcggtcctggactccctcccaggatccgtgggaggtctatcgccatctcaggcatccagcgccgcatcctcggcttcctcaagcccgggttcagggatctccgaagcactcagagctggagcaactaagtccaaggaattcctgctctacctggccggcttcgtggacggcgacggctccatcatcgcgtccatcaagccgcgccagtgctacaagttcaagcacgagctccgcctggagttcaccgtgacccagaagacgcagaggcgctggttcctcgacaagctggtcgacgagatcggggtgggctacgtctacgaccgcgggtcggtgtccgactaccgcctctcccagatcaagcccctgcacaacttcctcacccagctccagccgttcctcaagctgaagcagaagcaggcgaacctcgtcctgaagatcatcgagcagctcccctcggccaaggagtccccggacaagttcctggaggtgtgcacgtgggtcgaccagatcgcggccctcaacgacagcaagacccgcaagacgacctcggagacggtgcgggcggtcctggactccctcagcgagaagaagaagtcgtccccctga

SEQ ID NO: 113 - DNA, 22 bp, Zea mays
gatggtgacgtacgtgccctac

SEQ ID NO: 114 - DNA, 1053 bp, Zea mays
atgaacaccaagtacaacaaggagttcctcctctacctggcaggtttcgtggacggcgatgggtctatcatcgcccagattaccccgcaacagtcctacaagttcaagcacgccctgcggctgaggttcacggtcactcagaagacgcagcgcaggtggttcctcgataagctggtcgacgaaatcggagtcggcaaggtgcgggacaggggctctgtcagcgactacatcctctcccagaagaagccgctccacaacttcctgacccagctgcagcccttcctcaagctcaagcagaagcaggccaacctggtgctcaagatcatcgagcagctgccatctgccaaggagtcaccagacaagttccttgaggtctgcacctgggtcgatcagatcgctgccctgaacgactccaagacgaggaagaccacctccgagaccgtcagggctgtgctggactcactcccaggatccgttggcggtctcagcccttctcaggctagctcggctgcttcctcagccagcagctcacctggctccggtatcagcgaggctctcagagcaggtgccaccaagtccaaggagttcctcctgtacctggcaggcttcgttgacggcgacggctcgatcatggcgtccattaccccgaaccagtcgtgtaagttcaagcatcagctgcgcctgcgctttaccgtcacgcagaagacccagaggcgctggttcctggacaaactggtggacgagatcggggtcgggaaggtgtacgacagagggagcgttagcgactaccggctgtcccagaagaagccgctccacaacttcctgacgcagctccaacccttcctgaagctgaagcagaagcaggcgaaccttgtgctgaagatcattgagcagctgccgagcgccaaggagagccctgacaagttcctggaggtctgcacctgggtcgaccagatcgctgccctcaacgactccaagaccaggaagaccacgagcgagaccgttcgggctgtcctggacagcctctccgagaagaagaagtcgagcccgtag

SEQ ID NO: 115 - DNA, 4104 bp, Artificial sequence
  soybean codon optimized Cas9
atggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgac

SEQ ID NO: 116 - DNA, 1503 bp, Glycine max
ccgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatc

SEQ ID NO: 117 - DNA, 33 bp, Artificial sequence
  linker SV40 NLS
tctagagccgatcccaagaagaagagaaaggtg

SEQ ID NO: 118 - protein, 1379 aa, Artificial sequence
  Cas9 with a SV40 NLS
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 119 - DNA, 8519 bp, Artificial sequence
  QC782
ccgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgctcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccggtac

SEQ ID NO: 120 - DNA, 434 bp, Glycine max
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaa

SEQ ID NO: 121 - DNA, 104 bp, Artificial sequence
  Guide RNA for DD43CR1
gtcccttgtacttgtacgtagttttagagctagaaatagcaagttaaaataaggctagtccgttatcaacttgaaaaagtggcaccgagtcggtgctttttttt

SEQ ID NO: 122 - DNA, 3098 bp, Artificial sequence
  QC783
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttgtcccttgtacttgtacgtagttttagagctagaaatagcaagttaaaataaggctagtccgttatcaacttgaaaaagtggcaccgagtcggtgcttttttttgcggccgctcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccggtac

SEQ ID NO: 123 - DNA, 9093 bp, Artificial sequence
  QC815
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttgtcccttgtacttgtacgtagttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccggtac

SEQ ID NO: 124 - DNA, 4107 bp, S. pyogenes
atggataagaaatactcaataggcttagatatcggcacaaatagcgtcggatgggcggtgatcactgatgattataaggttccgtctaaaaagctcaagggtctgggaaatacagaccgccacggtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagtggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtagagtagatgctaaagcgattctttctgcacgattgagtaaatcaagacgattagaaaatctcattgctcagctccccggtgagaagaaaaatggattgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctactttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggcgaaactaaatcgtgaagatttgctgcgcaagcaacggacctttgacaacggctctattccctatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaagtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaacgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgtgagcgtatgaaacgtattgaagaaggaataaaagaactaggaagtgatattctaaaggagtatcctgttgaaaacactcaattacaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacaaaagctgaacgtggaggtttgagtgaacttgataaagttggttttatcaaacgccaattggttgaaactcgccaaatcactaagcatgtggcacaaattttggatagtcgcatgaatactaaatacgatgaaaatgataaacttattcgagaggttagagtgattaccttaaaatctaaattagtttctgacttccgaaaagatttccaattctataaagtacgtgagattaacaattaccatcatgcccatgatgcgtatcttaatgccgtcgttggaactgctttgattaagaaatatccaaaacttgaatcggagtttgtctatggtgattataaagtttatgatgttcgtaaaatgattgctaagtctgagcaggaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaataatggaaagaagctcttttgaaaaagatccgattgactttttagaagctaaaggatataaggaagttagaaaagacttaatcattaaactacctaaatatagtctttttgagttagaaaacggtcgtaaacggatgctggctagtgccggagaattgcaaaaaggaaatgagctagctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 125 - DNA, 20 bp, Glycine max
ggaactgacacacgacatga

SEQ ID NO: 126 - DNA, 20 bp, Glycine max
gacatgatggaacgtgacta

SEQ ID NO: 127 - DNA, 20 bp, Glycine max
gtcccttgtacttgtacgta

SEQ ID NO: 128 - DNA, 20 bp, Glycine max
gtattctagaaaagaggaat

SEQ ID NO: 129 - DNA, 70 bp, Glycine max
atcaaaattcggaactgacacacgacatgatggaacgtgactaaggtgggtttttgactttgcatgtcga

SEQ ID NO: 130 - DNA, 70 bp, Glycine max
tcgacatgcaaagtcaaaaacccaccttagtcacgttccatcatgtcgtgtgtcagttccgaattttgat

SEQ ID NO: 131 - DNA, 70 bp, Glycine max
ggcagactccaattcctcttttctagaataccctccgtacgtacaagtacaagggacttgtgagttgtaa

SEQ ID NO: 132 - DNA, 70 bp, Glycine max
ttacaactcacaagtcccttgtacttgtacgtacggagggtattctagaaaagaggaattggagtctgcc

SEQ ID NO: 133 - DNA, 71 bp, Glycine max
ctacactctttccctacacgacgctcttccgatctggaatttacagcacaagtagatcacttgtacttatc

SEQ ID NO: 134 - DNA, 59 bp, Glycine max
caagcagaagacggcatacgagctcttccgatctaaatcactctcacttcgacatgcaa

SEQ ID NO: 135 - DNA, 71 bp, Glycine max
ctacactctttccctacacgacgctcttccgatctttcctttacagcacaagtagatcacttgtacttatc

SEQ ID NO: 136 - DNA, 68 bp, Glycine max
ctacactctttccctacacgacgctcttccgatctagctgtaaatacagccttacaactcacaagtcc

SEQ ID NO: 137 - DNA, 63 bp, Artificial sequence
  Primer, DD43-A
caagcagaagacggcatacgagctcttccgatctttaatttaggactaaaagaagaggcagac

SEQ ID NO: 138 - DNA, 68 bp, Artificial sequence
  Primer, DD43-S4
ctacactctttccctacacgacgctcttccgatctctaggtaaatacagccttacaactcacaagtcc

SEQ ID NO: 139 - DNA, 68 bp, Artificial sequence
  Primer, DD43-S5
ctacactctttccctacacgacgctcttccgatctgatcgtaaatacagccttacaactcacaagtcc

SEQ ID NO: 140 - DNA, 43 bp, Artificial sequence; identical to SEQ ID NO: 53
  Primer, JKY557

SEQ ID NO: 141 - DNA, 18 bp, Artificial sequence
  primer, JKY558
caagcagaagacggcata

SEQ ID NO: 142 - DNA, 117 bp, Artificial sequence
  DD20CR1 PCR amplicon
ggaatttacagcacaagtagatcacttgtacttatcaaaattcggaactgacacacgacatgatggaacgtgactaaggtgggtttttgactttgcatgtcgaagtgagagtgattt

SEQ ID NO: 143 - DNA, 117 bp, Artificial sequence
  DD20CR2 PCR amplicon
ttcctttacagcacaagtagatcacttgtacttatcaaaattcggaactgacacacgacatgatggaacgtgactaaggtgggtttttgactttgcatgtcgaagtgagagtgattt

SEQ ID NO: 144 - DNA, 108 bp, Artificial sequence
  DD43CR1 PCR amplicon
agctgtaaatacagccttacaactcacaagtcccttgtacttgtacgtacggagggtattctagaaaagaggaattggagtctgcctcttcttttagtcctaaattaa

SEQ ID NO: 145 - DNA, 108 bp, Artificial sequence
  DD43CR2 PCR amplicon
ctaggtaaatacagccttacaactcacaagtcccttgtacttgtacgtacggagggtattctagaaaagaggaattggagtctgcctcttcttttagtcctaaattaa

SEQ ID NO: 146 - DNA, 108 bp, artificial sequence; identical to SEQ ID NO: 145
  amplicon

SEQ ID NO: 147 - DNA, 101 bp, Artificial sequence
  DD20CR1 mutant target site
ggaatttacagcacaagtagatcacttgtacttatcaaaattcggaactgacacacgatgatggaacgtgactaaggtgggtttttgactttgcatgtcga

SEQ ID NO: 148 - DNA, 101 bp, Artificial sequence
  DD20CR1 mutant target site
ggaatttacagcacaagtagatcacttgtacttatcaaaattcggaactgacacacgatggaacgtgactaaggtgggtttttgactttgcatgtcgaagt

SEQ ID NO: 149 - DNA, 101 bp, Artificial sequence
  DD20CR1 mutant target site
ggaatttacagcacaagtagatcacttgtacttatcaaaattcggaactgacacacgactgatggaacgtgactaaggtgggtttttgactttgcatgtcg

SEQ ID NO: 150 - DNA, 101 bp, Artificial sequence
  DD20CR1 mutant target site
ggaatttacagcacaagtagatcacttgtacttatcaaaattcggaactgacacacatggaacgtgactaaggtgggtttttgactttgcatgtcgaagtg

SEQ ID NO: 151 - DNA, 101 bp, Artificial sequence
  DD20CR1 mutant target site
ggaatttacagcacaagtagatcacttgtacttatcaaaattcggaactgacacatgatggaacgtgactaaggtgggtttttgactttgcatgtcgaagt

SEQ ID NO: 152 - DNA, 101 bp, Artificial sequence
  DD20CR1 mutant target site
ggaatttacagcacaagtagatcacttgtacttatcaaaattcggaactgacacagacatgatggaacgtgactaaggtgggtttttgactttgcatgtcg

SEQ ID NO: 153 - DNA, 101 bp, Artificial sequence
  DD20CR1 mutant target site
ggaatttacagcacaagtagatcacttgtacttatcaaaattcggaactgacacgacatgatggaacgtgactaaggtgggtttttgactttgcatgtcga

SEQ ID NO: 154 - DNA, 101 bp, Artificial sequence
  DD20CR1 mutant target site i
ggaatttacagcacaagtagatcacttgtacttatcaaaattcggaactgacacaagaaatgatggaacgtgactaaggtgggtttttgactttgcatgtc

SEQ ID NO: 155 - DNA, 101 bp, Artificial sequence
  DD20CR1 mutant target site
ggaatttacagcacaagtagatcacttgtacttatcaaaattcggaactgacacacgattgaacgtgactaaggtgggtttttgactttgcatgtcgaagt

SEQ ID NO: 156 - DNA, 101 bp, Artificial sequence
  DD20CR1 mutant target site
ggaatttacagcacaagtagatcacttgtacttatcaaaattcggaactgacacacattgaacgtgactaaggtgggtttttgactttgcatgtcgaagtg

SEQ ID NO: 157 - DNA, 101 bp, Artificial sequence
  DD20CR2 mutant target site
ttcctttacagcacaagtagatcacttgtacttatcaaaattcggaactgacacacgacatgatggaacgtctaaggtgggtttttgactttgcatgtcga

SEQ ID NO: 158 - DNA, 101 bp, Artificial sequence
  DD20CR2 mutant target site
ttcctttacagcacaagtagatcacttgtacttatcaaaattcggaactgacacacgacatgatggaacctaaggtgggtttttgactttgcatgtcgaag

SEQ ID NO: 159 - DNA, 101 bp, Artificial sequence
  DD20CR2 mutant target site
ttcctttacagcacaagtagatcacttgtacttatcaaaattcggaactgacacacgacatgatggaacgtgactaggtgggtttttgactttgcatgtcg

SEQ ID NO: 160 - DNA, 101 bp, Artificial sequence
  DD20CR2 mutant target site
ttcctttacagcacaagtagatcacttgtacttatcaaaattcggaactgacacacgacatgatggaactaaggtgggtttttgactttgcatgtcgaagt

SEQ ID NO: 161 - DNA, 101 bp, Artificial sequence
  DD20CR2 mutant target site
ttcctttacagcacaagtagatcacttgtacttatcaaaattcggaactgacacacgacatgatggaacgaaggtgggtttttgactttgcatgtcgaagt

SEQ ID NO: 162 - DNA, 101 bp, Artificial sequence
  DD20CR2 mutant target site
ttcctttacagcacaagtagatcacttgtacttatcaaaattcggaactgacacacgacatgatggaaggtgggtttttgactttgcatgtcgaagtgaga

SEQ ID NO: 163 - DNA, 101 bp, Artificial sequence
  DD20CR2 mutant target site i
ttcctttacagcacaagtagatcacttgtacttatcaaaattcggaactgacacacgacatgatggacgtgactaaggtgggtttttgactttgcatgtcg

SEQ ID NO: 164 - DNA, 101 bp, Artificial sequence
  DD20CR2 mutant target site
ttcctttacagcacaagtagatcacttgtacttatcaaaattcggaactgacacacgacatgatggaactttactaaggtgggtttttgactttgcatgtc

SEQ ID NO: 165 - DNA, 101 bp, Artificial sequence
  DD20CR2 mutant target site
ttcctttacagcacaagtagatcacttgtacttatcaaaattcggaactgacacacgacatgatggaacgtgacaaggtgggtttttgactttgcatgtcg

SEQ ID NO: 166 - DNA, 101 bp, Artificial sequence
  DD20CR2 mutant target site
ttcctttacagcacaagtagatcacttgtacttatcaaaattcggaactgacacactacattatttaactttactaaggtgggtttttgactttgcatgtc

SEQ ID NO: 167 - DNA, 108 bp, Artificial sequence; identical to SEQ ID NO: 144
  DD43CR1 mutant target site

SEQ ID NO: 168 - DNA, 101 bp, Artificial sequence
  DD43CR1 mutant target site
agctgtaaatacagccttacaactcacaagtcccttgtacggagggtattctagaaaagaggaattggagtctgcctcttcttttagtcctaaattaaaga

SEQ ID NO: 169 - DNA, 101 bp, Artificial sequence
  DD43CR1 mutant target site
agctgtaaatacagccttacaactcacaagtcccttgtacttgtacggagggtattctagaaaagaggaattggagtctgcctcttcttttagtcctaaat

SEQ ID NO: 170 - DNA, 101 bp, Artificial sequence
  DD43CR1 mutant target site
agctgtaaatacagccttacaactcacaagtcccttacggagggtattctagaaaagaggaattggagtctgcctcttcttttagtcctaaattaaagatc

SEQ ID NO: 171 - DNA, 101 bp, Artificial sequence
  DD43CR1 mutant target site
agctgtaaatacagccttacaactcacaagtcccttgtacttgtaccgtacggagggtattctagaaaagaggaattggagtctgcctcttcttttagtcc

SEQ ID NO: 172 - DNA, 101 bp, Artificial sequence
  DD43CR1 mutant target site
agctgtaaatacagccttacaactcacaagtcccttgtactgtacggagggtattctagaaaagaggaattggagtctgcctcttcttttagtcctaaatt

SEQ ID NO: 173 - DNA, 101 bp, Artificial sequence
  DD43CR1 mutant target site
agctgtaaatacagccttacaactcacaagtcccttgtagtacggagggtattctagaaaagaggaattggagtctgcctcttcttttagtcctaaattaa

SEQ ID NO: 174 - DNA, 101 bp, Artificial sequence
  DD43CR1 mutant target site
agctgtaaatacagccttacaactcacaagtcccttgtacttgtacgtagggtattctagaaaagaggaattggagtctgcctcttcttttagtcctaaat

SEQ ID NO: 175 - DNA, 100 bp, Artificial sequence
  DD43CR1 mutant target site
agctgtaaatacagccttacaactcacaagtcctacactctttccctacacgacgctcttcttttagtcctaaattaaagatcggaagatctcgtatgcc

SEQ ID NO: 176 - DNA, 101 bp, Artificial sequence
  DD43CR1 mutant target site
agctgtaaatacagccttacaactcacaagtcccttgtacttgtaccttacggagggtattctagaaaagaggaattggagtctgcctcttcttttagtcc

SEQ ID NO: 177 - DNA, 101 bp, Artificial sequence
  DD43CR2 mutant target site
ctaggtaaatacagccttacaactcacaagtcccttgtacttgtacgtacggagggtattctagaaaattggagtctgcctcttcttttagtcctaaatta

SEQ ID NO: 178 - DNA, 101 bp, Artificial sequence
  DD43CR2 mutant target site
ctaggtaaatacagccttacaactcacaagtcccttgtacttgtacgtacggagggtattctagaaaagaattggagtctgcctcttcttttagtcctaaa

SEQ ID NO: 179 - DNA, 101 bp, Artificial sequence
  DD43CR2 mutant target site
ctaggtaaatacagccttacaactcacaagtcccttgtacttgtacgtacggagggtattctagaattggagtctgcctcttcttttagtcctaaattaaa

SEQ ID NO: 180 - DNA, 101 bp, Artificial sequence
  DD43CR2 mutant target site
ctaggtaaatacagccttacaactcacaagtcccttgtacttgtacgtacggagggtattctagaaaagaaattggagtctgcctcttcttttagtcctaa

SEQ ID NO: 181 - DNA, 101 bp, Artificial sequence
  DD43CR2 mutant target site
ctaggtaaatacagccttacaactcacaagtcccttgtacttgtacgtacggagggtattctagaaaaattggagtctgcctcttcttttagtcctaaatt

SEQ ID NO: 182 - DNA, 101 bp, Artificial sequence
  DD43CR2 mutant target site
ctaggtaaatacagccttacaactcacaagtcccttgtacttgtacgtacggagggtattctagaaaagaggattggagtctgcctcttcttttagtccta

SEQ ID NO: 183 - DNA, 101 bp, Artificial sequence
  DD43CR2 mutant target site
ctaggtaaatacagccttacaactcacaagtcccttgtacttgtacgtacggagggtattctagaaattggagtctgcctcttcttttagtcctaaattaa

SEQ ID NO: 184 - DNA, 101 bp, Artificial sequence
  DD43CR2 mutant target site
ctaggtaaatacagccttacaactcacaagtcccttgtacttgtacgtacggagggtattctattggagtctgcctcttcttttagtcctaaattaaagat

SEQ ID NO: 185 - DNA, 101 bp, Artificial sequence
  DD43CR2 mutant target site
ctaggtaaatacagccttacaactcacaagtcccttgtacttgtacgtacggagggtattctagtctgcctcttcttttagtcctaaattaaagatcggaa

SEQ ID NO: 186 - DNA, 101 bp, Artificial sequence
  DD43CR2 mutant target site
ctaggtaaatacagccttacaactcacaagtcccttgtacttgtacgtacggagggtattctagaaaagtctgcctcttcttttagtcctaaattaaagat

SEQ ID NO: 187 - DNA, 101 bp, Artificial sequence
  DD43CR2 mutant target site
ctaggtaaatacagccttacaactcacaagtcccttgtacttgtacgtacggagggtattctagaaaagagaattggagtctgcctcttcttttagtccta

SEQ ID NO: 188 - DNA, 101 bp, Artificial sequence
  DD43CR2 mutant target site
ctaggtaaatacagccttacaactcacaagtcccttgtacttgtacgtacggagggtattctagaaaagaggagtctgcctcttcttttagtcctaaatta

SEQ ID NO: 189 - DNA, 101 bp, Artificial sequence
  DD43CR2 mutant target site
ctaggtaaatacagccttacaactcacaagtcccttgtacttgtacgtacggagggtattctaattggagtctgcctcttcttttagtcctaaattaaaga

SEQ ID NO: 190 - DNA, 101 bp, Artificial sequence
  DD43CR2 mutant target site
ctaggtaaatacagccttacaactcacaagtcccttgtacttgtacgtacggagggtattctagaaaagaggaaattggagtctgcctcttcttttagtcc

SEQ ID NO: 191 - DNA, 101 bp, Artificial sequence
  DD43CR2 mutant target site
ctaggtaaatacagccttacaactcacaagtcccttgtacttgtacgtacggagggtattctagaaagaggaattggagtctgcctcttcttttagtccta

SEQ ID NO: 192 - protein, 1377 aa, Artificial sequence
  maize optimized moCAS9 endonuclease
MAPKKKRKVMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 193 - DNA, 6677 bp, Artificial sequence
  maize optimized moCAS9 endonuclease
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccccatggccccgaagaagaagaggaaggtgcacatggataagaagtacagcatcggcctcgacatcgggaccaacagcgtcggctgggccgtcatcaccgacgaatataaggtgcccagcaagaagttcaaggtgctcgggaatacagaccgccacagcatcaagaagaacctgatcggcgccctcctgttcgactcgggcgagaccgctgaggccaccagactaaagaggaccgctcgccgccgctacacccgccgcaagaaccgcatatgctacctccaggagatcttcagcaacgagatggccaaggtggacgacagcttcttccaccgccttgaggagtcgttcctcgtggaggaggacaagaagcatgagaggcacccgatcttcgggaacatcgtggacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcgtaccacgagaagtacccgacgatctaccacctccgcaagaagctggtcgactccacagacaaggccgacctcagactgatctacctggccctcgcgcacatgatcaagttccgcgggcacttcctcatcgagggcgacctgaacccggacaactccgacgtcgacaagctcttcatccagctggtccagacctacaatcaactgttcgaggagaacccgatcaacgcgtccggcgtggacgcgaaggccatcctcagcgcgaggctcagcaaatcaagacggctggagaacctgatcgcccagctcccaggcgagaagaaaaacggcttgttcggcaacctgatcgcgctctcgctcggcctcacgcccaacttcaaatcaaacttcgacctggccgaggacgcgaaactgcagctgtccaaggacacttacgacgacgacctcgacaacctgctggcgcaaatcggtgaccagtacgcagacctcttcctggccgccaagaacctctcggacgccatcctgctgtccgatatcctgagagtgaatacggagatcaccaaggcgccgctcagcgcctccatgattaaaaggtacgacgagcaccaccaggacctgacgctgctcaaggccctggtgcgccagcagctccccgagaagtacaaggagatcttcttcgaccaatcaaaaaacggctacgccggctacatcgacgggggcgcctcccaggaggagttctacaagttcatcaaaccaattctcgagaagatggacggcacggaggagcttctcgtgaagctcaaccgggaggacctcctgaggaagcagaggacgttcgacaacggctcgataccgcatcagatccacctgggcgagctccacgccatcctgcgccggcaggaggatttctatccgttcctcaaggacaacagggagaagatcgagaaaattctgacgttccgcatcccgtactacgtgggccctctcgcgcgcgggaacagccggttcgcctggatgactcggaagtcggaggagacgatcacgccgtggaacttcgaggaggtggtggacaagggcgcctccgcccagtcgttcatcgagcgcatgacgaacttcgataaaaatctgcccaatgaaaaagtgctcccgaagcacagcctcctctacgagtacttcacggtgtacaacgagctcacgaaggtgaagtacgtgaccgagggtatgcggaagccggcgttcctgagcggcgagcagaagaaggccatcgtggacctcctcttcaagacgaaccggaaagtcaccgtgaagcaattaaaggaggactacttcaagaaaatagagtgcttcgacagcgtcgagatctcgggcgtcgaggacaggttcaacgcgtcgctgggcacataccacgacctcctcaagatcattaaagacaaggacttcctggacaacgaggagaacgaggacatcctcgaggacatcgtgctgaccctcaccctgtttgaggaccgggagatgatcgaggagcgcctcaagacgtacgctcaccttttcgacgacaaggtgatgaaacagctgaagcggcgccgctacaccggatggggccggctctcccgcaagctcattaatgggatcagggacaagcagtccggcaagaccatactcgatttcctgaagagcgacggcttcgccaaccggaacttcatgcagctcatccacgacgactccctcactttcaaggaggacatccagaaggcccaggtcagcggacagggcgactcgctccacgaacacatcgccaacctggccgggtcgcctgcgattaaaaagggaatccttcagaccgtcaaggtcgtggacgagctggtgaaggtgatgggcaggcacaagcccgaaaatatcgtcattgagatggcccgggagaaccagaccacgcagaaaggccagaagaacagccgggagcgcatgaaacggatcgaggagggtatcaaggagctgggctcgcagatcctcaaggagcaccctgtggaaaatacccagctgcagaatgaaaagctctacctctactacctccagaacggccgcgacatgtacgtggaccaggagctggacattaatcgcctctcggactacgacgtcgaccacatcgtcccgcagtccttcctgaaggacgacagcatcgacaacaaggtcttgacccgctccgataaaaatcgcgggaagtccgacaacgtgccgtcggaggaggtggtcaagaagatgaaaaactactggcgccagctgctcaacgccaagctaatcacgcagcgcaagttcgacaacctcaccaaggccgaacgcggcggtctctccgagcttgataaggctgggttcatcaagagacagctggtggagacccggcagatcaccaagcatgtcgcccagatcctggactcgcgcatgaatactaagtacgatgaaaacgacaagctcatccgcgaggtgaaggtgatcaccctgaagagcaagctggtctcggacttccggaaggacttccagttctacaaggtccgggagatcaacaactaccaccacgcgcacgacgcctacctgaacgcggtggtgggcacagcccttataaagaagtaccctaagctcgagtccgagttcgtgtacggcgactacaaggtgtacgacgtccgcaagatgatcgcgaagagcgagcaggagatcgggaaggccaccgcaaaatacttcttctactccaacatcatgaacttcttcaagaccgagatcaccctggccaacggggagatccgcaagcgcccgctgattgagacgaacggagagacaggcgagatagtctgggacaagggcagggacttcgccaccgtgcgcaaggttctgtccatgccgcaggtgaacatcgtgaagaagactgaggtgcagacaggcggcttctcgaaggagtccatcctgcccaagcggaacagcgacaagctcatcgcgcggaagaaggactgggaccctaaaaaatatggcgggttcgactcgcccaccgtggcttactcggtcctcgtggtggccaaggtcgagaagggcaaaagcaagaagctgaagagcgtcaaggagctcctcggcatcaccatcatggagcggtccagcttcgagaagaacccgatcgacttcctcgaggcgaagggatataaggaggtgaagaaggacctcatcattaaactgccgaagtactcgctattcgaactggagaatggtcgcaagaggatgctcgcgagcgctggcgagctgcagaaagggaacgagctggctctcccgagcaagtacgtcaacttcctctacctggcctcccactatgaaaagctcaagggctcgccggaggacaacgagcagaagcagctgttcgtcgagcagcacaagcattacctcgacgagatcatcgagcagatctcggagttcagcaagcgcgtgatcctggccgacgccaacctcgacaaggtgctgtccgcatataacaagcaccgcgacaaaccaatacgggagcaggccgaaaatatcatccacctgttcaccctcacgaacctgggcgcccccgccgcgttcaagtacttcgacacaaccatcgaccgcaagcggtacacgagcacgaaggaggtgctggacgccacgttgattcaccagtccatcacgggcctgtatgaaacaaggatcgatctcagccagctcggcggcgactaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggc

SEQ ID NO: 194 - DNA, 100 bp, Artificial sequence
  DNA version of guide RNA (EPSPS sgRNA)
gcagtaacagctgctgtcaagttttagagctagaaatagcaagttaaaataaggctagtccgttatcaacttgaaaaagtggcaccgagtcggtgctttt

SEQ ID NO: 195 - DNA, 3708 bp, Artificial sequence
  EPSPS polynucleotide template
ctgcagcccatcaaggagatctccggcaccgtcaagctgccggggtccaagtcgctttccaacaggatcctcctgctcgccgccctgtccgaggtgagcgattttggtgcttgctgcgctgccctgtctcactgctacctaaatgttttgcctgtcgaataccatggattctcggtgtaatccatctcacgatcagatgcaccgcatgtcgcatgcctagctctctctaatttgtctagtagtttgtatacggattaagattgataaatcggtaccgcaaaagctaggtgtaaataaacactacaaaattggatgttcccctatcggcctgtactcggctactcgttcttgtgatggcatgttatttcttcttggtgtttggtgaactcccttatgaaatttgggcgcaaagaaatcgccctcaagggttgatcttatgccatcgtcatgataaacagtgaagcacggatgatcctttacgttgtttttaacaaactttgtcagaaaactagcaatgttaacttcttaatgatgatttcacaacaaaaaaggtaaccttgctactaacataacaaaagacttgttgcttattaattatatgtttttttaatctttgatcaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggatgctaaagaggaagtgcagctcttcttggggaatgctggaatcgcaatgcggtcattgacagcagctgttactgctgctggtggaaatgcaacgtatgtttcctctctctctctacaatacttgttggagttagtatgaaacccatgtgtatgtctagtggcttatggtgtattggtttttgaacttcagttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggttagttactaagggccacatgttacattcttctgtaaatggtacaactattgtcgagcttttgcatttgtaaggaaaacattgattgatctgaatttgatgctacaccacaaaatatctacaaatggtcatccctaactagcaaaccatgtctccattaagctcaatgaagtaatacttggcatgtgtttatcaacttaatttccatcttctggggtattgcctgttttctagtctaatagcatttgtttttagaattagctcttacaactgttatgttctacaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtaagctctgtaatgtatttcactactttgatgccaatgtttcagttttcagttttccaaacagtcgcatcaatatttgaatagatgcactgtagaaaaaaatcattgcagggaaaaactagtactgagtattttgactgtaaattatttaaccagtcggaatatagtcagtctattggagtcaagagcgtgaaccgaaatagccagttaattatcccattatacagaggacaaccatgtatactattgaaacttggtttaagagaatctaggtagctggactcgtagctgcttggcatggataccttcttatctttaggaaaagacacttgattttttttctgtggccctctatgatgtgtgaacctgcttctctattgctttagaaggatatatctatgtcgttatgcaacatgcttcccttagtcatttgtactgaaatcagtttcataagttcgttagtggttccctaaacgaaaccttgtttttctttgcaatcaacaggtcccctaaaaatgcctatgttgaaggtgatgcctcaagcgcaagctatttcttggctggtgctgcaattactggagggactgtgactgtggaaggttgtggcaccaccagtttgcaggtaaagatttcttggctggtgctacgataactgcttttgtctttttggtttcagcattgttctcagagtcactaaataacattatcatctgcaaacgtcaaatagacatacttaggtgaatggatattcatgtaaccgtttccttacaaatttgctgaaacctcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacggtaaaacattctcagccctacaaccatgcctcttctacatcactacttgacaagactaaaaactattggctcgttggcagtggcttcctggagagtaaaggagaccgagaggatggttgcgatccggacggagctaaccaaggtaaggctacatacttcacatgtctcacgtcgtctttccatagctcgctgcctcttagcggcttgcctgcggtcgctccatcctcggttgctgtctgtgttttccacagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtgaccatccgggaccctgggtgcacccggaagaccttccccgactacttcgatgtgctgagcactttcgtcaagaattaataaagcgtgcgatactaccacgcagcttgattgaagtgataggcttgtgctgaggaaatacatttcttttgttctgttttttctctttcacgggattaagttttgagtctgtaacgttagttgtttgtagcaagtttctatttcggatcttaagtttgtgcactgtaagccaaatttcatttcaagagtggttcgttggaataataagaataataaattacgtttcagtggctgtcaagcctgctgctacgttttaggagatggcattagacattcatcatcaacaacaataaaaccttttagcctcaaacaataatagtgaagttattttttagtcctaaacaagttgcattaggatatagttaaaacacaaaagaagctaaagttagggtttagacatgtggatattgttttccatgtatagtatgttctttctttgagtctcatttaactacctctacacataccaactttagttttttttctacctcttcatgttactatggtgccttcttatcccactgagcattggtatatttagaggtttttgttgaacatgcctaaatcatctcaatcaacgatggacaatcttttcttcgattgagctgaggtacgtcatctaga

SEQ ID NO: 196 - DNA, 15 bp, Artificial sequence
  TIPS nucleotide modifications
atcgcaatgcggtca

SEQ ID NO: 197 - DNA, 19 bp, Artificial sequence
  Primer Seqeunce-1 F-E2
ccgaggagatcgtgctgca

SEQ ID NO: 198 - DNA, 20 bp, Artificial sequence
  Primer Seqeunce-2 F-E2
caatggccgcattgcagttc

SEQ ID NO: 199 - DNA, 19 bp, Artificial sequence
  Primer Seqeunce-1 F-T
ccgaggagatcgtgctgca

SEQ ID NO: 200 - DNA, 20 bp, Artificial sequence
  Primer Seqeunce-2 F-T
tgaccgcattgcgattccag

SEQ ID NO: 201 - DNA, 24 bp, Artificial sequence
  Primer Seqeunce-1 H-T
tccaagtcgctttccaacaggatc

SEQ ID NO: 202 - DNA, 20 bp, Artificial sequence
  Primer Seqeunce-2 H-T
tgaccgcattgcgattccag

SEQ ID NO: 203 - DNA, 19 bp, Artificial sequence
  Primer Seqeunce-1 F-E3
ccgaggagatcgtgctgca

SEQ ID NO: 204 - DNA, 24 bp, Artificial sequence
  Primer Seqeunce-2 F-E3
accaagctgcttcaatccgacaac

SEQ ID NO: 205 - DNA, 63 bp, Artificial sequence
  DNA fragment with intact Cas target sequence
ggggaatgctggaactgcaatgcggccattgacagcagctgttactgctgctggtggaaatgc

SEQ ID NO: 206 - DNA, 60 bp, Artificial sequence
  DNA fragment with mutated Cas target sequence
ggggaatgctggaactgcaatgcggccattggcagctgttactgctgctggtggaaatgc

SEQ ID NO: 207 - DNA, 50 bp, Artificial sequence
  DNA fragment with mutated Cas target sequence
ggggaatgctggaactgcacagcagctgttactgctgctggtggaaatgc

SEQ ID NO: 208 - DNA, 33 bp, Artificial sequence
  DNA fragment with mutated Cas target sequence
ggggaatgctgttactgctgctggtggaaatgc

SEQ ID NO: 209 - DNA, 51 bp, Artificial sequence
  TIPS edited EPSPS nucleotide sequence fragment
aatgctggaatcgcaatgcggtcattgacagcagctgttactgctgctggt

SEQ ID NO: 210 - DNA, 51 bp, Artificial sequence
  Wild-type epsps nucleotide sequence  fragment
aatgctggaactgcaatgcggccattgacagcagctgttactgctgctggt

SEQ ID NO: 211 - DNA, 5124 bp, Zea mays
atggcggccatggcgaccaaggccgccgcgggcaccgtgtcgctggacctcgccgcgccgccggcggcggcagcggcggcggcggtgcaggcgggtgccgaggagatcgtgctgcagcccatcaaggagatctccggcaccgtcaagctgccggggtccaagtcgctttccaacaggatcctcctgctcgccgccctgtccgaggtgagcgattttggtgcttgctgcgctgccctgtctcactgctacctaaatgttttgcctgtcgaataccatggattctcggtgtaatccatctcacgatcagatgcaccgcatgtcgcatgcctagctctctctaatttgtctagtagtttgtatacggattaagattgataaatcggtaccgcaaaagctaggtgtaaataaacactacaaaattggatgttcccctatcggcctgtactcggctactcgttcttgtgatggcatgttatttcttcttggtgtttggtgaactcccttatgaaatttgggcgcaaagaaatcgccctcaagggttgatcttatgccatcgtcatgataaacagtgaagcacggatgatcctttacgttgtttttaacaaactttgtcagaaaactagcaatgttaacttcttaatgatgatttcacaacaaaaaaggtaaccttgctactaacataacaaaagacttgttgcttattaattatatgtttttttaatctttgatcaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggatgctaaagaggaagtgcagctcttcttggggaatgctggaactgcaatgcggccattgacagcagctgttactgctgctggtggaaatgcaacgtatgtttcctctctctctctacaatacttgttggagttagtatgaaacccatgtgtatgtctagtggcttatggtgtattggtttttgaacttcagttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggttagttactaagggccacatgttacattcttctgtaaatggtacaactattgtcgagcttttgcatttgtaaggaaaacattgattgatctgaatttgatgctacaccacaaaatatctacaaatggtcatccctaactagcaaaccatgtctccattaagctcaatgaagtaatacttggcatgtgtttatcaacttaatttccatcttctggggtattgcctgttttctagtctaatagcatttgtttttagaattagctcttacaactgttatgttctacaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtaagctctgtaatgtatttcactactttgatgccaatgtttcagttttcagttttccaaacagtcgcatcaatatttgaatagatgcactgtagaaaaaaatcattgcagggaaaaactagtactgagtattttgactgtaaattatttaaccagtcggaatatagtcagtctattggagtcaagagcgtgaaccgaaatagccagttaattatcccattatacagaggacaaccatgtatactattgaaacttggtttaagagaatctaggtagctggactcgtagctgcttggcatggataccttcttatctttaggaaaagacacttgattttttttctgtggccctctatgatgtgtgaacctgcttctctattgctttagaaggatatatctatgtcgttatgcaacatgcttcccttagtcatttgtactgaaatcagtttcataagttcgttagtggttccctaaacgaaaccttgtttttctttgcaatcaacaggtcccctaaaaatgcctatgttgaaggtgatgcctcaagcgcaagctatttcttggctggtgctgcaattactggagggactgtgactgtggaaggttgtggcaccaccagtttgcaggtaaagatttcttggctggtgctacgataactgcttttgtctttttggtttcagcattgttctcagagtcactaaataacattatcatctgcaaacgtcaaatagacatacttaggtgaatggatattcatgtaaccgtttccttacaaatttgctgaaacctcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacggtaaaacattctcagccctacaaccatgcctcttctacatcactacttgacaagactaaaaactattggctcgttggcagtggcttcctggagagtaaaggagaccgagaggatggttgcgatccggacggagctaaccaaggtaaggctacatacttcacatgtctcacgtcgtctttccatagctcgctgcctcttagcggcttgcctgcggtcgctccatcctcggttgctgtctgtgttttccacagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtgaccatccgggaccctgggtgcacccggaagaccttccccgactacttcgatgtgctgagcactttcgtcaagaattaataaagcgtgcgatactaccacgcagcttgattgaagtgataggcttgtgctgaggaaatacatttcttttgttctgttttttctctttcacgggattaagttttgagtctgtaacgttagttgtttgtagcaagtttctatttcggatcttaagtttgtgcactgtaagccaaatttcatttcaagagtggttcgttggaataataagaataataaattacgtttcagtggctgtcaagcctgctgctacgttttaggagatggcattagacattcatcatcaacaacaataaaaccttttagcctcaaacaataatagtgaagttattttttagtcctaaacaagttgcattaggatatagttaaaacacaaaagaagctaaagttagggtttagacatgtggatattgttttccatgtatagtatgttctttctttgagtctcatttaactacctctacacataccaactttagttttttttctacctcttcatgttactatggtgccttcttatcccactgagcattggtatatttagaggtttttgttgaacatgcctaaatcatctcaatcaacgatggacaatcttttcttcgattgagctgaggtacgtcatctagaggataggaccttgagaatatgtgtccgtcaatagctaaccctctactaattttttcaatcaagcaacctattggcttgactttaattcgtaccggcttctactacttctacagtattttgtctctataaattgcagctacaacagtcagaacggctggctttaaaatcaaatggcctaaggatcattgaaaggcatcttagcaatgtctaaaattattaccttctctagacgttgatatctttgctccggattcgatcccttgttgtatgaccacaaatccaacaccaaatacgcatttctgcaacacacccaaacaccccttccaaataagtggaatggttgagaaatttgctattttgattaaatattggtgaaggggcaaggctgaggaaacgagacgaaggttccttgacagctgaaaaatggaacactctagaggcggagggagcgaggcgagctgtgtgaattgccacccattgattaagaatccaacaacttgactagcaaatgccgacatgggtagcctacaaaggcgagttttggagctggtttcgtaataaggaaatttctcaaccaactactttccttagaaaagagttgcttgaccggatcaacatctccccctaaaccccttggagggggagggggctaagattttaatctacaagttagatctaactgtccacctcaatccccctcaaggaggtttttgtattatttgttagtgtagaatgataaagtggatgtattgataggagatggggtacacatatttatagggactcaaccctaaccctaatgggtcggcagcccaacagtggtgtccggcccacacacacactcacacacacagtctaacatcccccgcagtcgcaacggggacaccacacacgatgagactggagtagaggccgaaggtaggagccgacgggttgaaatcccccctagtcgcagcgtcgtgatagtacgaatgttgcggctggagtagagaccggtgtgtgctccaagaagacgatagcccctagatgccgaggtagccgaagtcgaggtggtcgcggtcggaagacgcgcagcaaaagcctgatcttcgggatggtcgacgttcgagcgtcaacgatcggtagggcgacacaataaaagggcaccagcaggtcgaccttcctgcttcttcgatcgtccagacgtcaaggagcctcgctagggaggccgacggcagcgcacgcggctacgccggtcatggtgtcctcacccgcggcagaaaagaaggggaatgtcggatccgaccgagaaggccacggcagcga

SEQ ID NO: 212 - DNA, 3387 bp, S. thermophilus
atgagtgacttagttttaggacttgatatcggtataggttctgttggtgtaggtatccttaacaaagtgacaggagaaattatccataaaaactcacgcatcttcccagcagctcaagcagaaaataacctagtacgtagaacgaatcgtcaaggaagacgcttgacacgacgtaaaaaacatcgtatagttcgtttaaatcgtctatttgaggaaagtggattaatcaccgattttacgaagatttcaattaatcttaacccatatcaattacgagttaagggcttgaccgatgaattgtctaatgaagaactgtttatcgctcttaaaaatatggtgaaacaccgtgggattagttacctcgatgatgctagtgatgacggaaattcatcagtaggagactatgcacaaattgttaaggaaaatagtaaacaattagaaactaagacaccgggacagatacagttggaacgctaccaaacatatggtcaattacgtggtgattttactgttgagaaagatggcaaaaaacatcgcttgattaatgtctttccaacatcagcttatcgttcagaagccttaaggatactgcaaactcaacaagaatttaatccacagattacagatgaatttattaatcgttatctcgaaattttaactggaaaacggaaatattatcatggacccggaaatgaaaagtcacggactgattatggtcgttacagaacgagtggagaaactttagacaatatttttggaattctaattgggaaatgtacattttatccagaagagtttagagcagcaaaagcttcctacacggctcaagaattcaatttgctaaatgatttgaacaatctaacagttcctactgaaaccaaaaagttgagcaaagaacagaagaatcaaatcattaattatgtcaaaaatgaaaaggcaatggggccagcgaaactttttaaatatatcgctaagttactttcttgtgatgttgcagatatcaagggataccgtatcgacaaatcaggtaaggctgagattcatactttcgaagcctatcgaaaaatgaaaacgcttgaaaccttagatattgaacaaatggatagagaaacgcttgataaattagcctatgtcttaacattaaacactgagagggaaggtattcaagaagccttagaacatgaatttgctgatggtagctttagccagaagcaagttgacgaattggttcaattccgcaaagcaaatagttccatttttggaaaaggatggcataatttttctgtcaaactgatgatggagttaattccagaattgtatgagacgtcagaagagcaaatgactatcctgacacgacttggaaaacaaaaacgacttcgtcttcaaataaaacaaaatatttcaaataaaacaaaatatatagatgagaaactattaactgaagaaatctataatcctgttgttgctaagtctgttcgccaggctataaaaatcgtaaatgcggcgattaaagaatacggagactttgacaatattgtcatcgaaatggctcgtgaaacaaatgaagatgatgaaaagaaagctattcaaaagattcaaaaagccaacaaagatgaaaaagatgcagcaatgcttaaggctgctaaccaatataatggaaaggctgaattaccacatagtgttttccacggtcataagcaattagcgactaaaatccgcctttggcatcagcaaggagaacgttgcctttatactggtaagacaatctcaatccatgatttgataaataatcctaatcagtttgaagtagatcatattttacctctttctatcacattcgatgatagccttgcaaataaggttttggtttatgcaactgctaaccaagaaaaaggacaacgaacaccttatcaggctttagatagtatggatgatgcgtggtctttccgtgaattaaaagcttttgtacgtgagtcaaaaacactttcaaacaagaaaaaagaatacctccttacagaagaagatatttcaaagtttgatgttcgaaagaaatttattgaacgaaatcttgtagatacaagatacgcttcaagagttgtcctcaatgcccttcaagaacactttagagctcacaagattgatacaaaagtttccgtggttcgtggccaatttacatctcaattgagacgccattggggaattgagaagactcgtgatacttatcatcaccatgctgtcgatgcattgattattgccgcctcaagtcagttgaatttgtggaaaaaacaaaagaatacccttgtaagttattcagaagaacaactccttgatattgaaacaggtgaacttattagtgatgatgagtacaaggaatctgtgttcaaagccccttatcaacattttgttgatacattgaagagtaaagaatttgaagacagtatcttattctcatatcaagtggattctaagtttaatcgtaaaatatcagatgccactatttatgcgacaagacaggctaaagtgggaaaagataagaaggatgaaacttatgtcttagggaaaatcaaagatatctatactcaggatggttatgatgcctttatgaagatttataagaaggataagtcaaaattcctcatgtatcgtcacgacccacaaacctttgagaaagttatcgagccaattttagagaactatcctaataagcaaatgaatgaaaaaggaaaagaggtaccatgtaatcctttcctaaaatataaagaagaacatggctatattcgtaaatatagtaaaaaaggcaatggtcctgaaatcaagagtcttaaatactatgatagtaagcttttaggtaatcctattgatattactccagagaatagtaaaaataaagttgtcttacagtcattaaaaccttggagaacagatgtctatttcaataaggctactggaaaatacgaaatccttggattaaaatatgctgatctacaatttgagaaagggacaggaacatataagatttcccaggaaaaatacaatgacattaagaaaaaagagggtgtagattctgattcagaattcaagtttacactttataaaaatgatttgttactcgttaaagatacagaaacaaaagaacaacagcttttccgttttctttctcgaactttacctaaacaaaagcattatgttgaattaaaaccttatgataaacagaaatttgaaggaggtgaggcgttaattaaagtgttgggtaacgttgctaatggtggtcaatgcataaaaggactagcaaaatcaaatatttctatttataaagtaagaacagatgtcctaggaaatcagcatatcatcaaaaatgagggtgataagcctaagctagatttttaa

SEQ ID NO: 213 - DNA, 3369 bp, S. thermophilus
atgagtgacttagttttaggacttgatatcggtataggttctgttggtgtaggtatccttaacaaagtgacaggagaaattatccataaaaactcacgcatcttcccagcagctcaagcagaaaataacctagtacgtagaacgaatcgtcaaggaagacgcttgacacgacgtaaaaaacatcgtatagttcgtttaaatcgtctatttgaggaaagtggattaatcaccgattttacgaagatttcaattaatcttaacccatatcaattacgagttaagggcttgaccgatgaattgtctaatgaagaactgtttatcgctcttaaaaatatggtgaaacaccgtgggattagttacctcgatgatgctagtgatgacggaaattcatcagtaggagactatgcacaaattgttaaggaaaatagtaaacaattagaaactaagacaccgggacagatacagttggaacgctaccaaacatatggtcaattacgtggtgattttactgttgagaaagatggcaaaaaacatcgcttgattaatgtctttccaacatcagcttatcgttcagaagccttaaggatactgcaaactcaacaagaatttaattcacagattacagatgaatttattaatcgttatctcgaaattttaactggaaaacggaaatattatcatggacccggaaatgaaaagtcacggactgattatggtcgttacagaacgaatggagaaactttagacaatatttttggaattctaattgggaaatgtacattttatccagacgagtttagagcagcaaaagcttcctacacggctcaagaattcaatttgctaaatgatttgaacaatctaacagttcctactgaaaccaaaaagttgagcaaagaacagaagaatcaaatcattaattatgtcaaaaatgaaaaggtaatggggccagcgaaactttttaaatatatcgctaaattactttcttgtgatgttgcagatatcaagggacaccgtatcgacaaatcaggtaaggctgagattcatactttcgaagcctatcgaaaaatgaaaacgcttgaaaccttagatattgagcaaatggatagagaaacgcttgataaattagcctatgtcttaacattaaacactgagagggaaggtattcaagaagctttagaacatgaatttgctgatggtagctttagccagaagcaagttgacgaattggttcaattccgcaaagcaaatagttccatttttggaaaaggatggcataatttttctgtcaaactgatgatggagttaattccagaattgtatgagacgtcagaagagcaaatgactatcctgacacgacttggaaaacaaaaaacaacttcgtcttcaaataaaacaaaatatatagatgagaaactattaactgaagaaatctataatcctgttgttgctaagtctgttcgccaggctataaaaatcgtaaatgcggcgattaaagaatacggagactttgacaatattgtcatcgaaatggctcgtgaaacaaatgaagatgatgaaaagaaagctattcaaaagattcaaaaagccaacaaagatgaaaaagatgcagcaatgcttaaggctgctaaccaatataatggaaaggctgaattaccacatagtgttttccacggtcataagcaattagcgactaaaatccgcctttggcatcagcaaggagaacgttgcctttatactggtaagacaatctcaatccatgatttgataaataatcctaatcagtttgaagtagatcatattttacctctttctatcacattcgatgatagccttgcaaataaggttttggtttatgcaactgctaaccaagaaaaaggacaacgaacaccttatcaggctttagatagtatggatgatgcgtggtctttccgtgaattaaaagcttttgtacgtgagtcaaaaacactttcaaacaagaaaaaagaatacctccttacagaagaagatatttcaaagtttgatgttcgaaagaaatttattgaacgaaatcttgtagatacaagatacgcttcaagagttgtcctcaatgcccttcaagaacactttagagctcacaagattgatacaaaagtttccgtggttcgtggccaatttacatctcaattgagacgccattggggaattgagaagactcgtgatacttatcatcaccatgctgtcgatgcattgattattgccgcctcaagtcagttgaatttgtggaaaaaacaaaagaatacccttgtaagttattcagaagaacaactccttgatattgaaacaggtgaacttattagtgatgatgagtacaaggaatctgtgttcaaagccccttatcaacattttgttgatacattgaagagtaaagaatttgaagacagtatcttattctcatatcaagtggattctaagtttaatcgtaaaatatcagatgccactatttatgcgacaagacaggctaaagtgggaaaagataagaaggatgaaacttatgtcttagggaaaatcaaagatatctatactcaggatggttatgatgcctttatgaagatttataagaaggataagtcaaaattcctcatgtatcgtcacgacccacaaacctttgagaaagttatcgagccaattttagagaactatcctaataaggaaatgaatgaaaaagggaaagaagtaccatgtaatcctttcctaaaatataaagaagaacatggctatattcgtaaatatagtaaaaaaggcaatggtcctgaaatcaagagtcttaaatactatgatagtaagcttttaggtaatcctattgatattactccagagaatagtaaaaataaagttgtcttacagtcattaaaaccttggagaacagatgtctatttcaataaaaatactggtaaatatgaaattttaggactgaaatatgctgatttacaatttgaaaagaagacaggaacatataagatttcccaggaaaaatacaatggcattatgaaagaagagggtgtagattctgattcagaattcaagtttacactttataaaaatgatttgttactcgttaaagatacagaaacaaaagaacaacagcttttccgttttctttctcgaactatgcctaatgtgaaatattatgtagagttaaagccttattcaaaagataaatttgagaagaatgagtcacttattgaaattttaggttctgcagataagtcaggacgatgtataaaagggctaggaaaatcaaatatttctatttataaggtaagaacagatgtcctaggaaatcagcatatcatcaaaaatgagggtgataagcctaagctagatttttaa

SEQ ID NO: 214 - DNA, 4113 bp, S. agalactiae
atgaataagccatattcaataggccttgacatcggtactaattccgtcggatggagcattattacagatgattataaagtacctgctaagaagatgagagttttagggaacactgataaagaatatattaagaagaatctcataggtgctctgctttttgatggcgggaatactgctgcagatagacgcttgaagcgaactgctcgtcgtcgttatacacgtcgtagaaatcgtattctatatttacaagaaatttttgcagaggaaatgagtaaagttgatgatagtttctttcatcgattagaggattcttttctagttgaggaagataagagagggagcaagtatcctatctttgcaacattgcaggaagagaaagattatcatgaaaaattttcgacaatctatcatttgagaaaagaattagctgacaagaaagaaaaagcagaccttcgtcttatttatattgctctagctcatatcattaaatttagagggcatttcctaattgaggatgatagctttgatgtcaggaatacagacatttcaaaacaatatcaagattttttagaaatctttaatacaacttttgaaaataatgatttgttatctcaaaacgttgacgtagaggcaatactaacagataagattagcaagtctgcgaagaaagatcgtattttagcgcagtatcctaaccaaaaatctactggcatttttgcagaatttttgaaattgattgtcggaaatcaagctgacttcaagaaatatttcaatttggaggataaaacgccgcttcaattcgctaaggatagctacgatgaagatttagaaaatcttcttggacagattggtgatgaatttgcagacttattctcagcagcgaaaaagttatatgatagtgtccttttgtctggcattcttacagtaatcgacctcagtaccaaggcgccactttcagcttctatgattcagcgttatgatgaacatagagaggacttgaaacagttaaaacaattcgtaaaagcttcattgccggaaaaatatcaagaaatatttgctgattcatcaaaagatggctacgctggttatattgaaggtaaaactaatcaagaagctttttataaatacctgtcaaaattgttgaccaagcaagaagatagcgagaattttcttgaaaaaatcaagaatgaagatttcttgagaaaacaaaggacctttgataatggctcaattccacaccaagtccatttgacagagctgaaagctattatccgccgtcaatcagaatactatcccttcttgaaagagaatcaagataggattgaaaaaatccttacctttagaattccttattatatcgggccactagcacgtgagaagagtgattttgcatggatgactcgcaaaacagatgacagtattcgaccttggaattttgaagacttggttgataaagaaaaatctgcggaagcttttatccatcgtatgaccaacaatgatttttatcttcctgaagaaaaagttttaccaaagcatagtcttatttatgaaaaatttacggtctataatgagttgactaaggttagatataaaaatgagcaaggtgagacttatttttttgatagcaatattaaacaagaaatctttgatggagtattcaaggaacatcgtaaggtatccaagaagaagttgctagattttctggctaaagaatatgaggagtttaggatagtagatgttattggtctagataaagaaaataaagctttcaacgcctcattgggaacttaccacgatctcgaaaaaatactagacaaagattttctagataatccagataatgagtctattctggaagatatcgtccaaactctaacattatttgaagacagagaaatgattaagaagcgtcttgaaaactataaagatctttttacagagtcacaactaaaaaaactctatcgtcgtcactatactggctggggacgattgtctgctaagttaatcaatggtattcgagataaagagagtcaaaaaacaatcttggactatcttattgatgatggtagatctaatcgcaactttatgcagttgataaatgatgatggtctatctttcaaatcaattatcagtaaggcacaggctggtagtcattcagataatctaaaagaagttgtaggtgagcttgcaggtagccctgctattaaaaagggaattctacaaagtttgaaaattgttgatgagcttgttaaagtcatgggatacgaacctgaacaaattgtggttgagatggcgcgtgagaatcaaacaacaaatcaaggtcgtcgtaactctcgacaacgctataaacttcttgatgatggcgttaagaatctagctagtgacttgaatggcaatattttgaaagaatatcctacggataatcaagcgttgcaaaatgaaagacttttcctttactacttacaaaacggaagagatatgtatacaggggaagctctagatattgacaatttaagtcaatatgatattgaccacattattcctcaagctttcataaaagatgattctattgataatcgtgttttggtatcatctgctaaaaatcgtggaaagtcagatgatgttcctagccttgaaattgtaaaagattgtaaagttttctggaaaaaattacttgatgctaagttaatgagtcagcgtaagtatgataatttgactaaggcagagcgcggaggcctaacttccgatgataaggcaagatttatccaacgtcagttggttgagacacgacaaattaccaagcatgttgcccgtatcttggatgaacgctttaataatgagcttgatagtaaaggtagaaggatccgcaaagttaaaattgtaaccttgaagtcaaatttggtttcaaatttccgaaaagaatttggattctataaaattcgtgaagttaacaattatcaccatgcacatgatgcctatcttaatgcagtagttgctaaagctattctaaccaaatatcctcagttagagccagaatttgtctacggcgactatccaaaatataatagttacaaaacgcgtaaatccgctacagaaaagctatttttctattcaaatattatgaacttctttaaaactaaggtaactttagcggatggaaccgttgttgtaaaagatgatattgaagttaataatgatacgggtgaaattgtttgggataaaaagaaacactttgcgacagttagaaaagtcttgtcataccctcagaacaatatcgtgaagaagacagagattcagacaggtggtttctctaaggaatcaatcttggcgcatggtaactcagataagttgattccaagaaaaacgaaggatatttatttagatcctaagaaatatggaggttttgatagtccgatagtagcttactctgttttagttgtagctgatatcaaaaagggtaaagcacaaaaactaaaaacagttacggaacttttaggaattaccatcatggagaggtccagatttgagaaaaatccatcagctttccttgaatcaaaaggctatttaaatattagggctgataaactaattattttgcccaagtatagtctgttcgaattagaaaatgggcgtcgtcgattacttgctagtgctggtgaattacaaaaaggtaatgagctagccttaccaacacaatttatgaagttcttataccttgcaagtcgttataatgagtcaaaaggtaaaccagaggagattgagaagaaacaagaatttgtaaatcaacatgtctcttattttgatgacatccttcaattaattaatgatttttcaaaacgagttattctagcagatgctaatttagagaaaatcaataagctttaccaagataataaggaaaatatatcagtagatgaacttgctaataatattatcaatctatttacttttaccagtctaggagctccagcagcttttaaattttttgataaaatagttgatagaaaacgctatacatcaactaaagaagtacttaattctaccctaattcatcaatctattactggactttatgaaacacgtattgatttgggtaagttaggagaagattga

SEQ ID NO: 215 - DNA, 4134 bp, S. agalactiae
atgaataagccatattcaataggccttgacatcggtactaattccgtcggatggagcattattacagatgattataaagtacctgctaagaagatgagagttttagggaacactgataaagaatatattaagaagaatctcataggtgctctgctttttgatggcgggaatactgctgcagatagacgcttgaagcgaactgctcgtcgtcgttatacacgtcgtagaaatcgtattctatatttacaagaaatttttgcagaggaaatgagtaaagttgatgatagtttctttcatcgattagaggattcttttctagttgaggaagataagagaggtagcaagtatcctatctttgcaacaatgcaggaggagaaatattatcatgaaaaatttccgacaatctatcatttgagaaaagaattggctgacaagaaagaaaaagcagaccttcgtcttgtttatctggctctagctcatatcattaaattcagagggcatttcctaattgaggatgatagatttgatgtgaggaataccgatattcaaaaacaatatcaagcctttttagaaatttttgatactacctttgaaaataatcatttgttatctcaaaatgtagatgtagaagcaattctaacagataagattagcaagtctgcgaagaaggatcgcatcttagcgcagtatcctaaccaaaaatctactggtatttttgcagaatttttgaaattgattgtcggaaatcaagctgacttcaagaaacatttcaatttggaggataaaacaccgcttcaattcgctaaggatagctacgatgaagatttagaaaatcttcttggacagattggtgatgaatttgcagacttattctcagtagcgaaaaagctatatgatagtgttcttttatctggcattcttacagtaactgatctcagtaccaaggcgccactttctgcctctatgattcagcgttatgatgaacatcatgaggacttaaagcatctaaaacaattcgtaaaagcttcattacctgaaaattatcgggaagtatttgctgattcatcaaaagatggctacgctggctatattgaaggcaaaactaatcaagaagctttttataaatatctgttaaaattgttgaccaaacaagaaggtagcgagtattttcttgagaaaattaagaatgaagattttttgagaaaacagagaacctttgataatggctcaatcccgcatcaagtccatttgacagaattgagggctattattcgacgtcaatcagaatactatccattcttgaaagagaatcaagataggattgaaaaaatccttacctttagaattccttattatgtcgggccactagcacgtgagaagagtgattttgcatggatgactcgcaaaacagatgacagtattcgaccttggaattttgaagacttggttgataaagaaaaatctgcggaagcttttatccatcgcatgaccaacaatgacctctatcttccagaagaaaaagttttaccaaagcatagtcttatttatgaaaaatttactgtttacaatgaattaacgaaggttagatttttggcagaaggctttaaagattttcaatttttaaataggaagcaaaaagaaactatctttaacagcttgtttaaggaaaaacgtaaagtaactgaaaaggatattattagttttttgaataaagttgatggatatgaaggaattgcaatcaaaggaattgagaaacagtttaacgctagcctttcaacctatcatgatcttaaaaaaatacttggcaaggatttccttgataatacagataacgagcttattttggaagatatcgtccaaactctaaccttatttgaagatagagaaatgattaagaagtgtcttgacatctataaagatttttttacagagtcacagcttaaaaagctctatcgccgtcactatactggctggggacgattgtctgctaagctaataaatggcatccgaaataaagagaatcaaaaaacaatcttggactatcttattgatgatggaagtgcaaaccgaaacttcatgcagttgataaatgatgatgatctatcatttaaaccaattattgacaaggcacgaactggtagtcattcggataatctgaaagaagttgtaggtgaacttgctggtagccctgctattaaaaaagggattctacaaagtttgaaaatagttgatgagctggttaaagtcatgggctatgaacctgaacaaatcgtggttgaaatggcacgtgagaaccaaacgacagcaaaaggattaagtcgttcacgacaacgcttgacaaccttgagagaatctcttgctaatttgaagagtaatattttggaagagaaaaagcctaagtatgtgaaagatcaagttgaaaatcatcatttatctgatgaccgtcttttcctttactacttacaaaacggaagagatatgtatacaaaaaaggctctggatattgataatttaagtcaatatgatattgaccacattattcctcaagctttcataaaagatgattctattgataatcgtgttttggtatcatctgctaaaaatcgtggaaaatcagatgatgttcctagcattgaaattgtaaaagctcgcaaaatgttctggaaaaatttactggatgctaagttaatgagtcagcgtaagtatgataatttgactaaggcagagcgcggaggcctaacttccgatgataaggcaagatttatccaacgtcagttggttgagactcgacaaattaccaagcatgtagctcgtatcttggatgaacgcttcaataatgaagttgataatggtaaaaagatttgcaaggttaaaattgtaaccttgaagtcaaatttggtttcaaatttccgaaaagaatttggattctataaaattcgtgaagttaatgattatcaccatgcacacgatgcttatcttaatgcagtagttgccaaagctattctaaccaaatatccacagttagagccagagtttgtctacggaatgtatagacagaaaaaactttcgaaaatcgttcatgaggataaggaagaaaaatatagtgaagcaaccaggaaaatgtttttctactccaacttgatgaatatgttcaaaagagttgtgaggttagcagatggttctattgttgtaagaccagtaatagaaactggtagatatatgagaaaaactgcatgggataaaaagaaacactttgcgacagttagaaaagtcttgtcataccctcagaacaatatcgtgaagaagacagagattcagacaggtggtttctctaaggaatcaatcttggcgcatggtaactcagataagttgattccaagaaaaacgaaggatatttatttagatcctaagaaatatggaggttttgatagtccgatagtagcttactctgttttagttgtagctgatatcaaaaaaggtaaagcacaaaaactaaaaacagttacggaacttttaggaattaccatcatggagaggtccagatttgagaaaaatccatcagctttccttgaatcaaaaggttatttaaatattagggacgataaattaatgattttaccgaagtatagtctgttcgaattagaaaatgggcgtcgtcgattacttgctagtgctggtgaattacaaaaaggtaacgagctagccttaccaacacaatttatgaagttcttataccttgcaagtcgttataatgagtcaaaaggtaaaccagaggagattgagaagaaacaagaatttgtaaatcaacatgtctcttattttgatgacatccttcaattaattaatgatttttcaaaacgagttattctagcagatgctaatttagagaaaatcaataagctttaccaggataataaggaaaatataccagtagatgaacttgctaataatattatcaatctatttacttttaccagtctaggagctccagcagcttttaaattttttgataaaatagttgatagaaaacgctatacatcaactaaagaagtacttaattctactctaatccatcaatctattactggactttatgaaacacgtattgatttgggtaaattaggagaagattga

SEQ ID NO: 216 - DNA, 4038 bp, S. mutans
atgaaaaaaccttactctattggacttgatattggaaccaattctgttggttgggctgttgtgacagatgactacaaagttcctgctaagaagatgaaggttctgggaaatacagataaaagtcatatcgagaaaaatttgcttggcgctttattatttgatagcgggaatactgcagaagacagacggttaaagagaactgctcgccgtcgttacacacgtcgcagaaatcgtattttatatttgcaagagattttttcagaagaaatgggcaaggtagatgatagtttctttcatcgtttagaggattcttttcttgttactgaggataaacgaggagagcgccatcccatttttgggaatcttgaagaagaagttaagtatcatgaaaattttccaaccatttatcatttgcggcaatatcttgcggataatccagaaaaagttgatttgcgtttagtttatttggctttggcacatataattaagtttagaggtcattttttaattgaaggaaagtttgatacacgcaataatgatgtacaaagactgtttcaagaatttttagcagtctatgataatacttttgagaatagttcgcttcaggagcaaaatgttcaagttgaagaaattctgactgataaaatcagtaaatctgctaagaaagatagagttttgaaactttttcctaatgaaaagtctaatggccgctttgcagaatttctaaaactaattgttggtaatcaagctgattttaaaaagcattttgaattagaagagaaagcaccattgcaattttctaaagatacttatgaagaagagttagaagtactattagctcaaattggagataattacgcagagctctttttatcagcaaagaaactgtatgatagtatccttttatcagggattttaacagttactgatgttggtaccaaagcgcctttatctgcttcgatgattcagcgatataatgaacatcagatggatttagctcagcttaaacaattcattcgtcagaaattatcagataaatataacgaagttttttctgatgtttcaaaagacggctatgcgggttatattgatgggaaaacaaatcaagaagctttttataaataccttaaaggtctattaaataagattgagggaagtggctatttccttgataaaattgagcgtgaagattttctaagaaagcaacgtacctttgacaatggctctattccacatcagattcatcttcaagaaatgcgtgctatcattcgtagacaggctgaattttatccgtttttagcagacaatcaagataggattgagaaattattgactttccgtattccctactatgttggtccattagcgcgcggaaaaagtgattttgcttggttaagtcggaaatcggctgataaaattacaccatggaattttgatgaaatcgttgataaagaatcctctgcagaagcttttatcaatcgtatgacaaattatgatttgtacttgccaaatcaaaaagttcttcctaaacatagtttattatacgaaaaatttactgtttacaatgaattaacaaaggttaaatataaaacagagcaaggaaaaacagcattttttgatgccaatatgaagcaagaaatctttgatggcgtatttaaggtttatcgaaaagtaactaaagataaattaatggatttccttgaaaaagaatttgatgaatttcgtattgttgatttaacaggtctggataaagaaaataaagtatttaacgcttcttatggaacttatcatgatttgtgtaaaattttagataaagattttctcgataattcaaagaatgaaaagattttagaagatattgtgttgaccttaacgttatttgaagatagagaaatgattagaaaacgtctagaaaattacagtgatttattgaccaaagaacaagtgaaaaagctggaaagacgtcattatactggttggggaagattatcagctgagttaattcatggtattcgcaataaagaaagcagaaaaacaattcttgattatctcattgatgatggcaatagcaatcggaactttatgcaactgattaacgatgatgctctttctttcaaagaagagattgctaaggcacaagttattggagaaacagacaatctaaatcaagttgttagtgatattgctggcagccctgctattaaaaaaggaattttacaaagcttgaagattgttgatgagcttgtcaaaattatgggacatcaacctgaaaatatcgtcgtggagatggcgcgtgaaaaccagtttaccaatcagggacgacgaaattcacagcaacgtttgaaaggtttgacagattctattaaagaatttggaagtcaaattcttaaagaacatccggttgagaattcacagttacaaaatgatagattgtttctatattatttacaaaacggcagagatatgtatactggagaagaattggatattgattatctaagccagtatgatatagaccatattatcccgcaagcttttataaaggataattctattgataatagagtattgactagctcaaaggaaaatcgtggaaaatcggatgatgtaccaagtaaagatgttgttcgtaaaatgaaatcctattggagtaagctactttcggcaaagcttattacacaacgtaaatttgataatttgacaaaagctgaacgaggtggattgaccgacgatgataaagctggattcatcaagcgtcaattagtagaaacacgacaaattaccaaacatgtagcacgtattctggacgaacgatttaatacagaaacagatgaaaacaacaagaaaattcgtcaagtaaaaattgtgaccttgaaatcaaatcttgtttccaatttccgtaaagagtttgaactctacaaagtgcgtgaaattaatgactatcatcatgcacatgatgcctatctcaatgctgtaattggaaaggctttactaggtgtttacccacaattggaacctgaatttgtttatggtgattatcctcattttcatggacataaagaaaataaagcaactgctaagaaatttttctattcaaatattatgaacttctttaaaaaagatgatgtccgtactgataaaaatggtgaaattatctggaaaaaagatgagcatatttctaatattaaaaaagtgctttcttatccacaagttaatattgttaagaaagtagaggagcaaacgggaggattttctaaagaatctatcttgccgaaaggtaattctgacaagcttattcctcgaaaaacgaagaaattttattgggataccaagaaatatggaggatttgatagcccgattgttgcttattctattttagttattgctgatattgaaaaaggtaaatctaaaaaattgaaaacagtcaaagccttagttggtgtcactattatggaaaagatgacttttgaaagggatccagttgcttttcttgagcgaaaaggctatcgaaatgttcaagaagaaaatattataaagttaccaaaatatagtttatttaaactagaaaacggacgaaaaaggctattggcaagtgctagggaacttcaaaagggaaatgaaatcgttttgccaaatcatttaggaaccttgctttatcacgctaaaaatattcataaagttgatgaaccaaagcatttggactatgttgataaacataaagatgaatttaaggagttgctagatgttgtgtcaaacttttctaaaaaatatactttagcagaaggaaatttagaaaaaatcaaagaattatatgcacaaaataatggtgaagatcttaaagaattagcaagttcatttatcaacttattaacatttactgctataggagcaccggctacttttaaattctttgataaaaatattgatcgaaaacgatatacttcaactactgaaattctcaacgctaccctcatccaccaatccatcaccggtctttatgaaacgcggattgatctcaataagttaggaggagactaa

SEQ ID NO: 217 - DNA, 20 bp, Artificial Sequence
  Mprimer qADH-F
caagtcgcggttttcaatca

SEQ ID NO: 218 - DNA, 21 bp, Artificial Sequence
  Primer qADH-R
tgaaggtggaagtcccaacaa

SEQ ID NO: 219 - DNA, 19 bp, Artificial Sequence
  probe ADH-VIC
tgggaagcctatctaccac

SEQ ID NO: 220 - DNA, 15 bp, Artificial Sequence
  Probe wtEPSPS
cggccattgacagca

SEQ ID NO: 221 - DNA, 20 bp, Artificial Sequence
  Forward primer qEPSPS-F
tcttggggaatgctggaact

SEQ ID NO: 222 - DNA, 21 bp, Artificial Sequence
  reverse primer qEPSPSR
caccagcagcagtaacagctg

SEQ ID NO: 223 - DNA, 17 bp, Artificial Sequence
  FAM-wtEPSPS R probe
tgctgtcaatggccgca

SEQ ID NO: 224 - DNA, 20 bp, Artificial Sequence
  forward primer qEPSPS-F
tcttggggaatgctggaact

SEQ ID NO: 225 - DNA, 20 bp, Artificial Sequence
  reverse primer q wtEPSPS RA
ccaccagcagcagtaacagc

SEQ ID NO: 226 - DNA, 21 bp, Artificial Sequence
  orward primer q epTIPS F
ggaagtgcagctcttcttggg

SEQ ID NO: 227 - DNA, 19 bp, Artificial Sequence
  reverse primer q epTIPS R
agctgctgtcaatgaccgc

SEQ ID NO: 228 - DNA, 15 bp, Artificial Sequence
  TIPS probe
aatgctggaatcgca

SEQ ID NO: 229 - DNA, 23 bp, Zea mays
  MHP14Cas1 target site (1)..(23)
gttaaatctgacgtgaatctgtt

SEQ ID NO: 230 - DNA, 21 bp, Zea mays
  MHP14Cas3 target site (1)..(21)
acaaacattgaagcgacatag

SEQ ID NO: 231 - DNA, 18 bp, Zea mays
  TS8Cas1 target site (1)..(18)
gtacgtaacgtgcagtac

SEQ ID NO: 232 - DNA, 20 bp, Zea mays
  TS8Cas2 target site (1)..(20)
gctcatcagtgatcagctgg

SEQ ID NO: 233 - DNA, 17 bp, Zea mays
  TS9Cas2 target site (1)..(17)
ggctgtttgcggcctcg

SEQ ID NO: 234 - DNA, 21 bp, Zea mays
  TS9Cas3 target site (1)..(21)
gcctcgaggttgcacgcacgt

SEQ ID NO: 235 - DNA, 20 bp, Zea mays
  TS10Cas1 target site (1)..(20)
gcctcgccttcgctagttaa

SEQ ID NO: 236 - DNA, 18 bp, Zea mays
  TS10Cas3 target site (1)..(18)
gctcgtgttggagataca

SEQ ID NO: 237 - DNA, 80 bp, Zea mays
gttaaatctgacgtgaatctgtttggaattgaaaaacaagtgcttcctttcatacaccactatgtcgcttcaatgtttgt

SEQ ID NO: 238 - DNA, 80 bp, Zea mays
acaaacattgaagcgacatagtggtgtatgaaaggaagcacttgtttttcaattccaaacagattcacgtcagatttaac

SEQ ID NO: 239 - DNA, 66 bp, Zea mays
ccagtactgcacgttacgtacgtacgaactaatatactccaccagctgatcactgatgagccgagc

SEQ ID NO: 240 - DNA, 66 bp, Zea mays
gctcggctcatcagtgatcagctggtggagtatattagttcgtacgtacgtaacgtgcagtactgg

SEQ ID NO: 241 - DNA, 35 bp, Zea mays
ccgacgtgcgtgcaacctcgaggccgcaaacagcc

SEQ ID NO: 242 - DNA, 35 bp, Zea mays
ggctgtttgcggcctcgaggttgcacgcacgtcgg

SEQ ID NO: 243 - DNA, 68 bp, Zea mays
gctcgtgttggagatacagggacagcaagtacttggcccttaactagcgaaggcgaggcggccatgga

SEQ ID NO: 244 - DNA, 68 bp, Zea mays
tccatggccgcctcgccttcgctagttaagggccaagtacttgctgtccctgtatctccaacacgagc

SEQ ID NO: 245 - DNA, 1108 bp, Artificial Sequence
  MHP14Cas-1 guideRNA cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgttaaatctgacgtgaatctgttgttttagagctagaaatagcaagttaaaataaggctagtccgttatcaacttgaaaaagtggcaccgagtcggtgcttttttttt

SEQ ID NO: 246 - DNA, 1106 bp, Artificial Sequence
  MHP14Cas-3 gRNA cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgcaaacattgaagcgacataggttttagagctagaaatagcaagttaaaataaggctagtccgttatcaacttgaaaaagtggcaccgagtcggtgcttttttttt

SEQ ID NO: 247 - DNA, 1103 bp, Artificial Sequence
  TS8Cas-1 guideRNA cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgtacgtaacgtgcagtacgttttagagctagaaatagcaagttaaaataaggctagtccgttatcaacttgaaaaagtggcaccgagtcggtgcttttttttt

SEQ ID NO: 248 - DNA, 1105 bp, Artificial Sequence
  TS8Cas-2 guideRNA cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgctcatcagtgatcagctgggttttagagctagaaatagcaagttaaaataaggctagtccgttatcaacttgaaaaagtggcaccgagtcggtgcttttttttt

SEQ ID NO: 249 - DNA, 1102 bp, Artificial Sequence
  TS9Cas-2 guideRNA cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattggctgtttgcggcctcggttttagagctagaaatagcaagttaaaataaggctagtccgttatcaacttgaaaaagtggcaccgagtcggtgcttttttttt

SEQ ID NO: 250 - DNA, 1106 bp, Artificial Sequence
  TS9Cas-3 guideRNA cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgcctcgaggttgcacgcacgtgttttagagctagaaatagcaagttaaaataaggctagtccgttatcaacttgaaaaagtggcaccgagtcggtgcttttttttt

SEQ ID NO: 251 - DNA, 1105 bp, Artificial Sequence
  TS10Cas-1 guideRNA cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgcctcgccttcgctagttaagttttagagctagaaatagcaagttaaaataaggctagtccgttatcaacttgaaaaagtggcaccgagtcggtgcttttttttt

SEQ ID NO: 252 - DNA, 1103 bp, Artificial Sequence
  TSCas-3 guideRNA cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgctcgtgttggagatacagttttagagctagaaatagcaagttaaaataaggctagtccgttatcaacttgaaaaagtggcaccgagtcggtgcttttttttt

SEQ ID NO: 253 - DNA, 4928 bp, Artificial Sequence
  MHP14Cas1 donor
gccatgtcatcttgtagttagggcttggagctagtcgaccgttggaggctttgtcctcatgcggcaccggacagtctggtgctacaccggacagtccggtgcccctctgaccatctgctctgacatctgaattgcactgttcactttgcagagtcgaccattgcgtgcaggtagccattgctccgctggtgcaccggacagtccagtggcacaccggacagtccgatgaattatagcggagctgcgcctgggaaacccgaagctgaggagtttgagctgattcaccctggtgcaccggacactgtccggtggcacactggacagtccggtgcgccggaccagggcacacttcggtttcctttttgctcctttcttttgaagcctaacttgttcttttgattggtttgtgttgaacctttagcacctgtagaatgtatgatctagagcaaactagttagtccaattatttgtgttgggcaattcaaccaccaaaaacatttaggaaaatgtttgatcttatttccctttcatattctcttattgctagttgtcggggtgaagttgagctcttgcttaggttttaattagtgttgatttttagaaaaacccaattcacccccctcttgggcatcgtgatccttttagcaacaaaatgtgcacacatcaaaacaagcgcttctaccatatgtagttgttgcacaataatggtcctccttaggatttgcaaccgtttaacaatagctatgtgaccacagatttatgtcggatgcacgaaaattgtaggattttacatttctttaccttggttcacaaacattgaagcgacatagtggtgtatgaaaggaagcacttgtttttcaattccaaaccgcggtaccatttaaatcttaagcctaggataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtaccgaggccggccccgcgggagctcggcgcgccagattcacgtcagatttaaccaaaactatattatgaggtacacatattacaatccaaaatgaattatctagttctcgagttgtacacagtttatcacgtgttttacacattccaaccctaaactccaaccgtgg

SEQ ID NO: 254 - DNA, 4570 bp, Artificial Sequence
  MHP14Cas3 donor
acacttcggtttcctttttgctcctttcttttgaagcctaacttgttcttttgattggtttgtgttgaacctttagcacctgtagaatgtatgatctagagcaaactagttagtccaattatttgtgttgggcaattcaaccaccaaaaacatttaggaaaatgtttgatcttatttccctttcatattctcttattgctagttgtcggggtgaagttgagctcttgcttaggttttaattagtgttgatttttagaaaaacccaattcacccccctcttgggcatcgtgatccttttagcaacaaaatgtgcacacatcaaaacaagcgcttctaccatatgtagttgttgcacaataatggtcctccttaggatttgcaaccgtttaacaatagctatgtgaccacagatttatgtcggatgcacgaaaattgtaggattttacatttctttaccttggttcacaaacattgaagcgacaggtaccatttaaatcttaagcctaggataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtaccgaggccggccccgcgggagctcggcgcgcctagtggtgtatgaaaggaagcacttgtttttcaattccaaacagattcacgtcagatttaaccaaaactatattatgaggtacacatattacaatccaaaatgaattatctagttctcgagttgtacacagtttatcacgtgttttacacattccaaccctaaact

SEQ ID NO: 255 - DNA, 5091 bp, Artificial Sequence
  TS8Cas-1 donor
cacacatgactgcctgagaatctgctgccgttgcctctcatattatattcgatcccctgactaaaaaaactcggggccggctaatacgtactgtacgtacgcagaatttacggtccagcacgggcatgccgcgcgggctgactttgctccactgactcgatcatgtgcggattccatcgcggcgtagcgtagccaaccgcaacgcaaaccgacttcatcttttttttttattatgaacaaaaggagatcgagagaaacgtgaacggtaaataatatatctgatcccatgcatgcacgctgcctgggtcgatctcgctctcgctccgcccagacgaacatgcatgctggtcaggctcaacgctcaggcgggcaagctgtgggaggacatgggatgggagaggaggacacatgcatgctggccagtcaggcactgtgctggcacatgaggtagggataggggggccctcggccagtgtccaggccgcatgcatgcatgccccccctgctgctcgaccgaacaacgttggatgcctggattgatgcaacagtttggacggacggaccatacgttatgtaccagtaggtaccatttaaatcttaagcctaggataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtaccgaggccggccccgcggactgcacgttacgtacgtacgaactaatatactccaccagctgatcactgatgagccgagccgccatgcattgtaatttataacatgtgcggctgtacgcttccatctcaaatacctttttatatatatattgtactttatagtctacgacataatctgccatggtaatttataagatgtgctttattgctcgttgttctgttctcatctgtgtccatggcatggcatggatacaaaatgtatgtatggccacgcatccaatctgtgacgttgtcaaggcagaggtccaaccgtccaagaccctcttgtgccgccctgtacttgcagtcagtgacgttgtgagaaaaagctgtgggtggtctccgcagagcgcgcgggccacgagagggagccccatctctcggccgaggggtacgggggctccagacacggtcctttggtttcttctgcctgtagcgagcggccccgccccccaccgcgctgctagcctagccgatgctgatccatccaccacccacaagggattgttccacgacttgtggacctgaccatgacgtgacttcacgccatgtacgctcagccgctcactagcttttttttc

SEQ ID NO: 256 - DNA, 5237 bp, Artificial Sequence
  TS8Cas-2 donor
tctctttcagggcttgttcgtttacgttggattgcacccggaatcgttacagctaatcaaagtttatataaattagagaagcaaccggataggaatcgttccgacccaccaattcgacacaaacgaacaaggcctcaatccttctcaatccacctccaacccaataagctcttggaggcggcggcgggagagcagccacacacatgactgcctgagaatctgctgccgttgcctctcatattatattcgatcccctgactaaaaaaactcggggccggctaatacgtactgtacgtacgcagaatttacggtccagcacgggcatgccgcgcgggctgactttgctccactgactcgatcatgtgcggattccatcgcggcgtagcgtagccaaccgcaacgcaaaccgacttcatcttttttttttattatgaacaaaaggagatcgagagaaacgtgaacggtaaataatatatctgatcccatgcatgcacgctgcctgggtcgatctcgctctcgctccgcccagacgaacatgcatgctggtcaggctcaacgctcaggcgggcaagctgtgggaggacatgggatgggagaggaggacacatgcatgctggccagtcaggcactgtgctggcacatgaggtagggataggggggccctcggccagtgtccaggccgcatgcatgcatgccccccctgctgctcgaccgaacaacgttggatgcctggattgatgcaacagtttggacggacggaccatacgttatgtaccagtactgcacgttacgtacgtacgaactaatatactccaccaggtaccatttaaatcttaagcctaggataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtaccgaggccggccccgcgggagctcgctgatcactgatgagccgagccgccatgcattgtaatttataacatgtgcggctgtacgcttccatctcaaatacctttttatatatatattgtactttatagtctacgacataatctgccatggtaatttataagatgtgctttattgctcgttgttctgttctcatctgtgtccatggcatggcatggatacaaaatgtatgtatggccacgcatccaatctgtgacgttgtcaaggcagaggtccaaccgtccaagaccctcttgtgccgccctgtacttgcagtcagtgacgttgtgagaaaaagctgtgggtggtctccgcagagcgcgcgggccacgagagggagccccatctctcggccgaggggtacgggggctccagacacggtcctttggtttcttctgcctgtagcgagcggccccgccccccaccgcgctgctagcctagccgatgctgatccatccaccacccacaagggattgttccacgacttgtggac

SEQ ID NO: 257 - DNA, 5427 bp, Artificial Sequence
  TS9Cas-2 donor
agcaaggaactaaactgttattggacgctaaagtttagtactttatctttaacatctttcagcatttctatgtagatatttaagggctaaattttagcaagtgtgctgataaattttagcctaaatgtttctgttgggctaaattttagcaagtgtactgttaaattttagcatattccttttagagtggtatgggtgtgcatagactaaatgtttccgttgggccctaatttaacgatgtgtacgcaggcctgtttagatgacttggtaccggcatatggcctcgtactgtttcatttgatgacgcgagcgtgcggcccatgcagcagcagcacgccgggaaggcagcggattttgaagtactattggacagcgcggcgcggggaccgggtcgttggcgcgcggtggagtgggggtgggtggtcctggcgtcctgccctgcgcgatggtcgatggatgccccatgcgcgtgtaaccgcccagccgtcgccatccgaccaggtgggcagacgtacgtacggtggcacgcccacggcccatcggccatcgcgatcgcgttcgtatcgtgtcctcaataacgaaagcgccaacggaaggcgctgtcgtcgtcagttcaccgcgcgccggcgccctgtgtcctcgtccctctcgacttctcgaccagtaagaactctcgcgagctgcggagctgctggcgatggccggccggtgggatccgacgtgcgtgcaacctcgaatttaaatcttaagcctaggataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtaccgaggccggccccgcgggagctcggccgcaaacagcctggtgacagacgaagccagcaagcacgtacgtacgcacgtctctgctggtctggatgtgtatggatatggacgtctcacgtctggacgtcgtcgtcgccgttgtattgtatcatgccaaccacttccgtaccgtaccccctcgcgtgccaacatgaccaccgccggtacgtctccatcgtcggccgtcggcgtctcaggcagctctcaattaagcggacgtgttttggtaatctggtggaacgccgcgcgcactgagggtttgggggccccggcggacgagcgagcgagagacggtgcatgcatgccaaatggcaacgagggcccgcccgcccatccaataaccaacccagacgtagcgcaaccaacgtacgagtcctgtgctggcgcgtacgactaccacgctagctgccgcgacatgcgaactacggtccaccaggcaccagccatgacaatatatactgtatatatatttttcttcttctttttgtttccgctctctcaagttcctgctctgctcctgcctgtccgcggtgccgatcggcgagagagcatgcatggacatggaccacgcgagatccaggaaccggcacgggcccatgcgtggcaggcggccgtttcgtcaggttccccgaaatgccccaactgcgcggctgcaggatggctcatggctggctgcctagctggcccgtgacaccgatcgatcggtaacgacgacgcacgcacctgaagcacaggaaggagcctccctctcgcatgcacgttagtact

SEQ ID NO: 258 - DNA, 5426 bp, Artificial Sequence
  TS9Cas-3 donor
agcaaggaactaaactgttattggacgcaaagtttagtactttatctttaacatctttcagcatttctatgtagatatttaagggctaaattttagcaagtgtgctgataaattttagcctaaatgtttctgttgggctaaattttagcaagtgtactgttaaattttagcatattccttttagagtggtatgggtgtgcatagactaaatgtttccgttgggccctaatttaacgatgtgtacgcaggcctgtttagatgacttggtaccggcatatggcctcgtactgtttcatttgatgacgcgagcgtgcggcccatgcagcagcagcacgccgggaaggcagcggattttgaagtactattggacagcgcggcgcggggaccgggtcgttggcgcgcggtggagtgggggtgggtggtcctggcgtcctgccctgcgcgatggtcgatggatgccccatgcgcgtgtaaccgcccagccgtcgccatccgaccaggtgggcagacgtacgtacggtggcacgcccacggcccatcggccatcgcgatcgcgttcgtatcgtgtcctcaataacgaaagcgccaacggaaggcgctgtcgtcgtcagttcaccgcgcgccggcgccctgtgtcctcgtccctctcgacttctcgaccagtaagaactctcgcgagctgcggagctgctggcgatggccggccggtgggatccgacgatttaaatcttaagcctaggataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtaccgaggccggccccgcgggagctctgcgtgcaacctcgaggccgcaaacagcctggtgacagacgaagccagcaagcacgtacgtacgcacgtctctgctggtctggatgtgtatggatatggacgtctcacgtctggacgtcgtcgtcgccgttgtattgtatcatgccaaccacttccgtaccgtaccccctcgcgtgccaacatgaccaccgccggtacgtctccatcgtcggccgtcggcgtctcaggcagctctcaattaagcggacgtgttttggtaatctggtggaacgccgcgcgcactgagggtttgggggccccggcggacgagcgagcgagagacggtgcatgcatgccaaatggcaacgagggcccgcccgcccatccaataaccaacccagacgtagcgcaaccaacgtacgagtcctgtgctggcgcgtacgactaccacgctagctgccgcgacatgcgaactacggtccaccaggcaccagccatgacaatatatactgtatatatatttttcttcttctttttgtttccgctctctcaagttcctgctctgctcctgcctgtccgcggtgccgatcggcgagagagcatgcatggacatggaccacgcgagatccaggaaccggcacgggcccatgcgtggcaggcggccgtttcgtcaggttccccgaaatgccccaactgcgcggctgcaggatggctcatggctggctgcctagctggcccgtgacaccgatcgatcggtaacgacgacgcacgcacctgaagcacaggaaggagcctccctctcgcatgcacgttagtact

SEQ ID NO: 259 - DNA, 5152 bp, Artificial Sequence
  TS10Cas-1 donor
ggtaccaaatagtaaacgggaggggaggtcgctagtagtaaacgctaggtagctaggataatccgtctcgtgttggacggaaggttttggacgcatctgcgtgcacagcccgctgatacagatctgatcgactagctagctagatgccgaggccccagagcaaggcccggatactcctgcacagtccctgagatttcagcacagcaggtgctgttgcatcaatatataaatccctgctttattaatttaatctctgtgcatgtatccatacatcgtcagcggctcagcgctatcacactgcagtgcacgcagctagttgagcgcctgggtcagtatatatatagctagtagggacaaaggggggcactgtacgttggtttggtttggcacgcacgcgatcgagagtggtggaatggactgcagatcatcgatcgctgcactgtacgcacgcgcaccggactgcatttgcatgcccctgaaggaggaaaggggaaggaaagaaaagaaataggagaaagaagaagaagcagagaaatacgtcacagtccaagaagagtgagccgccctagctagcttcaaccctgacgaacccggcagccacacttccggccatgtatgcatgcatgcatggcttagcttcagatgtccaatcgaatccatcaagacctggccggttttccatggccgcctcgccttcgctagtggtaccatttaaatcttaagcctaggataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtaccgaggccggccccgcgggagctcggcgcgcctaagggccaagtacttgctgtccctgtatctccaacacgagccttgattcctgccggccggtgatggcaatggccgctagtagtctccgctagctagggagcggcgatccgacgcgacgccaccatgtgtctagaaaagaagtttcttgctttgcatgcagacttattagcgcggtcgacacctgtggggaccccgtgtcttgagacaatgagactgcctgtccgcccaagacactacttgtagccatgaagccatcgactcctctccttgctctccagtaatccagtggatggatccatcatcgatagtttagtttatcagtcttcttgaggccggtgtcccccatgcataatgatgacagaaagcctgggccaggtaaaagccaaaaagtttgaccctctaggtactggggccagccctggcgtttgaacaaaaaaaaaatctgagcgtgtcgccccggcctgttttcgaactcctaaacgacgtcgcaactttttttatacacacactaccggtacatggcttt

SEQ ID NO: 260 - DNA, 5146 bp, Artificial Sequence
  TS10Cas-3 donor
aaatagtaaacgggaggggaggtcgctagtagtaaacgctaggtagctaggataatccgtctcgtgttggacggaaggttttggacgcatctgcgtgcacagcccgctgatacagatctgatcgactagctagctagatgccgaggccccagagcaaggcccggatactcctgcacagtccctgagatttcagcacagcaggtgctgttgcatcaatatataaatccctgctttattaatttaatctctgtgcatgtatccatacatcgtcagcggctcagcgctatcacactgcagtgcacgcagctagttgagcgcctgggtcagtatatatatagctagtagggacaaaggggggcactgtacgttggtttggtttggcacgcacgcgatcgagagtggtggaatggactgcagatcatcgatcgctgcactgtacgcacgcgcaccggactgcatttgcatgcccctgaaggaggaaaggggaaggaaagaaaagaaataggagaaagaagaagaagcagagaaatacgtcacagtccaagaagagtgagccgccctagctagcttcaaccctgacgaacccggcagccacacttccggccatgtatgcatgcatgcatggcttagcttcagatgtccaatcgaatccatcaagacctggccggttttccatggccgcctcgccttcgctagttaagggccaagtacttgctgtccctgtggtaccatttaaatcttaagcctaggataacttcgtatagcatacattatacgaagttatggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtaccgaggccggccccgcgggagctcggcgcgccatctccaacacgagccttgattcctgccggccggtgatggcaatggccgctagtagtctccgctagctagggagcggcgatccgacgcgacgccaccatgtgtctagaaaagaagtttcttgctttgcatgcagacttattagcgcggtcgacacctgtggggaccccgtgtcttgagacaatgagactgcctgtccgcccaagacactacttgtagccatgaagccatcgactcctctccttgctctccagtaatccagtggatggatccatcatcgatagtttagtttatcagtcttcttgaggccggtgtcccccatgcataatgatgacagaaagcctgggccaggtaaaagccaaaaagtttgaccctctaggtactggggccagccctggcgtttgaacaaaaaaaaaatctgagcgtgtcgccccggcctgttttcgaactcctaaacgacgtcgcaactttttttatacacacactaccggtacatggcttt

SEQ ID NO: 261 - DNA, 32 bp, Artificial Sequence; identical to SEQ ID NO: 100
  ubir primer from donor

SEQ ID NO: 262 - DNA, 31 bp, Artificial Sequence; identical to SEQ ID NO: 99
  psbf primer from dono

SEQ ID NO: 263 - DNA, 27 bp, Artificial Sequences
  MHP14 14-1HR1f primer
ctcacatgaggctcttctttgcttgct

SEQ ID NO: 264 - DNA, 26 bp, Artificial Sequence
  MHP14 14-1HR2r primer
aggatcctattccccaatttgtagat

SEQ ID NO: 265 - DNA, 21 bp, Artificial Sequence
  CHR1-8 8HR1f primer
cagtccgtggattgaagccat

SEQ ID NO: 266 - DNA, 22 bp, Artificial Sequence
  CHR1-8 8HR2r primer
ctctgtctccgagacgtgctta

SEQ ID NO: 267 - DNA, 26 bp, Artificial Sequence
  CHR1-9 9HR1f primer
ggagcaaatgttttaggtatgaaatg

SEQ ID NO: 268 - DNA, 28 bp, Artificial Sequence
  CHR1-9  9HR2r primer
cggattctaaagatcatacgtaaatgaa

SEQ ID NO: 269 - DNA, 21 bp, Artificial Sequence
  CHR1-10  10HR1f primer
tggcttgtctatgcgcatctc

SEQ ID NO: 270 - DNA, 20 bp, Artificial Sequence
  CHR1-10  10HR2r primer
ccagacccaaacagcaggtt

SEQ ID NO: 271 - DNA, 18 bp, Artificial Sequence
  MHP14Cas-1 probe
cagattcacgtcagattt

SEQ ID NO: 272 - DNA, 27 bp, Artificial Sequence
  MHP14cas-1 forward primer
catagtggtgtatgaaaggaagcactt

SEQ ID NO: 273 - DNA, 30 bp, Artificial Sequence
  MHP14cas-1 reverse primer
cattttggattgtaatatgtgtacctcata

SEQ ID NO: 274 - DNA, 17 bp, Artificial Sequence
  MHP14Cas-3 probe
caccactatgtcgcttc

SEQ ID NO: 275 - DNA, 22 bp, Artificial Sequence
  MHP14Cas-3 forward primer
cggatgcacgaaaattgtagga

SEQ ID NO: 276 - DNA, 24 bp, Artificial Sequence
  MHP14Cas-3 reverse primer
ctgacgtgaatctgtttggaattg

SEQ ID NO: 277 - DNA, 18 bp, Artificial Sequence
  TS8Cas-1 probe
tacgtaacgtgcagtact

SEQ ID NO: 278 - DNA, 21 bp, Artificial Sequence
  TS8Cas-1 forward primer
acggacggaccatacgttatg

SEQ ID NO: 279 - DNA, 26 bp, Artificial Sequence
  TS8Cas-1 reverse primer
tcagctggtggagtatattagttcgt

SEQ ID NO: 280 - DNA, 18 bp, Artificial Sequence
  TS8Cas-2 probe
ccagctgatcactgatga

SEQ ID NO: 281 - DNA, 21 bp, Artificial Sequence
  TS8Cas-2 forward primer
acggacggaccatacgttatg

SEQ ID NO: 282 - DNA, 26 bp, Artificial Sequence
  TS8Cas-2 reverse primer
cgcacatgttataaattacaatgcat

SEQ ID NO: 283 - DNA, 14 bp, Artificial Sequence
  TS9Cas-2 probe
ctgtttgcggcctc

SEQ ID NO: 284 - DNA, 19 bp, Artificial Sequence
  TS9Cas-2 forward primer
ctgcggagctgctggcgat

SEQ ID NO: 285 - DNA, 20 bp, Artificial Sequence
  TS9Cas-2 reverse primer
cttgctggcttcgtctgtca

SEQ ID NO: 286 - DNA, 15 bp, Artificial Sequence
  TS9Cas-3 probe
ccgacgtgcgtgcaa

SEQ ID NO: 287 - DNA, 19 bp, Artificial Sequence
  TS9Cas-3 forward primer
ctgcggagctgctggcgat

SEQ ID NO: 288 - DNA, 20 bp, Artificial Sequence
  TS9Cas-3 reverse primer
cttgctggcttcgtctgtca

SEQ ID NO: 289 - DNA, 17 bp, Artificial Sequence
  TS10Cas-1 probe
tcgccttcgctagttaa

SEQ ID NO: 290 - DNA, 20 bp, Artificial Sequence
  TS10Cas-1 forward primer
aagacctggccggttttcca

SEQ ID NO: 291 - DNA, 18 bp, Artificial Sequence
  TS10Cas-1 reverse primer
tagcggccattgccatca

SEQ ID NO: 292 - DNA, 19 bp, Artificial Sequence
  TS10Cas-3 probe
ctgtatctccaacacgagc

SEQ ID NO: 293 - DNA, 20 bp, Artificial Sequence
  TS10Cas-3 forward primer
aagacctggccggttttcca

SEQ ID NO: 294 - DNA, 18 bp, Artificial Sequence
  TS10Cas-3 reverse primer
tagcggccattgccatca

SEQ ID NO: 295 - DNA, 472 bp, Glycine max
  GM-U6-9.1 promoter (1)..(472)
cccgggttaagagaattgtaagtgtgcttttatatatttaaaattaatatattttgaaatgttaaaatataaaagaaaattcaatgtaaattaaaaataaataaatgtttaataaagataaattttaaaacataaaagaaaatgtctaacaagaggattaagatcctgtgctcttaaatttttaggtgttgaaatcttagccatacaaaatatattttattaaaaccaagcatgaaaaaagtcactaaagagctatataactcatgcagctagaaatgaagtgaagggaatccagtttgttctcagtcgaaagagtgtctatctttgttcttttctgcaaccgagttaagcaaaatgggaatgcgaggtatcttcctttcgttaggggagcaccagatgcatagttagtcccacattgatgaatataacaagagcttcacagaatatatagcccaggccacagtaaaagctt

SEQ ID NO: 296 - DNA, 5958 bp, Artificial sequence
  EF1A2-CAS9
gggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 297 - DNA, 573 bp, Artificial sequence
  U6-9.1-DD20CR1
ccgggttaagagaattgtaagtgtgcttttatatatttaaaattaatatattttgaaatgttaaaatataaaagaaaattcaatgtaaattaaaaataaataaatgtttaataaagataaattttaaaacataaaagaaaatgtctaacaagaggattaagatcctgtgctcttaaatttttaggtgttgaaatcttagccatacaaaatatattttattaaaaccaagcatgaaaaaagtcactaaagagctatataactcatgcagctagaaatgaagtgaagggaatccagtttgttctcagtcgaaagagtgtctatctttgttcttttctgcaaccgagttaagcaaaatgggaatgcgaggtatcttcctttcgttaggggagcaccagatgcatagttagtcccacattgatgaatataacaagagcttcacagaatatatagcccaggccacagtaaaagcttggaactgacacacgacatgagttttagagctagaaatagcaagttaaaataaggctagtccgttatcaacttgaaaaagtggcaccgagtcggtgctttttt

SEQ ID NO: 298 - DNA, 6611 bp, Artificial sequence
  U6-9.1-DD20CR1+EF1A2-CAS9
cgcgccggtacccgggttaagagaattgtaagtgtgcttttatatatttaaaattaatatattttgaaatgttaaaatataaaagaaaattcaatgtaaattaaaaataaataaatgtttaataaagataaattttaaaacataaaagaaaatgtctaacaagaggattaagatcctgtgctcttaaatttttaggtgttgaaatcttagccatacaaaatatattttattaaaaccaagcatgaaaaaagtcactaaagagctatataactcatgcagctagaaatgaagtgaagggaatccagtttgttctcagtcgaaagagtgtctatctttgttcttttctgcaaccgagttaagcaaaatgggaatgcgaggtatcttcctttcgttaggggagcaccagatgcatagttagtcccacattgatgaatataacaagagcttcacagaatatatagcccaggccacagtaaaagcttggaactgacacacgacatgagttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccgg

SEQ ID NO: 299 - DNA, 5686 bp, Artificial sequence
  DD20HR1-SAMSHPT-DD20HR2
cgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcgctatcaactttgtatagaaaagttgggccgaattcgagctcggtacggccagaatccggtaagtgactagggtcacgtgaccctagtcacttaaattcggccagaatggccatctggattcagcaggcctagaaggcccggaccgattaaactttaattcggtccgggttacctcgagcctagtaataattacacatctaagatatccccttctttttcaagtaaaataatatcatatgatctcattttagtgaaacaatactatttccctgataactctcttcaacattagggacttcatctaatcatctactttcaaggtataactagacgtatttgttcttttaaaaaaaacactagatgtactcgtcaactcaaaattcatcgttcatgcattttaattaaactttaattagctaatgagtagaaaaagatcatacgagtaaaatagaagaatcttcctagattttggaagaatggattggagtgtaagtgaattgatccattagtggaagatgctctttacaatggccaaactgttctaattgttagagcacatttgagatgaaacacttcagtagtggaggtaacctacaatcctaggatctgtatcctctatcactaatggagcaatgggtttgagattgacttactcctttccttgtctctcgtagtgcatatgcgcactttcaaaggctacacaaaagccgttaactttttgtttatttaagttacgaaagatagttgaattagagtaaatggtgatattgaattaggattttaaataattttaaaagaatttttttaataaaaaaaatattgtgttgttggatcaaaatttttaaataacatgaataaggaaatggattgcaatgaggttttaaacaattattttaacatataggattttagaaagacttttataatattttgttgaagtttagattttaatatatttatgttttaaaattttaaaaaaaacttcatgaatttataatatttgaaaaagacacgtgaatatttagaaaacatttaaaattacaataataaatcataatgagatagggtgtattcatgtgtagacgagacaccaagtatatggttcacaagtgaatcatcttttttttttacagcacaagtagatcacttgtacttatcaaaattcggaactgacacacactagtggtcacctaagtgactagggtcacgtgaccctagtcacttattcccaaacactagtaacggccgccagtgtgctggaattcgcccttcccaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgtttgactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataacaacagaagttcctattccgaagttcctattctctagaaagtataggaacttccactagtccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgacccgggccctaggaggccggcccagctgatgatcccggtgaagttcctattccgaagttcctattctccagaaagtataggaacttcactagagcttgcggccgcgcatgctgacttaatcagctaacgccactcgacctgcaggcatgcccgcggatatcgatgggccccggccgaagcttcaagtttgtacaaaaaagcaggctggcgccggaaccaattcagtcgactggatccggtaccgaattcgcggccgcactcgagatatctagacccagctttcttgtacaaagtggccgttaacggatcggccagaatccggtaagtgactagggtcacgtgaccctagtcacttaaattcggccagaatggccatctggattcagcaggcctagaaggcccggaccgattaaactttaattcggtccgggttacctctagaaagcttgtcgacctgcagacacgacatgatggaacgtgactaaggtgggtttttgactttgcatgtcgaagtgagagtgattttattgagagaataatagaagacctacaaaacaaatgatcccgacgctaaagtaagtacgagagttaagagaataaatgggaaaatatgcatacatgattaggtgtgtgttcgtctcaagaaagtacgaatgaatatggtgtgtttgtagtacatgaatgatgtgttttgagggttcaagggaaattgatatttatagagtgaaatggaaccagaggtctttgttgacaagggttgttatgactcttgcaaataattaatagcttataaataatagccaataacttattatagatagagttagagataatatatagctaaatttgaacaaggcatacaaaacaaaaatgctaaatatgaataagacaatcaaaattgtagtcgatgttcaactctttgtcgttgaagaacttgtttgcagtggtatagtaaatgggtgtgagtgcagtgtctcacccatctcacaccacacaaccaacttcatatctaaagatattgtcgctgaatacaaaattgagttatggaatatacaattcataatatagatacgaaaaatcatttcttacaaaacattcaatcaaaaattattcaaacataattctagattaagtaatccgaagtacaagttagtatcctagatccgttaatttaaaattatgtttgcataattttggatttggtgttctataagggcacaattttgttcattcttacaagtttgtcaattctaaaatatatgcaaatttgaagaaaaaaaatttacgaatgtgtctcaaacaataacttaatgggaggagaatgagggatgaagaagctcaaaattaccaacgccttctacctcaagaagctacttcacacaaaatatgactggcggaaggataggggacaaccgataacgagaaggagatacataaggtaatgtacgttgttgtgtgagggatccggtcacctaagtgactagggtcacgtgaccctagtcacttattcccgggcaactttattatacaaagttgatagatctcgaattcattccgattaatcgtgg

SEQ ID NO: 300 - DNA, 6611 bp, Artificial sequence
  U6-9.1-DD20CR2+EF1A2-CAS9
cgcgccggtacccgggttaagagaattgtaagtgtgcttttatatatttaaaattaatatattttgaaatgttaaaatataaaagaaaattcaatgtaaattaaaaataaataaatgtttaataaagataaattttaaaacataaaagaaaatgtctaacaagaggattaagatcctgtgctcttaaatttttaggtgttgaaatcttagccatacaaaatatattttattaaaaccaagcatgaaaaaagtcactaaagagctatataactcatgcagctagaaatgaagtgaagggaatccagtttgttctcagtcgaaagagtgtctatctttgttcttttctgcaaccgagttaagcaaaatgggaatgcgaggtatcttcctttcgttaggggagcaccagatgcatagttagtcccacattgatgaatataacaagagcttcacagaatatatagcccaggccacagtaaaagcttgacatgatggaacgtgactagttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccgg

SEQ ID NO: 301 - DNA, 6611 bp, Artificial sequence
  U6-9.1DD43CR1+EF1A2CAS9
cgcgccggtacccgggttaagagaattgtaagtgtgcttttatatatttaaaattaatatattttgaaatgttaaaatataaaagaaaattcaatgtaaattaaaaataaataaatgtttaataaagataaattttaaaacataaaagaaaatgtctaacaagaggattaagatcctgtgctcttaaatttttaggtgttgaaatcttagccatacaaaatatattttattaaaaccaagcatgaaaaaagtcactaaagagctatataactcatgcagctagaaatgaagtgaagggaatccagtttgttctcagtcgaaagagtgtctatctttgttcttttctgcaaccgagttaagcaaaatgggaatgcgaggtatcttcctttcgttaggggagcaccagatgcatagttagtcccacattgatgaatataacaagagcttcacagaatatatagcccaggccacagtaaaagcttgtcccttgtacttgtacgtagttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccgg

SEQ ID NO: 302 - DNA, 5719 bp, Artificial sequence
  DD43HR1-SAMSHPT-DD43HR2
cgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcgctatcaactttgtatagaaaagttgggccgaattcgagctcggtacggccagaatccggtaagtgactagggtcacgtgaccctagtcacttaaattcggccagaatggccatctggattcagcaggcctagaaggcccggaccgattaaactttaattcggtccgggttacctcgagatcttgttcccctccttggtttggcataaattgattttcatggctcttctcggtcgaaactggagctaattcacccttagtctctcttaaaattctggctgtaagaaacaccacagaacacataaattataaactaattataatttgaagagtaaaatatgtttttactcttatgatttaattagtgtagttttaattttctcctttttttaaaaaattttggtattcataaatttcaattttttaaaaataattgttgttacccgttaatgataacgggatatgttatgttaccactaaatcggacaaaaaaaattcaaaacttttataaggattaaaattaacaaaaatattttaaaaaaatctaacctcaataaagttaaatttataagcacaaaataatacttttaagcctaatttggcaagacacaagcaagctcacctgtagcattaatagaaaggaagcaaagcaagagaaaagcaaccagaaggaagcgtttgcttggtgacacagccatcttacttgaatttatggtattactgagaaaccttgatcttgcttcaaaatcttctagttaccctctttttataggcagaaagagaactagctagttgccaataggatatgaggacatgtggtgcaatgcactcactcttcaaggacaagaaaaacaatggctacaattgtggttcaaatcaatgtctcctgctctgtcctgcctgaaaatgacacccttttgcttggaaaagaggatcaaagctaagaacaggagtggcttcattcccttcatgtaaccaaacactttcgcattctgtcattcgtgaatcagcaaaatctgcaaccaaaaatatatggtgcctaaataaaagaaataaaataatttagagttgcggactaaaataataaacaaaagaaatatattataatctagaattaatttaggactaaaagaagaggcagactccaattcctcttttctagaataccctccgtacgtacactagtggtcacctaagtgactagggtcacgtgaccctagtcacttattcccaaacactagtaacggccgccagtgtgctggaattcgcccttcccaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgtttgactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataacaacagaagttcctattccgaagttcctattctctagaaagtataggaacttccactagtccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgacccgggccctaggaggccggcccagctgatgatcccggtgaagttcctattccgaagttcctattctccagaaagtataggaacttcactagagcttgcggccgcgcatgctgacttaatcagctaacgccactcgacctgcaggcatgcccgcggatatcgatgggccccggccgaagcttcaagtttgtacaaaaaagcaggctggcgccggaaccaattcagtcgactggatccggtaccgaattcgcggccgcactcgagatatctagacccagctttcttgtacaaagtggccgttaacggatcggccagaatccggtaagtgactagggtcacgtgaccctagtcacttaaattcggccagaatggccatctggattcagcaggcctagaaggcccggaccgattaaactttaattcggtccgggttacctctagaaagcttgtcgacctgcaggtacaagtacaagggacttgtgagttgtaaggctgtatttacaatagtgaaaagagaatcatctgggtgattgggtttttagtccccagtgacgaattaaaggtttgaattcttagtatgtttgggaatcaattaggaatttcgttttggactttccaaagcaattattcactttttcattcattaaatgtgactaaaaaattgttatttctccattggccaggatgcatcgtttatataaacataaccttagtgaaagcagtgttttcatgtgacagcggcagactatatcttaaacaaaattacttgtaaagaaagataccgttaggaaaaaaatgaaaagaaaattgaagctatcacttgtttactttcctaatatctttcaagaatacaatgtggtgaatttcaattttccctacatatgtataccgtcagcctgacgcaacttatgaaacttctctttctttcatttgatgtatatataaagacacattatatataaagaaactttatatatatctccatcatattttagtacttgctactatgtaaaattagctgttggaagtatctcaagaaacatttaatttattgaaccaagcattaaccattcatctacatttgagttctaaaataaatcttaaatgatgtggaggaagggaaattgttaattatttccctcttctcctacatggatatacctgaaacatgcaatggatggattagattttaacatttgcagcctgagaagttcactgactttcctccagctattttatgtgtgcccgccaccatttatagctcatgattgtagctgaactgcaaaaactgcatcgattgcaaactgaaattgagaatctcttttcaactttatatgctgattgatgcatgctgagcatgctatactagtactcgaagttcctatatgtagactttgttactgcctaatatactttgtgtttgttctcaagttcttattttatttcatattttttcctataaaaggttaatggctctataaaggttgagtgacggatccggtcacctaagtgactagggtcacgtgaccctagtcacttattcccgggcaactttattatacaaagttgatagatctcgaattcattccgattaatcgtgg

SEQ ID NO: 303 - DNA, 6611 bp, Artificial sequence
  U6-9.1DD43CR2+EF1A2CAS9
cgcgccggtacccgggttaagagaattgtaagtgtgcttttatatatttaaaattaatatattttgaaatgttaaaatataaaagaaaattcaatgtaaattaaaaataaataaatgtttaataaagataaattttaaaacataaaagaaaatgtctaacaagaggattaagatcctgtgctcttaaatttttaggtgttgaaatcttagccatacaaaatatattttattaaaaccaagcatgaaaaaagtcactaaagagctatataactcatgcagctagaaatgaagtgaagggaatccagtttgttctcagtcgaaagagtgtctatctttgttcttttctgcaaccgagttaagcaaaatgggaatgcgaggtatcttcctttcgttaggggagcaccagatgcatagttagtcccacattgatgaatataacaagagcttcacagaatatatagcccaggccacagtaaaagcttgtattctagaaaagaggaatgttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccgg

SEQ ID NO: 304 - DNA, 64 bp, Artificial sequence
  DD20 qPCR amplicon
attcggaactgacacacgacatgatggaacgtgactaaggtgggtttttgactttgcatgtcga

SEQ ID NO: 305 - DNA, 115 bp, Artificial sequence
  DD43 qPCR amplicon
aaagaagaggcagactccaattcctcttttctagaataccctccgtacgtacaagtacaagggacttgtgagttgtaaggctgtatttacaatagtgaaaagagaatcatctggg

SEQ ID NO: 306 - DNA, 20 bp, Artificial sequence
  primer, DD20-CR1
ggaactgacacacgacatga

SEQ ID NO: 307 - DNA, 20 bp, Artificial sequence
  primer, DD20-CR2
gacatgatggaacgtgacta

SEQ ID NO: 308 - DNA, 22 bp, Artificial sequence
  primer, DD20-F
attcggaactgacacacgacat

SEQ ID NO: 309 - DNA, 17 bp, Artificial sequence
  FAM-MGB probe, DD20-T
atggaacgtgactaagg

SEQ ID NO: 310 - DNA, 22 bp, Artificial sequence
  primer, DD20-R
tcgacatgcaaagtcaaaaacc

SEQ ID NO: 311 - DNA, 20 bp, Artificial sequence
  primer, DD43CR1
gtcccttgtacttgtacgta

SEQ ID NO: 312 - DNA, 20 bp, Artificial sequence
  primer, DD43CR2
gtattctagaaaagaggaat

SEQ ID NO: 313 - DNA, 26 bp, Artificial sequence
  primer, DD43-F
ttctagaataccctccgtacgtacaa

SEQ ID NO: 314 - DNA, 26 bp, Artificial sequence
  primer, DD43-F2
aaagaagaggcagactccaattcctc

SEQ ID NO: 315 - DNA, 19 bp, Artificial sequence
  FAM-MGB probe, DD43-T
caagggacttgtgagttgt

SEQ ID NO: 316 - DNA, 26 bp, Artificial sequence
  primer, DD43-R
cccagatgattctcttttcactattg

SEQ ID NO: 317 - DNA, 19 bp, Artificial sequence
  primer, Cas9-F
ccttcttccaccgccttga

SEQ ID NO: 318 - DNA, 19 bp, Artificial sequence
  FAM-MGB probe, Cas9-T
aatcattcctggtggagga

SEQ ID NO: 319 - DNA, 21 bp, Artificial sequence
  primer, Cas9-R
tgggtgtctctcgtgcttttt

SEQ ID NO: 320 - DNA, 22 bp, Artificial sequence
  primer, Sams-76F
aggcttgttgtgcagtttttga

SEQ ID NO: 321 - DNA, 22 bp, Artificial sequence
  FAM-MGB probe, FRT1I-63T
tggactagtggaagttcctata

SEQ ID NO: 322 - DNA, 21 bp, Artificial sequence
  primer, FRT1I-41F
gcggtgagttcaggctttttc

SEQ ID NO: 323 - DNA, 31 bp, Artificial sequence
  primer, DD20-LB
ggttataccttcttcttagtgtggtctatcc

SEQ ID NO: 324 - DNA, 31 bp, Artificial sequence
  primer, Sams-A1
cccaaaataattagtatgattggtaaggaag

SEQ ID NO: 325 - DNA, 23 bp, Artificial sequence
  primer, QC498A-S1
ggaacttcactagagcttgcggc

SEQ ID NO: 326 - DNA, 28 bp, Artificial sequence
  primer, DD20-RB
gccattacattcttcataagttcctctc

SEQ ID NO: 327 - DNA, 26 bp, Artificial sequence
  primer, DD43-LB
gtgtagtccattgtagccaagtcacc

SEQ ID NO: 328 - DNA, 24 bp, Artificial sequence
  primer, DD43-RB
caaaccggagagagaggaagaacc

SEQ ID NO: 329 - DNA, 2105 bp, Artificial Sequence
  DD20 HR1-HR2 PCR amplicon
ggttataccttcttcttagtgtggtctatcccctagtaataattacacatctaagatatccccttctttttcaagtaaaataatatcatatgatctcattttagtgaaacaatactatttccctgataactctcttcaacattagggacttcatctaatcatctactttcaaggtataactagacgtatttgttcttttaaaaaaaacactagatgtactcgtcaactcaaaattcatcgttcatgcattttaattaaactttaattagctaatgagtagaaaaagatcatacgagtaaaatagaagaatcttcctagattttggaagaatggattggagtgtaagtgaattgatccattagtggaagatgctctttacaatggccaaactgttctaattgttagagcacatttgagatgaaacacttcagtagtggaggtaacctacaatcctaggatctgtatcctctatcactaatggagcaatgggtttgagattgacttactcctttccttgtctctcgtagtgcatatgcgcactttcaaaggctacacaaaagccgttaactttttgtttatttaagttacgaaagatagttgaattagagtaaatggtgatattgaattaggattttaaataattttaaaagaatttttttaataaaaaaaatattgtgttgttggatcaaaatttttaaataacatgaataaggaaatggattgcaatgaggttttaaacaattattttaacatataggattttagaaagacttttataatattttgttgaagtttagattttaatatatttatgttttaaaattttaaaaaaaacttcatgaatttataatatttgaaaaagacacgtgaatatttagaaaacatttaaaattacaataataaatcataatgagatagggtgtattcatgtgtagacgagacaccaagtatatggttcacaagtgaatcatcttttttttttacagcacaagtagatcacttgtacttatcaaaattcggaactgacacacgacatgatggaacgtgactaaggtgggtttttgactttgcatgtcgaagtgagagtgattttattgagagaataatagaagacctacaaaacaaatgatcccgacgctaaagtaagtacgagagttaagagaataaatgggaaaatatgcatacatgattaggtgtgtgttcgtctcaagaaagtacgaatgaatatggtgtgtttgtagtacatgaatgatgtgttttgagggttcaagggaaattgatatttatagagtgaaatggaaccagaggtctttgttgacaagggttgttatgactcttgcaaataattaatagcttataaataatagccaataacttattatagatagagttagagataatatatagctaaatttgaacaaggcatacaaaacaaaaatgctaaatatgaataagacaatcaaaattgtagtcgatgttcaactctttgtcgttgaagaacttgtttgcagtggtatagtaaatgggtgtgagtgcagtgtctcacccatctcacaccacacaaccaacttcatatctaaagatattgtcgctgaatacaaaattgagttatggaatatacaattcataatatagatacgaaaaatcatttcttacaaaacattcaatcaaaaattattcaaacataattctagattaagtaatccgaagtacaagttagtatcctagatccgttaatttaaaattatgtttgcataattttggatttggtgttctataagggcacaattttgttcattcttacaagtttgtcaattctaaaatatatgcaaatttgaagaaaaaaaatttacgaatgtgtctcaaacaataacttaatgggaggagaatgagggatgaagaagctcaaaattaccaacgccttctacctcaagaagctacttcacacaaaatatgactggcggaaggataggggacaaccgataacgagaaggagatacataaggtaatgtacgttgttgtgtgaggtacacaattatggggatgaagaagttcaactttagtcgaaaaaatgtttgagaggaacttatgaagaatgtaatggc

SEQ ID NO: 330 - DNA, 1204 bp, Artificial Sequence
  DD20 HR1-SAMS PCR amplicon
ggttataccttcttcttagtgtggtctatcccctagtaataattacacatctaagatatccccttctttttcaagtaaaataatatcatatgatctcattttagtgaaacaatactatttccctgataactctcttcaacattagggacttcatctaatcatctactttcaaggtataactagacgtatttgttcttttaaaaaaaacactagatgtactcgtcaactcaaaattcatcgttcatgcattttaattaaactttaattagctaatgagtagaaaaagatcatacgagtaaaatagaagaatcttcctagattttggaagaatggattggagtgtaagtgaattgatccattagtggaagatgctctttacaatggccaaactgttctaattgttagagcacatttgagatgaaacacttcagtagtggaggtaacctacaatcctaggatctgtatcctctatcactaatggagcaatgggtttgagattgacttactcctttccttgtctctcgtagtgcatatgcgcactttcaaaggctacacaaaagccgttaactttttgtttatttaagttacgaaagatagttgaattagagtaaatggtgatattgaattaggattttaaataattttaaaagaatttttttaataaaaaaaatattgtgttgttggatcaaaatttttaaataacatgaataaggaaatggattgcaatgaggttttaaacaattattttaacatataggattttagaaagacttttataatattttgttgaagtttagattttaatatatttatgttttaaaattttaaaaaaaacttcatgaatttataatatttgaaaaagacacgtgaatatttagaaaacatttaaaattacaataataaatcataatgagatagggtgtattcatgtgtagacgagacaccaagtatatggttcacaagtgaatcatcttttttttttacagcacaagtagatcacttgtacttatcaaaattcggaactgacacacactagtggtcacctaagtgactagggtcacgtgaccctagtcacttattcccaaacactagtaacggccgccagtgtgctggaattcgcccttcccaagctttgctctagatcaaactcacatccaaacataacatggatatcttccttaccaatcatactaattattttggg

SEQ ID NO: 331 - DNA, 1459 bp, Artificial Sequence
  DD20 NOS-HR2 PCR amplicon
ggaacttcactagagcttgcggccgcgcatgctgacttaatcagctaacgccactcgacctgcaggcatgcccgcggatatcgatgggccccggccgaagcttcaagtttgtacaaaaaagcaggctggcgccggaaccaattcagtcgactggatccggtaccgaattcgcggccgcactcgagatatctagacccagctttcttgtacaaagtggccgttaacggatcggccagaatccggtaagtgactagggtcacgtgaccctagtcacttaaattcggccagaatggccatctggattcagcaggcctagaaggcccggaccgattaaactttaattcggtccgggttacctctagaaagcttgtcgacctgcagacacgacatgatggaacgtgactaaggtgggtttttgactttgcatgtcgaagtgagagtgattttattgagagaataatagaagacctacaaaacaaatgatcccgacgctaaagtaagtacgagagttaagagaataaatgggaaaatatgcatacatgattaggtgtgtgttcgtctcaagaaagtacgaatgaatatggtgtgtttgtagtacatgaatgatgtgttttgagggttcaagggaaattgatatttatagagtgaaatggaaccagaggtctttgttgacaagggttgttatgactcttgcaaataattaatagcttataaataatagccaataacttattatagatagagttagagataatatatagctaaatttgaacaaggcatacaaaacaaaaatgctaaatatgaataagacaatcaaaattgtagtcgatgttcaactctttgtcgttgaagaacttgtttgcagtggtatagtaaatgggtgtgagtgcagtgtctcacccatctcacaccacacaaccaacttcatatctaaagatattgtcgctgaatacaaaattgagttatggaatatacaattcataatatagatacgaaaaatcatttcttacaaaacattcaatcaaaaattattcaaacataattctagattaagtaatccgaagtacaagttagtatcctagatccgttaatttaaaattatgtttgcataattttggatttggtgttctataagggcacaattttgttcattcttacaagtttgtcaattctaaaatatatgcaaatttgaagaaaaaaaatttacgaatgtgtctcaaacaataacttaatgggaggagaatgagggatgaagaagctcaaaattaccaacgccttctacctcaagaagctacttcacacaaaatatgactggcggaaggataggggacaaccgataacgagaaggagatacataaggtaatgtacgttgttgtgtgaggtacacaattatggggatgaagaagttcaactttagtcgaaaaaatgtttgagaggaacttatgaagaatgtaatggc

SEQ ID NO: 332 - DNA, 2098 bp, Artificial Sequence
  DD43 HR1-HR2 PCR amplicon
gtgtagtccattgtagccaagtcaccaatatcttgttcccctccttggtttggcataaattgattttcatggctcttctcggtcgaaactggagctaattcacccttagtctctcttaaaattctggctgtaagaaacaccacagaacacataaattataaactaattataatttgaagagtaaaatatgtttttactcttatgatttaattagtgtagttttaattttctcctttttttaaaaaattttggtattcataaatttcaattttttaaaaataattgttgttacccgttaatgataacgggatatgttatgttaccactaaatcggacaaaaaaaattcaaaacttttataaggattaaaattaacaaaaatattttaaaaaaatctaacctcaataaagttaaatttataagcacaaaataatacttttaagcctaatttggcaagacacaagcaagctcacctgtagcattaatagaaaggaagcaaagcaagagaaaagcaaccagaaggaagcgtttgcttggtgacacagccatcttacttgaatttatggtattactgagaaaccttgatcttgcttcaaaatcttctagttaccctctttttataggcagaaagagaactagctagttgccaataggatatgaggacatgtggtgcaatgcactcactcttcaaggacaagaaaaacaatggctacaattgtggttcaaatcaatgtctcctgctctgtcctgcctgaaaatgacacccttttgcttggaaaagaggatcaaagctaagaacaggagtggcttcattcccttcatgtaaccaaacactttcgcattctgtcattcgtgaatcagcaaaatctgcaaccaaaaatatatggtgcctaaataaaagaaataaaataatttagagttgcggactaaaataataaacaaaagaaatatattataatctagaattaatttaggactaaaagaagaggcagactccaattcctcttttctagaataccctccgtacgtacaagtacaagggacttgtgagttgtaaggctgtatttacaatagtgaaaagagaatcatctgggtgattgggtttttagtccccagtgacgaattaaaggtttgaattcttagtatgtttgggaatcaattaggaatttcgttttggactttccaaagcaattattcactttttcattcattaaatgtgactaaaaaattgttatttctccattggccaggatgcatcgtttatataaacataaccttagtgaaagcagtgttttcatgtgacagcggcagactatatcttaaacaaaattacttgtaaagaaagataccgttaggaaaaaaatgaaaagaaaattgaagctatcacttgtttactttcctaatatctttcaagaatacaatgtggtgaatttcaattttccctacatatgtataccgtcagcctgacgcaacttatgaaacttctctttctttcatttgatgtatatataaagacacattatatataaagaaactttatatatatctccatcatattttagtacttgctactatgtaaaattagctgttggaagtatctcaagaaacatttaatttattgaaccaagcattaaccattcatctacatttgagttctaaaataaatcttaaatgatgtggaggaagggaaattgttaattatttccctcttctcctacatggatatacctgaaacatgcaatggatggattagattttaacatttgcagcctgagaagttcactgactttcctccagctattttatgtgtgcccgccaccatttatagctcatgattgtagctgaactgcaaaaactgcatcgattgcaaactgaaattgagaatctcttttcaactttatatgctgattgatgcatgctgagcatgctatactagtactcgaagttcctatatgtagactttgttactgcctaatatactttgtgtttgttctcaagttcttattttatttcatattttttcctataaaaggttaatggctctataaaggttgagtgacatatatatactataaaggttcttcctctctctccggtttg

SEQ ID NO: 333 - DNA, 1202 bp, Artificial Sequence
  DD43 HR1-SAMS PCR PCR amplicon
gtgtagtccattgtagccaagtcaccaatatcttgttcccctccttggtttggcataaattgattttcatggctcttctcggtcgaaactggagctaattcacccttagtctctcttaaaattctggctgtaagaaacaccacagaacacataaattataaactaattataatttgaagagtaaaatatgtttttactcttatgatttaattagtgtagttttaattttctcctttttttaaaaaattttggtattcataaatttcaattttttaaaaataattgttgttacccgttaatgataacgggatatgttatgttaccactaaatcggacaaaaaaaattcaaaacttttataaggattaaaattaacaaaaatattttaaaaaaatctaacctcaataaagttaaatttataagcacaaaataatacttttaagcctaatttggcaagacacaagcaagctcacctgtagcattaatagaaaggaagcaaagcaagagaaaagcaaccagaaggaagcgtttgcttggtgacacagccatcttacttgaatttatggtattactgagaaaccttgatcttgcttcaaaatcttctagttaccctctttttataggcagaaagagaactagctagttgccaataggatatgaggacatgtggtgcaatgcactcactcttcaaggacaagaaaaacaatggctacaattgtggttcaaatcaatgtctcctgctctgtcctgcctgaaaatgacacccttttgcttggaaaagaggatcaaagctaagaacaggagtggcttcattcccttcatgtaaccaaacactttcgcattctgtcattcgtgaatcagcaaaatctgcaaccaaaaatatatggtgcctaaataaaagaaataaaataatttagagttgcggactaaaataataaacaaaagaaatatattataatctagaattaatttaggactaaaagaagaggcagactccaattcctcttttctagaataccctccgtacgtacactagtggtcacctaagtgactagggtcacgtgaccctagtcacttattcccaaacactagtaacggccgccagtgtgctggaattcgcccttcccaagctttgctctagatcaaactcacatccaaacataacatggatatcttccttaccaatcatactaattattttggg

SEQ ID NO: 334 - DNA, 1454 bp, Artificial Sequence
  DD43 NOS-HR2 PCR PCR amplicon
ggaacttcactagagcttgcggccgcgcatgctgacttaatcagctaacgccactcgacctgcaggcatgcccgcggatatcgatgggccccggccgaagcttcaagtttgtacaaaaaagcaggctggcgccggaaccaattcagtcgactggatccggtaccgaattcgcggccgcactcgagatatctagacccagctttcttgtacaaagtggccgttaacggatcggccagaatccggtaagtgactagggtcacgtgaccctagtcacttaaattcggccagaatggccatctggattcagcaggcctagaaggcccggaccgattaaactttaattcggtccgggttacctctagaaagcttgtcgacctgcaggtacaagtacaagggacttgtgagttgtaaggctgtatttacaatagtgaaaagagaatcatctgggtgattgggtttttagtccccagtgacgaattaaaggtttgaattcttagtatgtttgggaatcaattaggaatttcgttttggactttccaaagcaattattcactttttcattcattaaatgtgactaaaaaattgttatttctccattggccaggatgcatcgtttatataaacataaccttagtgaaagcagtgttttcatgtgacagcggcagactatatcttaaacaaaattacttgtaaagaaagataccgttaggaaaaaaatgaaaagaaaattgaagctatcacttgtttactttcctaatatctttcaagaatacaatgtggtgaatttcaattttccctacatatgtataccgtcagcctgacgcaacttatgaaacttctctttctttcatttgatgtatatataaagacacattatatataaagaaactttatatatatctccatcatattttagtacttgctactatgtaaaattagctgttggaagtatctcaagaaacatttaatttattgaaccaagcattaaccattcatctacatttgagttctaaaataaatcttaaatgatgtggaggaagggaaattgttaattatttccctcttctcctacatggatatacctgaaacatgcaatggatggattagattttaacatttgcagcctgagaagttcactgactttcctccagctattttatgtgtgcccgccaccatttatagctcatgattgtagctgaactgcaaaaactgcatcgattgcaaactgaaattgagaatctcttttcaactttatatgctgattgatgcatgctgagcatgctatactagtactcgaagttcctatatgtagactttgttactgcctaatatactttgtgtttgttctcaagttcttattttatttcatattttttcctataaaaggttaatggctctataaaggttgagtgacatatatatactataaaggttcttcctctctctccggtttg

SEQ ID NO: 335 - DNA, 60 bp, Glycine max
  soybean genomic DD20CR1 target region (1)..(60)
acttgtacttatcaaaattcggaactgacacacgacatgatggaacgtgactaaggtggg

SEQ ID NO: 336 - DNA, 59 bp, Artificial sequence
  sequence of gRNA/Cas9 system mediated NHEJ
acttgtacttatcaaaattcggaactgacacacgactgatggaacgtgactaaggtggg

SEQ ID NO: 337 - DNA, 59 bp, Artificial sequence
  sequence of gRNA/Cas9 system mediated NHEJ
acttgtacttatcaaaattcggaactgacacacgaatgatggaacgtgactaaggtggg

SEQ ID NO: 338 - DNA, 58 bp, Artificial sequence
  sequence of gRNA/Cas9 system mediated NHEJ
acttgtacttatcaaaattcggaactgacacacgatgatggaacgtgactaaggtggg

SEQ ID NO: 339 - DNA, 58 bp, Artificial sequence
  sequence of gRNA/Cas9 system mediated NHEJ
acttgtacttatcaaaattcggaactgacacacgacgatggaacgtgactaaggtggg

SEQ ID NO: 340 - DNA, 58 bp, Artificial sequence
  sequence of gRNA/Cas9 system mediated NHEJ
acttgtacttatcaaaattcggaactgacacacggtgatggaacgtgactaaggtggg

SEQ ID NO: 341 - DNA, 57 bp, Artificial sequence
  sequence of gRNA/Cas9 system mediated NHEJ
acttgtacttatcaaaattcggaactgacacacatgatggaacgtgactaaggtggg

SEQ ID NO: 342 - DNA, 57 bp, Artificial sequence
  sequence of gRNA/Cas9 system mediated NHEJ
acttgtacttatcaaaattcggaactgacacacgtgatggaacgtgactaaggtggg

SEQ ID NO: 343 - DNA, 56 bp, Artificial sequence
  sequence of gRNA/Cas9 system mediated NHEJ
acttgtacttatcaaaattcggaactgacacactgatggaacgtgactaaggtggg

SEQ ID NO: 344 - DNA, 56 bp, Artificial sequence
  sequence of gRNA/Cas9 system mediated NHEJ
acttgtacttatcaaaattcggaactgacacacggatggaacgtgactaaggtggg

SEQ ID NO: 345 - DNA, 55 bp, Artificial sequence
  sequence of gRNA/Cas9 system mediated NHEJ
acttgtacttatcaaaattcggaactgacacacgatggaacgtgactaaggtggg

SEQ ID NO: 346 - DNA, 55 bp, Artificial sequence
  sequence of gRNA/Cas9 system mediated NHEJ
acttgtacttatcaaaattcggaactgacacatgatggaacgtgactaaggtggg

SEQ ID NO: 347 - DNA, 54 bp, Artificial sequence
  sequence of gRNA/Cas9 system mediated NHEJ
acttgtacttatcaaaattcggaactgacacacatggaacgtgactaaggtggg

SEQ ID NO: 348 - DNA, 54 bp, Artificial sequence
  sequence of gRNA/Cas9 system mediated NHEJ
acttgtacttatcaaaattcggaactgacactgatggaacgtgactaaggtggg

SEQ ID NO: 349 - DNA, 53 bp, Artificial sequence
  sequence of gRNA/Cas9 system mediated NHEJ
acttgtacttatcaaaattcggaactgacacgatggaacgtgactaaggtggg

SEQ ID NO: 350 - DNA, 51 bp, Artificial sequence
  sequence of gRNA/Cas9 system mediated NHEJ
acttgtacttatcaaaattcggaactgatgatggaacgtgactaaggtggg

SEQ ID NO: 351 - DNA, 50 bp, Artificial sequence
  sequence of gRNA/Cas9 system mediated NHEJ
acttgtacttatcaaaattcggaactgacatggaacgtgactaaggtggg

SEQ ID NO: 352 - DNA, 50 bp, Artificial sequence
  sequence of gRNA/Cas9 system mediated NHEJ
acttgtacttatcaaaattcggaactgtgatggaacgtgactaaggtggg

SEQ ID NO: 353 - DNA, 51 bp, Artificial sequence
  sequence of gRNA/Cas9 system mediated NHEJ
acttgtacttatcaaaattcggaactgacacacgaacgtgactaaggtggg

SEQ ID NO: 354 - DNA, 50 bp, Artificial sequence
  sequence of gRNA/Cas9 system mediated NHEJ
acttgtacttatcaaaattcggaactgacacggaacgtgactaaggtggg

SEQ ID NO: 355 - DNA, 49 bp, Artificial sequence
  sequence of gRNA/Cas9 system mediated NHEJ
acttgtacctatcaaaattcggaactgaatggaacgtgactaaggtggg

SEQ ID NO: 356 - DNA, 48 bp, Artificial sequence
  sequence of gRNA/Cas9 system mediated NHEJ
acttgtacttatcaaaattcggaactgatggaacgtgactaaggtggg

SEQ ID NO: 357 - DNA, 46 bp, Artificial sequence
  sequence of gRNA/Cas9 system mediated NHEJ
acttgtacttatcaaaattcggaactgagaacgtgactaaggtggg

SEQ ID NO: 358 - DNA, 38 bp, Artificial sequence
  sequence of gRNA/Cas9 system mediated target site modification
acttgtacttatcaaaattcggaactgacacacgacat

SEQ ID NO: 359 - DNA, 38 bp, Artificial sequence
  sequence of gRNA/Cas9 system mediated NHEJ
acttgtacttatcaaaattcggaactgacaaaggtggg

SEQ ID NO: 360 - DNA, 39 bp, Artificial sequence
  sequence of gRNA/Cas9 system mediated NHEJ
acttgtacttatcaaaattcggaacgtgactaaggtggg

SEQ ID NO: 361 - DNA, 24 bp, Artificial sequence
  sequence of gRNA/Cas9 system mediated NHEJ
actatggaacgtgactaaggtggg

SEQ ID NO: 362 - DNA, 211 bp, Artificial sequence
  sequence of gRNA/Cas9 system mediated target site modification
acttgtacttatcaaaattcggaactgacacacggccggtgatggattggtggatgagtgttgcgtcgagcacctccttggtggaggtgtatctcttcctgtcaatggtggtgtcgaagtacttgaaggcagcaggggctcccaagtttgtgagggtaaacaggtggataatgttttcagcctgctcgcgatggaacgtgactaaggtggg

SEQ ID NO: 363 - DNA, 90 bp, Artificial sequence
  sequence of gRNA/Cas9 system mediated target site modification
acttgtacttatcaaaaactacttgtgctgtaaaaaaaaagaggaacaatcttcactcatcaataagtgatggaacgcgactaaggtggg

SEQ ID NO: 364 - DNA, 57 bp, Glycine max
  soybean genomic DD20CR2 target region (1)..(57)
gacacacgacatgatggaacgtgactaaggtgggtttttgactttgcatgtcgaagt

SEQ ID NO: 365 - DNA, 61 bp, artificial sequence
  sequence of gRNA/Cas9 system mediated target site modification
actgacacacgacatgatggaacgtgaactaaggtgggtttttgactttgcatgtcgaagt

SEQ ID NO: 366 - DNA, 59 bp, Artificial sequence
  sequence of gRNA/Cas9 system mediated NHEJ
actgacacacgacatgatggaacgtactaaggtgggtttttgactttgcatgtcgaagt

SEQ ID NO: 367 - DNA, 58 bp, Artificial sequence
  sequence of gRNA/Cas9 system mediated NHEJ
actgacacacgacatgatggaacgtctaaggtgggtttttgactttgcatgtcgaagt

SEQ ID NO: 368 - DNA, 58 bp, Artificial sequence
  sequence of gRNA/Cas9 system mediated NHEJ
actgacacacgacatgatggaacgtgaaaggtgggtttttgactttgcatgtcgaagt

SEQ ID NO: 369 - DNA, 57 bp, Artificial sequence
  sequence of gRNA/Cas9 system mediated NHEJ
actgacacacgacatgatggaacgctaaggtgggtttttgactttgcatgtcgaagt

SEQ ID NO: 370 - DNA, 57 bp, Artificial sequence
  sequence of gRNA/Cas9 system mediated NHEJ
actgacacacgacatgatggaacgtgaaggtgggtttttgactttgcatgtcgaagt

SEQ ID NO: 371 - DNA, 56 bp, Artificial sequence
  sequence of gRNA/Cas9 system mediated NHEJ
actgacacacgacatgatggaacgtgaggtgggtttttgactttgcatgtcgaagt

SEQ ID NO: 372 - DNA, 56 bp, Artificial sequence
  sequence of gRNA/Cas9 system mediated NHEJ
actgacacacgacatgatggaacgtaaggtgggtttttgactttgcatgtcgaagt

SEQ ID NO: 373 - DNA, 56 bp, Artificial sequence
  sequence of gRNA/Cas9 system mediated NHEJ
actgacacacgacatgatggaacctaaggtgggtttttgactttgcatgtcgaagt

SEQ ID NO: 374 - DNA, 56 bp, Artificial sequence; identical to SEQ ID NO: 371
  sequence of gRNA/Cas9 system mediated NHEJ

SEQ ID NO: 375 - DNA, 55 bp, Artificial sequence
  sequence of gRNA/Cas9 system mediated NHEJ
actgacacacgacatgatggaactaaggtgggtttttgactttgcatgtcgaagt

SEQ ID NO: 376 - DNA, 54 bp, Artificial sequence
  sequence of gRNA/Cas9 system mediated NHEJ
actgacacacgacatgatggaataaggtgggtttttgactttgcatgtcgaagt

SEQ ID NO: 377 - DNA, 53 bp, Artificial sequence
  sequence of gRNA/Cas9 system mediated NHEJ
actgacacacgacatgatggctaaggtgggtttttgactttgcatgtcgaagt

SEQ ID NO: 378 - DNA, 53 bp, Artificial sequence
  sequence of gRNA/Cas9 system mediated NHEJ
actgacacacgacatgatggataaggtgggtttttgactttgcatgtcgaagt

SEQ ID NO: 379 - DNA, 51 bp, Artificial sequence
  sequence of gRNA/Cas9 system mediated NHEJ
actgacacacgacatgatggaaggtgggtttttgactttgcatgtcgaagt

SEQ ID NO: 380 - DNA, 50 bp, Artificial sequence
  sequence of gRNA/Cas9 system mediated NHEJ
actgacacacgacatgatggaggtgggtttttgactttgcatgtcgaagt

SEQ ID NO: 381 - DNA, 44 bp, Artificial sequence
  sequence of gRNA/Cas9 system mediated NHEJ
actgacacacgacatgatgggtttttgactttgcatgtcgaagt

SEQ ID NO: 382 - DNA, 43 bp, Artificial sequence
  sequence of gRNA/Cas9 system mediated NHEJ
actgacacacgacaggtgggtttttgactttgcatgtcgaagt

SEQ ID NO: 383 - DNA, 40 bp, Artificial sequence
  sequence of gRNA/Cas9 system mediated NHEJ
actgacactaaggtgggtttttgactttgcatgtcgaagt

SEQ ID NO: 384 - DNA, 25 bp, Artificial sequence
  sequence of gRNA/Cas9 system mediated NHEJ
actgacacacgacatgatggaacgt

SEQ ID NO: 385 - DNA, 20 bp, Artificial sequence
  sequence of gRNA/Cas9 system mediated NHEJ
actgacacacgacatgatgg

SEQ ID NO: 386 - DNA, 60 bp, Glycine max
  soybean genomic DD43CR1 target region (1)..(60)
agccttacaactcacaagtcccttgtacttgtacgtacggagggtattctagaaaagagg

SEQ ID NO: 387 - DNA, 59 bp, Artificial sequence
  sequence of gRNA/Cas9 system mediated NHEJ
agccttacaactcacaagtcccttgtacttgtactacggagggtattctagaaaagagg

SEQ ID NO: 388 - DNA, 59 bp, Artificial sequence
  sequence of gRNA/Cas9 system mediated NHEJ
agccttacaactcacaagtcccttgtacttgtagtacggagggtattctagaaaagagg

SEQ ID NO: 389 - DNA, 58 bp, Artificial sequence
  sequence of gRNA/Cas9 system mediated NHEJ
agccttacaactcacaagtcccttgtacttgtgtacggagggtattctagaaaagagg

SEQ ID NO: 390 - DNA, 58 bp, Artificial sequence
  sequence of gRNA/Cas9 system mediated NHEJ
agccttacaactcacaagtcccttgtacttgcgtacggagggtattctagaaaagagg

SEQ ID NO: 391 - DNA, 57 bp, Artificial sequence
  sequence of gRNA/Cas9 system mediated NHEJ
agccttacaactcacaagtcccttgtacttggtacggagggtattctagaaaagagg

SEQ ID NO: 392 - DNA, 57 bp, Artificial sequence
  sequence of gRNA/Cas9 system mediated NHEJ
agccttacaactcacaagtcccttgtacttgttacggagggtattctagaaaagagg

SEQ ID NO: 393 - DNA, 56 bp, Artificial sequence
  sequence of gRNA/Cas9 system mediated NHEJ
agccttacaactcacaagtcccttgtacttgtacggagggtattctagaaaagagg

SEQ ID NO: 394 - DNA, 55 bp, Artificial sequence
  sequence of gRNA/Cas9 system mediated NHEJ
agccttacaactcacaagtcccttgtactttacggagggtattctagaaaagagg

SEQ ID NO: 395 - DNA, 55 bp, Artificial sequence
  sequence of gRNA/Cas9 system mediated NHEJ
agccttacaactcacaagtcccttgtactgtacggagggtattctagaaaagagg

SEQ ID NO: 396 - DNA, 54 bp, Artificial sequence
  sequence of gRNA/Cas9 system mediated NHEJ
agccttacaactcacaagccccttgtacttacggagggtattctagaaaagagg

SEQ ID NO: 397 - DNA, 52 bp, Artificial sequence
  sequence of gRNA/Cas9 system mediated NHEJ
agccttacaactcacaagtcccttgtatacggagggtattctagaaaagagg

SEQ ID NO: 398 - DNA, 52 bp, Artificial sequence
  sequence of gRNA/Cas9 system mediated NHEJ
agccttacaactcacaagtcccttgtgtacggagggtattctagaaaagagg

SEQ ID NO: 399 - DNA, 50 bp, Artificial sequence
  sequence of gRNA/Cas9 system mediated NHEJ
agccttacaactcacaagtcccttgtacggagggtattctagaaaagagg

SEQ ID NO: 400 - DNA, 49 bp, Artificial sequence
  sequence of gRNA/Cas9 system mediated NHEJ
agccttacaactcacaagtccctttacggagggtattctagaaaagagg

SEQ ID NO: 401 - DNA, 48 bp, Artificial sequence
  sequence of gRNA/Cas9 system mediated NHEJ
agccttacaactcacaagtcccttacggagggtattctagaaaagagg

SEQ ID NO: 402 - DNA, 47 bp, Artificial sequence
  sequence of gRNA/Cas9 system mediated NHEJ
agccttacaactcacaagtccctacggagggtattctagaaaagagg

SEQ ID NO: 403 - DNA, 43 bp, Artificial sequence
  sequence of gRNA/Cas9 system mediated NHEJ
agccttacaactcacaagtcccttgtacttgtaagaaaagagg

SEQ ID NO: 404 - DNA, 49 bp, Artificial sequence
  sequence of gRNA/Cas9 system mediated target site modification
agccttacaactcacaagtcctaaattaaaggttattctagaaaagagg

SEQ ID NO: 405 - DNA, 227 bp, Artificial sequence
  sequence of gRNA/Cas9 system mediated target site modification
agccttacaactcacaagtcccttgtacttgtagaatccagttcataaaacaagtgacacacaacagatatgaactggactacgtcgaacccacaaatcccacaaagcgcgtgaaatcaaatcgctcaaaccacaaaaaagaacaacgcgtttgttacacgctaataccaaaattatacccaaatcttaagctatttatgcgtacggagggtattctagaaaagagg

SEQ ID NO: 406 - DNA, 97 bp, Artificial sequence
  sequence of gRNA/Cas9 system mediated target site modification
agccttacaactcacaagtcccttgtacttgtaatgctcccctctaaactcgtatcgcttcagagttgagagtacggagggtattctagaaaagagg

SEQ ID NO: 407 - DNA, 183 bp, Artificial sequence
  sequence of gRNA/Cas9 system mediated target site modification
agccttacaactcacaagtcccttgtatatagatacccacaaaataagtaaacccgatccaaaatcttaaatgatgtggaggaagggaaattgttaattattcccctcttctcctacatggatatacctgaaacatgcaatggatggattagattttgtacggagggtattctagaaaagagg

SEQ ID NO: 408 - DNA, 234 bp, Artificial sequence
  sequence of gRNA/Cas9 system mediated target site modification
agccttacaactcacaagtcccttgtacttgtaccaggggatgttttttatttacattcacgtcttttggaaagagccgctaaattaagttctcagttaggcgaaggaagtatgactgctttaccaatagttgaaactcaatcgggagatgtttcagcttatattcctactaatgtaatttccattacagatggccaaatattcttacgtacggagggtattctagaaaagagg

SEQ ID NO: 409 - DNA, 280 bp, Artificial sequence
  sequence of gRNA/Cas9 system mediated target site modification
agccttacaactcacaagtcccttgtacttgtaccgaaaatttcagccataaaaaaagttataatagaatttaaagcaaaagtttcattttttaaacatatatactgacacgctccgataaaaatagaggcaagtccgacaacgtcccctccgaggaggtcgtgaagaagatgaaaaactactggagacagctcttgaacgccaagctcatcacccagcgtaagctcgacaacctgactaaggctgagagaggtgtacggagggtattctagaaaagagg

SEQ ID NO: 410 - DNA, 250 bp, Artificial sequence
  sequence of gRNA/Cas9 system mediated target site modification
agccttacaactcacaagtcccttgtacttgtactggatttggtgagggatgcttccgttgtcgaaggttctctgcttcctcaacaggtcctctctgttcaacttcaccaacagctcctcggtaccgtccatcttctcaaggatgaagatcgagtgcttcgactccgtcgagatctctggtgtcgaggacaggttcaacgcctcccttgggacttgccacgatcgtacggagggtattctagaaaagagg

SEQ ID NO: 411 - DNA, 161 bp, Artificial sequence
  sequence of gRNA/Cas9 system mediated target site modification
agccttacaactcacaagtcccttatgacctcaaaaaaaagattcacctccaacacaccaaataactcgaaaatctctttcctattctctagaaagtataggaacttccactagtccatgaaaaagcctgaactcgtacggagggtattctagaaaagagg

SEQ ID NO: 412 - DNA, 185 bp, Artificial sequence
  sequence of gRNA/Cas9 system mediated target site modification
agccttacaactcacaagtcccttgtacttgtacacctggggcatggagagcaccttcctcacagtagcgaaatccctccctttgtcccacacgatctctccagtctcaccgttggtctcgatcagtgggcgcttcctgatctcaccgttggcgagagtgtacggagggtattctagaaaagagg

SEQ ID NO: 413 - DNA, 212 bp, Artificial sequence
  sequence of gRNA/Cas9 system mediated target site modification
agccttacaactcacaagtcccttgtacttgtgctaggttagccgaaagatggttatcggttcaaggacgcaaggtgcccctgctttttcagggtaataaggggtagagaaaatgcctcgagccaaagttcgagtaccaggcgctacagcgctgaagtaatccatgccatactcccaggaaaagccgtacggagggtattctagaaaagagg

SEQ ID NO: 414 - DNA, 231 bp, Artificial sequence
  sequence of gRNA/Cas9 system mediated target site modification
agccttacaactcacaagtcccttgtacttgtactcaagttcttattttatttcatattttttcctataaaaggttaatggctctataaaggttgagtgacggatccggtcacctaagtgactagggtcacgtgaccctagtcacttattcccgggcaactttattatacaaagttgatagatctcgaattcattccgattaatcgtggcgagggtattctagaaaagagg

SEQ ID NO: 415 - DNA, 98 bp, Artificial sequence
  LIGCas-1 mutation 1
tcctctgtaacgatttacgcacctgctgggaattgtaccgtacgtgccccggtcggaggatatatatacctcacacgtacgcgtacgcgtatatatac

SEQ ID NO: 416 - DNA, 98 bp, Artificial sequence
  LIGCas-1 mutation 2
tcctctgtaacgatttacgcacctgctgggaattgtaccgtacgtgccccggacggaggatatatatacctcacacgtacgcgtacgcgtatatatac

SEQ ID NO: 417 - DNA, 98 bp, Artificial sequence
  LIGCas-1 mutation 3
tcctctgtaacgatttacgcacctgctgggaattgtaccgtacgtgccccgggcggaggatatatatacctcacacgtacgcgtacgcgtatatatac

SEQ ID NO: 418 - DNA, 98 bp, Artificial sequence
  LIGCas-1 mutation 4
tcctctgtaacgatttacgcacctgctgggaattgtaccgtacgtgccccggccggaggatatatatacctcacacgtacgcgtacgcgtatatatac

SEQ ID NO: 419 - DNA, 99 bp, Artificial sequence
  LIGCas-1 mutation 5
tcctctgtaacgatttacgcacctgctgggaattgtaccgtacgtgccccggatcggaggatatatatacctcacacgtacgcgtacgcgtatatatac

SEQ ID NO: 420 - DNA, 94 bp, Artificial sequence
  LIGCas-1 mutation 6
tcctctgtaacgatttacgcacctgctgggaattgtaccgtacgtgccccggaggatatatatacctcacacgtacgcgtacgcgtatatatac

SEQ ID NO: 421 - DNA, 81 bp, Artificial sequence
  LIGCas-1 mutation 7
tcctctgtaacgatttacgcacctgctgggaattgtaccgtacgtgccccggttcacacgtacgcgtacgcgtatatatac

SEQ ID NO: 422 - DNA, 65 bp, Artificial sequence
  LIGCas-1 mutation 8
tcctctgtaacgatttacgcacctgctgggaattgtaccgtacgtacgcgtacgcgtatatatac

SEQ ID NO: 423 - DNA, 99 bp, Artificial sequence
  LIGCas-1 mutation 9
tcctctgtaacgatttacgcacctgctgggaattgtaccgtacgtgccccggttcggaggatatatatacctcacacgtacgcgtacgcgtatatatac

SEQ ID NO: 424 - DNA, 95 bp, Artificial sequence
  LIGCas-1 mutation 10
tcctctgtaacgatttacgcacctgctgggaattgtaccgtacgtgcccccggaggatatatatacctcacacgtacgcgtacgcgtatatatac

SEQ ID NO: 425 - DNA, 98 bp, Artificial sequence
  LIGCas-2 mutation 1
gaagctgtaacgatttacgcacctgctgggaattgtaccgtacgtgaccccggcggaggatatatatacctcacacgtacgcgtacgcgtatatatac

SEQ ID NO: 426 - DNA, 98 bp, Artificial sequence
  LIGCas-2 mutation 2
gaagctgtaacgatttacgcacctgctgggaattgtaccgtacgtgtccccggcggaggatatatatacctcacacgtacgcgtacgcgtatatatac

SEQ ID NO: 427 - DNA, 96 bp, Artificial sequence
  LIGCas-2 mutation 3
gaagctgtaacgatttacgcacctgctgggaattgtaccgtacgtccccggcggaggatatatatacctcacacgtacgcgtacgcgtatatatac

SEQ ID NO: 428 - DNA, 98 bp, Artificial sequence
  LIGCas-2 mutation 4
gaagctgtaacgatttacgcacctgctgggaattgtaccgtacgtggccccggcggaggatatatatacctcacacgtacgcgtacgcgtatatatac

SEQ ID NO: 429 - DNA, 99 bp, Artificial sequence
  LIGCas-2 mutation 5
gaagctgtaacgatttacgcacctgctgggaattgtaccgtacgtgcaccccggcggaggatatatatacctcacacgtacgcgtacgcgtatatatac

SEQ ID NO: 430 - DNA, 87 bp, Artificial sequence
  LIGCas-2 mutation 6
gaagctgtaacgatttacgcacctgctgggaattgtacccggcggaggatatatatacctcacacgtacgcgtacgcgtatatatac

SEQ ID NO: 431 - DNA, 92 bp, Artificial sequence
  LIGCas-2 mutation 7
gaagctgtaacgatttacgcacctgctgggaattgtaccgtccccggcggaggatatatatacctcacacgtacgcgtacgcgtatatatac

SEQ ID NO: 432 - DNA, 94 bp, Artificial sequence
  LIGCas-2 mutation 8
gaagctgtaacgatttacgcacctgctgggaattgtaccgtacccccggcggaggatatatatacctcacacgtacgcgtacgcgtatatatac

SEQ ID NO: 433 - DNA, 95 bp, Artificial sequence
  LIGCas-2 mutation 9
gaagctgtaacgatttacgcacctgctgggaattgtaccgtacgccccggcggaggatatatatacctcacacgtacgcgtacgcgtatatatac

SEQ ID NO: 434 - DNA, 88 bp, Artificial sequence
  LIGCas-2 mutation 10
gaagctgtaacgatttacgcacctgctgggaattgtaccccggcggaggatatatatacctcacacgtacgcgtacgcgtatatatac

SEQ ID NO: 435 - DNA, 98 bp, Artificial sequence; identical to SEQ ID NO: 77
  LIGCas-3 mutation 1

SEQ ID NO: 436 - DNA, 96 bp, Artificial sequence; identical to SEQ ID NO: 78
  LIGCas-3 mutation 2

SEQ ID NO: 437 - DNA, 95 bp, Artificial sequence; identical to SEQ ID NO: 80
  LIGCas-3 mutation 3

SEQ ID NO: 438 - DNA, 96 bp, Artificial sequence; identical to SEQ ID NO: 79
  LIGCas-3 mutation 4

SEQ ID NO: 439 - DNA, 68 bp, Artificial sequence; identical to SEQ ID NO: 81
  LIGCas-3 mutation 5

SEQ ID NO: 440 - DNA, 93 bp, Artificial sequence; identical to SEQ ID NO: 83
  LIGCas-3 mutation 6

SEQ ID NO: 441 - DNA, 89 bp, Artificial sequence
  LIGCas-3 mutation 7
aaggcgcaaatgagtagcagcgcacgtatatatacgcgtacgtgaggtatatatatcctccgccggggcacgtacggtacaattcccag

SEQ ID NO: 442 - DNA, 89 bp, Artificial sequence
  LIGCas-3 mutation 8
aaggcgcaaatgagtagcagcgcacgtatatatacgcgtacgcgtactatatatatcctccgccggggcacgtacggtacaattcccag

SEQ ID NO: 443 - DNA, 94 bp, Artificial sequence
  LIGCas-3 mutation 9
aaggcgcaaatgagtagcagcgcacgtatatatacgcgtacgcgtacggaggtatatatatcctccgccggggcacgtacggtacaattcccag

SEQ ID NO: 444 - DNA, 96 bp, Artificial sequence
  LIGCas-3 mutation 0
aaggcgcaaatgagtagcagcgcacgtatatatacgcgtacgcgtacgatgaggtatatatatcctccgccggggcacgtacggtacaattcccag

SEQ ID NO: 445 - DNA, 1051 bp, Artificial sequence
  LIGCas-1_crRNA_Expression_Cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgtaccgtacgtgccccggcgggttttagagctatgctgttttgtttttttt

SEQ ID NO: 446 - DNA, 1051 bp, Artificial sequence
  LIGCas-2_crRNA_Expression_Cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattggaattgtaccgtacgtgcccgttttagagctatgctgttttgtttttttt

SEQ ID NO: 447 - DNA, 1047 bp, Artificial sequence
  LIGCas-3_crRNA_Expression_Cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattgcgtacgcgtacgtgtggttttagagctatgctgttttgtttttttt

SEQ ID NO: 448 - DNA, 1087 bp, Artificial sequence
  tracrRNA_Expression_Cassette
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattggaaccattcaaaacagcatagcaagttaaaataaggctagtccgttatcaacttgaaaaagtggcaccgagtcggtgctttttttt

SEQ ID NO: 449 - DNA, 63 bp, Artificial Sequence
  LIGCas-2 forward primer for primary
ctacactctttccctacacgacgctcttccgatctgaagctgtaacgatttacgcacctgctg

SEQ ID NO: 450 - DNA, 60 bp, Artificial Sequence
  LIGCas-3 forward primer for primary PCR
ctacactctttccctacacgacgctcttccgatctttcccgcaaatgagtagcagcgcac

SEQ ID NO: 451 - DNA, 19 bp, Artificial sequence
  Zm-ARGOS8-CTS1, Cas9 target sequence 1
gcgtgcatcgatccatcgc

SEQ ID NO: 452 - DNA, 21 bp, Artificial sequence
  m-ARGOS8-CTS2, Cas9 target sequence 2
ggctacggatagatatgatgc

SEQ ID NO: 453 - DNA, 20 bp, Artificial sequence
  Zm-ARGOS8-CTS3, Cas9 target sequence 3
gttacttctctaagcacggc

SEQ ID NO: 454 - DNA, 21 bp, Artificial sequence
  P1, Forward_primer
gcgccattccctaaaggtaac

SEQ ID NO: 455 - DNA, 23 bp, Artificial sequence
  P2, Reverse_primer
gctaatcgtaagtgacgcttgga

SEQ ID NO: 456 - DNA, 31 bp, Artificial sequence; identical to SEQ ID NO: 99
  P3, Forward_primer

SEQ ID NO: 457 - DNA, 20 bp, Artificial sequence
  P4, Reverse_primer
ctgcgaactgcttgattccg

SEQ ID NO: 458 - DNA, 24 bp, Artificial sequence
  P5, Forward_primer
accgtccttatctctgcatcatct

SEQ ID NO: 459 - DNA, 31 bp, Artificial sequence; identical to SEQ ID NO: 99
  PBS, Primer Binding Site

SEQ ID NO: 460 - DNA, 1823 bp, Zea mays
  misc_feature (1)..(1823): Zm-GOS2 PRO-GOS2 INTRON, maize GOS2 promoter and GOS2 intron1 including the promoter, 5′-UTR1, INTRON1 and 5′-UTR2 sequence
taattattggctgtaggattctaaacagagcctaaatagctggaatagctctagccctcaatccaaactaatgatatctatacttatgcaactctaaatttttattctaaaagtaatatttcatttttgtcaacgagattctctactctattccacaatcttttgaagctatatttaccttaaatctgtactctataccaataatcatatattctattatttatttttatctctctcctaaggagcatccctctatgtctgcatggcccccgcctcgggtcccaatctcttgctctgctagtagcacagaagaaaacactagaaatgacttgcttgacttagagtatcagataaacatcatgtttacttaactttaatttgtatcggtttctactatttttataatatttttgtctctatagatactacgtgcaacagtataatcaacctagtttaatccagagcgaaggattttttactaagtacgtgactccatatgcacagcgttccttttatggttcctcactgggcacagcataaacgaaccctgtccaatgttttcagcgcgaacaaacagaaattccatcagcgaacaaacaacatacatgcgagatgaaaataaataataaaaaaagctccgtctcgataggccggcacgaatcgagagcctccatagccagttttttccatcggaacggcggttcgcgcacctaattatatgcaccacacgcctataaagccaaccaacccgtcggaggggcgcaagccagacagaagacagcccgtcagcccctctcgtttttcatccgccttcgcctccaaccgcgtgcgctccacgcctcctccaggaaagcgagaggtgagcgcagtcccctttcccctccttccaattcaattcgtcttctcgttcgcagccctaggatttgggggtctggaggggtttgatcgtttctcgccgtgaatctgctttggtgtaaaccaacggatctcggatcgtagtcttcagaagatcccggattttgcggtttggcccctcctggattcaattcgtcgtatcgttcgcagccctaggatttggggatctggaggggtttgatcgtttctcgccgcgaatctgctctggtgtaaaccaacggatctcgggtcgtagtcttcagaaggtcccggattttgcggtttggcccctcctggattcaattcgtcgtatcgttcgcagccctaggatttggggatctggaggggtttgatcctttctcgccgcgaatctgctctggtataaccaacggatctcgggtcgtagtcttcagaaggtcccggattttgcggtttggtggttcttgctctatgaatcagagggatggttcttcccggatttatgccttgcggccactctgtcgaatcatggggtttcgacccgattcgtaggcgtgctccctgttttggatgggaagtaggcgtgtttgtagtattcgtgcttcgattcgtcaacggagattagaagacctgggatgggatttgaggaaatctaggtatctgtctagcacgtttctagatctattcttcagctgttatatgagagtaattttggaaccctggtggggtatgtttgaccgagtattctgtagattattgtccgtgacttgctggctgttaccgtccttatctctgcatcatctatctgtgctagtttctgcgtgcttctcaaatatttccggcctgtgtagcatgtgactgataatatgattttggcagcttctgcataagaacaacaaatcaaaagcttgatcagctcggtgcctacaaaacctcaacaaccaagtttc

SEQ ID NO: 461 - DNA, 556 bp, Zea mays
  misc_feature (1)..(556): Zm-ARGOS8 promoter
gttacttctctaagcacggctggatttcaggcctctagtcctctactagtactagctacacgacgtgcacgcatgcatcacagcatcaacaactagacacgcacacgctgcacgcggccggggaacccactgattccccccttccccgcgcgcggtttgatttcctttcctggtacggatccatatctgagggcttgttcggttattcccaacacacatgtattggatgggattgaaaaaaaaatgagaagaagtttgacttgtttgggattcaaacccatccaatcccactcaatccacatggattgagagctaaccgaacaagccctcatagtacatacctggtacggatccatatcatagtacatagatccagtagaatagaaggtgatccgaccgccggcgcttgcgttgttttccccggtccattgaacctgccaaccctcctaaccacaggcacgccaaaccgcgggctccggccaccaccgccaccgccacctgccctgccgcacctctccaaccccaaatccaggggggggggggggcaccatgcgtg

SEQ ID NO: 462 - DNA, 155 bp, Zea mays
  misc_feature (1)..(155): Zm-ARGOS8 5′-UTR
catcgatccatcgctggcgcgcgggtccggcggggcggtctgtgagggcaaatttatataggtctagtgggtacccggctacggatagatatgatgctgcactgcacattggctatatctgaggctcctgcgcgcgccttggccaggtgtctgtc

SEQ ID NO: 463 - DNA, 285 bp, Zea mays
atgcgggcgatgccgcaggaagaggaagccgcggtggcgacgacgaccatggccgggggcaaggtggcggcgctgctggccacggcggccgcgctgctgctgctgctcccgctggcgctgccgccgctgccgccgccgcccacgcagctgttgttcgtccccgtggtcttgctgctcctcgtggcgtccctcgcgttctgccccgccgcgaccccctcgccgtcgccgatgcatgccgccgaccacgggtcgttcgggaccactggatcaccgcacctatgttga

SEQ ID NO: 464 - DNA, 2843 bp, Zea mays
  misc_feature (1)..(2843): Zm-GOS2 gene, including promoter, 5′-UTR, CDS, 3′-UTR and introns sequence
taattattggctgtaggattctaaacagagcctaaatagctggaatagctctagccctcaatccaaactaatgatatctatacttatgcaactctaaatttttattctaaaagtaatatttcatttttgtcaacgagattctctactctattccacaatcttttgaagctatatttaccttaaatctgtactctataccaataatcatatattctattatttatttttatctctctcctaaggagcatccctctatgtctgcatggcccccgcctcgggtcccaatctcttgctctgctagtagcacagaagaaaacactagaaatgacttgcttgacttagagtatcagataaacatcatgtttacttaactttaatttgtatcggtttctactatttttataatatttttgtctctatagatactacgtgcaacagtataatcaacctagtttaatccagagcgaaggattttttactaagtacgtgactccatatgcacagcgttccttttatggttcctcactgggcacagcataaacgaaccctgtccaatgttttcagcgcgaacaaacagaaattccatcagcgaacaaacaacatacatgcgagatgaaaataaataataaaaaaagctccgtctcgataggccggcacgaatcgagagcctccatagccagttttttccatcggaacggcggttcgcgcacctaattatatgcaccacacgcctataaagccaaccaacccgtcggaggggcgcaagccagacagaagacagcccgtcagcccctctcgtttttcatccgccttcgcctccaaccgcgtgcgctccacgcctcctccaggaaagcgagaggtgagcgcagtcccctttcccctccttccaattcaattcgtcttctcgttcgcagccctaggatttgggggtctggaggggtttgatcgtttctcgccgtgaatctgctttggtgtaaaccaacggatctcggatcgtagtcttcagaagatcccggattttgcggtttggcccctcctggattcaattcgtcgtatcgttcgcagccctaggatttggggatctggaggggtttgatcgtttctcgccgcgaatctgctctggtgtaaaccaacggatctcgggtcgtagtcttcagaaggtcccggattttgcggtttggcccctcctggattcaattcgtcgtatcgttcgcagccctaggatttggggatctggaggggtttgatcctttctcgccgcgaatctgctctggtataaccaacggatctcgggtcgtagtcttcagaaggtcccggattttgcggtttggtggttcttgctctatgaatcagagggatggttcttcccggatttatgccttgcggccactctgtcgaatcatggggtttcgacccgattcgtaggcgtgctccctgttttggatgggaagtaggcgtgtttgtagtattcgtgcttcgattcgtcaacggagattagaagacctgggatgggatttgaggaaatctaggtatctgtctagcacgtttctagatctattcttcagctgttatatgagagtaattttggaaccctggtggggtatgtttgaccgagtattctgtagattattgtccgtgacttgctggctgttaccgtccttatctctgcatcatctatctgtgctagtttctgcgtgcttctcaaatatttccggcctgtgtagcatgtgactgataatatgattttggcagcttctgcataagaacaacaaatcaaaagcttgatcagctcggtgcctacaaaacctcaacaaccaagtttcatgtctgatctcgacgtccagcttccatctgcctttggtatggctacttctcaattcatgatgccatgtttttttttatattgtggttttacataatacatagcatcttccagcttcctgaagagtattactgaatagattgataacatcatacacacgaagttcatcttgaacatgcttattagtgttctgtttgcatctgatggtatggcatcatctttgatagatccgtttgctgaggcaaatgctgaggactctggtgctggtcctggaacgaaggattatgtgcatgtgcgcatccagcagcgcaacggcagaaagagtctgactacagtccagggtctgaagaaagagttcagctataacaagatcctcaaggatctgaagaaggaattctgctgcaatggtactgtagttcaggacccagagctaggccaggtaagatacgagaacaatgcatttcaagcttgtaaaaatggtatctgccggttggtggatatactgatctgtttgtccgctgcaggtcattcagctccaaggtgaccagcgcaagaatgttgctactttcctagttcaggtattcagaatcttcagacctggccagctgaatactgttttaccataccgatagatgttcaatctgttaatactgatcgtgcaattattacttgtcttggtaggctgggattgcgaagaaagagaacatcaagattcacgggttctaagggacctgtaaatgcttgtgccctatattgtgtgcctccacatattggggagcttgaagcatcgacagttactagtcattgcttacttatataagaacataagtagtatttgctattgtcaagtgtgccttgcttgatgcaagttgtgttttcgtatcattattattatgcacggccatcgtacgtgtatggcttgtatgggttattgccaacttaataaaagcacactctgtttgcctataagcactgatgtttgcctcgtcatgcacatgttgagtcgggttttatttgtatt

SEQ ID NO: 465 - DNA, 800 bp, Zea mays
  misc_feature (1)..(800): Zm-GOS2 PRO, maize GOS2 promoter
taattattggctgtaggattctaaacagagcctaaatagctggaatagctctagccctcaatccaaactaatgatatctatacttatgcaactctaaatttttattctaaaagtaatatttcatttttgtcaacgagattctctactctattccacaatcttttgaagctatatttaccttaaatctgtactctataccaataatcatatattctattatttatttttatctctctcctaaggagcatccctctatgtctgcatggcccccgcctcgggtcccaatctcttgctctgctagtagcacagaagaaaacactagaaatgacttgcttgacttagagtatcagataaacatcatgtttacttaactttaatttgtatcggtttctactatttttataatatttttgtctctatagatactacgtgcaacagtataatcaacctagtttaatccagagcgaaggattttttactaagtacgtgactccatatgcacagcgttccttttatggttcctcactgggcacagcataaacgaaccctgtccaatgttttcagcgcgaacaaacagaaattccatcagcgaacaaacaacatacatgcgagatgaaaataaataataaaaaaagctccgtctcgataggccggcacgaatcgagagcctccatagccagttttttccatcggaacggcggttcgcgcacctaattatatgcaccacacgcctataaagccaaccaacccgtcggaggggcgcaagccagacagaagacagcccgtcagcccctctcgtt

SEQ ID NO: 466 - DNA, 1023 bp, Zea mays
  misc_feature (1)..(1023): GOS2 INTRON, maize GOS2 5′-UTR1 and intron1 and 5′-UTR2 sequence
tttcatccgccttcgcctccaaccgcgtgcgctccacgcctcctccaggaaagcgagaggtgagcgcagtcccctttcccctccttccaattcaattcgtcttctcgttcgcagccctaggatttgggggtctggaggggtttgatcgtttctcgccgtgaatctgctttggtgtaaaccaacggatctcggatcgtagtcttcagaagatcccggattttgcggtttggcccctcctggattcaattcgtcgtatcgttcgcagccctaggatttggggatctggaggggtttgatcgtttctcgccgcgaatctgctctggtgtaaaccaacggatctcgggtcgtagtcttcagaaggtcccggattttgcggtttggcccctcctggattcaattcgtcgtatcgttcgcagccctaggatttggggatctggaggggtttgatcctttctcgccgcgaatctgctctggtataaccaacggatctcgggtcgtagtcttcagaaggtcccggattttgcggtttggtggttcttgctctatgaatcagagggatggttcttcccggatttatgccttgcggccactctgtcgaatcatggggtttcgacccgattcgtaggcgtgctccctgttttggatgggaagtaggcgtgtttgtagtattcgtgcttcgattcgtcaacggagattagaagacctgggatgggatttgaggaaatctaggtatctgtctagcacgtttctagatctattcttcagctgttatatgagagtaattttggaaccctggtggggtatgtttgaccgagtattctgtagattattgtccgtgacttgctggctgttaccgtccttatctctgcatcatctatctgtgctagtttctgcgtgcttctcaaatatttccggcctgtgtagcatgtgactgataatatgattttggcagcttctgcataagaacaacaaatcaaaagcttgatcagctcggtgcctacaaaacctcaacaaccaagtttc

SEQ ID NO: 467 - DNA, 23 bp, Glycine max
gcgtcctttgacagcagctgtgg

SEQ ID NO: 468 - DNA, 23 bp, Glycine max
gcaaccacagctgctgtcaaagg

SEQ ID NO: 469 - DNA, 434 bp, Glycine max; identical to SEQ ID NO: 120

SEQ ID NO: 470 - DNA, 9093 bp, Artificial sequence
  QC878
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttgcgtcctttgacagcagctggttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccggtac

SEQ ID NO: 471 - DNA, 9093 bp, Artificial sequence
  QC879
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttgcaaccacagctgctgtcaagttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccggtac

SEQ ID NO: 472 - DNA, 1357 bp, Artificial sequence
  RTW1013A
ctagaagataaaccctcccccaaaacacaaattagaatgacatttcaagttccatgtatgtcactttcattctattatttttacaacttttagttacttaacagatgtcttgttcagcataaattataatttattctgtttttttttagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtattgcaatgagatctttgacagcagctgttgttgctgcaggtggaaatgcaaggtctgttttttttttttttgttcagcataatctttgaattgttcctcgtataactaatcacaacagagtacgtgttcttcttcctgttataatctaaaaatctcatccagattagtcatcctttcttcttaaaaggaacctttaattatcaatgtatttatttaatatttaaattagcttgtcaaagtctagcatatacatattttgattatattctgagaaatgcacctgagggtgttcctcatgatctacttcaacctctgttattattagattttctatcatgattactggtttgagtctctaagtagaccatcttgatgttcaaaatatttcagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtatggtttggatttcatttagaataaggtggagtaactttcctggatcaaaattctaatttaagaagcctccctgttttcctctctttagaataagactaagggtaggtttaggagttgggttttggagagaaatggaagggagagcaatttttttcttcttctaataaatattctttaatttgatacattttttaagtaaaagaatataaagatagattagcataacttaatgttttaatcttttatttatttttataaatattatatacctgtctatttaaaaatcaaatatttgtcctccattccctttcccttcaaaacctcagttccaaatataccgtagttgaattatattttggaaggcctattggttggagacttttccttttcagagattatccctcacctttattatagcctttctatttttaaacttcatatagacgccattcttggggcggccgcgat

SEQ ID NO: 473 - DNA, 1357 bp, Artificial sequence
  RTW1012A
ctagaagataaaccctcccccaaaacacaaattagaatgacatttcaagttccatgtatgtcactttcattctattatttttacaacttttagttacttaacagatgtcttgttcagcataaattataatttattctgtttttttttagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtattgcaatgagatctttgacagcagctgtggttgctgcaggtggaaatgcaaggtctgttttttttttttttgttcagcataatctttgaattgttcctcgtataactaatcacaacagagtacgtgttcttcttcctgttataatctaaaaatctcatccagattagtcatcctttcttcttaaaaggaacctttaattatcaatgtatttatttaatatttaaattagcttgtcaaagtctagcatatacatattttgattatattctgagaaatgcacctgagggtgttcctcatgatctacttcaacctctgttattattagattttctatcatgattactggtttgagtctctaagtagaccatcttgatgttcaaaatatttcagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtatggtttggatttcatttagaataaggtggagtaactttcctggatcaaaattctaatttaagaagcctccctgttttcctctctttagaataagactaagggtaggtttaggagttgggttttggagagaaatggaagggagagcaatttttttcttcttctaataaatattctttaatttgatacattttttaagtaaaagaatataaagatagattagcataacttaatgttttaatcttttatttatttttataaatattatatacctgtctatttaaaaatcaaatatttgtcctccattccctttcccttcaaaacctcagttccaaatataccgtagttgaattatattttggaaggcctattggttggagacttttccttttcagagattatccctcacctttattatagcctttctatttttaaacttcatatagacgccattcttggggcggccgcgat

SEQ ID NO: 474 - DNA, 30 bp, Artificial sequence
  primer, soy1-F1
ccactagtaaggaatctaaagatgaaatca

SEQ ID NO: 475 - DNA, 24 bp, Artificial sequence
  primer, soy1-R2
cctgcagcaaccacagctgctgtc

SEQ ID NO: 476 - DNA, 15 bp, Artificial sequence
  probe, soy1-T1(FAM-MGB
ctgcaatgcgtcctt

SEQ ID NO: 477 - DNA, 19 bp, Artificial sequence
  primer, cas9-F
ccttcttccaccgccttga

SEQ ID NO: 478 - DNA, 21 bp, Artificial sequence
  primer, Cas9-R
tgggtgtctctcgtgcttttt

SEQ ID NO: 479 - DNA, 19 bp, Artificial sequence
  probe, Cas9-T(FAM-MGB)
aatcattcctggtggagga

SEQ ID NO: 480 - DNA, 25 bp, Artificial sequence
  primer, pINII-99F
tgatgcccacattatagtgattagc

SEQ ID NO: 481 - DNA, 22 bp, Artificial sequence
  primer, pINII-13R
catcttctggattggccaactt

SEQ ID NO: 482 - DNA, 17 bp, Artificial sequence
  probe, pINII-69T(FAM-MGB)
actatgtgtgcatcctt

SEQ ID NO: 483 - DNA, 23 bp, Artificial sequence
  primer, SIP-130F
ttcaagttgggctttttcagaag

SEQ ID NO: 484 - DNA, 22 bp, Artificial sequence
  primer, SIP-198R
tctccttggtgctctcatcaca

SEQ ID NO: 485 - DNA, 15 bp, Artificial sequence
  probe, SIP-170T(VIC-MGB)
ctgcagcagaaccaa

SEQ ID NO: 486 - DNA, 24 bp, Artificial
  WOL569, Forward_primer
ggacccattaggtgagagcgtggg

SEQ ID NO: 487 - DNA, 19 bp, Artificial
  WOL876, Reverse_primer
cagctgctgtcaaagatct

SEQ ID NO: 488 - DNA, 29 bp, Artificial
  WOL570, Reverse_primer
tctaataataacagaggttgaagtagatc

SEQ ID NO: 489 - DNA, 4104 bp, Glycine max; identical to SEQ ID NO: 115

SEQ ID NO: 490 - DNA, 23 bp, Glycine max
gtttgtttgttgttgggtgtggg

SEQ ID NO: 491 - DNA, 22 bp, Glycine max
tgttgttgggtgtgggaatagg

SEQ ID NO: 492 - DNA, 9174 bp, Artificial sequence
  RTW1199
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttgtttgtttgttgttgggtgtgttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggcaccgaagaagaagcgcaaggtgatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgaagagaccacgggaccgccacgatggcgagctgggaggccgcaagcgggcaaggtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgctcgggcgcgccggtac

SEQ ID NO: 493 - DNA, 9174 bp, Artificial sequence
  RTW1200
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttgtgttgttgggtgtgggaatgttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggcaccgaagaagaagcgcaaggtgatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgaagagaccacgggaccgccacgatggcgagctgggaggccgcaagcgggcaaggtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgctcgggcgcgccggtac

SEQ ID NO: 494 - DNA, 3175 bp, Artificial sequence
  RTW1190A
cgaattctacaggtcactaataccatctaagtagttggttcatagtgactgcatatgtaaaaattatccttattttaaggaaattaaaaattatcatatatatataagttttaaattaattatcttatatatgtaccaaaaagttttaaagcaattattataaaaattaataaatttatcatataaaataatttataattaaattttaaattatcaattcattaaattaaattatttaaaatttttgaatgataatataataattttatcctctactaagtcccaacgtttcctattttattccacttttagcaataaattttgtcataaacacttataacaaaaaaagtaagtaaaaaataaaaaaaagtttttcaataaagtataaactaatttgtataaacttttagaaaaaataaagttatacattgataatataaattttttacataattatccgatcaactcattatatatgataaatttattgattttttaaaataattatcttaaaataatttaaacaatgatttgcaattagatgataatataaaattattttacacactacatgtattaaactcaaacttttatatattagtttttctaaaaactaatttttaactcaaaaaaaatgttacttataattttcttatcttctttttttataagtattttttaagaaatttattgaaacatgaccatgcttgggtcaataatactactctcttagacaccaaacaacccttcccaaactataatctaatccaaaagccatcattcattttccttggtaggtaaagttccaagaccttcaccaactttttcactcaattgttttggtgtaagcaattcgacatgtgttagtgttagttggcaaccaaaaatccctttatgtgactcaatccaacaaccactcacaccaccaacccccataaccatttctcacaatacccttcatttacacattatcatcaccaaaaataaataaaaaaaacctctcatttcagagagagagagagagacttcacagaccaaagtgcagagaacaacaaagttcacaactttaaggaaaattgaaatggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtattgcaatgagatctttgacagcagctgttgttgctgcaggtggaaatgcaaggtctgttttttttttttttgttcagcataatctttgaattgttcctcgtataactaatcacaacagagtacgtgttcttcttcctgttataatctaaaaatctcatccagattagtcatcctttcttcttaaaaggaacctttaattatcaatgtatttatttaatatttaaattagcttgtcaaagtctagcatatacatattttgattatattctgagaaatgcacctgagggtgttcctcatgatctacttcaacctctgttattattagattttctatcatgattactggtttgagtctctaagtagaccatcttgatgttcaaaatatttcagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtatggtttggatttcatttagaataaggtggagtaactttcctggatcaaaattctaatttaagaagcctccctgttttcctctctttagaataagactaagggtaggtttaggagttgggttttggagagaaatggaagggagagcaatttttttcttcttctaataaatattctttaatttgatacattttttaagtaaaagaatataaagatagattagcataacttaatgttttaatcttttatttatttttataaatattatatacctgtctatttaaaaatcaaatatttgtcctccattccctttcccttcaaaacctcagttccaaatataccgtagttgaattatattttggaaggcctattggttggagacttttccttttcagagattatccctcacctttattatagcctttctatttttaaacttcatatagacgccattcttggggcggccgcgat

SEQ ID NO: 495 - DNA, 23 bp, Artificial sequence
  primer, soy1-F3
gtttgtttgttgttgggtgtggg

SEQ ID NO: 496 - DNA, 25 bp, Artificial sequence
  primer, soy1-R3
gacatgatgcttcattttcacagaa

SEQ ID NO: 497 - DNA, 18 bp, Artificial sequence
  robe, soy1-T2(FAM-MGB)
tgtgtagagtggattttg

SEQ ID NO: 498 - DNA, 22 bp, Artificial sequence
  primer, soy1-F2
tgttgttgggtgtgggaatagg

SEQ ID NO: 499 - DNA, 31 bp, Artificial sequence
  WOL1001, Forward_primer
aggtttaattttatataatgttagcatacag

SEQ ID NO: 500 - DNA, 28 bp, Artificial sequence
  500 WOL1002, Reverse_primer
atcaacatcatgctgatgtagaacaaac

SEQ ID NO: 501 - DNA, 29 bp, Artificial sequence
  501 WOL1003, Forward_primer
attctgatttatcttgtgattgttgactc

SEQ ID NO: 502 - DNA, 27 bp, Artificial sequence
  WOL1004, Reverse_primer
atttactttggagagaataaggagggg

SEQ ID NO: 503 - DNA, 23 bp, Glycine mac
gaaacgttgggacttagtagagg

SEQ ID NO: 504 - DNA, 23 bp, Glycine max
ggaataaaataggaaacgttggg

SEQ ID NO: 505 - DNA, 9174 bp, Artificial sequence
  RTW1201
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttgaaacgttgggacttagtaggttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggcaccgaagaagaagcgcaaggtgatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgaagagaccacgggaccgccacgatggcgagctgggaggccgcaagcgggcaaggtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgctcgggcgcgccggtac

SEQ ID NO: 506 - DNA, 9174 bp, Artificial sequence
  RTW1202
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttggaataaaataggaaacgttgttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggcaccgaagaagaagcgcaaggtgatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgaagagaccacgggaccgccacgatggcgagctgggaggccgcaagcgggcaaggtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgctcgggcgcgccggtac

SEQ ID NO: 507 - DNA, 6113 bp, Artificial sequence
  RTW1192A
caagtagttctagtcttaatacaaatgtcaaatggcacaagtgagattttgaatttctgatgttgtaaaaatctcaggacatgaatactattgggaagcaattattcatacttcaccaatccaaactgacccaaaattctcaaatcacatgaaagcaaaaatgcatataacacgaagaataagaagaagaggaactaacctggggtttcgatgattaaagcgttgttgttgatgatgaaaacgatgattatggagagaaattgttgttgaatggtgaaattgttatagaaagagaacgaagatagagaaaaagatatatagatttttcaaggctcaaaccctaaaatcaccatgagagagaacaaagattgagaaacctacaaccactatgagagagaatgagcagaacagaagcgtgagatagagaacgagagttaaggtgcgagaggacacgaagaacaaaaggtgtgagagaaagaacaaaggagcctacggtgtgagatgagagaatttgaaattcttaccatttaggtggaatttcaattctacaattttattctattaaaattattttaaaaaatgatgtcattttaaattctttaaaatctcatatccaacaactgaattatgatagaggtatttcaaattcacttaaaaaaattatcttatttaaataccccatccaaacatagcgaaatgttcatgagaaggatcaagtggtttggaaacatagtactaatggtgtttatacagttcatggaatccttgatagataatttaaaggttgctggaaattggatgaaggtgtggagattaaatattcttccaaaaataaagcgttttatttggagagtgttgtgtggttgtctcccctgtaggcaaaagcttcgatgtaaaggagttcaatgtccaataacctatgctttctatccctcgattattgaaaatgaatgacacattttatttggttgaaatcaagaaataagcatgtggcaagcaacgggtatttgacaattcatagaacaaaaggtgaatgcagcaaaagaattaatgaactccttttcgatctacttggatcactacatggagatattatcaacaaatttgatgttactttatggagcagttggaattcttggaatgacaagatatgaaatgaacataccaaccctcctcttgtttctgtttcggtttctatgcagtattttgttgaatggcaaagtgcaaggtaatatgctcctcaacatcaattaacaaatgttcatgacatctcttaccagctccaacttggggacgtttgacaaacaccaccgtcaagtttccttaaatgcaacattaatgttgctcatttcaaggaggagaatagttttggtgtcggcatgatactccatcaaggaagattcgtcaaagctcactcacgttttcgacatgggtcgacatgggttacctgacccaaaggctgaggcttaggcttgggtttgcttcaagtattgatctgggcccagactattggtttacataatatcatttttgaaaacctaacatctaaaactcaaggttgtttagaggtgcgccattccaaaataagattatcctatttgtgcatgaatgcgaccaactatctcctgtttcagcattataaagtataaacaacaaacttctttaatcaagggactaaaagatattggacatacaagctaaaagtgatagaatttgagaaaacaaatattgacaacaatattcaagaggacactaaaacataattctcaaattttttttgtttatttaaaataaagtggttcattaggtagctccgggtgattgcggttacatcatgtacggaaaaataattctaatccttgatttaaatttgaacttgactatttatttattctttatttcattttgtaaatcattttatgtatctcctggcaagcaattttatccaccttgcaccaacaccttcgggttccataatcaaaccaccttaacttcacaccatgctgtaactcacaccgcccagcatctccaatgtgaaagaagctaaaatttaataaacaatcatacgaagcagtgacaaaataccagatggtattaatgcttcgataaaattaattggaaagtataaaatggtagaaaataataaattataattaatttaagtaagataaaaaataattaaaaactaaaatgttaaaattttaaaaaaattattttaaataatatttaaaaacattaaaaatcattttaaaaaatttatttatagaacaattaaataaatatttcagctaataaaaaacaaaagcttacctagccttagaagacaacttgtccaacaattagatgatacccattgcccttacgttttctttaacatcaattattgtttttgtcaacaagctatcttttagttttattttattggtaaaaaatatgtcgccttcaagttgcatcatttaacacatctcgtcattagaaaaataaaactcttccctaaacgattagtagaaaaaatcattcgataataaataagaaagaaaaattagaaaaaaataacttcattttaaaaaaatcattaaggctatattttttaaatgactaattttatatagactgtaactaaaagtatacaatttattatgctatgtatcttaaagaattacttataaaaatctacggaagaatatcttacaaagtgaaaaacaaatgagaaagaatttagtgggatgattatgattttatttgaaaattgaaaaaataattattaaagactttagtggagtaagaaagctttcctattagtcttttcttatccataaaaaaaaaaaaaaaaatctagcgtgacagcttttccatagattttaataatgtaaaatactggtagcagccgaccgttcaggtaatggacactgtggtcctaacttgcaacgggtgcgggcccaatttaataacgccgtggtaacggataaagccaagcgtgaagcggtgaaggtacatctctgactccgtcaagattacgaaaccgtcaactacgaaggactccccgaaatatcatctgtgtcataaacaccaagtcacaccatacatgggcacgcgtcacaatatgattggagaacggttccaccgcatatgctataaaatgcccccacacccctcgaccctaatcgcacttcaattgcaatcaaattagttcattctctttgcgcagttccctacctctcctttcaaggttcgtagatttcttccgtttttttttcttcttctttattgtttgttctacatcagcatgatgttgatttgattgtgttttctatcgtttcatcgattataaattttcataatcagaagattcagcttttattaatgcaagaacgtccttaattgatgattttataaccgtaaattaggtctaattagagtttttttcataaagattttcagatccgtttacaacaagccttaattgttgattctgtagtcgtagattaaggtttttttcatgaactacttcagatccgttaaacaacagccttatttgttgatacttcagtcgtttttcaagaaattgttcagatccgttgataaaagccttattcgttgattctgtatggtatttcaagagatattgctcaggtcctttagcaactaccttatttgttgattctgtggccatagattaggattttttttcacgaaattgcttcttgaaattacgtgatggattttgattctgatttatcttgtgattgttgactctacagatggcccaagtgagcagagtgcacaatcttgctcaaagcactcaaatttttggccattcttccaactccaacaaactcaaatcggtgaattcggtttcattgaggccacgcctttggggggcctcaaaatctcgcatcccgatgcataaaaatggaagctttatgggaaattttaatgtggggaagggaaattccggcgtgtttaaggtttctgcatcggtcgccgccgcagagaagccgtcaacgtcgccggagatcgtgttggaacccatcaaagacttctcgggtaccatcacattgccagggtccaagtctctgtccaatcgaattttgcttcttgctgctctctctgaggtgaagtttatttatttatttatttgtttgtttgttgttgggtgtgggaataggagtttgatgtgtagagtggattttgaatatttgatttttttttgtattattctgtgaaaatgaagcatcatgtcccatgaaagaaatggacacgaaattaagtggcttatgatgtgaaatgaggatagaaatgtgtgtagggttttttaatgggtagcaataagcatattcaatatctggattgatttggacgtttctgtataaaggagtatgctagcaatgtgttaatgtatggcttgctaaaatactcctaaaaatcaagtgggagtagtatacatatctacagcaaatgtattaggtgaggcatttggcttctctattgtaaggaacaaataatatcagttaatgtgaaaatcaatggttgatattccaatacattcatgatgtgttatttatatgtacctaatattgactgttgtttttctccgcaatgaccaagattatttattttatcctctaaagtgactaattgagttgcttactttagagaagttggacccattaggtgagagcgtggggggaactaatcttgaatatacaatctgagtcttgattatccaagtatggttgtatgaacaatgttagctctagaagataaaccctcccccaaaacacaaattagaatgacatttcaagttccatgtatgtcactttcattctattatttttacaacttttagttacttaacagatgtcttgttcagcataaattataatttattctgtttttttttagggaacaactgttgtagacaacttgttgtatagtgaggatattcattacatgcttggtgcattaaggacccttggactgcgtgtggaagatgacaaaacaaccaaacaagcaattgttgaaggctgtgggggattgtttcccactagtaaggaatctaaagatgaaatcaatttattccttggaaatgctggtattgcaatgagatctttgacagcagctgttgttgctgcaggtggaaatgcaaggtctgttttttttttttttgttcagcataatctttgaattgttcctcgtataactaatcacaacagagtacgtgttcttcttcctgttataatctaaaaatctcatccagattagtcatcctttcttcttaaaaggaacctttaattatcaatgtatttatttaatatttaaattagcttgtcaaagtctagcatatacatattttgattatattctgagaaatgcacctgagggtgttcctcatgatctacttcaacctctgttattattagattttctatcatgattactggtttgagtctctaagtagaccatcttgatgttcaaaatatttcagctacgtacttgatggggtgccccgaatgagagagaggccaattggggatttggttgctggtcttaagcaacttggtgcagatgttgattgctttcttggcacaaactgtccacctgttcgtgtaaatgggaagggaggacttcctggcggaaaggtatggtttggatttcatttagaataaggtggagtaactttcctggatcaaaattctaatttaagaagcctccctgttttcctctctttagaataagactaagggtaggtttaggagttgggttttggagagaaatggaagggagagcaatttttttcttcttctaataaatattctttaatttgatacattttttaagtaaaagaatataaagatagattagcataacttaatgttttaatcttttatttatttttataaatattatatacctgtctatttaaaaatcaaatatttgtcctccattccctttcccttcaaaacctcagttccaaatataccgtagttgaattatattttggaaggcctattggttggagacttttccttttcagagattatccctcacctttattatagcctttctatttttaaacttcatatagacgccattcttggggcggccgcgat

SEQ ID NO: 508 - DNA, 32 bp, Artificial sequence
  primer, soy1-F4
tcaataatactactctcttagacaccaaacaa

SEQ ID NO: 509 - DNA, 23 bp, Artificial sequence
  primer, soy1-R4
caaggaaaatgaatgatggcttt

SEQ ID NO: 510 - DNA, 18 bp, Artificial sequence
  probe, soy1-T3(FAM-MGB)
ccttcccaaactataatc

SEQ ID NO: 511 - DNA, 27 bp, Artificial sequence
  WOL1005, Forward_primer
aaatgttatcagaggaacatgagctgc

SEQ ID NO: 512 - DNA, 28 bp, Artificial sequence
  WOL1006, Reverse_primer
attatttttccgtacatgatgtaaccgc

SEQ ID NO: 513 - DNA, 438 bp, Cauliflower mozaic virus
cccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgat

SEQ ID NO: 514 - DNA, 19 bp, Cauliflower mozaic virus
gtctcagaagaccaaaggg

SEQ ID NO: 515 - DNA, 25 bp, Cauliflower mozaic virus
tgccatcattgcgataaaggaaagg

SEQ ID NO: 516 - DNA, 20 bp, Cauliflower mozaic virus
gatgcctctgccgacagtgg

SEQ ID NO: 517 - DNA, 3708 bp, Zea mays
ctgcagcccatcaaggagatctccggcaccgtcaagctgccggggtccaagtcgctttccaacaggatcctcctgctcgccgccctgtccgaggtgagcgattttggtgcttgctgcgctgccctgtctcactgctacctaaatgttttgcctgtcgaataccatggattctcggtgtaatccatctcacgatcagatgcaccgcatgtcgcatgcctagctctctctaatttgtctagtagtttgtatacggattaagattgataaatcggtaccgcaaaagctaggtgtaaataaacactacaaaattggatgttcccctatcggcctgtactcggctactcgttcttgtgatggcatgttatttcttcttggtgtttggtgaactcccttatgaaatttgggcgcaaagaaatcgccctcaagggttgatcttatgccatcgtcatgataaacagtgaagcacggatgatcctttacgttgtttttaacaaactttgtcagaaaactagcaatgttaacttcttaatgatgatttcacaacaaaaaaggtaaccttgctactaacataacaaaagacttgttgcttattaattatatgtttttttaatctttgatcaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggatgctagagaggaagtgcagctcttcttggggaatgctggaatcgcaatgcggtcattgacagcagctgttactgctgctggtggaaatgcaacgtatgtttcctctctctctctacaatacttgttggagttagtatgaaacccatgtgtatgtctagtggcttatggtgtattggtttttgaacttcagttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggttagttactaagggccacatgttacattcttctgtaaatggtacaactattgtcgagcttttgcatttgtaaggaaaacattgattgatctgaatttgatgctacaccacaaaatatctacaaatggtcatccctaactagcaaaccatgtctccattaagctcaatgaagtaatacttggcatgtgtttatcaacttaatttccatcttctggggtattgcctgttttctagtctaatagcatttgtttttagaattagctcttacaactgttatgttctacaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtaagctctgtaatgtatttcactactttgatgccaatgtttcagttttcagttttccaaacagtcgcatcaatatttgaatagatgcactgtagaaaaaaatcattgcagggaaaaactagtactgagtattttgactgtaaattatttaaccagtcggaatatagtcagtctattggagtcaagagcgtgaaccgaaatagccagttaattatcccattatacagaggacaaccatgtatactattgaaacttggtttaagagaatctaggtagctggactcgtagctgcttggcatggataccttcttatctttaggaaaagacacttgattttttttctgtggccctctatgatgtgtgaacctgcttctctattgctttagaaggatatatctatgtcgttatgcaacatgcttcccttagtcatttgtactgaaatcagtttcataagttcgttagtggttccctaaacgaaaccttgtttttctttgcaatcaacaggtcccctaaaaatgcctatgttgaaggtgatgcctcaagcgcaagctatttcttggctggtgctgcaattactggagggactgtgactgtggaaggttgtggcaccaccagtttgcaggtaaagatttcttggctggtgctacgataactgcttttgtctttttggtttcagcattgttctcagagtcactaaataacattatcatctgcaaacgtcaaatagacatacttaggtgaatggatattcatgtaaccgtttccttacaaatttgctgaaacctcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacggtaaaacattctcagccctacaaccatgcctcttctacatcactacttgacaagactaaaaactattggctcgttggcagtggcttcctggagagtaaaggagaccgagaggatggttgcgatccggacggagctaaccaaggtaaggctacatacttcacatgtctcacgtcgtctttccatagctcgctgcctcttagcggcttgcctgcggtcgctccatcctcggttgctgtctgtgttttccacagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtgaccatccgggaccctgggtgcacccggaagaccttccccgactacttcgatgtgctgagcactttcgtcaagaattaataaagcgtgcgatactaccacgcagcttgattgaagtgataggcttgtgctgaggaaatacatttcttttgttctgttttttctctttcacgggattaagttttgagtctgtaacgttagttgtttgtagcaagtttctatttcggatcttaagtttgtgcactgtaagccaaatttcatttcaagagtggttcgttggaataataagaataataaattacgtttcagtggctgtcaagcctgctgctacgttttaggagatggcattagacattcatcatcaacaacaataaaaccttttagcctcaaacaataatagtgaagttattttttagtcctaaacaagttgcattaggatatagttaaaacacaaaagaagctaaagttagggtttagacatgtggatattgttttccatgtatagtatgttctttctttgagtctcatttaactacctctacacataccaactttagttttttttctacctcttcatgttactatggtgccttcttatcccactgagcattggtatatttagaggtttttgttgaacatgcctaaatcatctcaatcaacgatggacaatcttttcttcgattgagctgaggtacgtcatctaga

SEQ ID NO: 518 - DNA, 3714 bp, Zea mays
ctgcagcccatcaaggagatctccggcaccgtcaagctgccggggtccaagtcgctttccaacaggatcctcctgctcgccgccctgtccgaggtgagcgattttggtgcttgctgcgctgccctgtctcactgctacctaaatgttttgcctgtcgaataccatggattctcggtgtaatccatatctgcacgatcagatatgcaccgcatgtcgcatatctgagctctctctaatttgtctagtagtttgtatacggattaagattgataaatcggtaccgcaaaagctaggtgtaaataaacactacaaaattggatgttcccctatcggcctgtactcggctactcgttcttgtgatggcatgttatttcttcttggtgtttggtgaactcccttatgaaatttgggcgcaaagaaatcgccctcaagggttgatcttatgccatcgtcatgataaacagtgaagcacggatgatcctttacgttgtttttaacaaactttgtcagaaaactagcaatgttaacttcttaatgatgatttcacaacaaaaaaggtaaccttgctactaacataacaaaagacttgttgcttattaattatatgtttttttaatctttgatcaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggatgctaaagaggaagtgcagctcttcttggggaatgctggaatcgcaatgcggtcattgacagcagctgttactgctgctggtggaaatgcaacgtatgtttcctctctctctctacaatacttgttggagttagtatgaaacccatgtgtatgtctagtggcttatggtgtattggtttttgaacttcagttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggttagttactaagggccacatgttacattcttctgtaaatggtacaactattgtcgagcttttgcatttgtaaggaaaacattgattgatctgaatttgatgctacaccacaaaatatctacaaatggtcatccctaactagcaaaccatgtctccattaagctcaatgaagtaatacttggcatgtgtttatcaacttaatttccatcttctggggtattgcctgttttctagtctaatagcatttgtttttagaattagctcttacaactgttatgttctacaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtaagctctgtaatgtatttcactactttgatgccaatgtttcagttttcagttttccaaacagtcgcatcaatatttgaatagatgcactgtagaaaaaaatcattgcagggaaaaactagtactgagtattttgactgtaaattatttaaccagtcggaatatagtcagtctattggagtcaagagcgtgaaccgaaatagccagttaattatcccattatacagaggacaaccatgtatactattgaaacttggtttaagagaatctaggtagctggactcgtagctgcttggcatggataccttcttatctttaggaaaagacacttgattttttttctgtggccctctatgatgtgtgaacctgcttctctattgctttagaaggatatatctatgtcgttatgcaacatgcttcccttagtcatttgtactgaaatcagtttcataagttcgttagtggttccctaaacgaaaccttgtttttctttgcaatcaacaggtcccctaaaaatgcctatgttgaaggtgatgcctcaagcgcaagctatttcttggctggtgctgcaattactggagggactgtgactgtggaaggttgtggcaccaccagtttgcaggtaaagatttcttggctggtgctacgataactgcttttgtctttttggtttcagcattgttctcagagtcactaaataacattatcatctgcaaacgtcaaatagacatacttaggtgaatggatattcatgtaaccgtttccttacaaatttgctgaaacctcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacggtaaaacattctcagccctacaaccatgcctcttctacatcactacttgacaagactaaaaactattggctcgttggcagtggcttcctggagagtaaaggagaccgagaggatggttgcgatccggacggagctaaccaaggtaaggctacatacttcacatgtctcacgtcgtctttccatagctcgctgcctcttagcggcttgcctgcggtcgctccatcctcggttgctgtctgtgttttccacagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtgaccatccgggaccctgggtgcacccggaagaccttccccgactacttcgatgtgctgagcactttcgtcaagaattaataaagcgtgcgatactaccacgcagcttgattgaagtgataggcttgtgctgaggaaatacatttcttttgttctgttttttctctttcacgggattaagttttgagtctgtaacgttagttgtttgtagcaagtttctatttcggatcttaagtttgtgcactgtaagccaaatttcatttcaagagtggttcgttggaataataagaataataaattacgtttcagtggctgtcaagcctgctgctacgttttaggagatggcattagacattcatcatcaacaacaataaaaccttttagcctcaaacaataatagtgaagttattttttagtcctaaacaagttgcattaggatatagttaaaacacaaaagaagctaaagttagggtttagacatgtggatattgttttccatgtatagtatgttctttctttgagtctcatttaactacctctacacataccaactttagttttttttctacctcttcatgttactatggtgccttcttatcccactgagcattggtatatttagaggtttttgttgaacatgcctaaatcatctcaatcaacgatggacaatcttttcttcgattgagctgaggtacgtcatctaga

SEQ ID NO: 519 - DNA, 3708 bp, Zea mays
ctgcagcccatcaaggagatctccggcaccgtcaagctgccggggtccaagtcgctttccaacaggatcctcctgctcgccgccctgtccgaggtgagcgattttggtgcttgctgcgctgccctgtctcactgctacctaaatgttttgcctgtcgaataccatggattctcggtgtaatccatctcacgatcagatgcaccgcatgtcgcatgcctagctctctctaatttgtctagtagtttgtatacggattaagattgataaatcggtaccgcaaaagctaggtgtaaataaacactacaaaattggatgttcccctatcggcctgtactcggctactcgttcttgtgatggcatgttatttcttcttggtgtttggtgaactcccttatgaaatttgggcgcaaagaaatcgccctcaagggttgatcttatgccatcgtcatgataaacagtgaagcacggatgatcctttacgttgtttttaacaaactttgtcagaaaactagcaatgttaacttcttaatgatgatttcacaacaaaaaaggtaaccttgctactaacataacaaaagacttgttgcttattaattatatgtttttttaatctttgatcaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggatgctagaaaggaagtgcagctcttcttggggaatgctggaatcgcaatgcggtcattgacagcagctgttactgctgctggtggaaatgcaacgtatgtttcctctctctctctacaatacttgttggagttagtatgaaacccatgtgtatgtctagtggcttatggtgtattggtttttgaacttcaggtacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggttagttactaagggccacatgttacattcttctgtaaatggtacaactattgtcgagcttttgcatttgtaaggaaaacattgattgatctgaatttgatgctacaccacaaaatatctacaaatggtcatccctaactagcaaaccatgtctccattaagctcaatgaagtaatacttggcatgtgtttatcaacttaatttccatcttctggggtattgcctgttttctagtctaatagcatttgtttttagaattagctcttacaactgttatgttctacaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtaagctctgtaatgtatttcactactttgatgccaatgtttcagttttcagttttccaaacagtcgcatcaatatttgaatagatgcactgtagaaaaaaatcattgcagggaaaaactagtactgagtattttgactgtaaattatttaaccagtcggaatatagtcagtctattggagtcaagagcgtgaaccgaaatagccagttaattatcccattatacagaggacaaccatgtatactattgaaacttggtttaagagaatctaggtagctggactcgtagctgcttggcatggataccttcttatctttaggaaaagacacttgattttttttctgtggccctctatgatgtgtgaacctgcttctctattgctttagaaggatatatctatgtcgttatgcaacatgcttcccttagtcatttgtactgaaatcagtttcataagttcgttagtggttccctaaacgaaaccttgtttttctttgcaatcaacaggtcccctaaaaatgcctatgttgaaggtgatgcctcaagcgcaagctatttcttggctggtgctgcaattactggagggactgtgactgtggaaggttgtggcaccaccagtttgcaggtaaagatttcttggctggtgctacgataactgcttttgtctttttggtttcagcattgttctcagagtcactaaataacattatcatctgcaaacgtcaaatagacatacttaggtgaatggatattcatgtaaccgtttccttacaaatttgctgaaacctcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacggtaaaacattctcagccctacaaccatgcctcttctacatcactacttgacaagactaaaaactattggctcgttggcagtggcttcctggagagtaaaggagaccgagaggatggttgcgatccggacggagctaaccaaggtaaggctacatacttcacatgtctcacgtcgtctttccatagctcgctgcctcttagcggcttgcctgcggtcgctccatcctcggttgctgtctgtgttttccacagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtgaccatccgggaccctgggtgcacccggaagaccttccccgactacttcgatgtgctgagcactttcgtcaagaattaataaagcgtgcgatactaccacgcagcttgattgaagtgataggcttgtgctgaggaaatacatttcttttgttctgttttttctctttcacgggattaagttttgagtctgtaacgttagttgtttgtagcaagtttctatttcggatcttaagtttgtgcactgtaagccaaatttcatttcaagagtggttcgttggaataataagaataataaattacgtttcagtggctgtcaagcctgctgctacgttttaggagatggcattagacattcatcatcaacaacaataaaaccttttagcctcaaacaataatagtgaagttattttttagtcctaaacaagttgcattaggatatagttaaaacacaaaagaagctaaagttagggtttagacatgtggatattgttttccatgtatagtatgttctttctttgagtctcatttaactacctctacacataccaactttagttttttttctacctcttcatgttactatggtgccttcttatcccactgagcattggtatatttagaggtttttgttgaacatgcctaaatcatctcaatcaacgatggacaatcttttcttcgattgagctgaggtacgtcatctaga

SEQ ID NO: 520 - protein, 464 aa, zea mays
MQLDLNVAEAPPPVEMEASDSGSSVLNASEAASAGGAPAPAEEGSSSTPAVLEFSILIRSDSDAAGADEDEDATPSPPPRHRHQHQQQLVTRELFPAGAGPPAPTPRHWAELGFFRADLQQQQAPGPRIVPHPHAAPPPAKKSRRGPRSRSSQYRGVTFYRRTGRWESHIWDCGKQVYLGGFDTAHAAARAYDRAAIKFRGVDADINFNLSDYEDDMKQMGSLSKEEFVHVLRRQSTGFSRGSSRYRGVTLHKCGRWEARMGQFLGKKYIYLGLFDSEVEAARAYDKAAIKCNGREAVTNFEPSTYHGELPTEVADVDLNLSISQPSPQRDKNSCLGLQLHHGPFEGSELKKTKIDDAPSELPGRPRQLSPLVAEHPPAWPAQPPHPFFVFTNHEMSASGDLHRRPAGAVPSWAWQVAAAAPPPAALPSSAAASSGFSNTATTAATTAPSASSLRYCPPPPPPS

SEQ ID NO: 521 - DNA, 1413 bp, Zea mays
atgcagttggatctgaacgtggccgaggcgccgccgccggtggagatggaggcgagcgactcggggtcgtcggtgctgaacgcgtcggaagcggcgtcggcgggcggcgcgcccgcgccggcggaggagggatctagctcaacgccggccgtgctggagttcagcatcctcatccggagcgatagcgacgcggccggcgcggacgaggacgaggacgccacgccatcgcctcctcctcgccaccgccaccagcaccagcagcagctcgtgacccgcgagctgttcccggccggcgccggtccgccggccccgacgccgcggcattgggccgagctcggcttcttccgcgccgacctgcagcagcaacaggcgccgggccccaggatcgtgccgcacccacacgccgcgccgccgccggccaagaagagccgccgcggcccgcgctcccgcagctcgcagtaccgcggcgtcaccttctaccgccgcacaggccgctgggagtcccacatctgggattgcggcaagcaggtgtacctaggtggattcgacaccgctcacgccgctgcaagggcgtacgaccgggcggcgatcaagttccgcggcgtcgacgccgacatcaacttcaacctcagcgactacgaggacgacatgaagcagatggggagcctgtccaaggaggagttcgtgcacgtcctgcgccgtcagagcaccggcttctcgagaggcagctccaggtacagaggcgtcaccctgcacaagtgcggccgctgggaggcgcgcatggggcagttcctcggcaagaagtacatataccttgggctattcgacagcgaagtagaggctgcaagagcctacgacaaggccgccatcaaatgcaatggcagagaggccgtgacgaacttcgagccgagcacgtatcacggggagctgccgactgaagttgctgatgtcgatctgaacctgagcatatctcagccgagcccccaaagagacaagaacagctgcctaggtctgcagctccaccacggaccattcgagggctccgaactgaagaaaaccaagatcgacgatgctccctctgagctaccgggccgccctcgtcagctgtctcctctcgtggctgagcatccgccggcctggcctgcgcagccgcctcaccccttcttcgtcttcacaaaccatgagatgagtgcatcaggagatctccacaggaggcctgcaggggctgttcccagctgggcatggcaggtggcagcagcagctcctcctcctgccgccctgccgtcgtccgctgcagcatcatcaggattctccaacaccgccacgacagctgccaccaccgccccatcggcctcctccctccggtactgcccgccgccgccgccgccgtcgagccatcaccatccccgctga

SEQ ID NO: 522 - protein, 514 aa, Zea Mays
MTTSTTAKQLRRVRTLGRGASGAVVWLASDEASGELVAVKSARAAGAAAQLQREGRVLRGLSSPHIVPCLGSRAAAGGEYQLLLEFAPGGSLADEAARSGGGRLAERAIGAYAGDVARGLAYLHGRSLVHGDVKARNVVIGGDGRARLTDFGCARPAGGSTRPVGGTPAFMAPEVARGQEQGPAADVWALGCMVVELATGRAPWSDVEGDDLLAALHRIGYTDDVPEVPAWLSPEAKDFLAGCFERRAAARPTAAQPAAHPFVVASASAAAAIRGPAKQEVVPSPKSTLHDAFWDSDAEDEADEMSTGAAAERIGALACAASALPDWDTEEGWIDLQDDHSAGTADAPPAPVADYFISWAEPSDAELEPFVAVAAAAGLPHVAGVALAGATAVNLQGSYYYYPPMHLGVRGNEIPRPLLDHHGDGLEKGQGSHRVCNRETEKVTMKRISLKRRAAFLLDQHHVRSLDKLEYRPRHDRMLRRRQSIYRSNSVLGYDVSKGRQVRWRRAVCIAVAA

SEQ ID NO: 523 - DNA, 1545 bp, Zea mays
atgacgacgtcgaccacggcgaagcagctccggcgcgtgcgcacgctcggccgcggcgcgtcgggcgccgtggtgtggctggcctccgacgaggcctcgggcgagctggtggcggtcaagtcggcgcgcgccgccggggccgcggcgcagctgcagcgcgagggccgcgtcctccggggcctctcgtcgccgcacatcgtgccctgcctcggctcccgcgccgcggcgggcggcgagtaccagctcctgctggagttcgcgccgggcgggtcgctggccgacgaggccgccaggagcggcgggggccgcctcgcggagcgcgccatcggcgcctacgccggggacgtggcgcgcgggctggcgtacctccacggccggtcgctcgtgcacggggacgtcaaggcccggaacgtggtcatcggcggcgacgggcgcgccaggctgaccgacttcgggtgcgcgaggccggccggcgggtcgacgcgccccgtcgggggcaccccggcgttcatggcgcccgaggtggcgcgcggccaggagcagggccccgccgccgacgtctgggcgctcgggtgcatggtcgtcgagctggccacgggccgcgcgccctggagcgacgtggagggcgacgacctcctcgccgcgctccaccggatcgggtacacggacgacgtgccggaggtgcccgcgtggctgtcgcccgaggccaaggacttcctggccggctgcttcgagcgccgcgccgccgcccggcccacggccgcgcagcccgcggcgcacccgttcgtcgtcgcctccgcctccgccgccgccgccatccgcggcccggcgaagcaggaggtggtcccgtcacccaagagcacgctgcacgacgcgttctgggactcggacgccgaggacgaagcggacgagatgtcgacgggcgcggcggccgagaggatcggggcattggcgtgcgccgcctccgcgctgcctgactgggacaccgaggaaggctggatcgacctccaggacgaccactcggccggaactgccgacgcaccgccggcgcccgtcgcggactacttcatcagctgggcggagccgtcagacgcagagctggaaccattcgtcgccgtcgccgccgccgcaggtctcccgcacgttgcaggagttgcattagcaggcgccaccgccgttaacctgcagggcagttattattattacccgcctatgcatctaggcgtccgcggaaacgagattccacgcccgttgttggatcatcatggcgacgggttagaaaaggggcagggatcccaccgcgtttgtaacagagaaacagaaaaggtaacaatgaaacgaatttcgttaaaaagaagagctgctttccttctcgaccagcatcacgtgcgatcgctggacaaactggaatatcgtccacgtcacgaccgaatgctgcgtcgacggcaatctatatatcggagcaatagcgtccttggttacgacgttagcaaaggtaggcaggtccgttggcgccgtgcggtttgcattgccgttgctgcctga

SEQ ID NO: 524 - DNA, 671 bp, zea mays
cggatccactagtaacggccgccagtgtgctggaattcgcccttgacggcccgggctggtatttcaaaactatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcac

SEQ ID NO: 525 - protein, 245 aa, zea mays
MGRGKVQLKRIENKINRQVTFSKRRSGLLKKAHEISVLCDAEVALIIFSTKGKLYEYSTDSCMDKILERYERYSYAEKVLISAEYETQGNWCHEYRKLKAKVETIQKCQKHLMGEDLETLNLKELQQLEQQLESSLKHIRTRKSQLMVESISALQRKEKSLQEENKVLQKELAEKQKDQRQQVQRDQTQQQTSSSSTSFMLREAAPTTNVSIFPVAAGGRVVEGAAAQPQARVGLPPWMLSHLSC

SEQ ID NO: 526 - DNA, 738 bp, zea mays
atggggcgcgggaaggtgcagctgaagcggatcgagaacaagatcaaccgccaggtgacattctccaagcgccgctcggggctactcaagaaggcgcacgagatctccgtgctctgcgacgccgaggtcgcgctcatcatcttctccaccaagggcaagctctacgagtactctaccgattcatgtatggacaaaattcttgaacggtatgagcgctactcctatgcagaaaaggttctcatttccgcagaatatgaaactcagggcaattggtgccatgaatatagaaaactaaaggcgaaggtcgagacaatacagaaatgtcaaaagcacctcatgggagaggatcttgaaactttgaatctcaaagagcttcagcaactagagcagcagctggagagttcactgaaacatatcagaacaaggaagagccagcttatggtcgagtcaatttcagcgctccaacggaaggagaagtcactgcaggaggagaacaaggttctgcagaaggagctcgcggagaagcagaaagaccagcggcagcaagtgcaacgggaccaaactcaacagcagaccagttcgtcttccacgtccttcatgttaagggaagctgccccaacaacaaatgtcagcatcttccctgtggcagcaggcgggagggtggtggaaggggcagcagcgcagccgcaggctcgcgttggactgccaccatggatgcttagccatctgagctgctga

SEQ ID NO: 527 - DNA, 80 bp, zea mays
  misc_feature (1)..(80): sequence of Figure 34B
gctaaagaggaagtgcagctcttcttggggaatgctggaactgcaatgcggccattgacagcagctgttactgctgctgg

SEQ ID NO: 528 - DNA, 80 bp, zea mays
  misc_feature (1)..(80): sequence of Figure 34c
gctagagaggaagtgcagctcttcttggggaatgctggaatcgcaatgcggtcattgacagcagctgttactgctgctgg

SEQ ID NO: 529 - DNA, 37 bp, zea mays
  misc_feature (1)..(37): sequence of Figure 35b
catctcacgatcagatgcaccgcatgtcgcatgccta

SEQ ID NO: 530 - DNA, 42 bp, zea mays
  misc_feature (1)..(42): sequence of Figure 35c
catatctgcacgatcagatatgcaccgcatgtcgcatatctg

SEQ ID NO: 531 - DNA, 31 bp, zea mays
  misc_feature (1)..(31): sequence of Figure 37
gtttttgaacttcagttacgtgcttgatgga

SEQ ID NO: 532 - DNA, 31 bp, zea mays
  misc_feature (1)..(31): sequence of Figure 37
gtttttgaacttcaggtacgtgcttgatgga

SEQ ID NO: 533 - DNA, 459 bp, Artificial Sequence
  Southern genomic probe
agctttatccatccatccatcgcgctagctggctgcaggcacgggttatcttatcttgtcgtccagaggacgacacacggccggccggtgaagtaaaagggagtaatcttattttgccaggacgaggggcggtacatgatattacacacgtaccatgcatgcatatatgcatggacaaggtacgtcgtcgtcgatcgacgtcgatgcatatgtgtgtatgtatgtacgtgcataatgcatggtaccagctgctggcttatatatatttgtcaccgatcgatgcatgctgctgctctacacggtttgacactttaatttgactcatcgatgaccttgctagatagtagcggctcgtcaattaatgagccatcaagttaacaagagggcacgggcttgcgcgactgattccaccttattaacatacgccctgcgcccgcgcgtgctgtacgtacgagaatt

SEQ ID NO: 534 - DNA, 446 bp, Artificial Sequence
  Southern MoPAT probe
tcgaagtcgcgctgccagaagccgacgtcgtgccagccgccgtgcttgtagccggcggcgcggagggtgccgcgggcggtgtagccgagggcctcgtggaggcgcacggacgggtcgttcgggaggccgatcacggccaccacggacttgaagccctgggcctccatgctcttgaggaggtgggtgtagagggtggagccgaggccgaggcgctggtggcggtgggacacgtacacggtggactccacggtccagtcgtaggcgttgcgggccttccacgggccggcgtaggcgatgccggccaccacgccctccacctcggccacgagccacgggtagcggtcctggaggcgctccaggtcgtcgatccactcctgcggggtctgcggctcggtgcggaagttcacggtggaggtctcgatgtagtggttcacgatgtcgcacac

SEQ ID NO: 535 - DNA, 20 bp, Artificial Sequence
  RF-FPCas-1
gcaggtctcacgacggttgg

SEQ ID NO: 536 - DNA, 23 bp, Artificial Sequence
  RF-FPCas-2
gtaaagtacgcgtacgtgtgagg

SEQ ID NO: 537 - DNA, 23 bp, Artificial Sequence
  ALSCas-4
gctgctcgattccgtccccatgg

SEQ ID NO: 538 - DNA, 804 bp, Artificial Sequence
  ALS modification repair template 804
agcttacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgatccccgcaagggcgccgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgcggcctccggctacgcgcgctcctcgggccgcgtcggcgtctgcatcgccacctccggccccggcgccaccaaccttgtctccgcgctcgccgacgcgttgctcgactccgtccccattgtcgccatcacgggacaggtgtcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcagaagggcg

SEQ ID NO: 539 - DNA, 127 bp, Artificial Sequence
  ALS modification repair template 127
aaccttgtctccgcgctcgccgacgcgttgctcgactccgtccccattgtcgccatcacgggacaggtgtcgcgacgcatgattggcaccgacgccttccaggagacgcccatcgtcgaggtcaccc

SEQ ID NO: 540 - DNA, 25 bp, Artificial Sequence
  ALS Forward_primer;
ctacgcacatccccctttctcccac

SEQ ID NO: 541 - DNA, 36 bp, Artificial Sequence
  ALS Reverse_primer
atgcatacctagcatgcgcagagacagtgggtcgtc

SEQ ID NO: 542 - DNA, 22 bp, Artificial sequence
  soy ALS1-CR1, Cas9 target sequence
caccggccaggtcccccgccgg

SEQ ID NO: 543 - DNA, 22 bp, Artificial sequence
  soy ALS2-CR2, Cas9 target sequence
ggcgtcggtgccgatcatccgg

SEQ ID NO: 544 - DNA, 9093 bp, Artificial sequence
  QC880
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttgcaccggccaggtcccccgcgttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccggtac

SEQ ID NO: 545 - DNA, 9093 bp, Artificial sequence
  QC881
ccgggtgtgatttagtataaagtgaagtaatggtcaaaagaaaaagtgtaaaacgaagtacctagtaataagtaatattgaacaaaataaatggtaaagtgtcagatatataaaataggctttaataaaaggaagaaaaaaaacaaacaaaaaataggttgcaatggggcagagcagagtcatcatgaagctagaaaggctaccgatagataaactatagttaattaaatacattaaaaaatacttggatctttctcttaccctgtttatattgagacctgaaacttgagagagatacactaatcttgccttgttgtttcattccctaacttacaggactcagcgcatgtcatgtggtctcgttccccatttaagtcccacaccgtctaaacttattaaattattaatgtttataactagatgcacaacaacaaagcttgggcgtcggtgccgatcatcgttttagagctagaaatagcaagttaaaataaggctagtccgttatcaacttgaaaaagtggcaccgagtcggtgcttttttttgcggccgcaattggatcgggtttacttattttgtgggtatctatacttttattagatttttaatcaggctcctgatttctttttatttcgattgaattcctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatccatggacaaaaagtactcaatagggctcgacatagggactaactccgttggatgggccgtcatcaccgacgagtacaaggtgccctccaagaagttcaaggtgttgggaaacaccgacaggcacagcataaagaagaatttgatcggtgccctcctcttcgactccggagagaccgctgaggctaccaggctcaagaggaccgctagaaggcgctacaccagaaggaagaacagaatctgctacctgcaggagatcttctccaacgagatggccaaggtggacgactccttcttccaccgccttgaggaatcattcctggtggaggaggataaaaagcacgagagacacccaatcttcgggaacatcgtcgacgaggtggcctaccatgaaaagtaccctaccatctaccacctgaggaagaagctggtcgactctaccgacaaggctgacttgcgcttgatttacctggctctcgctcacatgataaagttccgcggacacttcctcattgagggagacctgaacccagacaactccgacgtggacaagctcttcatccagctcgttcagacctacaaccagcttttcgaggagaacccaatcaacgccagtggagttgacgccaaggctatcctctctgctcgtctgtcaaagtccaggaggcttgagaacttgattgcccagctgcctggcgaaaagaagaacggactgttcggaaacttgatcgctctctccctgggattgactcccaacttcaagtccaacttcgacctcgccgaggacgctaagttgcagttgtctaaagacacctacgacgatgacctcgacaacttgctggcccagataggcgaccaatacgccgatctcttcctcgccgctaagaacttgtccgacgcaatcctgctgtccgacatcctgagagtcaacactgagattaccaaagctcctctgtctgcttccatgattaagcgctacgacgagcaccaccaagatctgaccctgctcaaggccctggtgagacagcagctgcccgagaagtacaaggagatctttttcgaccagtccaagaacggctacgccggatacattgacggaggcgcctcccaggaagagttctacaagttcatcaagcccatccttgagaagatggacggtaccgaggagctgttggtgaagttgaacagagaggacctgttgaggaagcagagaaccttcgacaacggaagcatccctcaccaaatccacctgggagagctccacgccatcttgaggaggcaggaggatttctatcccttcctgaaggacaaccgcgagaagattgagaagatcttgaccttcagaattccttactacgtcgggccactcgccagaggaaactctaggttcgcctggatgacccgcaaatctgaagagaccattactccctggaacttcgaggaagtcgtggacaagggcgcttccgctcagtctttcatcgagaggatgaccaacttcgataaaaatctgcccaacgagaaggtgctgcccaagcactccctgttgtacgagtatttcacagtgtacaacgagctcaccaaggtgaagtacgtcacagagggaatgaggaagcctgccttcttgtccggagagcagaagaaggccatcgtcgacctgctcttcaagaccaacaggaaggtgactgtcaagcagctgaaggaggactacttcaagaagatcgagtgcttcgactccgtcgagatctctggtgtcgaggacaggttcaacgcctcccttgggacttaccacgatctgctcaagattattaaagacaaggacttcctggacaacgaggagaacgaggacatccttgaggacatcgtgctcaccctgaccttgttcgaagacagggaaatgatcgaagagaggctcaagacctacgcccacctcttcgacgacaaggtgatgaaacagctgaagagacgcagatataccggctggggaaggctctcccgcaaattgatcaacgggatcagggacaagcagtcagggaagactatactcgacttcctgaagtccgacggattcgccaacaggaacttcatgcagctcattcacgacgactccttgaccttcaaggaggacatccagaaggctcaggtgtctggacagggtgactccttgcatgagcacattgctaacttggccggctctcccgctattaagaagggcattttgcagaccgtgaaggtcgttgacgagctcgtgaaggtgatgggacgccacaagccagagaacatcgttattgagatggctcgcgagaaccaaactacccagaaagggcagaagaattcccgcgagaggatgaagcgcattgaggagggcataaaagagcttggctctcagatcctcaaggagcaccccgtcgagaacactcagctgcagaacgagaagctgtacctgtactacctccaaaacggaagggacatgtacgtggaccaggagctggacatcaacaggttgtccgactacgacgtcgaccacatcgtgcctcagtccttcctgaaggatgactccatcgacaataaagtgctgacacgctccgataaaaatagaggcaagtccgacaacgtcccctccgaggaggtcgtgaagaagatgaaaaactactggagacagctcttgaacgccaagctcatcacccagcgtaagttcgacaacctgactaaggctgagagaggaggattgtccgagctcgataaggccggattcatcaagagacagctcgtcgaaacccgccaaattaccaagcacgtggcccaaattctggattcccgcatgaacaccaagtacgatgaaaatgacaagctgatccgcgaggtcaaggtgatcaccttgaagtccaagctggtctccgacttccgcaaggacttccagttctacaaggtgagggagatcaacaactaccaccacgcacacgacgcctacctcaacgctgtcgttggaaccgccctcatcaaaaaatatcctaagctggagtctgagttcgtctacggcgactacaaggtgtacgacgtgaggaagatgatcgctaagtctgagcaggagatcggcaaggccaccgccaagtacttcttctactccaacatcatgaacttcttcaagaccgagatcactctcgccaacggtgagatcaggaagcgcccactgatcgagaccaacggtgagactggagagatcgtgtgggacaaagggagggatttcgctactgtgaggaaggtgctctccatgcctcaggtgaacatcgtcaagaagaccgaagttcagaccggaggattctccaaggagtccatcctccccaagagaaactccgacaagctgatcgctagaaagaaagactgggaccctaagaagtacggaggcttcgattctcctaccgtggcctactctgtgctggtcgtggccaaggtggagaagggcaagtccaagaagctgaaatccgtcaaggagctcctcgggattaccatcatggagaggagttccttcgagaagaaccctatcgacttcctggaggccaagggatataaagaggtgaagaaggacctcatcatcaagctgcccaagtactccctcttcgagttggagaacggaaggaagaggatgctggcttctgccggagagttgcagaagggaaatgagctcgcccttccctccaagtacgtgaacttcctgtacctcgcctctcactatgaaaagttgaagggctctcctgaggacaacgagcagaagcagctcttcgtggagcagcacaagcactacctggacgaaattatcgagcagatctctgagttctccaagcgcgtgatattggccgacgccaacctcgacaaggtgctgtccgcctacaacaagcacagggataagcccattcgcgagcaggctgaaaacattatccacctgtttaccctcacaaacttgggagcccctgctgccttcaagtacttcgacaccaccattgacaggaagagatacacctccaccaaggaggtgctcgacgcaacactcatccaccaatccatcaccggcctctatgaaacaaggattgacttgtcccagctgggaggcgactctagagccgatcccaagaagaagagaaaggtgtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgatatcaagcttatcgataccgtcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccggtac

SEQ ID NO: 546 - DNA, 1113 bp, Artificial sequence
  RTW1026A
agcttggtaccgagctcggatccactagtatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcagccgtcgcatgatcggtaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactggga

SEQ ID NO: 547 - DNA, 17 bp, Artificial
  WOL900, Forward_primer
atcaccggccaggtcag

SEQ ID NO: 548 - DNA, 25 bp, Artificial
  WOL578, Reverse_primer
acttaccctccactcctttctcctc

SEQ ID NO: 549 - DNA, 29 bp, Artificial
  WOL573, Forward_primer
atggcggccaccgcttccagaaccacccg

SEQ ID NO: 550 - protein, 638 aa, zea mays
MATAAAASTALTGATTAAPKARRRAHLLATRRALAAPIRCSAASPAMPMAPPATPLRPWGPTEPRKGADILVESLERCGVRDVFAYPGGASMEIHQALTRSPVIANHLFRHEQGEAFAASGYARSSGRVGVCIATSGPGATNLVSALADALLDSVPMVAITGQVPRRMIGTDAFQETPIVEVTRSITKHNYLVLDVDDIPRVVQEAFFLASSGRPGPVLVDIPKDIQQQMAVPVWDKPMSLPGYIARLPKPPATELLEQVLRLVGESRRPVLYVGGGCAASGEELRRFVELTGIPVTTTLMGLGNFPSDDPLSLRMLGMHGTVYANYAVDKADLLLALGVRFDDRVTGKIEAFASRAKIVHVDIDPAEIGKNKQPHVSICADVKLALQGMNALLEGSTSKKSFDFGSWNDELDQQKREFPLGYKTSNEEIQPQYAIQVLDELTKGEAIIGTGVGQHQMWAAQYYTYKRPRQWLSSAGLGAMGFGLPAAAGASVANPGVTVVDIDGDGSFLMNVQELAMIRIENLPVKVFVLNNQHLGMVVQWEDRFYKANRAHTYLGNPENESEIYPDFVTIAKGFNIPAVRVTKKNEVRAAIKKMLETPGPYLLDIIVPHQEHVLPMIPSGGAFKDMILDGDGRTVY

SEQ ID NO: 551 - DNA, 21 bp, Zea mays
gcgcgccggaccccagcgcgg

SEQ ID NO: 552 - DNA, 21 bp, Zea mays
gcctcgtcttgtgggggctgg

SEQ ID NO: 553 - DNA, 20 bp, Zea mays
gcttacagcagttggcttgg

SEQ ID NO: 554 - protein, 103 aa, Zea mays
MALEAATAPRALLAACLVLLVLGGSTGPSSVLRGAGTQAGGQCLPQLNRLLACRAYLVPGAPDPSADCCSALSAVSHECACSTMGIINSLPGRCHLAQANCSA